(19) United States
(12) Patent Application Publication
(10) Pub. No.: US 2009/0053227 A1
(43) Pub. Date: Feb. 26, 2009

(54) Prostate Specific Genes and The Use Thereof in Design of Therapeutics

(75) Inventor: Dennis GATELY, San Diego, CA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc., Cambridge, MA (US)

(21) Appl. No.: 12/118,455

(22) Filed: May 9, 2008

Related U.S. Application Data
(63) Continuation of application No. 10/367,978, filed on Feb. 19, 2003, now abandoned.
(60) Provisional application No. 60/357,140, filed on Feb. 19, 2002.
(60) Provisional application No. 60/396,082, filed on Jul. 17, 2002.
(60) Provisional application No. 60/386,759, filed on Jun. 10, 2002.

(51) Int. Cl. C07K 16/30; G01N 33/574; A61K 39/395; A61P 35/00

(52) U.S. Cl. 424/138.1; 530/387.7; 530/391.3; 435/7.23

(57) ABSTRACT

Genes that are upregulated in human prostate tumor tissues and the corresponding proteins are identified. These genes and the corresponding antigens are suitable targets for the treatment, diagnosis or prophylaxis of prostate cancer. A preferred target gene is Kv3.2.

### RELATED APPLICATIONS

This application claims priority to U.S. Provisional No. 60/357,140, filed on Feb. 19, 2002, U.S. Provisional No. 60/396,082, filed on Jul. 17, 2002, and U.S. Provisional No. 60/386,759, filed on Jun. 10, 2002, all of which are incorporated by reference in their entirety herein.

### BACKGROUND OF THE INVENTION

The present invention relates to the identification of human genes that are upregulated in prostate cancer. These genes or the corresponding proteins are to be targeted for the treatment, prevention and/or diagnosis of cancers wherein these genes are upregulated, particularly prostate cancer. In a preferred embodiment the invention provides antibodies directed against Kv3.2, a prostate antigen that is upregulated in prostate cancer that can be used to treat prostate cancer.

### DESCRIPTION OF THE RELATED ART

Genetic detection of human disease states is a rapidly developing field (Taparowsky et al., 1982; Slamon et al., 1989; Sidransky et al., 1992; Miki et al., 1994; Dong et al., 1995; Morahan et al., 1996; Lifton, 1996; Barinaga, 1996). However, some problems exist with this approach. A number of known genetic lesions merely predispose to development of specific disease states. Individuals carrying the genetic lesion may not develop the disease state, while other individuals may develop the disease state without possessing a particular genetic lesion. In human cancers, genetic defects may potentially occur in a large number of known tumor suppresser genes and proto-oncogenes.

The genetic detection of cancer has a long history. One of the earliest genetic lesions shown to predispose to cancer was transforming point mutations in the ras oncogenes (Taparowsky et al., 1982). Transforming ras point mutations may be detected in the stool of individuals with benign and malignant colorectal tumors (Sidransky et al., 1992). However, only 50% of such tumors contained a ras mutation (Sidransky et al., 1992). Similar results have been obtained with amplification of HER-2/neu in breast and prostate cancer (Slamon et al., 1989), deletion and mutation of p53 in bladder cancer (Sidransky et al., 1991), deletion of DCC in colorectal cancer (Fearon et al., 1990) and mutation of BRCAl in breast and prostate cancer (Miki et al., 1994).

None of these genetic lesions are capable of predicting a majority of individuals with cancer and most require direct sampling of a suspected tumor, making screening difficult.

Further, none of the markers described above are capable of distinguishing between metastatic and non-metastatic forms of cancer. In effective management of cancer patients, identification of those individuals whose tumors have already metastasized or are likely to metastasize is critical. Because metastatic cancer kills 560,000 people in the U.S. each year (ACS home page), identification of markers for metastatic prostate cancer would be an important advance.

A particular problem in cancer detection and diagnosis occurs with prostate cancer. Carcinoma of the prostate (PCA) is the most frequently diagnosed cancer among men in the United States (Veltri et al., 1996). Prostate cancer was diagnosed in approximately 189,500 men in 1998 and about 40,000 men succumbed to the malignancy (Landis et al, 1998). Although relatively few prostate tumors progress to clinical significance during the lifetime of the patient, those which are progressive in nature are likely to have metastasized by the time of detection. Survival rates for individuals with metastatic prostate cancer are quite low. Between these extremes are patients with prostate tumors that will metastasize but have not yet done so, for whom surgical prostate removal is curative. Determination of which group a patient falls within is critical in determining optimal treatment and patient survival.

The FDA approval of the serum prostate specific antigen (PSA) test in 1984 changed the way that prostate disease was managed (Allhoff et al., 1989; Cooner et al., 1990; Jacobson et al, 1995; Orozco et al., 1998). PSA is widely used as a serum biomarker to detect and monitor therapeutic response in prostate cancer patients (Badalament et al., 1996; O'Dowd et al., 1997). Several modifications in PSA assays (Partin and Oesterling, 1994; Babian et al., 1996; Zlotta et al, 1997) have resulted in earlier diagnoses and improved treatment.

Although PSA has been widely used as a clinical marker of prostate cancer since 1988 (Partin and Oesterling, 1994), screening programs utilizing PSA alone or in combination with digital rectal examination (DRE) have not been successful in improving the survival rate for men with prostate cancer (Partin and Oesterling, 1994). Although PSA is specific to prostate tissue, it is produced by normal and benign as well as malignant prostatic epithelium, resulting in a high false-positive rate for prostate cancer detection (Partin and Oesterling, 1994).

While an effective indicator of prostate cancer when serum levels are relatively high, PSA serum levels are more ambiguous indicators of prostate cancer when only modestly elevated, for example when levels are between 2-10 ng/ml. At these modest elevations, serum PSA may have originated from non-cancerous disease states such as BPH (benign prostatic hyperplasia), prostatitis or physical trauma (McCormack et al, 1995). Although application of the lower 2.0 ng/ml cancer detection cutoff concentration of serum PSA has increased the diagnosis of prostate cancer, especially in younger men with nonpalpable early stage tumors (Stage Tlc) (Soh et al., 1997; Carter and Coffey, 1997; Harris et al., 1997; Orozco et al., 1998), the specificity of the PSA assay for prostate cancer detection at low serum PSA levels remains a problem.

Several investigators have sought to improve upon the specificity of serologic detection of prostate cancer by examining a variety of other biomarkers besides serum PSA concentration (Ralph and Veltri, 1997). One of the most heavily investigated of these other biomarkers is the ratio of free versus total PSA (f/t PSA) in a patient's blood. Most PSA in serum is in a molecular form that is bound to other proteins such as .alpha.1-antichymotrypsin (ACT) or .alpha.2-macroglobulin (Christensson et al, 1993; Stenman et al., 1991; Lilja et al., 1991). Free PSA is not bound to other proteins. The ratio of free to total PSA (f/tPSA) is usually significantly higher in patients with BPH compared to those with organ confined prostate cancer (Marley et al., 1996; Oesterling et al., 1995; Pettersson et al., 1995). When an appropriate cutoff is determined for the f/tPSA assay, the f/tPSA assay can help distinguish patients with BPH from those with prostate cancer in cases in which serum PSA levels are only modestly elevated (Marley et al., 1996; Partin and Oesterling, 1996). Unfortunately, while f/tPSA may improve on the detection of prostate cancer, information in the f/tPSA ratio is insufficient to improve the sensitivity and specificity of serologic detection of prostate cancer to desirable levels.

Other markers that have been used for prostate cancer detection include prostatic acid phosphatase (PAP) and prostate secreted protein (PSP). PAP is secreted by prostate cells under hormonal control (Brawn et al., 1996). It has less specificity and sensitivity than does PSA. As a result, it is used much less now, although PAP may still have some applications for monitoring metastatic patients that have failed primary treatments. In general, PSP is a more sensitive biomarker than PAP, but is not as sensitive as PSA (Huang et al., 1993). Like PSA, PSP levels are frequently elevated in patients with BPH as well as those with prostate cancer.

Another serum marker associated with prostate disease is prostate specific membrane antigen (PSMA) (Horoszewicz et al., 1987; Carter and Coffey, 1996; Murphy et al., 1996). PSMA is a Type II cell membrane protein and has been identified as Folic Acid Hydrolase (FAH) (Carter and Coffey, 1996). Antibodies against PSMA react with both normal prostate tissue and prostate cancer tissue (Horoszewicz et al., 1987). Murphy et al. (1995) used ELISA to detect serum PSMA in advanced prostate cancer. As a serum test, PSMA levels are a relatively poor indicator of prostate cancer. However, PSMA may have utility in certain circumstances. PSMA is expressed in metastatic prostate tumor capillary beds (Silver et al., 1997) and is reported to be more abundant in the blood of metastatic cancer patients (Murphy et al., 1996). PSMA messenger RNA (mRNA) is down-regulated 8-10 fold in the LNCaP prostate cancer cell line after exposure to 5-.alpha.-dihydroxytestosterone(DHT) (Israeli et al., 1994).

Two relatively new potential biomarkers for prostate cancer are human kallekrein 2 (HK2) (Piironen et al., 1996) and prostate specific transglutaminase (pTGase) (Dubbink et al., 1996). HK2 is a member of the kallekrein family that is secreted by the prostate gland (Piironen et al., 1996). Prostate specific transglutaminase is a calcium-dependent enzyme expressed in prostate cells that catalyzes post-translational cross-linking of proteins (Dubbink et al., 1996). In theory, serum concentrations of HK2 or pTGase may be of utility in prostate cancer detection or diagnosis, but the usefulness of these markers is still being evaluated.

Interleukin 8 (IL-8) has also been reported as a marker for prostate cancer. (Veltri et al., 1999). Serum IL-8 concentrations were reported to be correlated with increasing stage of prostate cancer and to be capable of differentiating BPH from malignant prostate tumors. (Id.) The wide-scale applicability of this marker for prostate cancer detection and diagnosis is still under investigation.

In addition to these protein markers for prostate cancer, several genetic changes have been reported to be associated with prostate cancer, including: allelic loss (Bova, et al., 1993; Macoska et al., 1994; Carter et al., 1990); DNA hypermethylation (Isaacs et al., 1994); point mutations or deletions of the retinoblastoma (Rb), p53 and KAI1 genes (Bookstein et al., 1990a; Bookstein et al., 1990b; Isaacs et al., 1991; Dong et al., 1995); and aneuploidy and aneusomy of chromosomes detected by fluorescence in situ hybridization (FISH) (Macoska et al., 1994; Visakorpi et al., 1994; Takahashi et al., 1994; Alcaraz et al., 1994). None of these has been reported to exhibit sufficient sensitivity and specificity to be useful as general screening tools for asymptomatic prostate cancer.

A recent discovery was that differential expression of both full-length and truncated forms of HER2/neu oncogene receptor was correlated with prostate cancer. (An et al., 1998). Analysis by RT-PCR™ indicated that overexpression of the HER2/neu gene is associated with prostate cancer progression. (Id.)

In current clinical practice, the serum PSA assay and digital rectal exam (DRE) is used to indicate which patients should have a prostate biopsy (Lithrup et al., 1994; Orozco et al., 1998). Histological examination of the biopsied tissue is used to make the diagnosis of prostate cancer. Based upon the 189,500 cases of diagnosed prostate cancer in 1998 (Landis, 1998) and a known cancer detection rate of about 35% (Parker et al., 1996), it is estimated that in 1998 over one-half million prostate biopsies were performed in the United States (Orozco et al., 1998; Veltri et al., 1998). Clearly, there would be much benefit derived from a serological test that was sensitive enough to detect small and early stage prostate tumors that also had sufficient specificity to exclude a greater portion of patients with noncancerous or clinically insignificant conditions.

There remain deficiencies in the prior art with respect to the identification of the genes linked with the progression of prostate cancer and the development of diagnostic methods to monitor disease progression. Likewise, the identification of genes, which are differentially expressed in prostate cancer, would be of considerable importance in the development of a rapid, inexpensive method to diagnose cancer. Although a few prostate specific genes have been cloned (PSA, PSMA, HK2, pTGase, etc.), these are typically not upregulated in prostate cancer. The identification of a novel, prostate specific gene that is differentially expressed in prostate cancer, compared to non-malignant prostate tissue, would represent a major, unexpected advance for the diagnosis, prognosis and treatment of prostate cancer.

### OBJECTS OF THE INVENTION

It is an object of the invention to identify novel gene targets for treatment and diagnosis of prostate cancer.

It is a specific object of the invention to develop novel therapies for treatment of prostate cancer involving the administration of anti-sense oligonucleotides or interfering RNAs corresponding to novel gene targets that are specifically expressed by the prostate cancer.

It is another specific object of the invention to identify that an antigens specifically upregulated in prostate cancer cells.

It is another specific object of the invention to produce ligands that bind antigens expressed by certain prostate cancers, especially monoclonal antibodies and fragments thereof, e.g., domain-deleted antibodies.

It is another specific object of the invention to provide novel therapeutic regimens for the treatment of prostate cancer that involve the administration of antigens expressed by certain prostate cancers, alone or in combination with adjuvants that elicit an antigen-specific cytotoxic T-cell lymphocyte response against cancer cells that express such antigen.

It is another object of the invention to provide novel therapeutic regimens for the treatment of prostate cancer that involve the administration of ligands, especially monoclonal antibodies or fragments thereof that specifically bind novel antigens that are expressed by certain prostate cancers.

It is another object of the invention to provide a novel method for diagnosis of prostate cancer by using ligands, e.g., monoclonal antibodies or fragments, thereof that specifically bind to antigens that are specifically expressed by certain prostate cancers, in order to detect whether a subject has or is at increased risk of developing prostate cancer.

It is another object of the invention to provide a novel method of detecting persons having, or at increased risk of developing prostate cancer by use of labeled DNAs that hybridize to novel gene targets expressed by certain prostate cancers.

It is yet another object of the invention to provide diagnostic test kits for the detection of persons having or at increased risk of developing prostate cancer that comprise a ligand, e.g., monoclonal antibody or antibody fragment that specifically binds to an antigen expressed by prostate cancer cells, and a detectable label, e.g. a radiolabel or fluorophore.

It is another object of the invention to provide diagnostic kits for detection of persons having or at risk of developing prostate cancer that comprise DNA primers or probes specific for novel gene targets specifically expressed by prostate cancer cells, and a detectable label, e.g. radiolabel or fluorophore.

It is another object of the invention to identify genes that are expressed in altered form in prostate cancer cells, e.g. splice variants, and target such altered forms for therapy.

### BRIEF DESCRIPTION OF THE DRAWINGS

FIG. 1 contains visual representation of hybridization results using the fragment 147504 used to measure expression levels of the DWAN gene in prostate malignant and various normal tissue types.

FIG. 2 contains a schematic depiction of the DWAN gene.

FIG. 3 depicts schematically the translation of 147504 fragment including putative PKC and Tyr sites, extracellular and intracellular portions.

FIG. 4 contains the results of PCR hybridization experiment conducted using a primer that spans the intron of DWAN in various tissues including brain and heart that detected the expression of DWAN.

FIGS. 5 and 6 also contain PCR hybridization expression results using primers that span the intron in DWAN that detected the expression of DWAN in various tissues including the heart and brain.

FIG. 7 contains PCR hybridization results showing the expression of DWAN in normal prostate, prostate tumor, and prostate Clontech tissue.

FIG. 8 contains a visual representation of Enorthern results using the DNA fragment 117293 to detect the expression of Kv3.2 in prostate tumor and a variety of normal tissues.

FIGS. 9 and 10 contains PCR hybridization results using exon spanning primers to detect expression of Kv3.2 in various important normal tissues and prostate tumor.

FIG. 11 contains a visual representation of exon results using the fragment 159171 to amplify and assay MASP expression in malignant and non-malignant prostate and various normal tissues.

FIG. 12 is a schematic of the MASP gene.

FIG. 13 shows Kv3.2 and GAPDH expression in prostate samples and MTCI.

FIG. 14 shows Kv3.2 and GAPDH expression in prostate samples and MTC II.

FIG. 15 shows Kv3.2 and GAPDH expression in prostate samples and human heart.

FIG. 16 shows Kv3.2 and GAPDH expression in prostate samples and human brain.

FIG. 17 contains the gene expression profile determined using the Gene Logic datasuite for a DNA sequence overexpressed in prostate tumor tissue (AF116574 Enorthern)

FIG. 18 contains the gene expression profile determined using the Gene Logic datasuite for a DNA sequence overexpressed in prostate tumor tissue (AK024064 Enorthern)

FIG. 19 contains the gene expression profile determined using the Gene Logic datasuite for a DNA sequence overexpressed in prostate tumor tissue (A1640307/Protocadherin 10)

FIG. 20 contains the gene expression profile determined using the Gene Logic datasuite for a DNA sequence overexpressed in prostate tumor tissue (AU144598/Contactin associated Protein-like 2)

FIG. 21 contains the gene expression profile determined using the Gene Logic datasuite for a DNA sequence overexpressed in prostate tumor tissue (BC001186/Protocadherin 5

FIG. 22 contains the gene expression profile determined using the Gene Logic datasuite for a DNA sequence overexpressed in prostate tumor tissue (NM_{—}015392/Neural proliferation, differentiation and control 1)

FIG. 23 contains the gene expression profile determined using the Gene Logic datasuite for a DNA sequence overexpressed in prostate tumor tissue (AI832249/HS1-2)

FIG. 24 contains the gene expression profile determined using the Gene Logic datasuite for a DNA sequence overexpressed in prostate tumor tissue (AI832249/HS1-2)

FIG. 25 contains the gene expression profile determined using the Gene Logic datasuite for a DNA sequence overexpressed in prostate tumor tissue (AB033070/KIAA1244)

FIG. 26 contains the gene expression profile determined using the Gene Logic datasuite for a DNA sequence overexpressed in prostate tumor tissue (AB037765/KIAA 344)

FIG. 27 contains the gene expression profile determined using the Gene Logic datasuite for a DNA sequence overexpressed in prostate tumor tissue (AI742872/Hs6_{—}25897_{—}28_{—}16_{—}1426.a)

FIG. 28 contains the gene expression profile determined using the Gene Logic datasuite for a DNA sequence overexpressed in prostate tumor tissue (AW023227/Hs10_{—}8766_{—}28_{—}5_{—}2415)

FIG. 29 contains the gene expression profile determined using the Gene Logic datasuite for a DNA sequence overexpressed in prostate tumor tissue (BC005335/DKFZP564G2022)

FIG. 30 contains the gene expression profile determined using the Gene Logic datasuite for a DNA sequence overexpressed in prostate tumor tissue (BF055352/Hs18_{—}11087_{—}28_{—}3_t18_Hs18_{—}11087_{—}28_{—}4_{—}3064.a)

FIG. 31 contains the gene expression profile determined using the Gene Logic datasuite for a DNA sequence overexpressed in prostate tumor tissue (N62096/Hs2_{—}5396_{—}28_{—}4_{—}677)

FIG. 32 contains the gene expression profile determined using the Gene Logic datasuite for a DNA sequence overexpressed in prostate tumor tissue (NM_{—}018542/PRO2834)

FIG. 33 contains the gene expression profile determined using the Gene Logic datasuite for a DNA sequence overexpressed in prostate tumor tissue (AI1821426)

FIG. 34 contains the gene expression profile determined using the Gene Logic datasuite for a DNA sequence overexpressed in prostate tumor tissue (AI973051)

FIG. 35 contains the gene expression profile determined using the Gene Logic datasuite for a DNA sequence overexpressed in prostate tumor tissue (AI1979261/AW953116)

FIG. 36 contains the gene expression profile determined using the Gene Logic datasuite for a DNA sequence overexpressed in prostate tumor tissue (AW953116)

FIG. 37 contains the gene expression profile determined using the Gene Logic datasuite for a DNA sequence overexpressed in prostate tumor tissue (AW173166)

FIG. 38 contains the gene expression profile determined using the Gene Logic datasuite for a DNA sequence overexpressed in prostate tumor tissue (AW474960)

FIG. 39 contains the gene expression profile determined using the Gene Logic datasuite for a DNA sequence overexpressed in prostate tumor tissue (BE972639)

FIG. 40 contains the gene expression profile determined using the Gene Logic datasuite for a DNA sequence overexpressed in prostate tumor tissue (N74444)

FIG. 41 contains the gene expression profile determined using the Gene Logic datasuite for a DNA sequence overexpressed in prostate tumor tissue (AW242701)

FIG. 42 contains the gene expression profile determined using the Gene Logic datasuite for a DNA sequence overexpressed in prostate tumor tissue (AW07290)

FIG. 43 contains the gene expression profile determined using the Gene Logic datasuite for a DNA sequence overexpressed in prostate tumor tissue (BF513474)

FIG. 44 contains the gene expression profile determined using the Gene Logic datasuite for a DNA sequence overexpressed in prostate tumor tissue (BF969986)

FIG. 45 contains the gene expression profile determined using the Gene Logic datasuite for a DNA sequence overexpressed in prostate tumor tissue (NM_{—}020372)

FIG. 46 GLUT12 message in multi-tissue panel 1. 1 ng of cDNA from 1 no cDNA, 2 prostate tumor N1, 3 prostate tumor N2, 4, prostate tumor 0, 5 normal brain, 6 normal heart, 7 normal kidney, 8 normal liver, 9 normal lung, 10 normal skeletal muscle, 11 normal pancreas, 12 normal prostate, 13 positive control EST.

FIG. 47 GLUT12 message in multi-tissue panel 1. 5 ng of cDNA from 1 no cDNA, 2 prostate tumor N1, 3 normal brain, 4 normal heart, 5 normal kidney, 6 normal liver, 7 normal lung, 8 normal skeletal muscle, 9 normal pancreas, 10 normal prostate.

FIG. 48 GLUT12 message in multi-tissue panel 11. 5 ng of cDNA from 1 no cDNA, 2 prostate tumor N, 3 prostate tumor O, 4, normal colon, 5 normal heart, 6 normal peripheral blood lymphocytes, 7 normal small intestine, 8 normal ovary, 9 normal spleen, 10 normal testis, 11 normal thymus 12, EST positive control.

FIG. 49 GLUT12 message in brain tissue panel. 5 ng of cDNA from 1 no cDNA, 2 cerebral cortex, 3 cerebellum, 4 medulla oblongata, 5 pons, 6 frontal lobe, 7 occipital lobe, 8 parietal lobe, 9 temporal lobe, 10 placenta, 11 EST positive control.

FIG. 50 GLUT12 message in heart tissue panel. 5 ng of cDNA from 1 no cDNA, 2 prostate tumor N, 3 prostate tumor O, 4 adult heart, 5 fetal heart, 6 aorta, 7 apex, 8 left atrium, 9 right atrium, 10 left ventricle, 11 right ventricle, 12 dextra auricle, 13 sinistra auricle, 14 atrioventricular node, 15 septum intraven, 16 EST positive control.

FIG. 51 PSAT message in multi-tissue panel 1. 1 ng of cDNA from 1 no cDNA, 2 normal prostate N, 3 prostate tumor N, 4, prostate tumor O, 5 normal brain, 6 normal heart, 7 normal kidney, 8 normal liver, 9 normal lung, 10 normal skeletal muscle, 11 normal pancreas, 12 normal prostate, 13 positive control EST.

FIG. 52 PSAT message in multi-tissue panel II. 5 ng of cDNA from 1 no cDNA, 2 normal prostate N, 3 prostate tumor N, 4 prostate tumor O, 5 normal colon, 6 normal peripheral blood lymphocytes, 7 normal small intestine, 8 normal ovary, 9 normal spleen, 10 normal testis, 11 normal thymus 12, EST positive control.

FIG. 53 PSAT message in brain tissue panel. 5 ng of cDNA from 1 no cDNA, 2 cerebral cortex, 3 cerebellum, 4 medulla oblongata, 5 pons, 6 frontal lobe, 7 occipital lobe, 8 parietal lobe, 9 temporal lobe, 10 placenta, 11 EST positive control.

FIG. 54 PSAT message in heart tissue panel. 5 ng of cDNA from 1 no cDNA, 2 adult heart, 3 fetal heart, 4 aorta, 5 apex, 6 left atrium, 7 right atrium, 8 left ventricle, 9 right ventricle, 10 dextra auricle, 11 sinistra auricle, 12 atrioventricular node, 13 septum intraven, 14 EST positive control.

FIG. 55 contains the amino acid and nucleic acid of Kv3.2a and Kv3.2b.

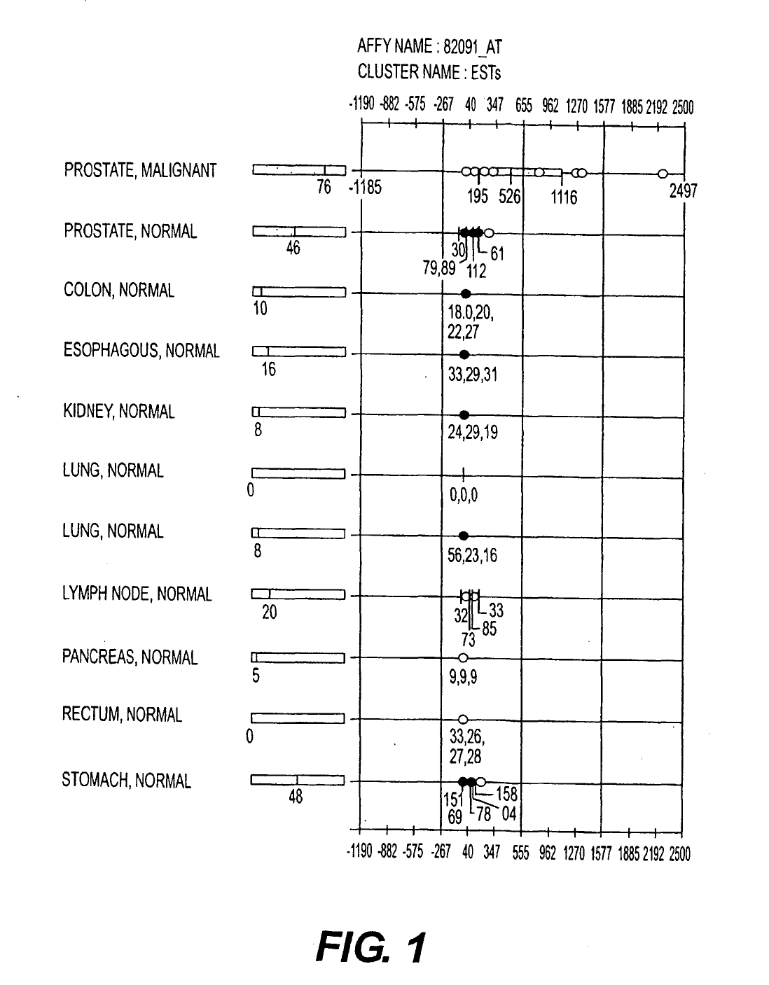

### DETAILED DESCRIPTION OF THE INVENTION

The present invention identifies genes (the sequences of which are provided in the examples infra) using the Gene Logic database that are specifically upregulated in malignant tissues obtained from subjects with prostate cancer. Specifically, the gene sequences which were identified by hybridization analysis are specifically upregulated in a substantial percentage of prostate cancer tissues in relation to various normal tissues screened using the same hybridization probes (prostate, kidney, lung, pancreas, stomach, prostate, esophagus, liver, lymph note and rectum) as well as relative to other normal tissues. The results of these hybridization analyses are set forth infra in the examples.

For example, the invention provides three genes identified and referred to herein as DWAN, Kv3.2 and MASP. The first gene DWAN, (comprising the nucleic acid sequence identified infra as SEQ ID NO: 1) was identified using the GeneLogic probe 147504 and is contained in EST IMAGE 2251589. As shown in FIG. 3, DWAN encodes a protein of 69 amino acids (followed by a step codon) that comprises a putative transmembrane domain and possible PKC and tyrosine phosphorylation sites. The predicted amino acid sequence for DWAN is comprised in SEQ ID NO: 2. As the protein is likely expressed on the surface of prostate cancer cells, DWAN is a potential target for antibody therapy, e.g. using naked antibodies or conjugated antibodies an effect or moiety, e.g. a radionuclide.

The second gene, Kv3.2, identified using as the probe 117293 is predicted to be an extension of the 3′ UTK of the potassium channel KV3.2a. This gene is in the public domain and exists in at least two alternatively spliced versions, KV3.2a and KV3.2b, both possessing the same extracellular domain and differing only in the C-terminal amino acids. As the polypeptide encoded by KV3.2 is also predicted to be expressed on the surface of prostate cancer cells (as evidence by the presence of extracellular domains) the corresponding protein is also an appropriate potential candidate for antibody therapy.

The DNA and protein Sequences for both splice variants are:

| | | |
|---|---|---|
| KV3.2a | (DNA) | AF268897 |
| KV3.2a | (protein) | AF26897_1 |
| KV3.2b | (DNA) | AF268896 |
| KV3.2b | (protein) | AF268896_1 |

The third gene which was found to be upregulated in prostate tumor tissues, MASP, which comprises the nucleic acid sequence identified infra as SEQ ID NO: 3 is contained on a single exon. This gene is also believed to be expressed on the surface of prostate tumor cells.

Based on the results disclosed in the examples, it is anticipated that these the disclosed genes and the corresponding proteins are suitable targets for prostate cancer therapy, prevention or diagnosis, e.g. for the development of antibodies, antibody fragments, small molecular inhibitors, anti-sense therapeutics, therapies, interfering RNA therapies and ribozymes. The potential therapies are described in greater detail below.

Such therapies will include the synthesis of oligonucleotides having sequences in the antisense orientation relative to the three genes identified to be unregulated in prostate cancer. Suitable therapeutic antisense oligonucleotides will typically vary in length from two to several hundred nucleotides in length, more typically about 50-70 nucleotides in length or shorter. These antisense oligonucleotides may be administered as naked DNAs or in protected forms, e.g., encapsulated in liposomes. The use of liposomal or other protected forms may be advantageous as it may enhance in vivo stability and delivery to target sites, i.e., prostate tumor cells.

Also, the subject novel genes may be used to design novel ribozymes that target the cleavage of the corresponding mRNAs in prostate tumor cells. Similarly, these ribozymes may be administered in free (naked) form or by the use of delivery systems that enhance stability and/or targeting, e.g., liposomes. Ribozymal and antisense therapies used to target genes that are selectively expressed by cancer cells are well known in the art.

Also, the invention embraces the use of short interfering RNAs, (RNA's). e.g., that may be single, double or triple stranded, that target the genes disclosed infra that are upregulated in prostate cancer.

Also, the present invention embraces the administration of use of DNAs that hybridize to the novel gene targets identified infra, attached to therapeutic effector moieties, e.g., radiolabels, e.g., yttrium, iodine, cytotoxins, cytokines, prodrugs or enzymes, in order to selectively target and kill cells that express these genes, i.e., prostate tumor cells.

Also, the present invention embraces the treatment and/or diagnosis of prostate cancer by targeting altered genes or the corresponding altered protein particularly splice variants that are expressed in altered form in prostate cells. These methods will provide for the selective detection of cells and/or eradication of cells that express such altered forms thereby avoiding adverse effects to normal cells.

Still further, the present invention encompasses non-nucleic acid based therapies. Particularly, the invention encompasses the use of an antigen encoded by the novel cDNAs disclosed in the examples of the corresponding antigens. It is anticipated that these antigens may be used as therapeutic or prophylactic anti-tumor vaccines. For example, a particular contemplated application of these antigens involves their administration with adjuvants that induce a cytotoxic T lymphocyte response. An especially preferred adjuvant developed by the Assignee of this application, IDEC Pharmaceuticals Corporation, is disclosed in U.S. Pat. Nos. 5,709,860, 5,695,770, and 5,585,103, the disclosures of which are incorporated by reference in their entirety. In particular, the use of this adjuvant to promote CTL responses against prostate and papillomavirus related human prostate cancer has been suggested.

Also, administration of the subject novel antigens in combination with an adjuvant may result in a humoral immune response against such antigens, thereby delaying or preventing the development of prostate cancer.

Essentially, these embodiments of the invention will comprise administration of one or both of the subject novel prostate cancer antigens, ideally in combination with an adjuvant, e.g., PROVAX®, which comprises a microfluidized adjuvant containing Squalene, Tween and Pluronic, in an amount sufficient to be therapeutically or prophylactically effective. A typical dosage will range from 50 to 20,000 mg/kg body weight, have typically 100 to 5000 mg/kg body weight.

Alternatively, the subject prostate tumor antigens may be administered with other adjuvants, e.g., ISCOM'S®, DETOX®, SAF, Freund's adjuvant, Alum®, Saponin®, among others.

However, the preferred embodiment of the invention will comprise the preparation of monoclonal antibodies or antibody fragments against the antigens encoded by the novel genes containing the nucleic acid sequences disclosed infra. Such monoclonal antibodies can be produced by conventional methods and include human monoclonal antibodies, antibody dimers or tetramers, humanized monoclonal antibodies, chimeric monoclonal antibodies, single chain antibodies, e.g., scFv's and antigen-binding antibody fragments such as Fabs, 2 Fabs, and Fab′ fragments, and domain deleted antibodies. Methods for the preparation of monoclonal antibodies and fragments thereof, e.g., by pepsin or papain-mediated cleavage are well known in the art. In general, this will comprise immunization of an appropriate (non-homologous) host with the subject prostate cancer antigens, isolation of immune cells therefrom, use of such immune cells to make hybridomas, and screening for monoclonal antibodies that specifically bind to either of such antigens. Methods for preparation of antibodies, including tetrameric antibodies and domain-deleted antibodies, in particular CH₂ domain-deleted antibodies are disclosed in commonly assigned PCT applications, PCT/US02/02373 and PCT/US02/02374 both filed on Jan. 29, 2002, which name Braslawsky et al., as the inventor.

These antibodies and fragments thereof, e.g., domain deleted antibodies fragments will be useful for passive anti-tumor immunotherapy, or may be attached to therapeutic effector moieties, e.g., radiolabels, cytotoxins, therapeutic enzymes, agents that induce apoptosis, in order to provide for targeted cytotoxicity, i.e., killing of human prostate tumor cells. Given the fact that the subject genes are apparently not significantly expressed by many normal tissues this should not result in significant adverse side effects (toxicity to non-target tissues).

In this embodiment, such antibodies or fragments will be administered in labeled or unlabeled form, alone or in combination with other therapeutics, e.g., chemotherapeutics such as cisplatin, methotrexate, adriamycin, and other chemotherapies suitable for prostate cancer therapy. The administered composition will include a pharmaceutically acceptable carrier, and optionally adjuvants, stabilizers, etc., used in antibody compositions for therapeutic use.

Preferably, such monoclonal antibodies will bind the target antigens with high affinity, e.g., possess a binding affinity (Kd) on the order of 10⁻⁶ to 10⁻¹² M.

As noted, the present invention also embraces diagnostic applications that provide for detection of the expression of prostate specific genes disclosed herein. Essentially, this will comprise detecting the expression of one or all of these genes at the DNA level or at the protein level.

At the DNA level, expression of the subject genes will be detected by known DNA detection methods, e.g., Northern blot hybridization, strand displacement amplification (SDA), catalytic hybridization amplification (CHA), and other known DNA detection methods. Preferably, a cDNA library will be made from prostate cells obtained from a subject to be tested for prostate cancer by PCR using primers corresponding to either or both of the novel genes disclosed in this application.

The presence or absence of prostate cancer will be determined based on whether PCR products are obtained, and the level of expression. The levels of expression of such PCR product may be quantified in order to determine the prognosis of a particular prostate cancer patient (as the levels of expression of the PCR product likely will increase as the disease progresses.) This may provide a method of monitoring the status of a prostate cancer patient. Of course, suitable controls will be effected.

Alternatively, the status of a subject to be tested for prostate cancer may be evaluated by testing biological fluids, e.g., blood, urine, lymph, with an antibody or antibodies or fragment that specifically binds to the novel prostate tumor antigens disclosed herein.

Methods for using antibodies to detect antigen expression are well known and include ELISA, competitive binding assays, etc. In general, such assays use an antibody or antibody fragment that specifically binds the target antigen directly or indirectly bound to a label that provides for detection, e.g., a radiolabel enzyme, fluorophore, etc.

Patients which test positive for the enhanced presence of the antigen on prostate cells will be diagnosed as having or being at increased risk of developing prostate cancer. Additionally, the levels of antigen expression may be useful in determining patient status, i.e., how far disease has advanced (stage of prostate cancer).

As noted, the present invention identified and provides the sequences of genes and corresponding antigens the overexpression of which correlates to human prostate cancer. The present invention also embraces variants thereof. By “variants” is intended sequences that are at least 75% identical thereto, more preferably at least 85% identical, and most preferably at least 90% identical when these DNA sequences are aligned to a nucleic acid sequence encoding the subject DNAs or a fragment thereof having a size of at least 50 nucleotides. This includes in particular allelic and splice variants of the subject genes.

Also, the present invention provides for primer pairs that result in the amplification DNAs encoding the subject novel genes or a portion thereof in an mRNA library obtained from a desired cell source, typically human prostate cell or tissue sample. Typically, such primers will be on the order of 12 to 50 nucleotides in length, and will be constructed such that they provide for amplification of the entire or most of the target gene.

Also, the invention embraces the antigens encoded by the subject DNAs or fragments thereof that bind to or elicits antibodies specific to the full-length antigens. Typically, such fragments will be at least 10 amino acids in length, more typically at least 25 amino acids in length.

As noted, the subject genes are expressed in a majority of prostate tumor samples tested. The invention further contemplates the identification of other cancers that express such genes and the use thereof to detect and treat such cancers. For example, the subject genes or variants thereof may be expressed on other cancers, e.g., breast, pancreas, lung or prostate cancers. Essentially, the present invention embraces the detection of any cancer wherein the expression of the subject novel genes or variants thereof correlate to a cancer or an increased likelihood of cancer.

“Isolated tumor antigen or tumor protein” refers to any protein that is not in its normal cellular millieu. This includes by way of example compositions comprising recombinant proteins encoded by the genes disclosed infra, pharmaceutical compositions comprising such purified proteins, diagnostic compositions comprising such purified proteins, and isolated protein compositions comprising such proteins. In preferred embodiments, an isolated prostate tumor protein according to the invention will comprise a substantially pure protein, in that it is substantially free of other proteins, preferably that is at least 90% pure, that comprises the amino acid sequence contained herein or natural homologues or mutants having essentially the same sequence. A naturally occurring mutant might be found, for instance, in tumor cells expressing a gene encoding a mutated protein according to the invention.

“Native tumor antigen or tumor protein” refers to a protein that is a non-human primate homologue of the protein having the amino acid sequence contained infra.

“Isolated prostate tumor gene or nucleic acid sequence” refers to a nucleic acid molecule that encodes a tumor antigen according to the invention which is not in its normal human cellular millieu, e.g., is not comprised in the human or non-human primate chromosomal DNA. This includes by way of example vectors that comprise a gene according to the invention, a probe that comprises a gene according to the invention, and a nucleic acid sequence directly or indirectly attached to a detectable moiety, e.g. a fluorescent or radioactive label, or a DNA fusion that comprises a nucleic acid molecule encoding a gene according to the invention fused at its 5′ or 3′ end to a different DNA, e.g. a promoter or a DNA encoding a detectable marker or effector moiety. Also included are natural homologues or mutants having substantially the same sequence. Naturally occurring homologies that are degenerate would encode the same protein including nucleotide differences that do not change the corresponding amino acid sequence. Naturally occurring mutants might be found in tumor cells, wherein such nucleotide differences may result in a mutant tumor antigen. Naturally occurring homologues containing conservative substitutions are also encompassed.

“Variant of prostate tumor antigen or tumor protein” refers to a protein possessing an amino acid sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to the corresponding native tumor antigen wherein sequence identity is as defined infra. Preferably, this variant will possess at least one biological property in common with the native protein.

“Variant of prostate tumor gene or nucleic acid molecule or sequence” refers to a nucleic acid sequence that possesses at least 90% sequence identity, more preferably at least 91%, more preferably at least 92%, even more preferably at least 93%, still more preferably at least 94%, even more preferably at least 95%, still more preferably at least 96%, even more preferably at least 97%, even more preferably at least 98% sequence identity, and most preferably at least 99% sequence identity, to the corresponding native human nucleic acid sequence, wherein “sequence identity” is as defined infra.

“Fragment of prostate antigen encoding nucleic acid molecule or sequence” refers to a nucleic acid sequence corresponding to a portion of the native human gene wherein said portion is at least about 50 nucleotides in length, or 100, more preferably at least 150 nucleotides in length.

“Antigenic fragments of prostate tumor antigen” refer to polypeptides corresponding to a fragment of a prostate protein or a variant or homologue thereof that when used itself or attached to an immunogenic carrier that elicits antibodies that specifically bind the protein. Typically such antigenic fragments will be at least 20 amino acids in length.

Sequence identity or percent identity is intended to mean the percentage of the same residues shared between two sequences, when the two sequences are aligned using the Clustal method [Higgins et al, Cabios 8:189-191 (1992)] of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 [Dayhoff et al., in Atlas of Protein Sequence and Structure, Dayhoff, Ed., NDRF, Washington, Vol. 5, suppl. 3, p. 345, (1978)].

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human Gene A or gene B when determining percent conservation with non-human Gene A or gene B, e.g. mgene A or gene B, when determining percent conservation. Conservative amino acid changes satisfying this requirement are: R-K; E-D, Y-F, L-M; V-I, Q-H.

### Polypeptide Fragments

The invention provides polypeptide fragments of the disclosed proteins. Polypeptide fragments of the invention can comprise at least 8, more preferably at least 25, still more preferably at least 50 amino acid residues of the protein or an analogue thereof. More particularly such fragment will comprise at least 75, 100, 125, 150, 175, 200, 225, 250, 275 residues of the polypeptide encoded by the corresponding gene. Even more preferably, the protein fragment will comprise the majority of the native protein, e.g. about 100 contiguous residues of the native protein.

### Biologically Active Variants

The invention also encompasses mutants of the novel prostate proteins disclosed infra which comprise an amino acid sequence that is at least 80%, more preferably 90%, still more preferably 95-99% similar to the native protein.

Guidance in determining which amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant.

Protein variants include glycosylated forms, aggregative conjugates with other molecules, and covalent conjugates with unrelated chemical moieties. Also, protein variants also include allelic variants, species variants, and muteins. Truncations or deletions of regions which do not affect the differential expression of the gene are also variants. Covalent variants can be prepared by linking functionalities to groups which are found in the amino acid chain or at the N- or C-terminal residue, as is known in the art.

It will be recognized in the art that some amino acid sequence of the prostate proteins of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the prostate proteins disclosed infra which show comparable expression patterns or which include antigenic regions. Such mutants include deletions, insertions, inversions, repeats, and site substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255: 306-312 (1992)).

As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

### Fusion Proteins

Fusion proteins comprising proteins or polypeptide fragments of the subject prostate tumor antigen can also be constructed. Fusion proteins are useful for generating antibodies against amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of a protein according to the invention or a fragment thereof can be used to target other protein domains to cellular locations in which the domains are not normally found, such as bound to a cellular membrane or secreted extracellularly.

A fusion protein comprises two protein segments fused together by means of a peptide bond. As noted, these fragments may range in size from about 8 amino acids up to the full length of the protein.

The second protein segment can be a full-length protein or a polypeptide fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP 16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence encoding a possible antigen according to the invention or a fragment thereof in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Proteins, fusion proteins, or polypeptides of the invention can be produced by recombinant DNA methods. For production of recombinant proteins, fusion proteins, or polypeptides, a sequence encoding the protein can be expressed in prokaryotic or eukaryotic host cells using expression systems known in the art. These expression systems include bacterial, yeast, insect, and mammalian cells.

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be necessary to modify a protein produced in yeast or bacteria, for example by phosphorylation or glycosylation of the appropriate sites, in order to obtain a functional protein. Such covalent attachments can be made using known chemical or enzymatic methods.

A protein or polypeptide of the invention can also be expressed in cultured host cells in a form which will facilitate purification. For example, a protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence disclosed herein can also be used to construct transgenic animals, such as mice, rats, guinea pigs, cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule.

Typically, homologous polynucleotide sequences can be confirmed by hybridization under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each, homologous sequences can be identified which contain at most about 25-30% basepair mismatches. More preferably, homologous nucleic acid strands contain 15-25% basepair mismatches, even more preferably 5-15% basepair mismatches.

The invention also provides polynucleotide probes which can be used to detect complementary nucleotide sequences, for example, in hybridization protocols such as Northern or Southern blotting or in situ hybridizations. Polynucleotide probes of the invention comprise at least 12, 13, 14, 15, 16, 17, 18, 19, 20, 30, or 40 or more contiguous nucleotides of the nucleic acid sequences provided herein. Polynucleotide probes of the invention can comprise a detectable label, such as a radioisotopic, fluorescent, enzymatic, or chemiluminescent label.

Isolated genes corresponding to the cDNA sequences disclosed herein are also provided. Standard molecular biology methods can be used to isolate the corresponding genes using the cDNA sequences provided herein. These methods include preparation of probes or primers from the nucleotide sequence disclosed herein for use in identifying or amplifying the genes from mammalian, including human, genomic libraries or other sources of human genomic DNA.

Polynucleotide molecules of the invention can also be used as primers to obtain additional copies of the polynucleotides, using polynucleotide amplification methods. Polynucleotide molecules can be propagated in vectors and cell lines using techniques well known in the art. Polynucleotide molecules can be on linear or circular molecules. They can be on autonomously replicating molecules or on molecules without replication sequences. They can be regulated by their own or by other regulatory sequences, as is known in the art.

### Polynucleotide Constructs

Polynucleotide molecules comprising the coding sequences disclosed herein can be used in a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Also included are polynucleotide molecules comprising the promoter and UTR sequences of the subject novel genes, operably linked to the associated protein coding sequence and/or other sequences encoding a detectable or selectable marker. Such promoter and/or UTR-based constructs are useful for studying the transcriptional and translational regulation of protein expression, and for identifying activating and/or inhibitory regulatory proteins.

### Host Cells

An expression construct can be introduced into a host cell. The host cell comprising the expression construct can be any suitable prokaryotic or eukaryotic cell. Expression systems in bacteria include those described in Chang et al., Nature 275:615 (1978); Goeddel et al., Nature 281: 544 (1979); Goeddel et al., Nucleic Acids Res. 8:4057 (1980); EP 36,776; U.S. Pat. No. 4,551,433; deBoer et al., Proc. Natl. Acad. Sci. USA 80: 21-25 (1983); and Siebenlist et al., Cell 20: 269 (1980).

Expression systems in yeast include those described in Hinnen et al., Proc. Natl. Acad. Sci. USA 75: 1929 (1978); Ito et al., J Bacteriol 153: 163 (1983); Kurtz et al., Mol. Cell. Biol. 6: 142 (1986); Kunze et al., J Basic Microbiol. 25: 141 (1985); Gleeson et al., J. Gen. Microbiol. 132: 3459 (1986), Roggenkamp et al., Mol. Gen. Genet. 202: 302 (1986)); Das et al., J. Bacteriol. 158: 1165 (1984); De Louvencourt et al., J. Bacteriol. 154:737 (1983), Van den Berg et al., Bio/Technology 8: 135 (1990); Kunze et al., J. Basic Microbiol. 25: 141 (1985); Cregg et al., Mol. Cell. Biol. 5: 3376 (1985); U.S. Pat. No. 4,837,148; U.S. Pat. No. 4,929,555; Beach and Nurse, Nature 300: 706 (1981); Davidow et al., Curr. Genet. 10: 380 (1985); Gaillardin et al., Curr. Genet. 10: 49 (1985); Ballance et al., Biochem. Biophys. Res. Commun. 112: 284-289 (1983); Tilburn et al., Gene 26: 205-22 (1983); Yelton et al., Proc. Natl. Acad, Sci. USA 81: 1470-1474 (1984); Kelly and Hynes, EMBO J. 4: 475-479 (1985); EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. 69: 765-776 (1988); Miller et al., Ann. Rev. Microbiol. 42: 177 (1988); Carbonell et al., Gene 73: 409 (1988); Maeda et al., Nature 315: 592-594 (1985); Lebacq-Verheyden et al., Mol. Cell Biol. 8: 3129 (1988); Smith et al., Proc. Natl. Acad. Sci. USA 82: 8404 (1985); Miyajima et al., Gene 58: 273 (1987); and Martin et al., DNA 7:99 (1988). Numerous baculoviral strains and variants and corresponding permissive insect host cells from hosts are described in Luckow et al., Bio/Technology (1988) δ: 47-55, Miller et al., in GENETIC ENGINEERING (Setlow, J. K. et al. eds.), Vol. 8, pp. 277-279 (Plenum Publishing, 1986); and Maeda et al., Nature, 315: 592-594 (1985).

Mammalian expression can be accomplished as described in Dijkema et al., EMBO J. 4: 761 (1985); Gorman et al., Proc. Natl. Acad. Sci. USA 79: 6777 (1982b); Boshart et al., Cell 41: 521 (1985); and U.S. Pat. No. 4,399,216. Other features of mammalian expression can be facilitated as described in Ham and Wallace, Meth Enz. 58: 44 (1979); Barnes and Sato, Anal. Biochem. 102: 255 (1980); U.S. Pat. No. 4,767,704; U.S. Pat. No. 4,657,866; U.S. Pat. No. 4,927,762; U.S. Pat. No. 4,560,655; WO 90/103430, WO 87/00195, and U.S. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a protein of the invention can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12, 15, 20, or 50 contiguous nucleotides of the nucleotide sequence shown in the figures herein. The transcription unit is located upstream to a coding sequence of the endogenous gene. The exogenous regulatory sequence directs transcription of the coding sequence of the endogenous gene.

The invention can also include hybrid and modified forms thereof including fusion proteins, fragments and hybrid and modified forms in which certain amino acids have been deleted or replaced, modifications such as where one or more amino acids have been changed to a modified amino acid or unusual amino acid.

Also included within the meaning of substantially homologous is any human or non-human primate protein which may be isolated by virtue of cross-reactivity with antibodies to proteins encoded by a gene described herein or whose encoding nucleotide sequences including genomic DNA, mRNA or cDNA may be isolated through hybridization with the complementary sequence of genomic or subgenomic nucleotide sequences or cDNA of a gene herein or fragments thereof. It will also be appreciated by one skilled in the art that degenerate DNA sequences can encode a tumor protein according to the invention and these are also intended to be included within the present invention as are allelic variants of the subject genes.

Preferred is a prostate protein according to the invention prepared by recombinant DNA technology. By “pure form” or “purified form” or “substantially purified form” it is meant that a protein composition is substantially free of other proteins which are not the desired protein.

The present invention also includes therapeutic or pharmaceutical compositions comprising a protein according to the invention in an effective amount for treating patients with disease, and a method comprising administering a therapeutically effective amount of the protein. These compositions and methods are useful for treating cancers associated with the subject proteins, e.g. prostate cancer. One skilled in the art can readily use a variety of assays known in the art to determine whether the protein would be useful in promoting survival or functioning in a particular cell type.

In certain circumstances, it may be desirable to modulate or decrease the amount of the protein expressed by a cell, e.g. ovary cell. Thus, in another aspect of the present invention, anti-sense oligonucleotides can be made and a method utilized for diminishing the level of expression a prostate antigen according to the invention by a cell comprising administering one or more anti-sense oligonucleotides. By anti-sense oligonucleotides reference is made to oligonucleotides that have a nucleotide sequence that interacts through base pairing with a specific complementary nucleic acid sequence involved in the expression of the target such that the expression of the gene is reduced. Preferably, the specific nucleic acid sequence involved in the expression of the gene is a genomic DNA molecule or mRNA molecule that encodes the gene. This genomic DNA molecule can comprise regulatory regions of the gene, or the coding sequence for the mature gene.

The term complementary to a nucleotide sequence in the context of antisense oligonucleotides and methods therefor means sufficiently complementary to such a sequence as to allow hybridization to that sequence in a cell, i.e., under physiological conditions. Antisense oligonucleotides preferably comprise a sequence containing from about 8 to about 100 nucleotides and more preferably the antisense oligonucleotides comprise from about 15 to about 30 nucleotides. Antisense oligonucleotides can also contain a variety of modifications that confer resistance to nucleolytic degradation such as, for example, modified internucleoside lineages [Uhlmann and Peyman, Chemical Reviews 90:543-548 (1990); Schneider and Banner, Tetrahedron Lett. 31:335, (1990) which are incorporated by reference], modified nucleic acid bases as disclosed in U.S. Pat. No. 5,958,773 and patents disclosed therein, and/or sugars and the like.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. [Branch, A. D., T.I.B.S. 23:45-50 (1998)].

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

Additionally, the subject prostate tumor proteins can also be linked or conjugated with agents that provide desirable pharmaceutical or pharmacodynamic properties. For example, the protein can be coupled to any substance known in the art to promote penetration or transport across the blood-brain barrier such as an antibody to the transferrin receptor, and administered by intravenous injection (see, for example, Friden et al., Science 259:373-377 (1993) which is incorporated by reference). Furthermore, the subject prostate antigens can be stably linked to a polymer such as polyethylene glycol to obtain desirable properties of solubility, stability, half-life and other pharmaceutically advantageous properties. [See, for example, Davis et al., Enzyme Eng. 4:169-73 (1978); Buruham, Am. J. Hosp. Pharm. 51:210-218 (1994) which are incorporated by reference].

The compositions are usually employed in the form of pharmaceutical preparations. Such preparations are made in a manner well known in the pharmaceutical art. See, e.g. Remington Pharmaceutical Science, 18th Ed., Merck Publishing Co. Eastern PA, (1990). One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. The subject prostate tumor antigens, fragments or variants thereof can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parental administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing the subject prostate or variant or fragment thereof are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, the protein may be therapeutically administered by implanting into patients vectors or cells capable of producing a biologically-active form of the protein or a precursor of protein, i.e., a molecule that can be readily converted to a biological-active form of the protein by the body. In one approach, cells that secrete the protein may be encapsulated into semipermeable membranes for implantation into a patient. The cells can be cells that normally express the protein or a precursor thereof or the cells can be transformed to express the protein or a precursor thereof. It is preferred that the cell be of human origin and that the protein be a human protein when the patient is human. However, it is anticipated that non-human primate homologues of the protein discussed infra may also be effective.

In a number of circumstances it would be desirable to determine the levels of protein or corresponding mRNA in a patient. Evidence disclosed infra suggests the subject prostate proteins may be expressed at different levels during some diseases, e.g., cancers, provides the basis for the conclusion that the presence of these proteins serves a normal physiological function related to cell growth and survival. Endogenously produced protein according to the invention may also play a role in certain disease conditions.

The term “detection” as used herein in the context of detecting the presence of protein in a patient is intended to include the determining of the amount of protein or the ability to express an amount of protein in a patient, the estimation of prognosis in terms of probable outcome of a disease and prospect for recovery, the monitoring of the protein levels over a period of time as a measure of status of the condition, and the monitoring of protein levels for determining a preferred therapeutic regimen for the patient, e.g. one with prostate cancer.

To detect the presence of a prostate protein according to the invention in a patient, a sample is obtained from the patient. The sample can be a tissue biopsy sample or a sample of blood, plasma, serum, CSF, urine or the like. It has been found that the subject proteins are expressed at high levels in some cancers. Samples for detecting protein can be taken from prostate tissues. When assessing peripheral levels of protein, it is preferred that the sample be a sample of blood, plasma or serum. When assessing the levels of protein in the central nervous system a preferred sample is a sample obtained from cerebrospinal fluid or neural tissue.

In some instances, it is desirable to determine whether the gene is intact in the patient or in a tissue or cell line within the patient. By an intact gene, it is meant that there are no alterations in the gene such as point mutations, deletions, insertions, chromosomal breakage, chromosomal rearrangements and the like wherein such alteration might alter production of the corresponding protein or alter its biological activity, stability or the like to lead to disease processes. Thus, in one embodiment of the present invention a method is provided for detecting and characterizing any alterations in the gene. The method comprises providing an oligonucleotide that contains the gene, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarity to the sequence from which it is derived to hybridize specifically to the gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, TaqI and AluI. Using the Southern blot protocol, which is well known in the art, this assay determines whether a patient or a particular tissue in a patient has an intact prostate gene according to the invention or a gene abnormality.

Hybridization to a gene would involve denaturing the chromosomal DNA to obtain a single-stranded DNA; contacting the single-stranded DNA with a gene probe associated with the gene sequence; and identifying the hybridized DNA-probe to detect chromosomal DNA containing at least a portion of a gene.

The term “probe” as used herein refers to a structure comprised of a polynucleotide that forms a hybrid structure with a target sequence, due to complementarily of probe sequence with a sequence in the target region. Oligomers suitable for use as probes may contain a minimum of about 8-12 contiguous nucleotides which are complementary to the targeted sequence and preferably a minimum of about 20.

A gene according to the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 25°-45° C., more preferably at 32°-40° C. and more preferably at 37°-38° C. The time required for hybridization is from about 0.25 to about 96 hours, more preferably from about one to about 72 hours, and most preferably from about 4 to about 24 hours.

Gene abnormalities can also be detected by using the PCR method and primers that flank or lie within the gene. The PCR method is well known in the art. Briefly, this method is performed using two oligonucleotide primers which are capable of hybridizing to the nucleic acid sequences flanking a target sequence that lies within a gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, a method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified.

After PCR amplification, the DNA sequence comprising the gene or a fragment thereof is then directly sequenced and analyzed by comparison of the sequence with the sequences disclosed herein to identify alterations which might change activity or expression levels or the like.

In another embodiment, a method for detecting a tumor protein according to the invention is provided based upon an analysis of tissue expressing the gene. Certain tissues such as prostate tissues have been found to overexpress the subject gene. The method comprises hybridizing a polynucleotide to mRNA from a sample of tissue that normally expresses the gene. The sample is obtained from a patient suspected of having an abnormality in the gene.

To detect the presence of mRNA encoding the protein, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques.

The mRNA of the sample is contacted with a DNA sequence serving as a probe to form hybrid duplexes. The use of a labeled probes as discussed above allows detection of the resulting duplex.

When using the cDNA encoding the protein or a derivative of the cDNA as a probe, high stringency conditions can be used in order to prevent false positives, that is the hybridization and apparent detection of the gene nucleotide sequence when in fact an intact and functioning gene is not present. When using sequences derived from the gene cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. [Sambrook et al. (1989), supra].

In order to increase the sensitivity of the detection in a sample of mRNA encoding the detected prostate antigen, the technique of reverse transcription/polymerization chain reaction (RT/PCR) can be used to amplify cDNA transcribed from mRNA encoding the prostate tumor antigen. The method of RT/PCR is well known in the art, and can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and gene A or gene B specific primers. [Belyavsky et al., Nucl. Acid Res. 17:2919-2932 (1989); Krug and Berger, Methods in Enzymology, 152:316-325, Academic Press, NY (1987) which are incorporated by reference].

The polymerase chain reaction method is performed as described above using two oligonucleotide primers that are substantially complementary to the two flanking regions of the DNA segment to be amplified. Following amplification, the PCR product is then electrophoresed and detected by ethidium bromide staining or by phosphoimaging.

The present invention further provides for methods to detect the presence of the protein in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. [Basic and Clinical Immunology, 217-262, Sites and Terr, eds., Appleton & Lange, Norwalk, Conn., (1991), which is incorporated by reference]. Preferred are binder-ligand immunoassay methods including reacting antibodies with an epitope or epitopes of the prostate tumor antigen protein and competitively displacing a labeled prostate antigen according to the invention or derivative thereof.

As used herein, a derivative of the subject prostate tumor antigen is intended to include a polypeptide in which certain amino acids have been deleted or replaced or changed to modified or unusual amino acids wherein the derivative is biologically equivalent to gene and wherein the polypeptide derivative cross-reacts with antibodies raised against the protein. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

Polyclonal or monoclonal antibodies to the subject protein or an epitope thereof can be made for use in immunoassays by any of a number of methods known in the art. By epitope reference is made to an antigenic determinant of a polypeptide. An epitope could comprise 3 amino acids in a spatial conformation which is unique to the epitope. Generally an epitope consists of at least 5 such amino acids. Methods of determining the spatial conformation of amino acids are known in the art, and include, for example, x-ray crystallography and 2 dimensional nuclear magnetic resonance.

One approach for preparing antibodies to a protein is the selection and preparation of an amino acid sequence of all or part of the protein, chemically synthesizing the sequence and injecting it into an appropriate animal, typically a rabbit, hamster or a mouse.

Oligopeptides can be selected as candidates for the production of an antibody to the protein based upon the oligopeptides lying in hydrophilic regions, which are thus likely to be exposed in the mature protein. Suitable additional oligopeptides can be determined using, for example, the Antigenicity Index, Welling, G. W. et al., FEBS Lett. 188:215-218 (1985), incorporated herein by reference.

The anti-prostate antibodies or fragments according to the invention may be administered in naked form, or can be conjugated to desired effective moieties. Examples thereof include therapeutic proteins such as lymphokines and cytokines, diagnostic and therapeutic enzymes, chemotherapeutic agents, radionuclides, prodrugs, cytotoxins, and the like.

In a preferred embodiment of the invention, the antibody or fragment will be conjugated directly or indirectly to a radionuclide, e.g., by use of a chelating agent. Examples of suitable radiolabels include by way of example ⁹⁰Y, ¹²⁵I, ¹³¹I, ¹¹¹In, ¹⁰⁵Rh, ¹⁵³Sm, ⁶⁷Cu, ⁶⁷Ga, ¹⁶⁶Ho, ¹⁷⁷Lo, ¹⁸⁶Re, ²¹³ Bi, ²¹¹At, ¹⁰⁹Pd, ²¹²Bi, and ¹⁸⁸Re.

Examples of therapeutic proteins include interferons, interleukins, colony stimulating factor, tumor necrosis factor, lymphotoxins, and the like.

Examples of chemotherapeutic agents include by way of example adriamycin, methotrexate, cisplatin, daunorubicin, doxorubicin, methopterin, caminomycin, mitheramycin, streptnigrin, chlorambucil, ifosfimide, et al. Examples of suitable toxins include diptheria toxin, cholera toxin, ricin, pseudomonas toxin, calicheamicin, euperamicin, dynemicin and variants thereof.

Additionally, the invention embraces the use of the subject targeted therapeutics, e.g., antibodies with hormones and hormone antagonists, such as corticosteroids, e.g., prednisone, progestions, anthestrogens, e.g., tamoxifin, andrrogenes, e.g., texosteroid and aromatase inhibitors.

Suitable prodrugs that may be attached to antibodies include e.g., phosphate-containing prodrugs, thiophosphate-containing prodrugs, sulfate containing prodrugs peptide containing prodrugs, and beta lactam containing prodrugs.

As noted, in a preferred embodiment radiolabeled antibodies will be prepared against one of the prostate antigens disclosed infra and used for the treatment of prostate cancer via radioimmunotherapy. Preferably these antibodies will not elicit an immunogenic response as effective therapy will typically comprise chronic, in multiple administrations of the particular antibody, either in whole or conjugated form.

Anti-Prostate Antigen Antibodies

As noted, the invention preferably includes the preparation and use of anti-prostate antigen antibodies and fragments for use as diagnostics and therapeutics. These antibodies may be polyclonal or monoclonal. Polyclonal antibodies can be prepared by immunizing rabbits or other animals by injecting antigen followed by subsequent boosts at appropriate intervals. The animals are bled and sera assayed against purified protein usually by ELISA or by bioassay based upon the ability to block the action of the corresponding gene. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. [Milstein and Kohler, Nature 256:495-497 (1975); Gulfre and Milstein, Methods in Enzymology: Immunochemical Techniques 73:1-46, Langone and Banatis eds., Academic Press, (1981) which are incorporated by reference]. The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of the protein by treatment of a patient with specific antibodies to the protein.

Specific antibodies, either polyclonal or monoclonal, to the protein can be produced by any suitable method known in the art as discussed above. For example, by recombinant methods, preferably in eukaryotic cells murine or human monoclonal antibodies can be produced by hybridoma technology or, alternatively, the protein, or an immunologically active fragment thereof, or an anti-idiotypic antibody, or fragment thereof can be administered to an animal to elicit the production of antibodies capable of recognizing and binding to the protein. Such antibodies can be from any class of antibodies including, but not limited to IgG, IgA, IgM, IgD, and IgE or in the case of avian species, IgY and from any subclass of antibodies.

Model systems are available that can be adapted for use in high throughput screening for compounds that inhibit the interaction of protein with its ligand, for example by competing with protein for ligand binding. Sarubbi et al., Anal. Biochem. 237:70-75 (1996) describe cell-free, non-isotopic assays for discovering molecules that compete with natural ligands for binding to the active site of IL-1 receptor. Martens, C. et al., Anal. Biochem. 273:20-31 (1999) describe a generic particle-based nonradioactive method in which a labeled ligand binds to its receptor immobilized on a particle; label on the particle decreases in the presence of a molecule that competes with the labeled ligand for receptor binding.

### Antibody Preparation

### (i) Starting Materials and Methods

Immunoglobulins (Ig) and certain variants thereof are known and many have been prepared in recombinant cell culture. For example, see U.S. Pat. No. 4,745,055; EP 256,654; EP 120,694; EP 125,023; EP 255,694; EP 266,663; WO 30 88/03559; Faulkner et al., Nature, 298: 286 (1982); Morrison, J. Immun., 123: 793 (1979); Koehler et al., Proc. Natl. Acad. Sci. USA, 77: 2197 (1980); Raso et al., Cancer Res., 41: 2073 (1981); Morrison et al., Ann. Rev. Immunol., 2: 239 (1984); Morrison, Science, 229: 1202 (1985); and Morrison et al., Proc. Natl. Acad. Sci. USA, 81: 6851 (1984). Reassorted immunoglobulin chains are also known. See, for example, U.S. Pat. No. 4,444,878; WO 88/03565; and EP 68,763 and references cited therein. The immunoglobulin moiety in the chimeras of the present invention may be obtained from IgG-1, IgG-2, IgG-3, or IgG-4 subtypes, IgA, IgE, IgD, or IgM, but preferably from IgG-1 or IgG-3.

### (ii) Polyclonal Antibodies

Polyclonal antibodies to the subject prostate antigens are generally raised in animals by multiple subcutaneous (sc) or intraperitoneal (ip) injections of the antigen and an adjuvant. It may be useful to conjugate the antigen or a fragment containing the target amino acid sequence to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde or succinic anhydride.

Animals are immunized against the polypeptide or fragment, immunogenic conjugates, or derivatives by combining 1 mg or 1 .mu.g of the peptide or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer to the antigen or a fragment thereof. Animals are boosted until the titer plateaus. Preferably, the animal is boosted with the conjugate of the same polypeptide or endothelin or fragment thereof, but conjugated to a different protein and/or through a different cross-linking reagent. Conjugates also can be made in recombinant cell culture as protein fusions. Also, aggregating agents such as alum are suitably used to enhance the immune response.

(iii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, monoclonal antibodies using for practicing this invention may be made using the hybridoma method first described by Kohler and Milstein, Nature, 256: 495 (1975), or may be made by recombinant DNA methods (Cabilly et al., supra).

In the hybridoma method, a mouse or other appropriate host animal, such as a hamster, is immunized as hereinabove described to elicit lymphocytes that produce or are capable of producing antibodies that will specifically bind to the antigen or fragment thereof used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 [Academic Press, 1986]).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Culture medium in which hybridoma cells are growing is assayed for production of monoclonal antibodies directed against the prostate antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson and Pollard, Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxyapatite chromatography, gel electrophoresis, dialysis, or affinity chromatography.

DNA encoding the monoclonal antibodies of the invention is readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5: 256-262 (1993) and Pluckthun, Immunol. Revs., 130: 151-188 (1992). A preferred expression system is the NEOSPLA (expression system of IDEC above-referenced).

The DNA also may be modified, for example, by substituting the coding sequence for human heavy- and light-chain constant domains in place of the homologous murine sequences (Morrison, et al., Proc. Natl. Acad. Sci. USA, 81: 6851 [1984]), or by covalently joining to the immunoglobulin coding sequence all or part of the coding sequence for a non-immunoglobulin polypeptide. In that manner, “chimeric” or “hybrid” antibodies are prepared that have the binding specificity of an anti-prostate antigen monoclonal antibody herein.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for prostate antigen according to the invention and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### (iv) Humanized Antibodies

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature 321, 522-525 [1986]; Riechmann et al., Nature 332, 323-327 [1988]; Verhoeyen et al., Science 239, 1534-1536 [1988]), by substituting rodent CDRs or CDR sequences for the corresponding sequences of a human antibody. Accordingly, such “humanized” antibodies are chimeric antibodies (Cabilly et al., supra), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151: 2296 [1993]; Chothia and Lesk, J. Mol. Biol., 196: 901 [1987]). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89: 4285 [1992]; Presta et al., J. Immunol., 151: 2623 [1993]).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

### (v) Human Antibodies

Human monoclonal antibodies can be made by the hybridoma method. Human myeloma and mouse-human heteromyeloma cell lines for the production of human monoclonal antibodies have been described, for example, by Kozbor, J. Immunol. 133, 3001 (1984); Brodeur, et al., Monoclonal Antibody Production Techniques and Applications, pp. 51-63 (Marcel Dekker, Inc., New York, 1987); and Boerner et al., J. Immunol., 147: 86-95 (1991).

It is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90: 2551 (1993); Jakobovits et al., Nature, 362: 255-258 (1993); Bruggermann et al., Year in Immuno., 7: 33 (1993).

Alternatively, the phage display technology (McCafferty et al., Nature, 348: 552-553 [1990]) can be used to produce human antibodies and antibody fragments in vitro, from immunoglobulin variable (V) domain gene repertoires from non-immunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson and Chiswell, Curr. Op. Struct. Biol., 3: 564-571 (1993). Several sources of V-gene segments can be used for phage display. Clackson et al., Nature, 352: 624-628 (1991) isolated a diverse array of anti-oxazolone antibodies from a small random combinatorial library of V genes derived from the spleens of immunized mice. A repertoire of V genes from non-immunized human donors can be constructed and antibodies to a diverse array of antigens (including self-antigens) can be isolated essentially following the techniques described by Marks et al., J. Mol. Biol., 222: 581-597 (1991), or Griffith et al., EMBO J., 12: 725-734 (1993).

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technology, 10: 779-783 [1992]). In this method, the affinity of “primary” human antibodies obtained by phage display can be improved by sequentially replacing the heavy and light chain V region genes with repertoires of naturally occurring variants (repertoires) of V domain genes obtained from non-immunized donors. This technique allows the production of antibodies and antibody fragments with affinities in the nM range. A strategy for making very large phage antibody repertoires has been described by Waterhouse et al., Nucl. Acids Res., 21: 2265-2266 (1993).

Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e., the epitope governs (imprints) the choice of partner. When the process is repeated in order to replace the remaining rodent V domain, a human antibody is obtained (see PCT WO 93/06213, published Apr. 1, 1993). Unlike traditional humanization of rodent antibodies by CDR grafting, this technique provides completely human antibodies, which have no framework or CDR residues of rodent origin.

### (vi) Bispecific Antibodies

Bispecific antibodies are monoclonal, preferably human or humanized, antibodies that have binding specificities for at least two different antigens. In the present case, one of the binding specificities will be to a prostate antigen according to the invention. Methods for making bispecific antibodies are known in the art.

Traditionally, the recombinant production of bispecific antibodies is based on the co-expression of two immunoglobulin heavy chain-light chain pairs, where the two heavy chains have different specificities (Milstein and Cuello, Nature, 305: 537-539 [1983]). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in WO 93/08829 published May 13, 1993, and in Traunecker et al., EMBO J., 10: 3655-3659 (1991).

According to a different and more preferred approach, antibody-variable domains with the desired binding specificities (antibody-antigencombining sites) are fused to immunoglobulin constant-domain sequences. The fusion preferably is with an immunoglobulin heavy-chain constant domain, comprising at least part of the hinge, CH2, and CH3 regions. It is preferred to have the first heavy-chain constant region (CH1), containing the site necessary for light-chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the production of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation.

For further details of generating bispecific antibodies, see, for example, Suresh et al., Methods in Enzymology, 121: 210 (1986).

(vii) Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/00373; and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

(viii) Domain-Deleted Antibodies

Methods for producing domain-deleted antibodies are disclosed in PCT/US02/02373 and PCT/US02/02374, both filed on Jan. 29, 2002.

Domain deleted antibodies are antibodies wherein a portion of one or more of the constant region domains has been deleted or otherwise altered so as to provide desired biochemical characteristics, e.g., increased tumor localized or reduced serum half-like. The modified antibodies may comprise alterations or modifications to one or more of the three heavy chain constant domains (C_{H}1, C_{H}2, or C_{H}3) and/or to the light chain constant domain (C_{L}). In a preferred embodiment the domain deleted antibody will have the entire C_{H}2 domain removed and/or an amino acid spacer substituted for a deleted domain to provide flexibility and freedom of movement to the variable region.

As discussed supra, because humanized and human antibodies are far less immunogenic in humans than other species monoclonal antibodies, e.g., murine antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

### Small Molecule Antagonists

The availability of isolated protein also allows for the identification of small molecules and low molecular weight compounds that inhibit the binding of protein to binding partners, through routine application of high-throughput screening methods (HTS). HTS methods generally refer to technologies that permit the rapid assaying of lead compounds for therapeutic potential. HTS techniques employ robotic handling of test materials, detection of positive signals, and interpretation of data. Lead compounds may be identified via the incorporation of radioactivity or through optical assays that rely on absorbance, fluorescence or luminescence as read-outs. [Gonzalez, J. E. et al., Curr. Opin. Biotech. 9:624-631 (1998)].

Model systems are available that can be adapted for use in high throughput screening for compounds that inhibit the interaction of protein A or protein B with its ligand, for example by competing with protein A or protein B for ligand binding. Sarubbi et al., Anal. Biochem. 237:70-75 (1996) describe cell-free, non-isotopic assays for discovering molecules that compete with natural ligands for binding to the active site of IL-1 receptor. Martens, C. et al., Anal. Biochem. 273:20-31 (1999) describe a generic particle-based nonradioactive method in which a labeled ligand binds to its receptor immobilized on a particle; label on the particle decreases in the presence of a molecule that competes with the labeled ligand for receptor binding.

### Gene Therapy

The polynucleotides and polypeptides of the present invention may be utilized in gene delivery vehicles. The gene delivery vehicle may be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic according to the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs may be readily prepared (see PCT publications WO 95/3 0763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (MV) vectors. Representative examples include the MV vectors disclosed by Srivastava in WO 93/09239, Samulski et al., J. Vir. 63: 3822-3828 (1989); Mendelson et al., Virol. 166: 154-165 (1988); and Flotte et al., P.N.A.S. 90: 10613-10617 (1993).

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Bisler et al., P.N.A.S. 90: 11498-11502 (1993); Guzman et al., Circulation 88: 2838-2848 (1993); Guzman et al., Cir. Res. 73: 1202-1207 (1993); Zabner et al., Cell 75: 207-216 (1993); Li et al., Hum. Gene Ther. 4: 403-409 (1993); Cailaud et al., Eur. J. Neurosci. 5: 1287-1291 (1993); Vincent et al., Nat. Genet. 5: 130-134 (1993); Jaffe et al., Nat. Genet. 1: 372-378 (1992); and Levrero et al., Gene 101: 195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to kill adenovirus as described in Curiel, Hum. Gene Ther. 3: 147-154 (1992) may be employed.

Other gene delivery vehicles and methods may be employed; including polycationic condensed DNA linked or unlinked to kill adenovirus alone, for example Curiel, Hum. Gene Ther. 3: 147-154 (1992); ligand-linked DNA, for example see Wu, J. Biol. Chem. 264: 16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994), and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13 796, WO 94/23697, and WO 91/14445, and EP No. 0 524 968.

Further non-viral delivery suitable for use includes mechanical delivery systems such as the approach described in Woffendin et al., Proc. Natl. Acad. Sci. USA 91(24): 11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

### Interfering RNA

The invention further embraces the use of interfering RNA (RNAi) to disrupt the expression of prostate cancer associated genes according to the invention. This can be accomplished by various means.

For example, in one method all or a portion of the targeted gene can be incorporated into a vector and used to target desired cells, e.g., prostate cancer cells. By the phenomena of “co-suppression” first observed in plants, the expression of the endogenous gene is thereby inhibited in the target cell. This phenomena has also been observed in animals, e.g., C. elegans and Drosophila. The interfering RNA interferes with expression of the unlinked endogenous gene by molecular phenomena yet to be fully understood. It is hypothesized that the interfering RNA results in the synthesis of an RNA intermediate which is synthesized at the transgenic locus that disrupts expression of the endogenous gene.

Alternatively, interfering RNA approaches include the use of double or triple helical structures that are homologus to the targeted gene, in this case a prostate cancer associated gene according to the invention. Delivery of the double or stranded nucleic acid structure similarly results in the inhibition of the expression of the endogenous gene, similar to antisense oligonucleotides. A review of these RNA interference methods is disclosed in U.S. Pat. No. 6,506,559, incorporated by reference in its entirety herein.

While the invention has been described supra, including preferred embodiments, the following examples are provided to further illustrate the invention.

### EXAMPLE 1

### Identification of DWAN Nucleic Acid Sequence

A prostate specific gene referred to as DWAN was identified by hybridization analysis with the GeneLogic database using the fragment 147504 as an Enorthern probe (which probe contains a portion of the DWAN gene). The data obtained from this hybridization analysis are summarized below in Table 1 wherein the “present score” represents the number of patient samples that gave a hybridization score considered significant by the GeneLogic database and the “median score” refers to the median hybridization score for all samples of the particular tissue type.

TABLE 1

| Prostate, | Prostate, | Prostate, | Prostate, |
|---|---|---|---|
| Malignant: | Malignant: | Normal: | Normal: |
| Present Score | Median | Present Score | Median |
| (10/13) | 526.08 | (7/15) | 89.71 |

| Colon, | Colon, | Esophagus, | Esophagus, |
|---|---|---|---|
| Normal: | Normal: | Normal: | Normal: |
| Present Score | Median | Present Score | Median |
| (3/28) | 22.57 | (3/18) | 31.44 |

| Kidney, | Kidney, | Liver, | Liver, |
|---|---|---|---|
| Normal: | Normal: | Normal: | Normal: |
| Present Score | Median | Present Score | Median |
| (2/25) | 19.55 | (0/21) | 0 |

| Lung, | Lung, | Lymph Node, | Lymph Node, |
|---|---|---|---|
| Normal: | Normal: | Normal: | Normal: |
| Present Score | Median | Present Score | Median |
| (2/32) | 16.55 | (2/10) | 85.94 |

| Pancreas, | Pancreas, | Rectum, | Rectum, |
|---|---|---|---|
| Normal: | Normal: | Normal: | Normal: |
| Present Score | Median | Present Score | Median |
| (1/17) | 9.23 | (2/22) | 27.58 |

| Stomach, Normal: | Stomach, Normal: |
|---|---|
| Present Score | Median |
| (12/25) | 78.63 |

Upon analysis of the above results, it can be seen that DWAN is substantially upregulated in prostate tumor tissues relative to normal tissues. Based on these results, the inventors obtained and sequenced EST IMAGE 2251589 that contains the fragment 147504 which comprises the DWAN coding sequence. The 221589 sequence is set forth below:

(SEQ ID NO: 1)

agttactcat ttttcaggcc tgagttgatc gttaatcatc
ttaattatgt tcattctgaa gccaacagga gaaccaagac
caaaacttta ttgtctctgc tttcatttct tgatgaaacc
tctggactaa gcacacatct tccttgttta tctctctcaa
aggagtgtgg agtgcttcat ctggacatcc acgggaagaa
ggaagacatg agggaatgct ggaagaggag acaggcccca
gatttgggca ggaagtaaac agttttcagg ctgaggccaa
tctgagcagg aacattccaa tatttcttca gctacgttgt
cccagcactt cactggttaa ccttttatgt ccaccatttg
tggatttcac agctacttgt caatggtgaa tattgatcat
catcattatc tactgagctg ctaccatatc ccagctactc
cttgcatgtt gttcattatt ttctcaacac tcagcatatt
tgcaatatgt tatgtaatat cacagacaag gaaactgaac
gcagaaatgt tttatttctt gccaaacatc acatgaggat
gaacaatgaa accgatttga aaccaggatt gtctgattcc
aacatctctg ggtccttttt cactctgata tgctgcaatt
aaaaagccat ttctaagact gtaaaaaaaa aaaaaaaaaa
cacctgcggc cgcaagctta ttcccttagg aggtat

As shown above, nucleotides 1-212 in SEQ ID NO: 1 corresponds to the first exon of DWAN and nucleotides 212-663 correspond to the second exon. The coding sequence is in bold, and comprises to bases 347-556.

### Identification of DWAN Coding Sequence

The DWAN coding putative sequence is predicted to encode a protein of 69 amino acids followed by a stop codon. The predicted amino acid sequence for DWAN is set forth below:

(SEQ ID NO: 2)

msticgfhsy lsmvnidhhh yllscyhipa tpcmlfiifs
tlsifaicyv isqtrklnae mfyflpnit

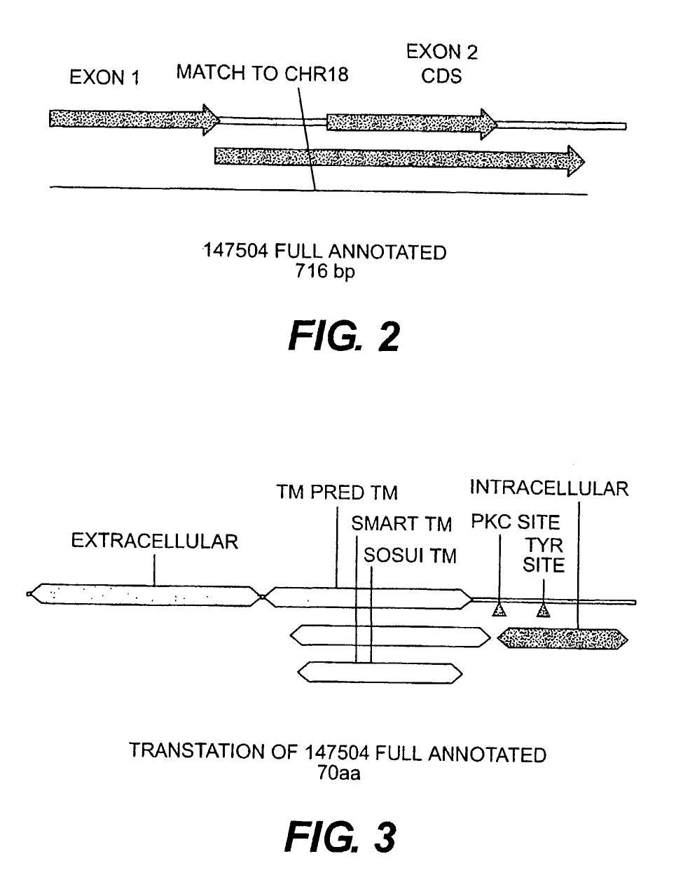

Further analysis of this sequence using three different programs commonly used to identify transmembrane domains (TM Pred, SOSUI, and SMART) reveals that the DWAN protein comprises a putative transmembrane domain in the DWAN coding sequence. Also identified were putative PKC and Tyrosine phosphorylation sites using the Motif Scan web site. The predicted structure of the DWAN protein is contained in FIGS. 2 and 3.

### Expression of DWAN in Other Normal Tissues

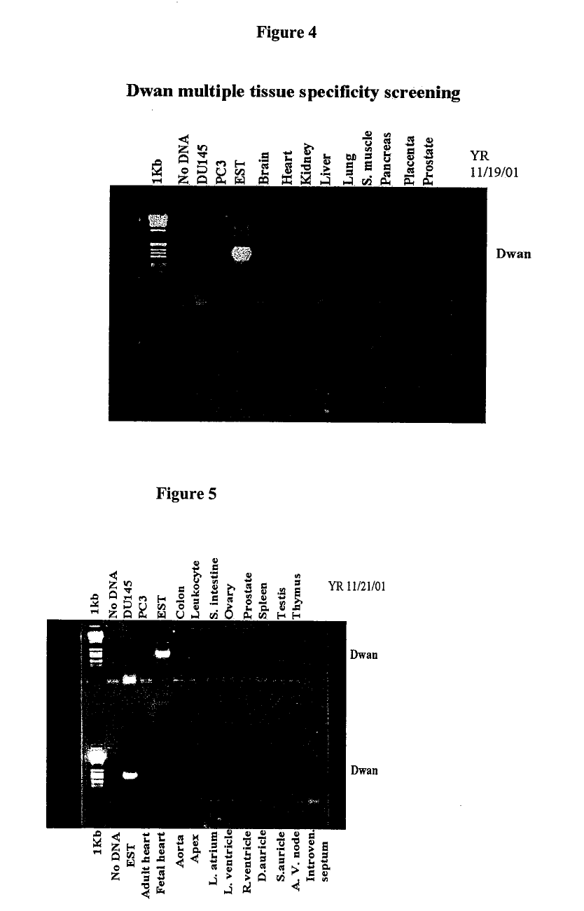

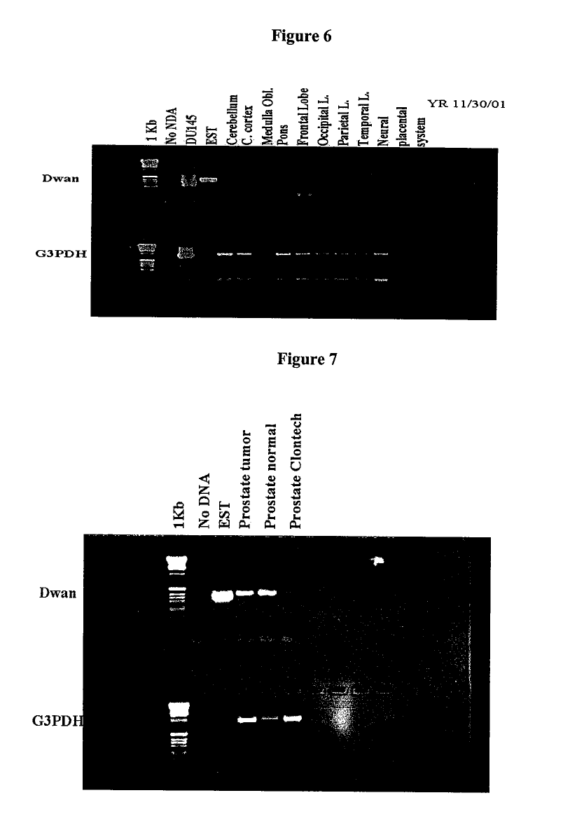

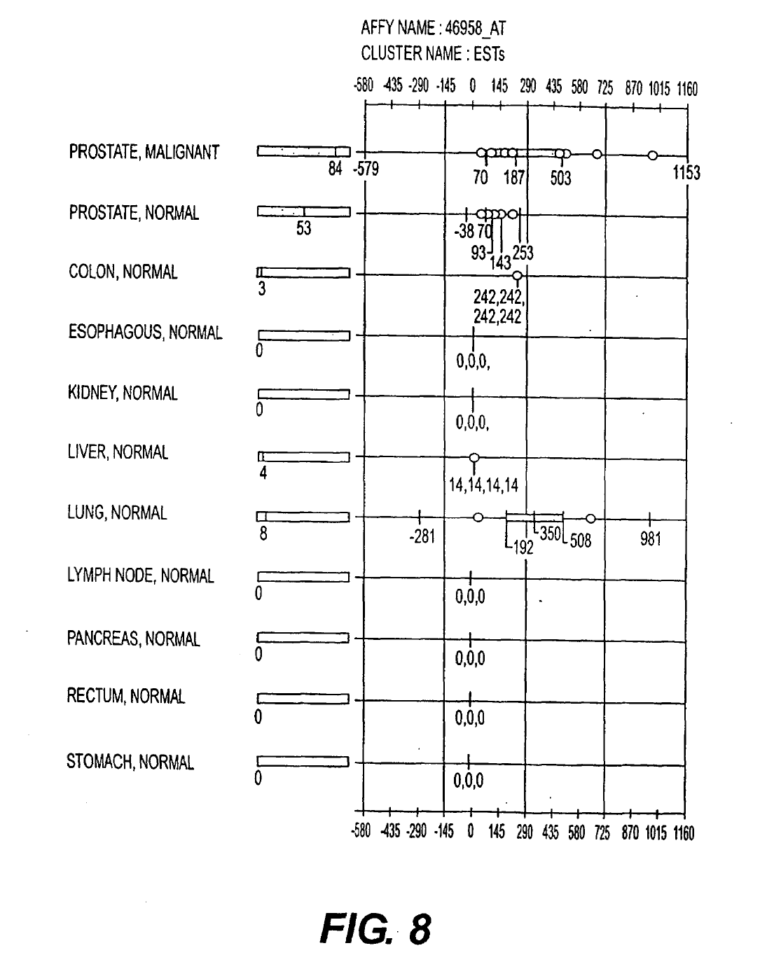

The GeneLogic database lacks DNA expression data corresponding to a number of important tissues including brain and heart. Accordingly, to establish that DWAN is not significantly expressed in our other normal tissues, the inventors designed primers that spanned the intron in DWAN and investigated the presence or absence of DWAN message in cDNAs from multiple tissue panels obtained from Clontech. These results are contained in FIGS. 4-6 and show that the DWAN message is only significantly expressed in prostate.

### Expression of DWAN IN Normal Versus Cancerous Prostate Tissues

Another round of PCR hybridization experiments were conducted using the sub-primer to detect DWAN expression in normal versus cancerous prostate tissues. These results are in FIG. 7. In FIG. 7, EST refers to IMAGE clone 2251589 that encodes the full length DWAN and G3PDH was used as a standard to ensure that there are equal amounts of cDNA in each sample. Du145 and PC-3 are prostate cancer cell lines. Suprisingly, these cell lines do not appear to express DWAN. Although the Enorthern suggest that the tumor should have more DWAN message than the paired normal, in this particular patient, the results suggest that it does not. This could just be an aberrational; result or it may be that the “normal” prostate tissue may be malignant.

### EXAMPLE 2

### Identification of Kv3.2 Gene

Using similar methods it was observed that Kv3.2 is substantially and specifically upregulated in malignant prostate tissues in relation to the same normal tissues identified in Example 1. Set forth below in Table 2 are the results of an Enorthern using the GeneLogic database and the fragment 117293 as a probe. (This probe contains a portion of the Kv3.2 gene). The present score again represents the number of patient samples that gave a hybridization score considered significant by the GeneLogic database, and the median is the median hybridization score for that all of the tissue type.

TABLE 2

| Prostate, | Prostate, | Prostate, | Prostate, |
|---|---|---|---|
| Malignant: | Malignant: | Normal: | Normal: |
| Present Score | Median | Present Score | Median |
| (11/13) | 187.43 | (8/15) | 93.12 |

| Colon, | Colon, | Esophagus, | Esophagus, |
|---|---|---|---|
| Normal: | Normal: | Normal: | Normal: |
| Present Score | Median | Present Score | Median |
| (1/28) | 242.22 | (0/18) | 0 |

| Kidney, | Kidney, | Liver, | Liver, |
|---|---|---|---|
| Normal: | Normal: | Normal: | Normal: |
| Present Score | Median | Present Score | Median |
| (0/25) | 0 | (1/21) | 14.83 |

| Lung, | Lung, | Lymph Node, | Lymph Node, |
|---|---|---|---|
| Normal: | Normal: | Normal: | Normal: |
| Present Score | Median | Present Score | Median |
| (2/32) | 350.13 | (0/10) | 0 |

| Pancreas, | Pancreas, | Rectum, | Rectum, |
|---|---|---|---|
| Normal: | Normal: | Normal: | Normal: |
| Present Score | Median | Present Score | Median |
| (0/17) | 0 | (0/22) | 0 |

| Stomach, Normal: | Stomach, Normal: |
|---|---|
| Present Score | Median |
| (0/25) | 0 |

After obtaining these results the inventor queried the public database and determined that this sequence likely is an extension of the 3′UTR of the potassium channel Kv3.2a.

As reported in a public database of human gene sequences, the gene comprises at least two alternatively spliced variants, Kv3.2a and Kv3.2b. Both have the same extracellular domains and differ only by the C-terminal 19 amino acids. According to the literature, these sequences play a role in the trafficking of these proteins to different parts of the polarized cells. The sequence of both Kv3.2 gene variants is in the public domain and have the following accessing number:

| | |
|---|---|
| Kv3.2a DNA | AF268897 |
| Kv3.2a protein | AF268897_1 |
| Kv3.2b DNA | AF268896 |
| Kv3.2b protein | AF268896_1 |

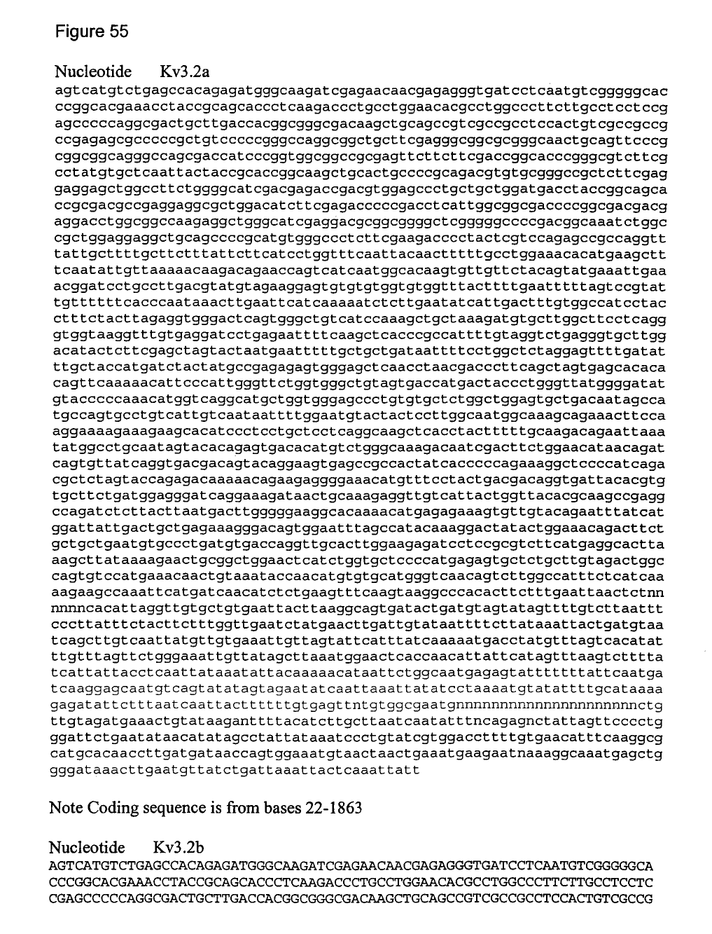

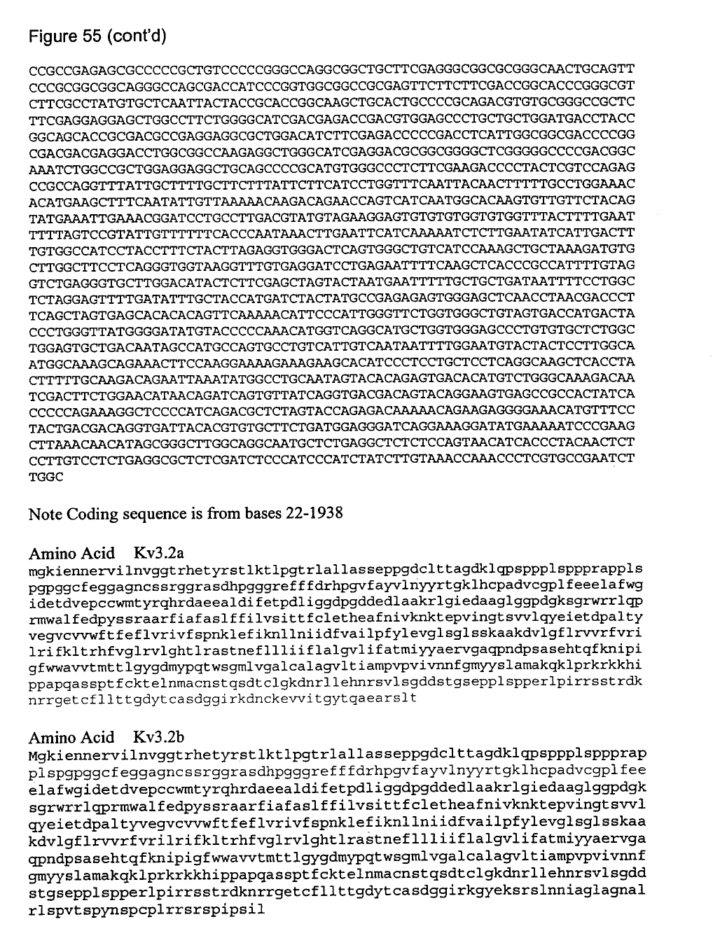

Additionally, the amino acid and nucleic acid sequences for Kv3.2a and Kv3.2b are contained in sequence FIG. 55.

### Expression of Kv3.2 in Other Normal Tissues

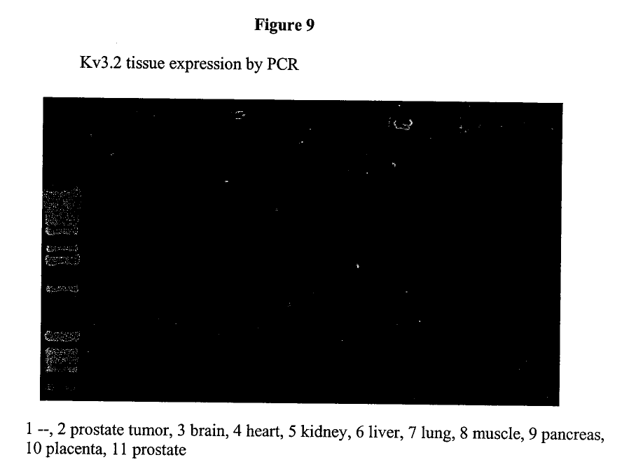

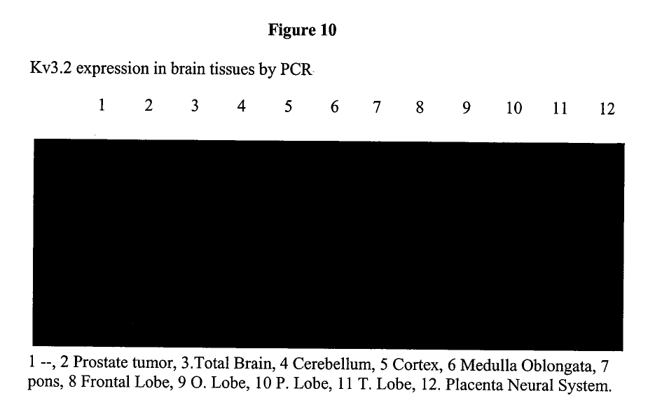

Since the GeneLogic database lacks a number of important tissues including brain and heart, the inventor again designed intron-spanning primers in order to detect expression in cDNAs from multiple tissue panels obtained from Clontech. These results are contained in FIGS. 9 and 10 and show that the Kv3.2 message is only significantly expressed in brain (as predicted in the literature) and the malignant prostate. Based thereon, Kv3.2 should be an appropriate target for treatment of prostate cancer as it is not significantly expressed in most normal tissues.

PCR from Multiple Tissue Panels

As described above, we identified fragment 117293 on the Hu_{—}95 Affymetrix chip as hybridizing specifically to samples from normal and malignant prostate. This fragment corresponded to the 3′ untranslated region of Shaker-Shaw related potassium channel Kv3.2a (KCNC2, transcript variant 1). Using reverse transcriptase PCR (RT-PCR), we confirmed that Kv3.2 RNA was present in two different surgical resections of malignant prostate but not in other normal human tissues with the exception of the brain.

We further obtained additional data contained therein expanding the number of tissues examined by RT-PCR, cloning Kv3.2a, expression and detection of Kv3.2a in Chinese Hamster Ovary cells (CHO) and African Green Monkey Kidney cells (COS-7), and generation of murine monoclonal antibodies against the extracellular domain of this protein.

To confirm that Kv3.2 mRNA was present in malignant prostate and absent in most other tissues, we assayed Kv3.2 expression using cDNA from two primary prostate tumors and from commercially available cDNA panels of normal tissue. Malignant and adjacent normal prostate samples were obtained from Analytical Pathology Medical Group and frozen within thirty minutes of surgery. RNA was extracted from the samples using RNeasy Maxi Kit (Qiagen) according to the manufacture's instructions and reverse transcribed into cDNA using Superscript II Kit (Invitrogen). MTC I, MTC II and Human Heart cDNA panels were obtained from Clontech and Human Brain cDNA panels were obtained from BioChain. The Kv3.2 message was amplified using the following primers:

(SEQ ID NO: 3)

5′ Primer: gaagctttcaatattgttaaaaacaagac

(SEQ ID NO: 4)

3′ Primer: atgtgtcactctgtgtactattgcaggcc
using

standard PCR conditions.

These primers span an intron to prevent the amplification of genomic DNA in the event of contamination with genomic DNA.

These data demonstrate that Kv3.2 message is expressed in the malignant prostate and in the cortex, the pons and the frontal lobe of the brain. Although expression in the brain has been documented (Rudy et al. Annals of the New York Academy of Sciences, 868: 304-343, 1999, Chow et al. J Neurosci 19: 9332-9345, 1999, Rudy et al. Proc Natl Acad Sci, USA, 89: 4603-4607, 1992, Weiser et al. J Neurosci, 14: 949-972, 1994 Moreno et al. J Neurosci 15: 5486-5501, 1995), this is the first report of Kv3.2 expression in the malignant prostate.

### Expression and Localization of Kv3.2

Full length Kv3.2a was assembled from commercially available ESTs and by PCR products generated using cDNA from the prostate tumors N and O as templates. The full length Kv3.2 was ligated into an expression vector under the control of a cytomegalovirus promoter and a bovine growth hormone poly adenylation signal. This vector also contains the neomycin phosphotransferase gene that confers resistance to neomycin (G418) that has been engineered to contain an intron. This NEOSPLA vector has been previously described (U.S. Pat. No. 6,159,730). This vector also contains a cassette encoding the extracellular domain of human B7.1 (CD80, amino acids 1-243) fused to the human IgG1 constant domain (amino acids 226-478 EU in Kabot, with the following mutations to prevent dimerization, 230 (Cys to Ala), 239 (Cys to Ser) and 242 (Cys to Ser). The vector was prepared using Qiagen Endofree Plasmid Maxi Kit and dissolved in 10 mM Tris-HCl, 1 mM EDTA pH 8 buffer. Plasmid DNA was linearized with PacI restriction endonuclease prior to transfection into CHO cell line DG44.

DG44 CHO cells were maintained in CHO-S-SFMII media (Gibco) supplemented with HT supplement. The DG44 cell line has been adapted for suspension growth in culture (Urlaub et. al., Som. Cell. Mol. Gen., 12:555-566, 1985). Briefly, DG44 cells were washed, counted and resuspended in ice cold PBS buffer. 4×10⁶ cells were mixed with 0.5 μg of linear plasmid DNA and pulsed at 350 volts, 600 μF using Gene Pulser II (Bio-Rad). Cells were seeded into 96-well microtiter tissue culture plates at approximately 4×10⁴ cells/well. After two days, cells were selected in media containing G418. The resistant clones appeared after 3 weeks. 61 clones were assayed for B7Ig expression in ELISA.

In short, Immunolon II 96-well microtiter plates were coated overnight with 200 ng per well unlabeled goat anti-human IgG antibody (Southern Biotechnology Associates, Inc.) in 50 mM carbonate buffer pH 9.4. Plates were blocked for 2 hours at room temperature with Phosphate buffered saline (PBS), 0.5% Nonfat Dry Milk, 0.01% Thimerosal (Blocking buffer/sample diluent). Culture supernatants containing test samples were diluted in Blocking buffer/sample diluent and incubated for 1 h at 37° C. The plates were washed 5 times and incubated with goat anti-human IgG-HRP antibody (Southern Biotechnology Associates, Inc.) in Blocking buffer/sample diluent for 1 h at 37° C. Plates were once again washed and developed with HRPO substrate derived from 1:1 mixture of TMB Peroxidase Substrate:Peroxidase Solution B (Kirdgaard and Perry Labs). Reactions were terminated with the addition of 2M H₂SO₄ and absorbance measured on a microtiter plate reader (Molecular Devices) at 450 nm. Stable clone 1A5 produced the most soluble B7Ig and was selected for further characterization.

The presence of KV3.2a mRNA in clone 1A5 was confirmed by RT-PCR. Total RNA was isolated from clone 1A5 using RNeasy Mini Kit (Qiagen) and cDNA prepared according to manufacturer's directions using the cDNA Cycle Kit (Invitrogen). The PCR reaction was performed using a standard protocol with the following primers:

(SEQ ID NO: 5)

5′ Primer XC-23 GCGGCGAAGCTTTCAATATTGTTAAAAACAAGAC

(SEQ ID NO: 6)

3′ Primer SC-24 ATGTGTCACTCTGTGTACTATTGCAGGCC

The appearance of the expected 810 bp KV3.2a fragment by agarose gel electrophoresis demonstrated the expression of KV3.2a mRNA in the 1A5 cell line.

Analysis of Kv3.2a expression and cell surface localization in the 1A5 cell line was performed by immunofluorescence microscopy. Cells grown on coverslips were washed with PBS, and fixed by exposure to 4% paraformaldehyde for 15 min at room temperature. The fixed cells were permeabilized by incubation with 0.5% Triton X-100, 1% goat serum in PBS for 10 min. Subsequently, the cells were incubated for 4 hrs at room temperature with rabbit anti-Kv3.2 primary antibody (Chemicon) at a dilution of 1:250 in PBS supplemented with 3% goat serum (blocking buffer). After washing with PBS, the cells were incubated for 45 min at room temperature with Alexa488-conjugated goat-anti-rabbit IgG secondary antibody (Molecular Probes) at 1:2,000 and 1 μg/ml DAPI stain (Sigma) in blocking buffer. The cells were washed with PBS, mounted on glass slides using ProLong Antifade Kit (Molecular Probes) and examined using an Olympus IX 70 microscope (40× objective) with a Delta Vision deconvolution system. Approximately 30% of the 1A5 cells expressed detectable Kv3.2 protein; Kv3.2 demonstrated surface localization only in 10% of these stability transfected cells.

### Generation of Antibodies Against the Extracellular Domain of Kv3.2

Female Balb/c mice were immunized twice with DNA encoding the Kv3.2a protein under the control a CMV promoter. The mice were boosted twice with COS-7 cells transiently transfected with a plasmid encoding Kv3.2a. COS-7 cells were seeded at 800,000 cells per 100 mm dish the night before transfection and transfected with 3.5 □g Kv3.2a expressing plasmid and 20 μl Lipofectamine (Invitrogen) diluted in OptiMEM (Invitrogen) as per manufacture's instructions. Forty-eight hours after transfection, these cells were harvested. The mice were boosted twice with these cells. The mice were bleed and titers of anti-Kv3.2 antibodies were determined by binding to the Kv3.2 expressing CHO cell 1A5 relative to wild type CHO cells (WT-CHO). Spleens from mice exhibiting the highest titer were removed and fused to mouse myeloma Sp2/0 cells following standard immunological techniques (Kohler, G. and Milstein, C. 1975. Nature 256, p 495.) The resulting hybridoma cells were plated in 96-well flat bottom plates (Corning) and cultured in Iscove's Modified Dulbecco's Medium (IMDM, Irvine Scientific) containing 10% FBS, 4 mM L-Glutamine (Gibco), 1× non-essential amino acids (Sigma), 1 mM sodium pyruvate (Sigma), 5 ug/ml gentamicin (Gibco) supplemented with HAT (5×10⁻³ M hypoxanthine, 2×10⁻⁵M aminopterin, 8×10⁻³M thymidine, Sigma) and 1% Origen hybridoma cloning factor (Igen International.) After 5 days in culture, the medium was replaced with IMDM containing the above supplements plus HT (Gibco) in place of HAT. Supernatants were screened by whole cell sandwich ELISA comparing Kv3.2 expressing CHO 1A5 cells to WT-CHO.

Briefly, Immulon-II plates (Thermo Labsystems) were coated with Poly L Lysine. 1A5 or WT-CHO at 10⁵ cells per well were bound to the Poly L lysine and fixed with paraformaldehyde. Fifty □l of hybridoma supernatant was added to the fixed cells and incubated for an hour to allow binding. The plates were washed and binding was detected with goat anti-mouse IgG-HRP antibody (Southern Biotechnology Associates, Inc.) and developed with HRPO substrate derived from 1:1 mixture of TMB Peroxidase Substrate:Peroxidase Solution B (Kirdgaard and Perry Labs). Reactions were terminated with the addition of 2M H₂SO₄ and absorbance measured on a microtiter plate reader (Molecular Devices) at 450 nm. The twenty-one clones demonstrating binding to the 1A5 cell line with minimal binding to WT-CHO were selected for further study.

TABLE 3
ELISA results from twenty-one Kv3.2 reactive clones.

| Clone | Kv3.2% | WT % |
|---|---|---|
| 1B8 | 0.82 | 0.02 |
| 4C12 | 0.66 | 0.04 |
| 5C9 | 0.94 | 0.03 |
| 5E1 | 0.66 | 0.01 |
| 9B9 | 0.85 | 0.02 |
| 16E6 | 0.81 | 0.01 |
| 17C1 | 0.46 | 0.01 |
| 18H10 | 0.33 | 0.00 |
| 21D7 | 0.96 | 0.00 |
| 21E10 | 0.45 | 0.00 |
| 21G6 | 0.40 | 0.00 |
| 23D8 | 0.39 | 0.00 |
| 24E6 | 0.75 | 0.01 |
| 34B5 | 0.55 | 0.01 |
| 37E12 | 0.54 | 0.03 |
| 37F10 | 0.70 | 0.05 |
| 38D12 | 0.52 | 0.01 |
| 42B9 | 0.74 | 0.03 |
| 442G4 | 0.51 | 0.00 |
| 43D3 | 0.83 | 0.04 |

Optical Densities were recorded and are reported as the percentage of positive control (1:100 dilution of positive bleed).

To determine if these antibodies are reacting with an epitope expressed on the extracellular surface of Kv3.2, these antibodies were tested by flow cytometry analysis of binding to unpermeabilized cells. In short, 2*10⁵ 1A5 or WT-CHO cells (at 4*10⁶ cells/ml) were incubated in with 50 □l of hybridoma supernatant and incubated for an hour to allow binding. The cells were washed and the antibody was detected with a 1:2000 dilution of goat anti-Mouse IgG (H+L)-RPE (Southern Biotechnology). The cells were washed and stained with aminoactinomycin D (Molecular Probes) at a 1:1000 dilution. The cells were analyzed on a FACSCalibur (Becton Dickinson).

TABLE 4
Percentage shift of into gate observed with binding of hybridoma
supernatants.

| | Kv3.2a- | WT- |
|---|---|---|
| Clone | CHO | CHO |
| Neg | 0.01 | 0.03 |
| control | | |
| 1B8 | 3.28 | 0.01 |
| 4C12 | 0.32 | 0.01 |
| 5C9 | 8.79 | 0.01 |
| 5E1 | 7.44 | 0.03 |
| 9B9 | 6.29 | 0.02 |
| 16E6 | 5.69 | 0.05 |
| 17C1 | 7.47 | 0 |
| 18H10 | 0.44 | 0 |
| 21D7 | 0.12 | 0.01 |
| 21E10 | 7.35 | 0 |
| 21G6 | 4.71 | 0.02 |
| 23D8 | 3.48 | 0.01 |
| 24E6 | 4.25 | 0.15 |
| 25C6 | 3.26 | 0.01 |
| 34B5 | 3.46 | 0.24 |
| 37E12 | 12.65 | 0.3 |
| 37F10 | 0.33 | 0.34 |
| 38D12 | 0.33 | 0.18 |
| 42B9 | 2.28 | 0.1 |
| 42G4 | 3.64 | 0.1 |
| 43D3 | 4.48 | 0.38 |

Note approximately 10% of 1A5 CHO cells express Kv3.2a on the surface of the cell.

Sixteen clones (1B8, 5C9, 5E1, 9B9, 16E6, 17C1, 21E10, 21G6, 23D8, 24E6, 25C6, 34B5, 37E12, 42B9, 42G4, 43D3) were identified that bound to unpermeabilized 1A5 cells and not to WT-CHO cells; 37E12 and 5C9 demonstrating the best binding.

Based on these results, we have demonstrated that Kv3.2a message is expressed in the malignant prostate and in the brain. Moreover, we demonstrate that the Kv3.2 is expressed on the surface of transfected cells and that we can raise antibodies against the extracellular portion of this protein. Antibodies against the extracellular of the protein can be used for the treatment of prostate cancer.

### EXAMPLE 3

### Identification of MASP (159171)

A third prostate specification gene was identified using the same methods using the GeneLogic database and the fragment 159171 to detect gene expression. This probes a portion of the MASP gene and was used therefor to detect MASP expression in a variety of tissues including malignant prostate. The results of the Enorthern experiments are summarized in Table 3 below. Again, the score again represents the number of patient samples that gave a hybridization score considered significant by the GeneLogic database, and the median refer to the median hybridization score for all of the particular tissue type.

TABLE 5

| Prostate, | Prostate, | Prostate, | Prostate, |
|---|---|---|---|
| Malignant: | Malignant: | Normal: | Normal: |
| Present Score | Median | Present Score | Median |
| (12/13) | 133.94 | (9/15) | 112.34 |

| Colon, | Colon, | Esophagus, | Esophagus, |
|---|---|---|---|
| Normal: | Normal: | Normal: | Normal: |
| Present Score | Median | Present Score | Median |
| (1/28) | 7.81 | (1/18) | 56.51 |

| Kidney, | Kidney, | Liver, | Liver, |
|---|---|---|---|
| Normal: | Normal: | Normal: | Normal: |
| Present Score | Median | Present Score | Median |
| (6/25) | 33.07 | (0/21) | 0 |

| Lung, | Lung, | Lymph Node, | Lymph Node, |
|---|---|---|---|
| Normal: | Normal: | Normal: | Normal: |
| Present Score | Median | Present Score | Median |
| (3/32) | 23.14 | (2/10) | 29.81 |

| Pancreas, | Pancreas, | Rectum, | Rectum, |
|---|---|---|---|
| Normal: | Normal: | Normal: | Normal: |
| Present Score | Median | Present Score | Median |
| (1/17) | 10.56 | (1/22) | 33.27 |

| Stomach, Normal: | Stomach, Normal: |
|---|---|
| Present Score | Median |
| (2/25) | 37.73 |

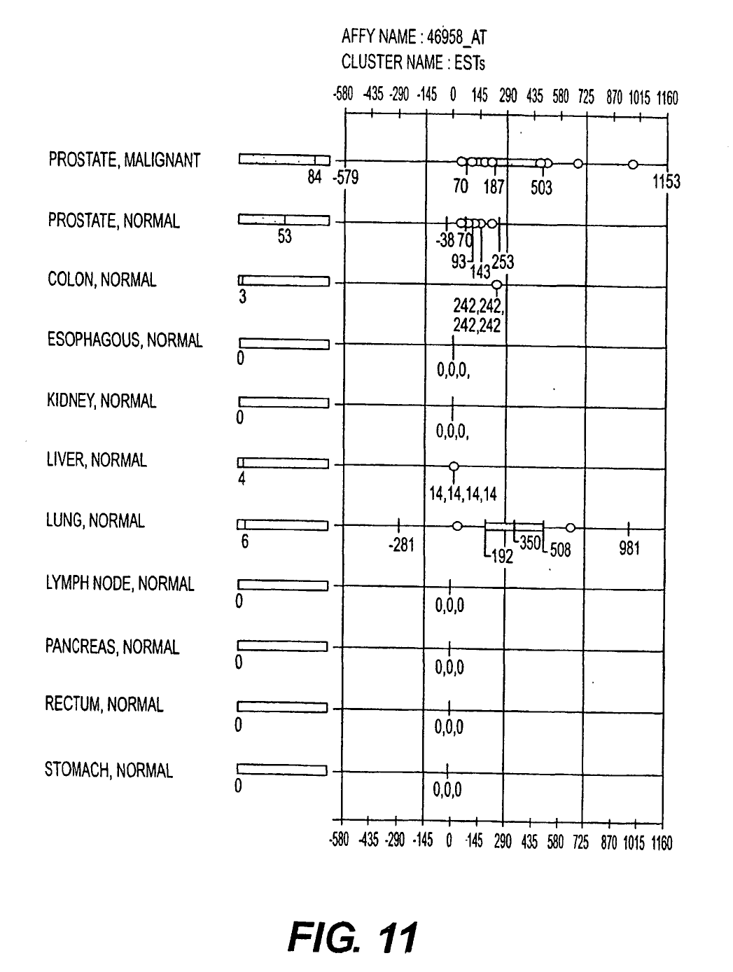

Based on these Enorthern results, it appears that the MASP gene is significantly upregulated in prostate cancer tissues. The inventor thereupon obtained and sequenced EST IMAGE 2490796 (which contains the fragment 147504). The sequence of MASP is set forth below as SEQ ID NO: 7. These results are also depicted visually in FIG. 11.

(SEQ ID NO: 7)

Ggaaagcgaagagcgcccaatacgcaaaccgcntctccccgcgngtgggc
gattcattatgcagctggcacgacagggtttcccgactggaaagcngggc
agtgagnggcaacgcaattaatgtgagttagctcactcattaggcccccc
caggctttacactttatgcttcccggctcgtatgttgtgtggaattgtga
gcggataacaatttcacacaggaaacagctatgacatgattacgaattta
atacgactcactatagggaatttggccctcgaggccaagaattcggcacg
aggtgctttcatggtgaccaaactaatgagcagcacccttctgcagaggt
aaactttgccttgctgagaaaccaattgttggcgtgtttatttcatttat
gactttgagctttatttctaacatggcccaaagtaatcctcttttcttga
acacatggtagaatgccctaggtgaatccctccagtcttccagtaccatc
cttgactcctctctctgatgacacatgaactttatgcttttgcacacttc
aggcaacaccaaaagaaaggaaaagaacagcttagcttcttaatgtgtgt
aagaaaccacagtgaaaaaaaatcaggtgtgttgttgaggctgctaaaag
ctttccttttttttctgtgccagttctcgctgcctcattggttgagatgg
gatgtcttttttgatgtcctctttagagagtgttatcctcacctttttgc
atagtcctaccaaaagacacctcacatgcaaagtgtaacagaaaattaca
gtcatgactttagttttaaaaacaggacgtatattcatgaagaatgtttg
ctgttttcccagtgggttaatcatatgaatataaaacagactaaaaatat
caagttgtttttgcatttatttattgtagaaataaaatggattgctacct
ctgagcttctgaaaaaaaaaaaaaaaaaaa

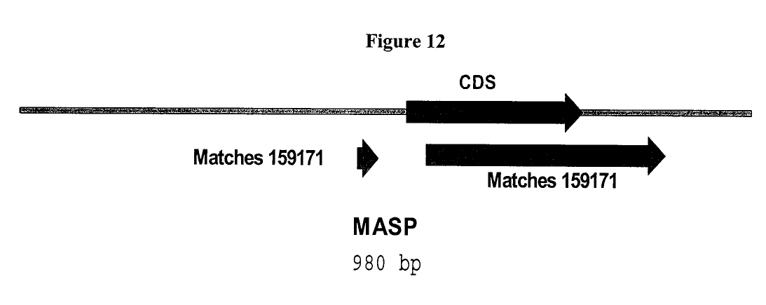

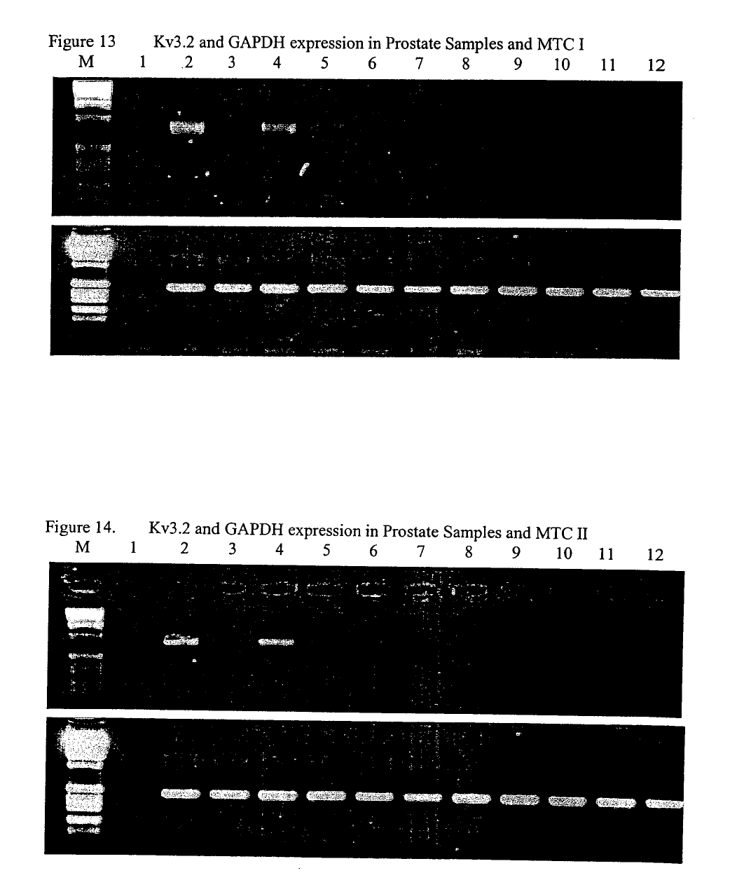

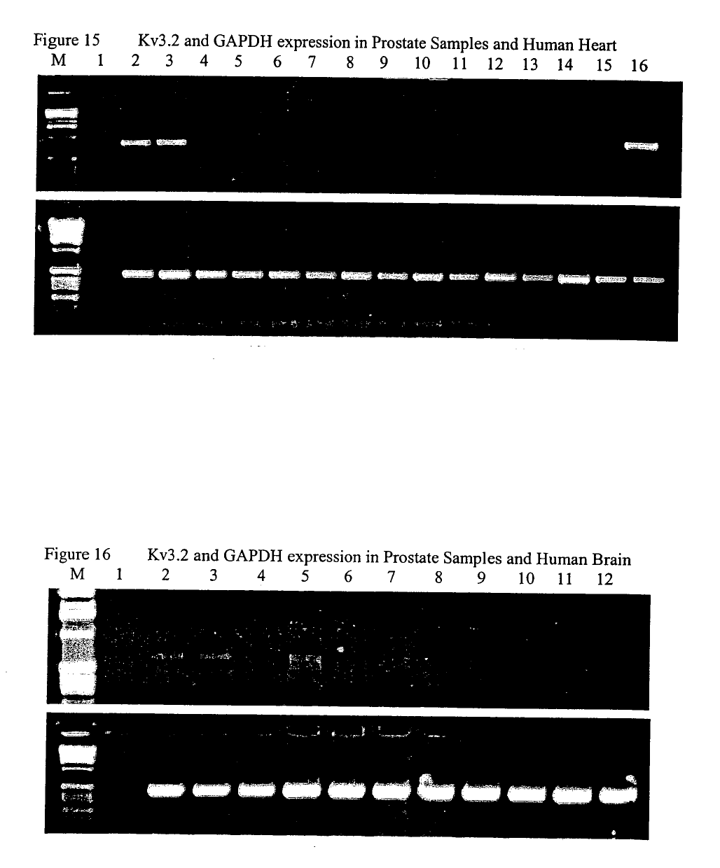

As depicted schematically in FIG. 12, the MASP gene comprises a single exon. The coding sequence of the MASP antigen is contained in SEQ ID NO: 7 and is set forth in bold, and corresponds to nucleotides 518-754.

### EXAMPLE 4

### AF116574, AK024064/Astrotactin

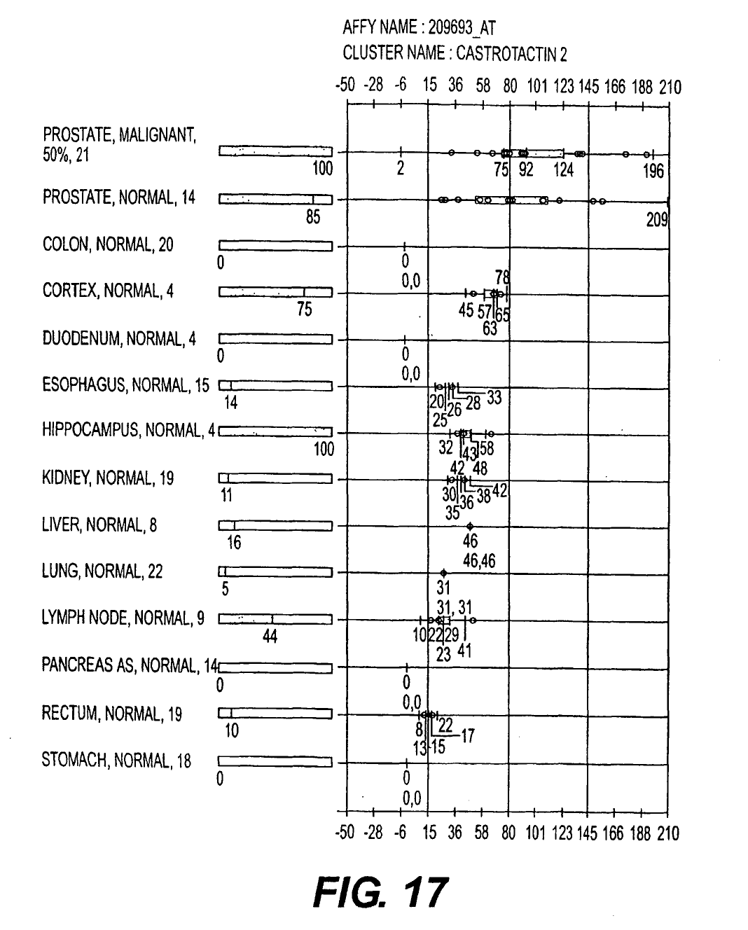

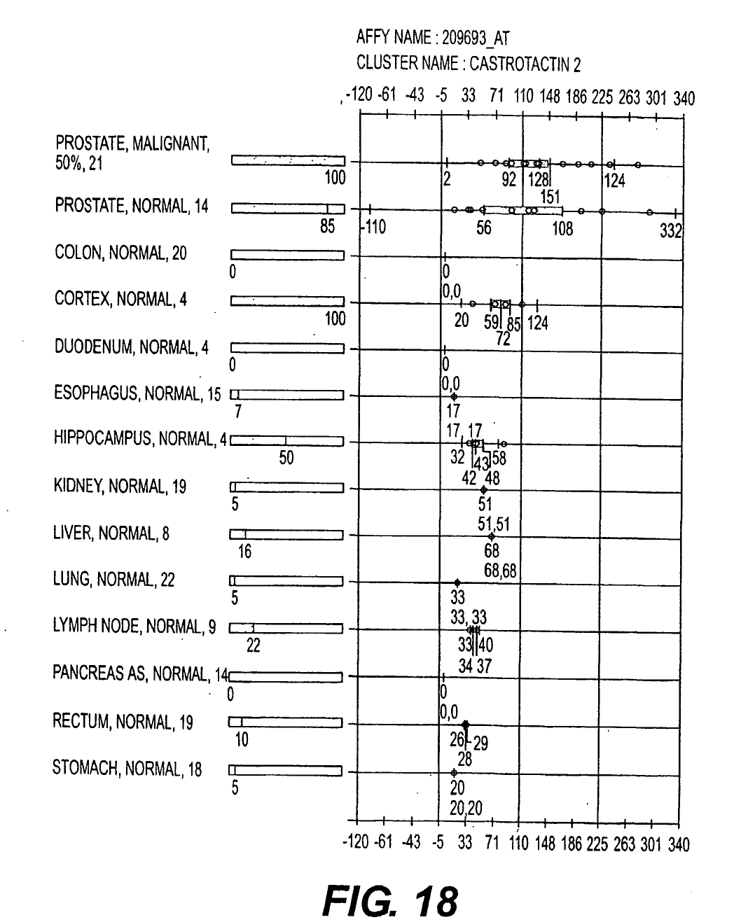

Using the GeneLogic database, we found fragment AF116574 was upregulated 7.01 fold and fragment AK024064 was upregulated 7.54 fold in the malignant prostate samples compared to mixed normal tissue without normal prostate and female specific organs. Enorthern analysis of these fragments demonstrates that they are expressed in 100% of the prostate tumors with greater than 50% malignant cells with very little expression in normal tissues (FIGS. 17 and 18). This protein contains two putative transmembrane domains (TMs) and a signal sequence by SMART™, and three TMs by SOSUI™ prediction programs.

The DNA sequence of these fragments are below:

### AF116574

(SEQ ID NO:8)

TGGGGGACAGCTGAGGATGGGCCTAGCAGATGAAGCTTGCCAGCAAGGCC
AAAGCAAACGGTTTCTCCTGTGGATAGTGGACAGAGACCTTTGTAACCAA
TGGAATTA

### AK024064

(SEQ ID NO:9)

ATTCTACGGCGACTGGAGAGGGTGAGTAGCCACTGCTCCAGCCTCCTGCG
GAGNGCCTACATCCANANCCGNGTGNAANCAGTGCCNTATCTTTTCTGCC
NCANCNANGANGGTCCGGCCTGCANGGCATGGTGTGGTATAGCATCCTCA
AGGNCACCAAAATCACGTGTGAGGAGAAGATGGTGTCAATGGCCCGAAAC
ACATATTATTTGACTCTATCAAAAGTCTCTCCTTTTTAAACCTTTTCTTA
TGGATGGCTGTCAATCCCGAGGCAGAAGTTTTCAGGTGGAGACCAAGCGG
CCTTTGCTCTTCTTCCTTCTTCCTGCCACACTCTGCTTTCTTCCTGCCAT
GGACCCCTGGAGGAGACCTATGGAGGGACAGTTTTGACCTGACCCCTAGA
GGAGACAGTTTTGACCTCTTCAGCACCAGGAAGGAAGCTCTGAGGATGGT
TGCAGTGAGGAAGCATGGGTCTTTAAGGACTTCTCTCTCTTTTTTGCTGG
ACATTATTG

The GeneLogic database calls this protein astrotactin.

### Nucleotide Sequence:

(SEQ ID NO: 10)

CTGTACGCCCAGCGACGTTGGCAGAAGCGTCGCCGCATCCCCCAGAAGAG
CGCAAGCACAGAAGCCACTCATGAGATCCACTACATCCCATCTGTGCTGC
TGGGTCCCCAGGCGCGGGAGAGCTTCCGTTCATCCCGGCTGCAAACCCAC
AATTCCGTCATTGGCGTGCCCATCCGGGAGACTCCCATCCTGGATGACTA
TGACTGTGAGGAGGATGAGGAGCCACCTAGGCGGGCCAACCATGTCTCCC
GCGAGGACGAGTTTGGCAGCCAGGTGACCCACACTCTGGACAGTCTGGGA
CATCCAGGGGAAGAGAAGGTGGACTTTGAGAAGAAAGGAGGAATCAGCTT
TGGGAGAGCCAAGGGGACGTCGGGCTCAGAGGCAGACGATGAAACTCAGC
TGACATTCTACACGGAGCAGTACCGCAGTCGCCGCCGCAGCAAAGGTTTG
CTGAAAAGCCCAGTGAACAAGACAGCCCTGACACTGATTGCTGTGAGTTC
CTGCATCCTGGCCATGGTGTGTGGCAGCCAGATGTCTTGTCCACTCACTG
TGAAGGTGACTCTGCATGTGCCCGAGCACTTCATAGCAGATGGAAGCAGC
TTCGTGGTGAGTGAAGGGAGCTACCTGGACATCTCCGACTGGTTAAACCC
AGCCAAGCTTTCCCTGTATTACCAGATCAATGCCACCTCGCCATGGGTGA
GGGACCTCTGTGGACAAAGGACGACAGATGCCTGTGAGCAGCTCTGCGAC
CCAGAAACCGGAGAGTGCAGCTGTCATGAAGGCTATGCCCCTGACCCTGT
TCACAGACACCTGTGTGTGCGCAGTGACTGGGGACAGAGTGAAGGACCTT
GGCCCTACACGACACTTGAGAGGGGCTATGATCTGGTGACAGGGGAGCAA
GCCCCTGAAAAGATTCTCAGGTCTACTTTCAGCTTGGGCCAAGGCCTCTG
GCTTCCTGTCAGCAAAAGCTTTGTGGTTCCGCCTGTGGAGCTGTCCATCA
ACCCCCTGGCCAGCTGCAAGACCGATGTGCTCGTCACGGAAGACCCTGCA
GATGTCAGGGAAGAAGCGATGCTGTCCACATACTTTGAAACCATCAATGA
CCTGCTGTCTTCCTTCGGGCCAGTTCGTGACTGCTCTCGGAACAATGGGG
GCTGCACTCGCAACTTCAAGTGTGTGTCTGACCGGCAGGTGGATTCCTCG
GGATGTGTGTGCCCTGAGGAGCTGAAACCCATGAAGGATGGCTCTGGCTG
CTACGACCACTCCAAAGGCATTGACTGCTCTGATGGCTTTAATGGCGGCT
GTGAGCAGCTGTGCCTGCAGCAGACGCTGCCCCTGCCCTACGATGCCACT
TCGAGCACCATCTTCATGTTCTGCGGTTGCGTGGAGGAGTACAAACTGGC
TCCTGATGGAAAATCCTGCTTAATGCTCTCAGATGTCTGCGAGGGCCCCA
AGTGCCTCAAACCTGACTCCAAATTCAATGATACCCTCTTTGGAGAGATG
CTACATGGTTACAACAACCGGACCCAGCATGTGAACCAAGGCCAAGTCTT
CCAGATGACCTTTAGGGAGAACAACTTCATCAAGGACTTTCCCCAGCTGG
CCGATGGGCTGTTGGTGATCCCGCTGCCGGTGGAGGAGCAGTGCCGGGGG
GTCCTCTCCGAGCCCCTTCCGGACCTCCAACTGCTCACTGGAGATATCAG
GTATGATGAGGCCATGGGTTACCCCATGGTGCAGCAGTGGCGGGTCCGGA
GCAACCTCTACCGTGTGAAGCTCAGCACCATCACCCTCGCAGCAGGCTTC
ACTAATGTTCTCAAGATCCTGACCAAGGAGAGCAGTCGGGAGGAGCTGCT
GTCCTTCATCCAGCACTATGGCTCCCACTACATCGCAGAGGCCCTCTATG
GCTCAGAGCTCACCTGCATCATCCACTTTCCCAGCAAGAAGGTCCAGCAG
CAGCTGTGGCTCCAGTATCAGAAAGAGACCACAGAGCTGGGCAGCAAGAA
GGAGCTCAAGTCCATGCCCTTCATCACCTACCTCTCAGGTTTGCTGACAG
CCCAGATGCTGTCAGATGACCAGCTCATTTCAGGTGTGGAGATTCGCTGT
GAGGAGAAGGGGCGCTGTCCATCTACCTGTCACCTTTGCCGCCGGCCAGG
CAAGGAGCAGCTGAGCCCCACACCAGTGCTGCTGGAAATCAACCGTGTGG
TGCCACTTTATACCCTCATCCAAGACAATGGCACAAAGGAGGCCTTCAAG
AGTGCACTGATGAGTTCCTACTGGTGCTCAGGGAAAGGGGATGTGATCGA
TGACTGGTGCAGGTGTGACCTCAGCGCCTTTGATGCCAATGGGCTCCCCA
ACTGCAGCCCCCTTCTGCAGCCGGTGCTGCGGCTGTCCCCAACAGTGGAG
CCCTCCAGTACTGTGGTCTCCTTGGAGTGGGTGGATGTTCAGCCAGCTAT
TGGGACCAAGGTCTCCGACTATATTCTGCAGCATAAGAAAGTGGATGAAT
ACACAGACACTGACCTGTACACAGGAGAATTCCTGAGTTTTGCTGATGAC
TTACTCTCTGGCCTGGGCACATCTTGTGTAGCAGCTGGTCGAAGCCATGG
AGAGGTCCCTGAAGTCAGTATCTACTCAGTCATCTTCAAGTGTCTGGAGC
CCGACGGTCTCTACAAGTTCACTCTGTATGCTGTGGATACACGAGGGAGG
CACTCAGAGCTAAGCACGGTGACCCTGAGGACGGCCTGTCCACTGGTAGA
TGACAACAAGGCAGAAGAAATAGCTGACAAGATCTACAATCTGTACAATG
GGTACACAAGTGGAAAGGAGCAGCAGATGGCCTACAACACACTGATGGAG
GTCTCAGCCTCGATGCTGTTCCGAGTCCAGCACCACTACAACTCTCACTA
TGAAAAGTTTGGCGACTTCGTCTGGAGAAGTGAGGATGAGCTGGGGCCCA
GGAAGGCCCACCTGATTCTACGGCGACTGGAGAGGGTGAGTAGCCACTGC
TCCAGCCTCCTGCGGAGTGCCTACATCCAGAGCCGCGTGGAAACAGTGCC
CTATCTTTTCTGCCGCAGCGAGGAGGTCCGGCCTGCAGGCATGGTGTGGT
ATAGCATCCTCAAGGACACCAAAATCACGTGTGAGGAGAAGATGGTGTCA
ATGGCCCGAAACACGTACGGGGAGTCCAAGGGCCGGTGAGGGAGGGTATT
GCCCTCCGTGAGCACAGAGACTCTCCATGGGAGGGGGAGCAGTATTCTCC
TGGATCCTGGGGCCTGGGTGGGCTGGGGGACAGCTGAGGATGGGCCTAGC
AGATGAAGCTTGCCAGCAAGGCCAAAGCAAACGGTTTCTCCTGTGGATAG
TGGACAGAGACCTTTGTAACCAATGGAATTATTCATTTTTCTCTATCTTT
TATTTTTTCAAAGATATTATTTGACTCTATCAAAAGTCTCTCCTTTTTAA
ACCTTTTCTTATGGATGGCTGTCAATCCCGAGGCAGAAGTTTTCAGGTGG
AGACCAAGCGGCCTTTGCTCTTCTTCCTTCTTCCTGCCACACTCTGCTTT
CTTCCTGCCATGGACCCCTGGAGGAGACCTATGGAGGGACAGTTTTGACC
TGACCCCTAGAGGAGACAGTTTTGACCTCTTCAGCACCAGGAAGGAAGCT
CTGAGGATGGTTGCAGTGAGGAAGCATGGGTCTTTAAGGACTTCTCTCTC
TTTTTTGCTGGACATTATTGAGTTTGTGGAACCCTGCCTCTTCCTGCTAC
CTGTGGGTCTGCCCAGAGTCCCTGCAGGCCTGTCCATGCATTAAAAATTC
CTATTGTCTCTCAAAAAAAAAAAAAAAAAAAAAAAAA

### Protein Sequence

(SEQ ID NO: 11)

FASASAVSAAASSSSFATAATAAAARSTAAPPAMAAAGARLSPGPGSGLR
GRPRLCFHPGPPPLLPLLLLFLLLLPPPPLLAGATAAASREPDSPCRLKT
VTVSTLPALRESDIGWSGARAGAGAGTGAGAAAAAASPGSPGSAGTAAES
RLLLFVRNELPGRIAVQDDLDNTELPFGTLEMSGTAADISLVHWRQQWLE
NGTLYFHVSMSSSGQLAQATAPTLQEPSEIVEEQMHILHISVMGGLIALL
LLLLVFTVALYAQRRWQKRRRIPQKSASTEATHEIHYIPSVLLGPQARES
FRSSRLQTHNSVIGVPIRETPILDDYDCEEDEEPPRRANHVSREDEFGSQ
VTHTLDSLGHPGEEKVDFEKKGGISFGRAKGTSGSEADDETQLTFYTEQY
RSRRRSKGLLKSPVNKTALTLIAVSSCILAMVCGSQMSCPLTVKVTLHVP
EHFIADGSSFVVSEGSYLDISDWLNPAKLSLYYQINATSPWVRDLCGQRT
TDACEQLCDPETGECSCHEGYAPDPVHRHLCVRSDWGQSEGPWPYTTLER
GYDLVTGEQAPEKILRSTFSLGQGLWLPVSKSFVVPPVELSINPLASCKT
DVLVTEDPADVREEAMLSTYFETINDLLSSFGPVRDCSRNNGGCTRNFKC
VSDRQVDSSGCVCPEELKPMKDGSGCYDHSKGIDCSDGFNGGCEQLCLQQ
TLPLPYDATSSTIFMFCGCVEEYKLAPDGKSCLMLSDVCEGPKCLKPDSK
FNDTLFGEMLHGYNNRTQHVNQGQVFQMTFRENNFIKDFPQLADGLLVIP
LPVEEQCRGVLSEPLPDLQLLTGDIRYDEAMGYPMVQQWRVRSNLYRVKL
STITLAAGFTNVLKILTKESSREELLSFIQHYGSHYIAEALYGSELTCII
HFPSKKVQQQLWLQYQKETTELGSKKELKSMPFITYLSGLLTAQMLSDDQ
LISGVEIRCEEKGRCPSTCHLCRRPGKEQLSPTPVLLEINRVVPLYTLIQ
DNGTKEAFKSALMSSYWCSGKGDVIDDWCRCDLSAFDANGLPNCSPLLQP
VLRLSPTVEPSSTVVSLEWVDVQPAIGTKVSDYILQHKKVDEYTDTDLYT
GEFLSFADDLLSGLGTSCVAAGRSHGEVPEVSIYSVIFKCLEPDGLYKFT
LYAVDTRGRHSELSTVTLRTACPLVDDNKAEEIADKIYNLYNGYTSGKEQ
QMAYNTLMEVSASMLFRVQHHYNSHYEKFGDFVWRSEDELGPRKAHLILR
RLERVSSHCSSLLRSAYIQSRVETVPYLFCRSEEVRPAGMVWYSILKDTK
ITCEEKMVSMARNTYGESKGR

This protein contains two TMs and a signal sequence by SMART™, and three TMs by SOSUI™ prediction programs.

### AI640307/Protocadherin 10

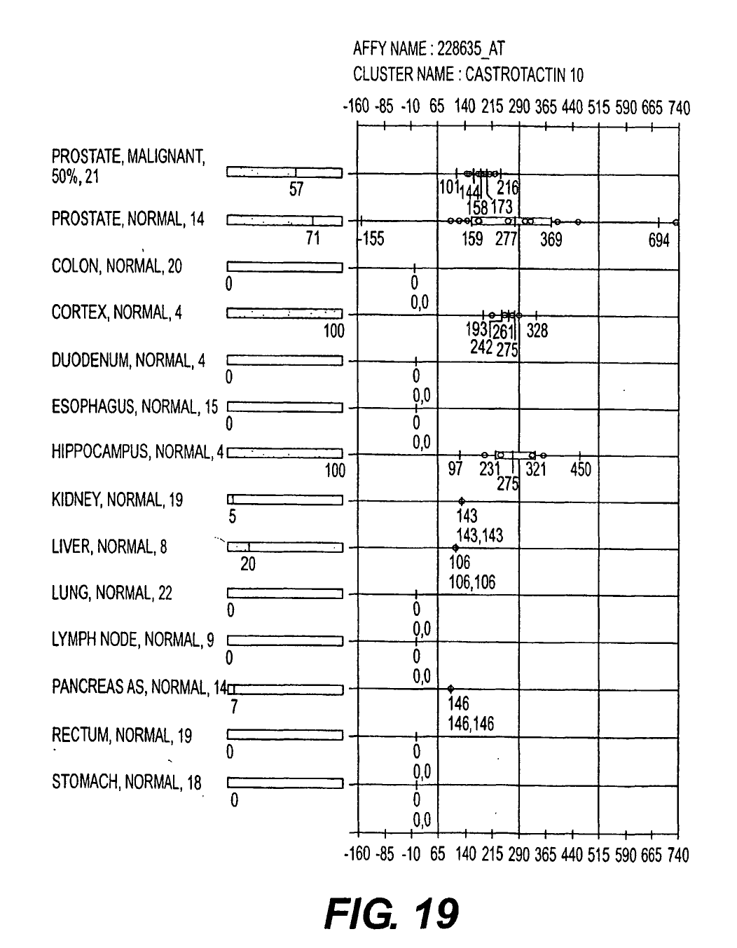

Using the GeneLogic database, we found fragment A1640307 was upregulated 7.69 fold in the malignant prostate samples compared to mixed normal tissue without normal prostate and female specific organs. Enorthern analysis of this fragment (FIG. 19) demonstrates that it is expressed in 87% of the prostate tumors with greater than 50% malignant cells with very little expression in normal tissues other than the prostate and the brain.

The nucleotide sequence of A1640307

(SEQ ID NO: 12)

AACTTCATTATCTTGGCCATCCAGTTAGTCATGTGTAACTGAGTATTAGA
TTTCGGATGGAGTCATCATGGCCAATTATAGGACCTAATTGCTCTCAGCA
GGCCTGAGAAATGAGTTGAAATGTGCAGAACTGTAGAAACTTTAGAGGCA
ACAGATTTTGCCTCCCCGATCAGTGTGTGCCTGTTTACAGCACTATCTAT
CTTTCTCTCTCCAAATGTCACTGAGCCCTTTAGATGTTTATATTCACCAC
GAGAAGCCAGTCATAAAGATAAAGGAAATTTGTGCATTATAAATGCAATA
TCACTGTTTTAAACTTGACTGTTTTATATTATTTTTGTGTGATCAAGTGT
TCCGCAAGCTATTCCAACTTTACAAGAGAAATTGTGATTATGTTCTTTTC
ACCTGTGGGTTATAAAAAATGTTGTATTCTGAAGACCCACAAAATATCAA
AGACATTCTGTAGTTTATACACCGTG

This sequence corresponds to protocadherin 10.

Nucleotide Sequence of Protocadherin 10:

(SEQ ID NO: 13)

CAGGCTCAGAGGCTGAAGCAGGAGGAAGGAAGGACTGGAAGGAAAAAGAG
ACAGGTTAGAGGGAAAGAGGCTTGGGAAGAAAACAGCAGAAAAGAAACTG
CTCATTACACTTACAGAGAGGCAAGTAACGGTGGAGATGAGGACAGAGGG
AACCAAGACTCTGAAAGACAAAAAATACAAATAGAGCGAAAGAGGAAAAA
AATGTCAAGAAGAACATCCATCCGGAGAAATGAAGAGAATGAAAGTTTTA
AACTGCAGAGCCGTTCTGTGCTTTTCCGGCACAAAATTATATCGCTGATT
TTAAGCCCTTTTGCATTTGCCAGCCGTTGACATTAAGAGGCATGTTTAAC
GGTGCCAACAGCATCTCCTTTTCCTTCTCCTCTTCCTCTTCTTCTTCTTC
CTCCTCCTCCTCCTCTTTTTCCTCCTCCTCGTTCTCCTCCCATCAGCAAG
AAGACAAACCGAGGACAGTCTTGAAATATCGAAATTTCCTCTTTGGGATT
TGCCAGCGCCAAGACTGTCGGAATAAAGGACGCTGACTATTGTATTATTG
TTATTTTATTAATTAGTCAGTGGAAAGATTACAGATGAGGAAAGGGGACG
CCTGTCACCCTTCCTTGTGCTAAGATTTAAAAAAAAAGAGGCTGGATTGC
GGGAAGCTCTAAAATGAAGCAAAAGGAGTAAGATTTTTAAAGACAGAAAG
CCACAGGAGCCCCCACGTAGCGCACTTTTATTTGTATTTTTTCAGATTTT
TTTTTGTTTCGTGGTGGTGGGGGAGGTGATTGGGTGGCTGACTGGCTGCG
GGAAGCTACTTCCTTTCCTTTTGGAGATGATTGTGCTATTATTGTTTGCC
TTGCTCTGGATGGTGGAAGGAGTCTTTTCCCAGCTTCACTACACGGTACA
GGAGGAGCAGGAACATGGCACTTTCGTGGGGAATATCGCTGAAGATCTGG
GTCTGGACATTACAAAACTTTCGGCTCGCGGGTTTCAGACGGTGCCCAAC
TCAAGGACCCCTTACTTAGACCTCAACCTGGAGACAGGGGTGCTGTACGT
GAACGAGAAAATAGACCGCGAACAAATCTGCAAACAGAGCCCCTCCTGTG
TCCTGCACCTGGAGGTCTTTCTGGAGAACCCCCTGGAGCTGTTCCAGGTG
GAGATCGAGGTGCTGGACATTAATGACAACCCCCCCTCTTTCCCGGAGCC
AGACCTGACGGTGGAAATCTCTGAGAGCGCCACGCCAGGCACTCGCTTCC
CCTTGGAGAGCGCATTCGACCCAGACGTGGGCACCAACTCCTTGCGCGAC
TACGAGATCACCCCCAACAGCTACTTCTCCCTGGACGTGCAGACCCAGGG
GGATGGCAACCGATTCGCTGAGCTGGTGCTGGAGAAGCCACTGGACCGAG
AGCAGCAAGCGGTGCACCGCTACGTGCTGACCGCGGTGGACGGAGGAGGT
GGGGGAGGAGTAGGAGAAGGAGGGGGAGGTGGCGGGGGAGCAGGCCTGCC
CCCCCAGCAGCAGCGCACCGGCACGGCCCTACTCACCATCCGAGTGCTGG
ACTCCAATGACAATGTGCCCGCTTTCGACCAACCCGTCTACACTGTGTCC
CTACCAGAGAACTCTCCCCCAGGCACTCTCGTGATCCAGCTCAACGCCAC
CGACCCGGACGAGGGCCAGAACGGTGAGGTCGTGTACTCCTTCAGCAGCC
ACATTTCGCCCCGGGCGCGGGAGCTTTTCGGACTCTCGCCGCGCACTGGC
AGACTGGAGGTAAGCGGCGAGTTGGACTATGAAGAGAGCCCAGTGTACCA
AGTGTACGTGCAAGCCAAGGACCTGGGCCCCAACGCCGTGCCTGCGCACT
GCAAGGTGCTAGTGCGAGTACTGGATGCTAATGACAACGCGCCAGAGATC
AGCTTCAGCACCGTGAAGGAAGCGGTGAGTGAGGGCGCGGCGCCCGGCAC
TGTGGTGGCCCTTTTCAGCGTGACTGACCGCGACTCAGAGGAGAATGGGC
AGGTGCAGTGCGAGCTACTGGGAGACGTGCCTTTCCGCCTCAAGTCTTCC
TTTAAGAATTACTACACCATCGTTACCGAAGCCCCCCTGGACCGAGAGGC
GGGGGACTCCTACACCCTGACTGTAGTGGCTCGGGACCGGGGCGAGCCTG
CGCTCTCCACCAGTAAGTCGATCCAGGTACAAGTGTCGGATGTGAACGAC
AACGCGCCGCGTTTCAGCCAGCCGGTCTACGACGTGTATGTGACTGAAAA
CAACGTGCCTGGCGCCTACATCTACGCGGTGAGCGCCACCGACCGGGATG
AGGGCGCCAACGCCCAGCTTGCCTACTCTATCCTCGAGTGCCAGATCCAG
GGCATGAGCGTCTTCACCTACGTTTCTATCAACTCTGAGAACGGCTACTT
GTACGCCCTGCGCTCCTTCGACTATGAGCAGCTGAAGGACTTCAGTTTTC
AGGTGGAAGCCCGGGACGCTGGCAGCCCCCAGGCGCTGGCTGGTAACGCC
ACTGTCAACATCCTCATAGTGGATCAAAATGACAACGCCCCTGCCATCGT
GGCGCCTCTACCAGGGCGCAACGGGACTCCAGCGCGTGAGGTGCTGCCCC
GCTCGGCGGAGCCGGGTTACCTGCTCACCCGCGTGGCCGCCGTGGACGCG
GACGACGGCGAGAACGCCCGGCTCACTTACAGCATCGTGCGTGGCAACGA
AATGAACCTCTTTCGCATGGACTGGCGCACCGGGGAGCTGCGCACAGCAC
GCCGAGTCCCGGCCAAGCGCGACCCCCAGCGGCCTTATGAGCTGGTGATC
GAGGTGCGCGACCATGGGCAGCCGCCCCTTTCCTCCACCGCCACCCTGGT
GGTTCAGCTGGTGGATGGCGCCGTGGAGCCCCAGGGCGGGGGCGGGAGCG
GAGGCGGAGGGTCAGGAGAGCACCAGCGCCCCAGTCGCTCTGGCGGCGGG
GAAACCTCGCTAGACCTCACCCTCATCCTCATCATCGCGTTGGGCTCGGT
GTCCTTCATCTTCCTGCTGGCCATGATCGTGCTGGCCGTGCGTTGCCAAA
AAGAGAAGAAGCTCAACATCTATACTTGTCTGGCCAGCGATTGCTGCCTC
TGCTGCTGCTGCTGCGGTGGCGGAGGTTCGACCTGCTGTGGCCGCCAAGC
CCGGGCGCGCAAGAAGAAACTCAGCAAGTCAGACATCATGCTGGTGCAGA
GCTCCAATGTACCCAGTAACCCGGCCCAGGTGCCGATAGAGGAGTCCGGG
GGCTTTGGCTCCCACCACCACAACCAGAATTACTGCTATCAGGTATGCCT
GACCCCTGAGTCCGCCAAGACCGACCTGATGTTTCTTAAGCCCTGCAGCC
CTTCGCGGAGTACGGACACTGAGCACAACCCCTGCGGGGCCATCGTCACC
GGTTACACCGACCAGCAGCCTGATATCATCTCCAACGGAAGCATTTTGTC
CAACGAGGTAAGGCTGAAGCGAAAGGACCACCATCTCTCATCTCCTCCAT
CAGAAAGCCTCCTCTAGCCCGGCCCTTGTATCTCTGGTGCACTGTATCTA
TTTTTAGGATATTAGCTTATGTGTATCGTTGTGGGAGCAGAGATGGGCGG
TCACCTTCTCCCACTCCTTCGTGTGTAACCTAACTTTCGCGTTGTTCCAC
CCTTTCACATTTATTTTCATTCCGTCCCCTTGGTACTTTGCCACCTTGGA
GCTCCCTCCTTTGCTCTTCCATCCTGTCAGTCCTTTCCCTTCTCAGTAAC
CTGGGCATGAAGGGAAACTGCGTGAAGGGAGAGGGAAATGTGGAGGAGGG
ACTTACTTTCTAGCACTGGCAAAGGTCTTTTTTCTTTGCGTCTGTCCCAG
GCATTAATAAAGTTGGCTCTATTTTGCTTTGTTTAACGATGCTTTTAGTC
GCGTGTACAAGTAAGCTATAGATTGTTTAACTTTA

Amino Acid Sequence of Protocadherin 10:

(SEQ ID NO: 14)

MIVLLLFALLWMVEGVFSQLHYTVQEEQEHGTFVGNIAEDLGLDITKLSA
RGFQTVPNSRTPYLDLNLETGVLYVNEKIDREQICKQSPSCVLHLEVFLE
NPLELFQVEIEVLDINDNPPSFPEPDLTVEISESATPGTRFPLESAFDPD
VGTNSLRDYEITPNSYFSLDVQTQGDGNRFAELVLEKPLDREQQAVHRYV
LTAVDGGGGGGVGEGGGGGGGAGLPPQQQRTGTALLTIRVLDSNDNVPAF
DQPVYTVSLPENSPPGTLVIQLNATDPDEGQNGEVVYSFSSHISPRAREL
FGLSPRTGRLEVSGELDYEESPVYQVYVQAKDLGPNAVPAHCKVLVRVLD
ANDNAPEISFSTVKEAVSEGAAPGTVVALFSVTDRDSEENGQVQCELLGD
VPFRLKSSFKNYYTIVTEAPLDREAGDSYTLTVVARDRGEPALSTSKSIQ
VQVSDVNDNAPRFSQPVYDVYVTENNVPGAYIYAVSATDRDEGANAQLAY
SILECQIQGMSVFTYVSINSENGYLYALRSFDYEQLKDFSFQVEARDAGS
PQALAGNATVNILIVDQNDNAPAIVAPLPGRNGTPAREVLPRSAEPGYLL
TRVAAVDADDGENARLTYSIVRGNEMNLFRMDWRTGELRTARRVPAKRDP
QRPYELVIEVRDHGQPPLSSTATLVVQLVDGAVEPQGGGGSGGGGSGEHQ
RPSRSGGGETSLDLTLILIIALGSVSFIFLLAMIVLAVRCQKEKKLNIYT
CLASDCCLCCCCCGGGGSTCCGRQARARKKKLSKSDIMLVQSSNVPSNPA
QVPIEESGGFGSHHHNQNYCYQVCLTPESAKTDLMFLKPCSPSRSTDTEH
NPCGAIVTGYTDQQPDIISNGSILSNEVRLKRKDHHLSSPPSESLL

This protein has 1 TM domain by SMART™ and SOSUI™.

### AU144598/Contactin Associated Protein-Like 2

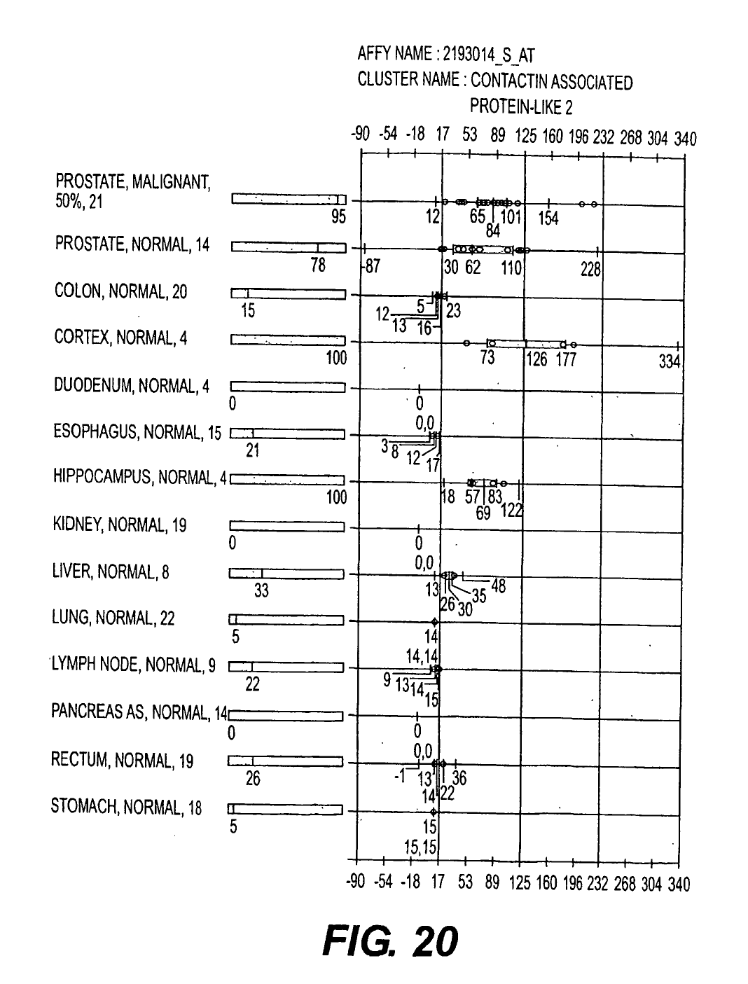

Using the GeneLogic database, we found fragment AU144598 was upregulated 9.19 fold in the malignant prostate samples compared to mixed normal tissue without normal prostate and female specific organs. Enorthern analysis of this fragment demonstrates that it is expressed in 47% of the prostate tumors with greater than 50% malignant cells with very little expression in normal tissues other than normal prostate and brain (FIG. 20).

Sequence of AU144598

(SEQ ID NO: 15)

ACAGCTGTGGGACTTGAACATGCAAGTGTTCAGGTTGTGTCAAGAAGCTT
TTCTTTCCTTCTATGATGGAATCNGTTCTTTTCNATCNNNCTTTTTTCTN
TCTNCNTNTCCTCNCCNCATTATACCNNGNTCTTACGCAGTAAACGTTTT
AATGGCCNGTTTATGTCTCATGCCTCCAANCAACACTGAATTTGAAACCC
CCCATTTTTTCTTTTCACCACCCTGTTGAGCAATTTTCCCAAAAAAAGGG
CAGCAATTATTAAATTNNNNTCAAGTNNNNNNNNNNNNNNTTCNTAGATT
TTACTAAGTTTTATTTTGTCNAGGTTTTTTAAATTTTTTCAGTGAGCGTG
GTGACTGCAGAGGTTAGTGCTGTGAAAAGCTGGGCTAAATATTCTTTCTG
TAAAGTCAAACAGGATTCCATCCCCTGTGAAATAACACAAAATTTCACTC
TCTAAAAGCAACAGCATGTAAACTAGAATGAAAGAAGGAAATTATGTACG
TATGCCTAATATTCTTTGTGAATGTCTTTCATTTAAC

This corresponds to contactin associated protein-like 2

Nucleic Acid Sequence of Contactin Associated Protein-Like 2:

(SEQ ID NO 16)

TGAGGGAAGAAGAGGAAGCGGGAGGAGCTTGGCTTCCTCGCGTATTTGAG
GACAGCCCATCTCCCTTCAAGAACCCTACGGAGAGTCGGACTGCATCTCC
GCAGCGAGCTCTTGGAGCGCCGCCGGCCGGGAGGCGAAGGATGCAGGCGG
CTCCGCGCGCCGGCTGCGGGGCAGCGCTCCTGCTGTGGATTGTCAGCAGC
TGCCTCTGCAGAGCCTGGACGGCTCCCTCCACGTCCCAAAAATGTGATGA
GCCACTTGTCTCTGGACTCCCCCATGTGGCTTTCAGCAGCTCCTCCTCCA
TCTCTGGTAGCTATTCTCCCGGCTATGCCAAGATAAACAAGAGAGGAGGT
GCTGGGGGATGGTCTCCATCAGACAGCGACCATTATCAATGGCTTCAGGT
TGACTTTGGCAATCGGAAGCAGATCAGTGCCATTGCAACCCAAGGAAGGT
ATAGCAGCTCAGATTGGGTGACCCAATACCGGATGCTCTACAGCGACACA
GGGAGAAACTGGAAACCCTATCATCAAGATGGGAATATCTGGGCATTTCC
CGGAAACATTAACTCTGACGGTGTGGTCCGGCACGAATTACAGCATCCGA
TTATTGCCCGCTATGTGCGCATAGTGCCTCTGGATTGGAATGGAGAAGGT
CGCATTGGACTCAGAATTGAAGTTTATGGCTGTTCTTACTGGGCTGATGT
TATCAACTTTGATGGCCATGTTGTATTACCATATAGATTCAGAAACAAGA
AGATGAAAACACTGAAAGATGTCATTGCCTTGAACTTTAAGACGTCTGAA
AGTGAAGGAGTAATCCTGCACGGAGAAGGACAGCAAGGAGATTACATTAC
CTTGGAACTGAAAAAAGCCAAGCTGGTCCTCAGTTTAAACTTAGGAAGCA
ACCAGCTTGGCCCCATATATGGCCACACATCAGTGATGACAGGAAGTTTG
CTGGATGACCACCACTGGCACTCTGTGGTCATTGAGCGCCAGGGGCGGAG
CATTAACCTCACTCTGGACAGGAGCATGCAGCACTTCCGTACCAATGGAG
AGTTTGACTACCTGGACTTGGACTATGAGATAACCTTTGGAGGCATCCCT
TTCTCTGGCAAGCCCAGCTCCAGCAGTAGAAAGAATTTCAAAGGCTGCAT
GGAAAGCATCAACTACAATGGCGTCAACATTACTGATCTTGCCAGAAGGA
AGAAATTAGAGCCCTCAAATGTGGGAAATTTGAGCTTTTCTTGTGTGGAA
CCCTATACGGTGCCTGTCTTTTTCAACGCTACAAGTTACCTGGAGGTGCC
CGGACGGCTTAACCAGGACCTGTTCTCAGTCAGTTTCCAGTTTAGGACAT
GGAACCCCAATGGTCTCCTGGTCTTCAGTCACTTTGCGGATAATTTGGGC
AATGTGGAGATTGACCTCACTGAAAGCAAAGTGGGTGTTCACATCAACAT
CACACAGACCAAGATGAGCCAAATCGATATTTCCTCAGGTTCTGGGTTGA
ATGATGGACAGTGGCACGAGGTTCGCTTCCTAGCCAAGGAAAATTTTGCT
ATTCTCACCATCGATGGAGATGAAGCATCAGCAGTTCGAACTAATAGTCC
CCTTCAAGTTAAAACTGGCGAGAAGTACTTTTTTGGAGGTTTTCTGAACC
AGATGAATAACTCAAGTCACTCTGTCCTTCAGCCTTCATTCCAAGGATGC
ATGCAGCTCATTCAAGTGGACGATCAACTTGTAAATTTATACGAAGTGGC
ACAAAGGAAGCCGGGAAGTTTCGCGAATGTCAGCATTGACATGTGTGCGA
TCATAGACAGATGTGTGCCCAATCACTGTGAGCATGGTGGAAAGTGCTCG
CAAACATGGGACAGCTTCAAATGCACTTGTGATGAGACAGGATACAGTGG
GGCCACCTGCCACAACTCTATCTACGAGCCTTCCTGTGAAGCCTACAAAC
ACCTAGGACAGACATCAAATTATTACTGGATAGATCCTGATGGCAGCGGA
CCTCTGGGGCCTCTGAAAGTTTACTGCAACATGACAGAGGACAAAGTGTG
GACCATAGTGTCTCATGACTTGCAGATGCAGACGCCTGTGGTCGGCTACA
ACCCAGAAAAATACTCAGTGACACAGCTCGTTTACAGCGCCTCCATGGAC
CAGATAAGTGCCATCACTGACAGTGCCGAGTACTGCGAGCAGTATGTCTC
CTATTTCTGCAAGATGTCAAGATTGTTGAACACCCCAGATGGAAGCCCTT
ACACTTGGTGGGTTGGCAAAGCCAACGAGAAGCACTACTACTGGGGAGGC
TCTGGGCCTGGAATCCAGAAATGTGCCTGCGGCATCGAACGCAACTGCAC
AGATCCCAAGTACTACTGTAACTGCGACGCGGACTACAAGCAATGGAGGA
AGGATGCTGGTTTCTTATCATACAAAGATCACCTGCCAGTGAGCCAAGTG
GTGGTTGGAGATACTGACCGTCAAGGCTCAGAAGCCAAATTGAGCGTAGG
TCCTCTGCGCTGCCAAGGAGACAGGAATTATTGGAATGCCGCCTCTTTCC
CAAACCCATCCTCCTACCTGCACTTCTCTACTTTCCAAGGGGAAACTAGC
GCTGACATTTCTTTCTACTTCAAAACATTAACCCCCTGGGGAGTGTTTCT
TGAAAATATGGGAAAGGAAGATTTCATCAAGCTGGAGCTGAAGTCTGCCA
CAGAAGTGTCCTTTTCATTTGATGTGGGAAATGGGCCAGTAGAGATTGTA
GTGAGGTCACCAACCCCTCTCAACGATGACCAGTGGCACCGGGTCACTGC
AGAGAGGAATGTCAAGCAGGCCAGCCTACAGGTGGACCGGCTACCGCAGC
AGATCCGCAAGGCCCCAACAGAAGGCCACACCCGCCTGGAGCTCTACAGC
CAGTTATTTGTGGGTGGTGCTGGGGGCCAGCAGGGCTTCCTGGGCTGCAT
CCGCTCCTTGAGGATGAATGGGGTGACACTTGACCTGGAGGAAAGAGCAA
AGGTCACATCTGGGTTCATATCCGGATGCTCGGGCCATTGCACCAGCTAT
GGAACAAACTGTGAAAATGGAGGCAAATGCCTAGAGAGATACCACGGTTA
CTCCTGCGATTGCTCTAATACTGCATATGATGGAACATTTTGCAACAAAG
ATGTTGGTGCATTTTTTGAAGAAGGGATGTGGCTACGATATAACTTTCAG
GCACCAGCAACAAATGCCAGAGACTCCAGCAGCAGAGTAGACAACGCTCC
CGACCAGCAGAACTCCCACCCGGACCTGGCACAGGAGGAGATCCGCTTCA
GCTTCAGCACCACCAAGGCGCCCTGCATTCTCCTCTACATCAGCTCCTTC
ACCACAGACTTCTTGGCAGTCCTCGTCAAACCCACTGGAAGCTTACAGAT
TCGATACAACCTGGGTGGCACCCGAGAGCCATACAATATTGACGTAGACC
ACAGGAACATGGCCAATGGACAGCCCCACAGTGTCAACATCACCCGCCAC
GAGAAGACCATCTTTCTCAAGCTCGATCATTATCCTTCTGTGAGTTACCA
TCTGCCAAGTTCATCCGACACCCTCTTCAATTCTCCCAAGTCGCTCTTTC
TGGGAAAAGTTATAGAAACAGGGAAAATTGACCAAGAGATTCACAAATAC
AACACCCCAGGATTCACTGGTTGCCTCTCCAGAGTCCAGTTCAACCAGAT
CGCCCCTCTCAAGGCCGCCTTGAGGCAGACAAACGCCTCGGCTCACGTCC
ACATCCAGGGCGAGCTGGTGGAGTCCAACTGCGGGGCCTCGCCGCTGACC
CTCTCCCCCATGTCGTCCGCCACCGACCCCTGGCACCTGGATCACCTGGA
TTCAGCCAGTGCAGATTTTCCATATAATCCAGGACAAGGCCAAGCTATAA
GAAATGGAGTCAACAGAAACTCGGCTATCATTGGAGGCGTCATTGCTGTG
GTGATTTTCACCATCCTGTGCACCCTGGTCTTCCTGATCCGGTACATGTT
CCGCCACAAGGGCACCTACCATACCAACGAAGCAAAGGGGGCGGAGTCGG
CAGAGAGCGCGGACGCCGCCATCATGAACAACGACCCCAACTTCACAGAG
ACCATTGATGAAAGCAAAAAGGAATGGCTCATTTGAGGGGTGGCTACTTG
GCTATGGGATAGGGAGGAGGGAATTACTAGGGAGGAGAGAAAGGGACAAA
AGCACCCTGCTTCATACTCTTGAGCACATCCTTAAAATATCAGCACAAGT
TGGGGGAGGCAGGCAATGGAATATAATGGAATATTCTTGAGACTGATCAC
AAAAAAAAAAAAAACCTTTTTAATATTTCTTTATAGCTGAGTTTTCCCTT
CTGTATCAAAACAAAATAATACAAAAAATGCTTTTAGAGTTTAAGCAATG
GTTGAAATTTGTAGGTACTATCTGTCTTATTTTGTGTGTGTTTAGAGGTG
TTCTAAAGACCCGTGGTAACAGGGCAAGTTTTCTACGTTTTTAAGAGCCC
TTAGAACGTGGGTATTTTTTTTCTTGAGAAAAGCTAATGCACCTACAGAT
GGCCCCCAACATTCTCTTCCTTTTGCTTCTAGTCAACCTTAATGGGCTGT
TACAGAAACTAGTTCGTGTTTATATACTATTTCCTTTGATGTCCTATAAG
TCGGAAAAGAAAGGGGCAAAGAGAACCTATTATTTGCCAGTTTTTAAGCA
GAGCTCAATCTATGCCAGCTCTCTGGCATCTGGGGTTCCTGACTGATACC
AGCAGTTGAAGGAAGAGAGTGCATGGCACCTGGTGTGTAACGACACAATC
AGCACAACTGGAGAGAGGCATTAAAGAACCAGGGAAGGTAGTTTGATTTT
TCATTGAATTCTACAAGCTAATATTGTTCCACGTATGTAGTCTTAGACCA
ATAGCTGTAACTATCAGCTGCAATACCATGGTGACCAGCTGTTACAAAAG
ATTTTTTCCTGTTTTATCTGAAACATACTGGATTTATATATGTATAAGCG
CCTCAATGGGGAATTAGAGCCAGATGTTATGATTTGTTTGCTCTTTTTCT
TTTATAGTTATAGCAAAAATATGGATAATTTCTAGTGAATGCATAAATTA
GGTTGCGTTTCTTATTTTGCTTTAAATCTCTGGTAGTTTTTCCACCCCTG
TGACACAATCCTAATAGACAGTGTCCTGTAAATGGACACAACACAATAAA
GTCAAGTTATTATTGCTGTTACTCTGGATGATATGGAAAACACTGCCATA
TTTTAAATCAACTACTCCACGTGTTTTTCCATCCAATCACACTGCTGTGA
TTCAGGGATCTTTCTTCTAAAACGGACACATTTGAACCTCAGGTTCATCA
CAAACCTGGTACCTGTTGCTTCCCAGAGGATGGAGAAGTGTAGTTAATCA
CACCTCTTAGTTTAATCTGAAATCTTGACCCAGTTATTTAACAAATAAAT
ACCTCATTGATTATATTTAAAAGTAATACACTTCCTGTAAACAAATGGGG
ACAATGCATCCAAAAAATCTTTTTAAACAGATTACACAAAAATTATTTCC
AGAAAGGCTACCATTTATCATCATTATATTTCAAGCCTCTTATACTTAAT
AAGCACTTTCTAAAAAGTCTTGAGATCCCACCATTCTGAGGAATTCAATA
TGATCACTTTTTCCTTCTTTGCCTGGGAGAGGTTAAGAGGCGGTTTCGAA
GGTATAGATGCTATTGTTCTGATGGCCCGGCTGAATAAAATGGAAATTCT
AGTTTGTTAGAATTATGCATTCTTTTTCAAGATTCTCAGTGTGCCTAACT
TATTGGAGCACATCAGTTTCTTGGGTAATGGAAAACATTACCTAGAGTTG
CCAGTGGCACATTACACCAGTACAGAGCACATTCCAAAGGAGACATTGGA
CCAGTTAATTCCCATACAAGTCAAGGTAACAGAACAAAAGGGAATCCTGA
TGCCCTTTTACCATTGCTGGTTGAGCTCAGGCACTGTCATGGACACCCTT
AATTTTAAAAGGTTTTAATCATTCTTCTATAAAATACATTTAAAATGGAA
AAATACTTAATATCACTAAATATCAGAACAATGTAACATTTACAAATGAC
ATATTGAAAGCAAAGGCTGTTTTATTTAGCCAAGATGATTACCATTAGGA
GTTACTTTATGTATTGTTGAAAGCAAATTTTAAACATGATGTTTTAGAAG
TGTTTCTGATTTTTAAACCTGGTTTACAGGTATTACTTCTGCACTTACCA
AATAATGCCAGATGGAAATTTATTATTTCTTGCAATTCCCGTGATAGCTC
TGTTCTTTATGCATTGTCTCAACACTTTCCCTTTTTTCCCAAAATGAGTA
GAGAATTAAAGCCACCCAAAACAGCTTCTGCTACTAAAATGTTCTCATCC
TTTCTCCTCCCTCTCCTTTTCCTGCCACAAAAGGTGAAAAATGAGATCCA
ATCCTCTCACCAAAATTTCAAACCTAGGACACTGGAATGACTGCAGGGAT
CAGTGGTTCTCCCATATCACCATCAATTAAGACATATAGGACACTGTCTT
CCTTCAAGAGGGTTACAATGTGGCCATCAGACAGGAAACCAAACGGTGGA
TAAAGTATTAAGTAACTAAGTGCCAAATAAATGCTGGAAATCTTGACCTC
TCCTTGGGATTATGGGTGTAACAAAAATCCCTACATCTGTTTATGAAGGC
CATATTCAGTACATTTTAAATGGTAAATAATCTGTTTATGTGAAGAAAAA
GAATTAAGTCTTTCTTCCAACTCTCTCCTTGGATAGCCTAGCACAGTGCA
GCCTCCATAACCATGACATTCCCGCCCAAGCTCTCAGTGCCTAATCCTGC
TTTGTCATTCACATCTCACAAAATCTTGACATCTTACATTCCAATACATT
ATCAAGCAAGCACAAGTATGCTGGTAGTAGCCTCTTTAAATAATATGTAT
AGACAACAACAACGACAAAAAATAGACTGTTTTAAAGTTTCAGGGAAAGT
TGGTGGCTGATTTAAAGTTGTGCAGGAAACATCTTCTGTGTATGAAGCAA
ATGTCGATGTTTTGAAAAGCTAGGAGATGACTTTGAATGAATGCAAGGTT
AGTGAGATCCTAAGCTCTCAAAATAGCATATTCCCTAGAGCTCAAGAAAG
CTGGTCCAGGAGGTTGAAAAAGCTATTTTGTTGTTAAATTATTTTCTGGC
CCTTCTTAATATTTAAAAATGTATTTCCCCTTGTGGCTTTCAACCACCTG
CTCAAAAAAAGAGACTTGTTACATGAAAGTTTTCATTAAAGAGCTGAAAA
CAAGAATTTAGAGAGCCATTCCTAGAAAATGTCCTACTGCCCTGCATTTG
ACAAACAAGCATCCTTTACTAACAAGAGCAGGAATTCAGAGGCACAAGAA
AAAGCATTGGCATGAGCCAAAGAGTCTGTCTTAATGTTACTTTTGAAAAT
CTGCTGAGCGGCCACCATATGCAGGCTGAGAGCTGGGCACAGGCGAAGCC
ATTGGAAGCACTTCAGGAACAAGCACACAGCTGTGGGACTTGAACATGCA
AGTGTTCAGGTTGTGTCAAGAAGCTTTTCTTTCCTTCTATGATGGAATCT
GTTCTTTTCTATCCTACTTTTTTCTCTCTTCCTCTCCTCACCACATTATA
CCCTGCTCTTACGCAGTAAACGTTTTAATGGCCCGTTTATGTCTCATGCC
TCCAAACAACACTGAATTTGAAACCCCCCATTTTTTCTTTTCACCACCCT
GTTGAGCAATTTTCCCAAAAAAAGGGCAGCAATTATTAAATTGAATTCAA
GTTTCTAGATTTTACTAAGTTTTATTTTGTCAGGTTTTTTAAATTTTTTC
AGTGAGCGTGGTGACTGCAGAGGTTAGTGCTGTGAAAAGCTGGGCTAAAT
ATTCTTTCTGTAAAGTCAAACAGGATTCCATCCCCTGTGAAATAACACAA
AATTTCACTCTCTAAAAGCAACAGCATGTAAACTAGAATGAAAGAAGGAA
ATTATGTACGTATGCCTAATATTCTTTGTGAATGTCTTTCATTTAACTAA
AATTATATTAGAAACCAGATTGATAAATAAAAAATTCAAAGTAGTTTTAA
TTATCCT

Amino Acid Sequence of Contactin Associated Protein-Like 2:

(SEQ ID NO 17)

MQAAPRAGCGAALLLWIVSSCLCRAWTAPSTSQKCDEPLVSGLP
HVAFSSSSSISGSYSPGYAKINKRGGAGGWSPSDSDHYQWLQVDFGNRKQ
ISAIATQGRYSSSDWVTQYRMLYSDTGRNWKPYHQDGNIWAFPGNINSDG
VVRHELQHPIIARYVRIVPLDWNGEGRIGLRIEVYGCSYWADVINFDGHV
VLPYRFRNKKMKTLKDVIALNFKTSESEGVILHGEGQQGDYITLELKKAK
LVLSLNLGSNQLGPIYGHTSVMTGSLLDDHHWHSVVIERQGRSINLTLDR
SMQHFRTNGEFDYLDLDYEITFGGIPFSGKPSSSSRKNFKGCMESINYNG
VNITDLARRKKLEPSNVGNLSFSCVEPYTVPVFFNATSYLEVPGRLNQDL
FSVSFQFRTWNPNGLLVFSHFADNLGNVEIDLTESKVGVHINITQTKMSQ
IDISSGSGLNDGQWHEVRFLAKENFAILTIDGDEASAVRTNSPLQVKTGE
KYFFGGFLNQMNNSSHSVLQPSFQGCMQLIQVDDQLVNLYEVAQRKPGSF
ANVSIDMCAIIDRCVPNHCEHGGKCSQTWDSFKCTCDETGYSGATCHNSI
YEPSCEAYKHLGQTSNYYWIDPDGSGPLGPLKVYCNMTEDKVWTIVSHDL
QMQTPVVGYNPEKYSVTQLVYSASMDQISAITDSAEYCEQYVSYFCKMSR
LLNTPDGSPYTWWVGKANEKHYYWGGSGPGIQKCACGIERNCTDPKYYCN
CDADYKQWRKDAGFLSYKDHLPVSQVVVGDTDRQGSEAKLSVGPLRCQGD
RNYWNAASFPNPSSYLHFSTFQGETSADISFYFKTLTPWGVFLENMGKED
FIKLELKSATEVSFSFDVGNGPVEIVVRSPTPLNDDQWHRVTAERNVKQA
SLQVDRLPQQIRKAPTEGHTRLELYSQLFVGGAGGQQGFLGCIRSLRMNG
VTLDLEERAKVTSGFISGCSGHCTSYGTNCENGGKCLERYHGYSCDCSNT
AYDGTFCNKDVGAFFEEGMWLRYNFQAPATNARDSSSRVDNAPDQQNSHP
DLAQEEIRFSFSTTKAPCILLYISSFTTDFLAVLVKPTGSLQIRYNLGGT
REPYNIDVDHRNMANGQPHSVNITRHEKTIFLKLDHYPSVSYHLPSSSDT
LFNSPKSLFLGKVIETGKIDQEIHKYNTPGFTGCLSRVQFNQIAPLKAAL
RQTNASAHVHIQGELVESNCGASPLTLSPMSSATDPWHLDHLDSASADFP
YNPGQGQAIRNGVNRNSAIIGGVIAVVIFTILCTLVFLIRYMFRHKGTYH
TNEAKGAESAESADAAIMNNDPNFTETIDESKKEWLI

SOSUI and TmPred predict 2 TM domains.

### BC001186/Protocadherin 5

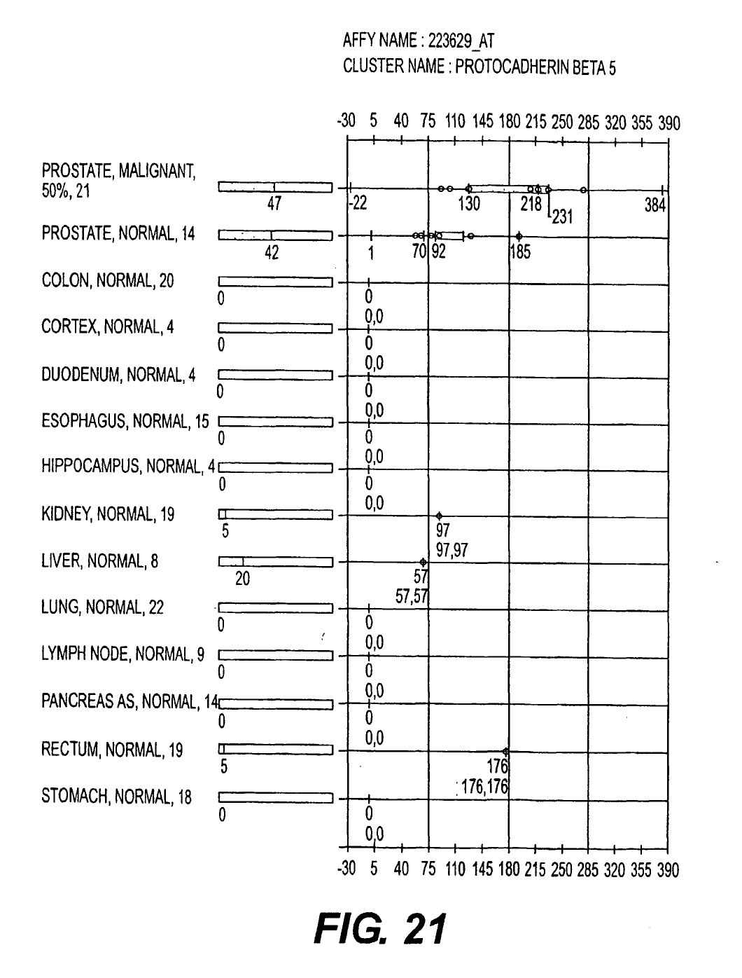

Using GeneLogic database, we found fragment BC001186 was upregulated 6.34 fold in the malignant prostate samples compared to mixed normal tissue without normal prostate and female specific organs. Enorthern analysis of this fragment demonstrates that it is expressed in 47% of the prostate tumors with greater than 50% malignant cells with very little expression in normal tissues (FIG. 21)

The sequence of BC001186

(SEQ ID NO 18)

GCTACCACTACGAGGTGTGTTTGACCGGAGACTCAGGGGCCGGCGAGTTC
AAGTTCCTGAAGCCGATTATTCCTAACCTTTTGCCCCAGGGCGCTGGTGA
AGAAATAGGGAAAACTGCTGCCTTCCGGAATAGCTTTGGATTAAATTAGA
GATCTCGTGATGACGCGTTGTTTTCTGCCATTTATCCCAAACTTTTTCAG
ATCTAGAATTCGAGAGTGTCATGGACAAAAATTTCACCTTGAGATTGAGC
TTTTATTTCCCTTTTTAATGGATTTGTCTGTTGAACTTCATGCTGTCCAA
GTGTTGAAAAGTCAATTTTATTTCATTGCATTTATTTACATAGTGTCATT
CCAAATCCATGCATGCTGTTGATTTTCCTGAGATTTTTTTCTCTTCTTGT
TGGTATTTGTT

This sequence corresponds to Protocadherin 5 beta:

Nucleic Acid Sequence of Protocadherin 5 Beta:

(SEQ ID NO 19)

GCGGATAACTCAGACGCCATTAAGCTGGGGAATCCAAACTCTAAAAGAAG
GACGCATTTTAGGTAAGATCTAGTGGCTAGATCTTCAGGGTGGGCTTCGT
TCTTGTGGAAATCAGTCAAGAAAGATCGGATTCGCGGTTATTTATGCAAA
TCATCTGGGTGGATTGTGTACGGAGTTAAACTGCGCCTTCTGGACCGGGT
CTGAACAATGGAGACTGCGCTAGCAAAAACGCCACAGAAAAGGCAAGTTA
TGTTTCTTGCTATATTGTTGCTTTTGTGGGAGGCTGGCTCTGAGGCAGTT
AGGTATTCCATACCAGAAGAAACAGAAAGTGGCTATTCTGTGGCCAACCT
GGCAAAAGACCTGGGTCTTGGGGTGGGGGAACTGGCCACTCGGGGCGCGC
GAATGCATTACAAAGGAAACAAAGAGCTCTTGCAGCTTGATATAAAGACC
GGCAATTTGCTTCTATATGAAAAACTAGACCGGGAGGTGATGTGCGGGGC
GACAGAACCCTGTATATTGCATTTCCAGCTCTTACTAGAAAATCCAGTGC
AGTTTTTTCAAACTGATCTGCAGCTCACAGATATAAATGACCATGCCCCA
GAGTTCCCAGAGAAGGAAATGCTCCTAAAAATCCCAGAGAGCACCCAGCC
AGGGACTGTGTTTCCCTTAAAAATAGCCCAGGACTTTGACATAGGTAGCA
ACACTGTTCAGAACTACACAATCAGCCCAAATTCACACTTTCATGTTGCT
ACGCATAATCGCGGAGATGGCAGAAAATACCCAGAGCTGGTGCTGGACAA
AGCGCTGGACCGGGAGGAGCGGCCTGAGCTCAGCTTAACACTCACTGCAC
TGGACGGTGGGGCTCCGCCCAGGTCCGGGACCACCACAATTCGCATTGTC
GTCTTGGATAATAATGACAACGCCCCCGAATTTTTACAATCATTCTATGA
GGTACAGGTGCCCGAGAACAGCCCCCTTAACTCCTTAGTTGTCGTTGTCT
CCGCTCGAGATTTAGATGCAGGAGCATATGGGAGTGTAGCCTATGCTCTA
TTCCAAGGCGATGAAGTTACTCAACCATTTGTAATAGACGAGAAAACAGC
AGAAATTCGCCTGAAAAGGGCATTGGATTTCGAGGCAACTCCATATTATA
ACGTGGAAATTGTAGCCACAGATGGTGGGGGCCTTTCAGGAAAATGCACT
GTGGCTATAGAAGTGGTGGATGTGAATGACAACGCCCCTGAACTCACCAT
GTCTACGCTCTCCAGCCCTACCCCAGAAAATGCCCCGGAAACTGTAGTTG
CCGTTTTCAGTGTTTCTGATCCAGACTCCGGGGACAACGGTAGGATGATT
TGCTCCATCCAGAATGATCTCCCCTTTCTTTTGAAGCCCACATTAAAAAA
CTTTTACACCCTAGTGACACAGAGAACACTGGACAGAGAGAGCCAAGCCG
AGTACAACATCACCATCACTGTCACCGACATGGGGACACCCAGGCTGAAA
ACCGAGCACAACATAACGGTCCTGGTCTCCGACGTCAATGACAACGCCCC
CGCCTTCACCCAAACCTCCTACACCCTGTTCGTCCGAGAGAACAACAGCC
CCGCCCTGCACATCGGCAGTGTCAGCGCCACAGACAGAGACTCAGGCACC
AACGCCCAGGTCACCTACTCGCTGCTGCCGCCCCAGAATCCACACCTGCG
CCTCGCCTCCCTGGTCTCCATCAACGCGGACAACGGCCACCTGTTTGCCC
TCAGGTCGCTGGACTACGAGGCCCTGCAGGCGTTCGAGTTCCGCGTGGGA
GCCACAGACCGCGGCTCCCCGGCGCTGAGCAGCGAGGCGCTGGTGCGCGT
GCTGGTGCTGGACGCCAACGACAACTCGCCCTTCGTGCTGTATCCGCTGC
AGAACGGCTCGGCGCCTTGCACCGAGCTGGTGCCCCGGGCGGCCGAGCCG
GGCTACCTGGTGACCAAGGTGGTGGCGGTGGACGGTGACTCGGGCCAGAA
CGCCTGGCTGTCGTACCAGCTGCTCAAGGCCACGGAGCCCGGGCTGTTCA
GCATGTGGGCGCACAATGGCGAGGTGCGCACCGCCAGGCTGCTGAGCGAG
CGCGACGCGGCCAAGCACAGGCTGGTGGTGCTGGTCAAGGACAATGGCGA
GCCTCCGCGCTCGGCCACCGCCACGCTGCACGTGCTCCTGGTGGACGGCT
TCTCCCAGCCCTACCTGCCGCTGCCGGAGGCGGCCCCGGCCCAGGCCCAG
GCCGACTCGCTCACTGTCTACCTGGTGGTGGCATTGGCCTCGGTGTCGTC
GCTCTTCCTCTTTTCGGTGCTCCTGTTCGTGGCAGTGCGGCTGTGCAGGA
GGAGCAGGGCGGCCCCGGTCGGTCGCTGCTCGGTGCCCGAGGGCCCCTTT
CCAGGGCATCTGGTGGACGTGAGCGGCACCGGGACCCTATCCCAGAGCTA
CCACTACGAGGTGTGTTTGACCGGAGACTCAGGGGCCGGCGAGTTCAAGT
TCCTGAAGCCGATTATTCCTAACCTTTTGCCCCAGGGCGCTGGTGAAGAA
ATAGGGAAAACTGCTGCCTTCCGGAATAGCTTTGGATTAAATTAGAGATC
TCGTGATGACGCGTTGTTTTCTGCCATTTATCCCAAACTTTTTCAGATCT
AGAATTCGAGAGTGTCATGGACAAAAATTTCACCTTGAGATTGAGCTTTT
ATTTCCCTTTTTAATGGATTTGTCTGTTGAACTTCATGCTGTCCAAGTGT
TGAAAAGTCAATTTTATTTCATTGCATTTATTTACATAGTGTCATTCCAA
ATCCATGCATGCTGTTGATTTTCCTGAGATTTTTTTCTCTTCTTGTTGGT
ATTTGTTGTGATAAACCACCTTAATAAAATCAAGTATTAATTTTAAAAAA
AAAAAAAAAAAAAAA

Amino Acid of Protocadherin 5 Beta

(SEQ ID NO 20)

MCGATEPCILHFQLLLENPVQFFQTDLQLTDINDHAPEFPEKEMLLKIPE
STQPGTVFPLKIAQDFDIGSNTVQNYTISPNSHFHVATHNRGDGRKYPEL
VLDKALDREERPELSLTLTALDGGAPPRSGTTTIRIVVLDNNDNAPEFLQ
SFYEVQVPENSPLNSLVVVVSARDLDAGAYGSVAYALFQGDEVTQPFVID
EKTAEIRLKRALDFEATPYYNVEIVATDGGGLSGKCTVAIEVVDVNDNAP
ELTMSTLSSPTPENAPETVVAVFSVSDPDSGDNGRMICSIQNDLPFLLKP
TLKNFYTLVTQRTLDRESQAEYNITITVTDMGTPRLKTEHNITVLVSDVN
DNAPAFTQTSYTLFVRENNSPALHIGSVSATDRDSGTNAQVTYSLLPPQN
PHLRLASLVSINADNGHLFALRSLDYEALQAFEFRVGATDRGSPALSSEA
LVRVLVLDANDNSPFVLYPLQNGSAPCTELVPRAAEPGYLVTKVVAVDGD
SGQNAWLSYQLLKATEPGLFSMWAHNGEVRTARLLSERDAAKHRLVVLVK
DNGEPPRSATATLHVLLVDGFSQPYLPLPEAAPAQAQADSLTVYLVVALA
SVSSLFLFSVLLFVAVRLCRRSRAAPVGRCSVPEGPFPGHLVDVSGTGTL
SQSYHYEVCLTGDSGAGEFKFLKPIIPNLLPQGAGEEIGKTAAFRNSFGL
N

This protein has 1 TM by both SMART and SOSUI prediction programs.

### NM 015392/Neural Proliferation, Differentiation and Control 1

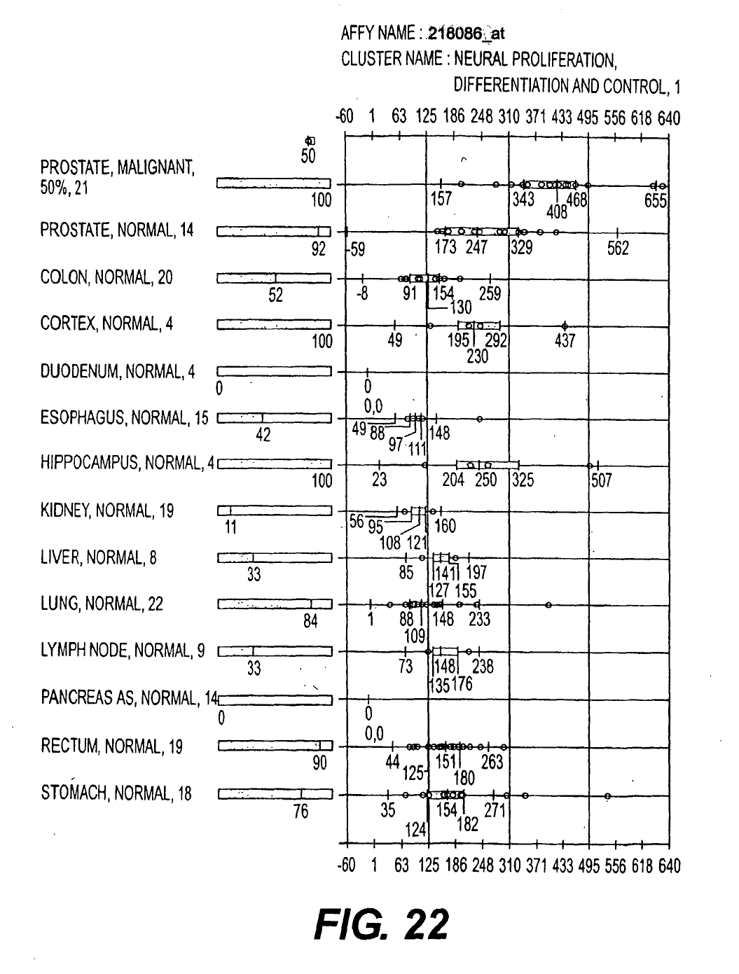

Using the GeneLogic database, we found fragment NM_{—}015392 was upregulated 4.53 fold in the malignant prostate samples compared to mixed normal tissue without normal prostate and female specific organs. Enorthern analysis of this fragment demonstrates that it is expressed in 100% of the prostate tumors with greater than 50% malignant cells with very little expression in normal tissues except for the brain (FIG. 22).

### Sequence of NM_{—}_015392

(SEQ ID NO 21)

GGCACAGAGCGCGGAGATGTACCACTACCAGCACCAACGGCAACAGATGC
TGTGCCTGGAGCGGCATAAAGAGCCACCCAAGGAGCTGGACACGGCCTCC
TCGGATGAGGAGAATGAGGACGGAGACTTCACGGTGTACGAGTGCCCGGG
CCTGGCCCCGACCGGGGAAATGGAGGTGCGCAACCCTCTGTTCGACCACG
CCGCACTGTCCGCGCCCCTGCCGGCCCCCAGCTCACCGCCTGCACTGCCA
TGACCTGGAGGCAGACAGACGCCCACCTGCTCCCCGACCTCGAGGCCCCC
GGGGAGGGGCAGGGCCTGGAGCTTCCCACTAAAAACATGTTTTGATGCTG
TGTGCTTTTGGCTGGGCCTCGGGCTCCAGGCCCTGGGACCCCTTGCCAGG
GAGACCCCCGAACCTTTGTGCCAGGACACCTCCTGGTCCCCTGCACCTCT
CCTGTTCGGTTTAGACCCCCAAACTGGAGGGGGCATGGAGAACCGTAGAG
CGCAGGAACGGGTGGGTAATT

This corresponds to neural proliferation, differentiation and control 1:

### Nucleic Acid Sequence

(SEQ ID NO 22)

GGCACGAGGGCCTCTTCTTCCTCCTGCGTCCTCCCCCGCTGCCTCCGCTG
CTCCCGACGCGGAGCCCGGAGCCCGCGCCGAGCCCCTGGCCTCGCGGTGC
CATGCTGCCCCGGCGGCGGCGCTGAAGGATGGCGACGCCGCTGCCTCCGC
CCTCCCCGCGGCACCTGCGGCTGCTGCGGCTGCTGCTCTCCGGCCTCGTC
CTCGGCGCCGCCCTGCGTGGAGCCGCCGCCGGCCACCCGGATGTAGCCGC
CTGTCCCGGGAGCCTGGACTGTGCCCTGAAGAGGCGGGCAAGGTGTCCTC
CTGGTGCACATGCCTGTGGGCCCTGCCTTCAGCCCTTCCAGGAGGACCAG
CAAGGGCTCTGTGTGCCCAGGATGCGCCGGCCTCCAGGCGGGGGCCGGCC
CCAGCCCAGACTGGAAGATGAGATTGACTTCCTGGCCCAGGAGCTTGCCC
GGAAGGAGTCTGGACACTCAACTCCGCCCCTACCCAAGGACCGACAGCGG
CTCCCGGAGCCTGCCACCCTGGGCTTCTCGGCACGGGGGCAGGGGCTGGA
GCTGGGCCTCCCCTCCACTCCAGGAACCCCCACGCCCACGCCCCACACCT
CCCTGGGCTCCCCTGTGTCATCCGACCCGGTGCACATGTCGCCCCTGGAG
CCCCGGGGAGGGCAAGGCGACGGCCTCGCCCTTGTGCTGATCCTGGCGTT
CTGTGTGGCCGGTGCAGCCGCCCTCTCCGTAGCCTCCCTCTGCTGGTGCA
GGCTGCAGCGTGAGATCCGCCTGACTCAGAAGGCCGACTACGCCACTGCG
AAGGCCCCTGGCTCACCTGCAGCTCCCCGGATCTCGCCTGGGGACCAGCG
GCTGGCACAGAGCGCGGAGATGTACCACTACCAGCACCAACGGCAACAGA
TGCTGTGCCTGGAGCGGCATAAAGAGCCACCCAAGGAGCTGGACACGGCC
TCCTCGGATGAGGAGAATGAGGACGGAGACTTCACGGTGTACGAGTGCCC
GGGCCTGGCCCCGACCGGGGAAATGGAGGTGCGCAACCCTCTGTTCGACC
ACGCCGCACTGTCCGCGCCCCTGCCGGCCCCCAGCTCACCGCCTGCACTG
CCATGACCTGGAGGCAGACAGACGCCCACCTGCTCCCCGACCTCGAGGCC
CCCGGGGAGGGGCAGGGCCTGGAGCTTCCCACTAAAAACATGTTTTGATG
CTGTGTGCTTTTGGCTGGGCCTCGGGCTCCAGGCCCTGGGACCCCTTGCC
AGGGAGACCCCCGAACCTTTGTGCCAGGACACCTCCTGGTCCCCTGCACC
TCTCCTGTTCGGTTTAGACCCCCAAACTGGAGGGGGCATGGAGAACCGTA
GAGCGCAGGAACGGGTGGGTAATTCTAGAGACAAAAGCCAATTAAAGTCC
ATTTCAGACCTGCGGCTTCTGAAAAAAAAAAAAAAAAAAAA

### Amino Acid Sequence of Neural Proliferation, Differentiation and Control 1:

(SEQ ID NO 23)

MATPLPPPSPRHLRLLRLLLSGLVLGAALRGAAAGHPDVAACPGSLDCAL
KRRARCPPGAHACGPCLQPFQEDQQGLCVPRMRRPPGGGRPQPRLEDEID
FLAQELARKESGHSTPPLPKDRQRLPEPATLGFSARGQGLELGLPSTPGT
PTPTPHTSMGSPVSSDPVHMSPLEPRGGQGDGLALVLILAFCVAGAAALS
VASLCWCRLHREIRLTQKADYATAKAPGSPAAPRISPGDQRLAQSAEMYH
YQHARQQMLCLERHKEPPKELDTASSDEENEDGDFTVYECPGLAPTGEME
VRNPLFDHAALSAPLPAPSSPPALP

This protein contains one TM and a signal sequence by SMART and two TMs by SOSUI prediction programs.

### AI832249/HS1-2

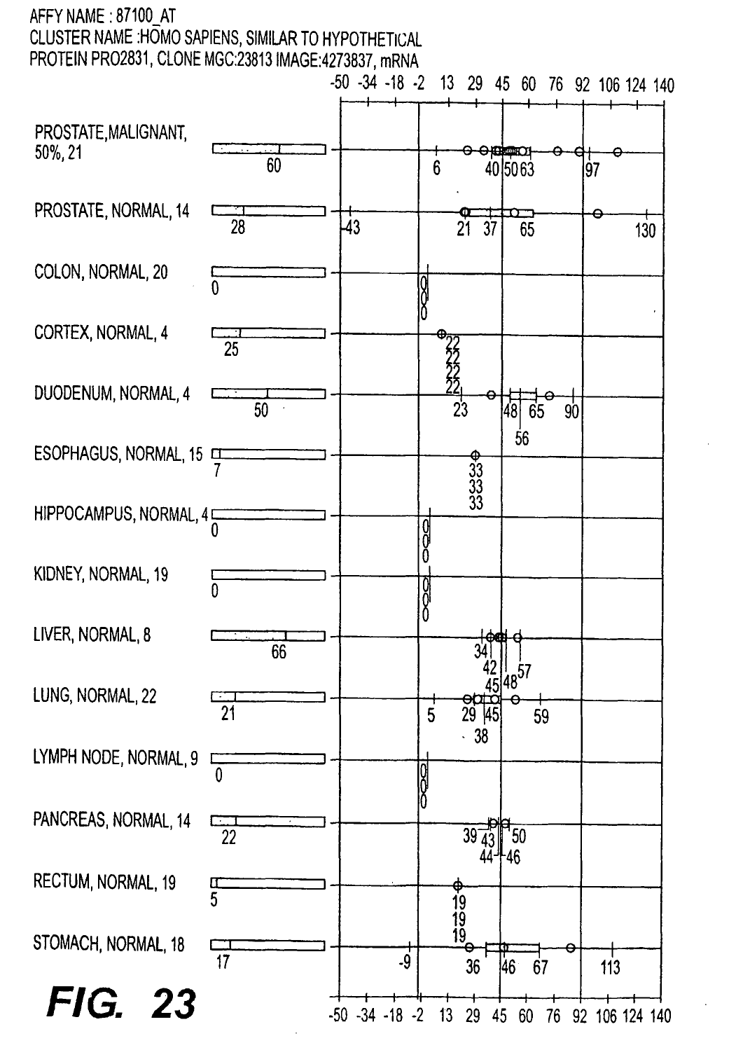

We found fragment AI832249 was upregulated 3.87 fold in the malignant prostate samples from the Jun. 7, 2002 update of GeneLogic compared to mixed normal tissue without normal prostate and female specific organs. Enorthern analysis of this fragment demonstrates that it is expressed in 60% of the prostate tumors with greater than 50% malignant cells with low expression in normal tissues other than the prostate and the liver (FIG. 23).

### Sequence of AB832249

(SEQ ID NO 24)

GAAATCCTTCCTGCTCAGGCTTTCATTCTAAAACTACAGTCTTCATTAAA
GCTGAACTTTCTGGGTAGCTGAGCTTATATGCCCGGCATCTGAATGAGAG
CTCTCTTTGTAACTGTGTGACTTGAGATCTAGTTTGCNAGNTCCNGGNAA
ACAATACATGTGTTNTTNNNTTTGTGTTTGCTCAGCAAGCAGATGTCTGA
GATGTAAGAAGCTTTTCTTTTCCTGTGGCATTGATTCTGACTTAGAGCTG
AAGTAAAGATCACTGAAACATCACGTCAAGTTGAAGTCACTCATAGGTCT
TTGTCCTTTAGGCAGGACAGGAGAGTCATTAAGAAGCATTTCACTGTAGC
ATTCTATCACAATATCATCTGGAATTNTTTTCTTTGCCCAGAAAGCCTTA
ACTTGCCTCTAGAGAATCCCTGGNNNNNNNNNNNNNNNNNNNNNNNNNNN
NTNCAACTCTTCTGCTGTGGAAGTTTGAAGCGACNGNCNAGGCANANCCA
GAGAATTTCCTCAAGTNGCCTNTAGGTNCCNTGTTATCTTATGCCCCCAC
CCCTCCCTCAACAATATGAGTGATCCAG

### This AB832249 Sequence Corresponds to a Novel 3′UTR of HS1-2:

(SEQ ID NO 25)

gaattcgggcggggagctgcaggaaccagactgggggcgagctgagcacc
tgtagtcaatcacacgcagcttttaggtttgtttgaataagagatctgac
ctgaccggcccaactgtacaactcttcaaggaaaattcgtatttgcagtg
ggaagaataagtaacattgatcaagatgaatgccatgctggagactcccg
aactcccagccgtgtttgatggagtgaagctggctgcagtggctgctgtg
ctgtacgtgatcgtccggtgtttgaacctgaagagccccacagccccacc
tgacctctacttccaggactcggggctctcacgctttctgctcaagtcct
gtcctcttctgaccaaagaatacattccaccgttgatctgggggaaaagt
ggacacatccagacagccttgtatgggaagatgggaagggtgaggtcgcc
acatccttatgggcaccggaagttcatcactatgtctgatggagccactt
ctacattcgacctcttcgagcccttggctgagcactgtgttggagatgat
atcaccatggtcatctgccctggaattgccaatcacagcgagaagcaata
catccgcactttcgttgactacgcccagaaaaatggctatcggtgcgccg
tgctgaaccacctgggtgccctgcccaacattgaattgacctcgccacgc
atgttcacctatggctgcacgtgggaatttggagccatggtgaactacat
caagaagacatatcccctgacccagctggtcgtcgtgggcttcagcctgg
gtggtaacattgtgtgcaaatacttgggggagactcaggcaaaccaagag
aaggtcctgtgctgcgtcagcgtgtgccaggggtacagtgcactgagggc
ccaggaaaccttcatgcaatgggatcagtgccggcggttctacaacttcc
tcatggctgacaacatgaagaagatcatcctctcgcacaggcaagctctt
tttggagaccatgttaagaaaccccagagcctggaagacacggacttgag
ccggctctacacagcaacatccctgatgcagattgatgacaatgtgatga
ggaagtttcacggctataactccctgaaggaatactatgaggaagaaagt
tgcatgcggtacctgcacaggatttatgttcctctcatgctggttaatgc
agctgacgatccgttggtgcatgaaagtcttctaaccattccaaaatctc
tttcagagaaacgagagaacgtcatgtttgtgctgcctctgcatgggggc
cacttgggcttctttgagggctctgtgctgttccccgagcccctgacatg
gatggataagctggtggtggagtacgccaacgccatttgccaatgggagc
gtaacaagttgcagtgctctgacacggagcaggtggaggccgacctggag
tgaggcctccggactctggcacgctccagcagccctcctctggaagctgc
gtcccctcaccccctgtttcaggtctcccatctccctcagtgacctggat
ctgacctcacaccatcagcagggggcacccaccatgcacacctgtctcgg
agtaggcagctcttcctgggagctccaggctatttttgtgcttagttact
ggttttctccattgcattgttaggcatggtgacaagtgacagagttcttg
ccctctgtccagtttcagcatctggttgcttttaagccaagtacatctag
tttccctattaaaaatgtgtctgaatccccccgaattc

### Amino Acid Sequence of HS1-2

(SEQ ID NO 26)

MNAMLETPELPAVFDGVKLAAVAAVLYVIVRCLNLKSPTAPPDLYFQDSG
LSRFLLKSCPLLTKEYIPPLIWGKSGHIQTALYGKMGRVRSPHPYGHRKF
ITMSDGATSTFDLFEPLAEHCVGDDITMVICPGIANHSEKQYIRTFVDYA
QKNGYRCAVLNHLGALPNIELTSPRMFTYGCTWEFGAMVNYIKKTYPLTQ
LVVVGFSLGGNIVCKYLGETQANQEKVLCCVSVCQGYSALRAQETFMQWD
QCRRFYNFLMADNMKKIILSHRQALFGDHVKKPQSLEDTDLSRLYTATSL
MQIDDNVMRKFHGYNSLKEYYEEESCMRYLHRIYVPLMLVNAADDPLVHE
SLLTIPKSLSEKRENVMFVLPLHGGHLGFFEGSVLFPEPLTWMDKLVVEY
ANAICQWERNKLQCSDTEQVEADLE

SOSUI and TmPred predict 1 ™.

### AB033007/KIAA1181

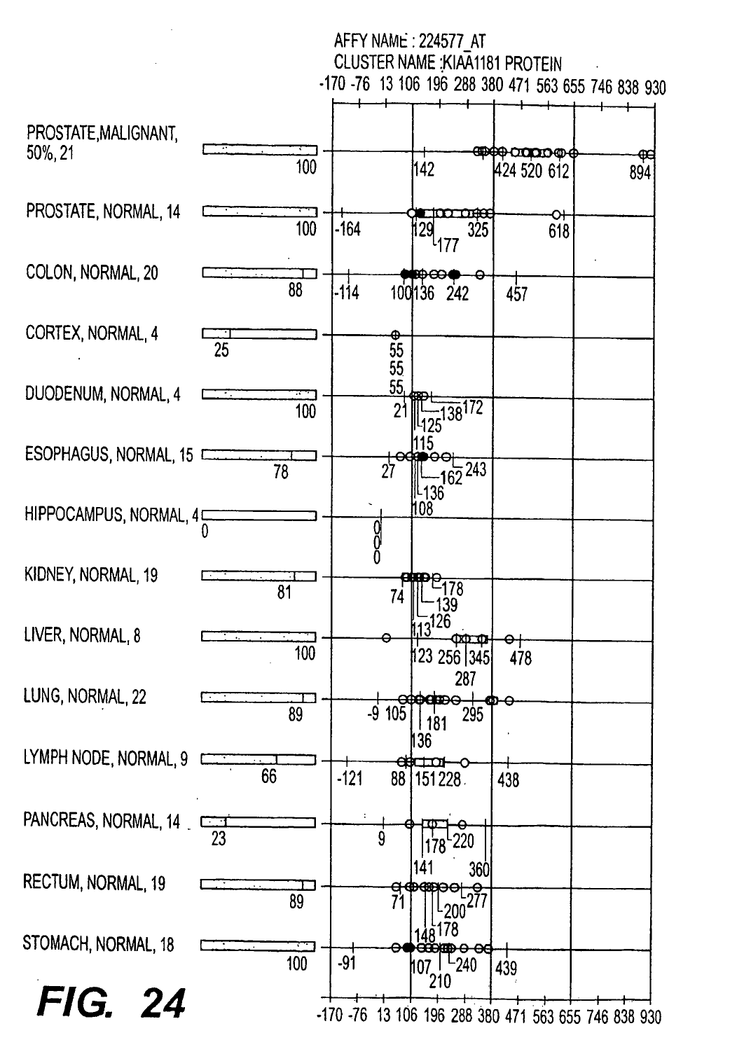

Using GeneLogic database, we found fragment AB033007 was upregulated 4.06 fold in the malignant prostate samples compared to mixed normal tissue without normal prostate and female specific organs. Enorthern analysis of this fragment (FIG. 24) demonstrates that it is expressed in 100% of the prostate tumors with greater than 50% malignant cells with low expression in normal tissues other than the prostate.

### Sequence of AB033007:

(SEQ ID NO 27)

GGAAGTCATCTTTTGAGATCCAGATAGACATGGTTTGTGCACTTACGTCC
AGATGGGAAGCATCCTTCCTGCAACCCTAAAATAATCATGCAGCCTCTCA
GACGGACGCCATCGGTCCCAAGGCCTTAGGTGGAGGAAGCAAAGCAGGCC
AGGCCTGTCCTGTCCGTGGACCTCTACCTTCTGGACTCCCTACGGGTGCA
GAGCACTTGGGTTTCTCTACAGCCATCGTGGCCCACTTGACACTGTGCTC
CTCCATCAGCTGGTCACATGCCAACACGTTCCCAGCCCCTGAGGCAGCTC
CAGGGTGCCCCACCTGCTCCTGAGGTGGGTCCCTACCCTGCTGCTCCTCT
TCATCCTTTCCCTTTTGTCCTGAAAGGGAGGAGCAATGGTCCAGGCATTA
ATTCCACCCAGGGAATTTTAGCTATGCCCTCATGTC

### This Sequence Corresponds to the Hypothetical Gene KIAA1181:

(SEQ ID NO 28)

GGCGAGTGGCGAGTGGCGAGTGTCAGGGGGGCGGCCGGCGGGGGCGGGGC
GGCCGGAGGAGGCGTTGGCAGCGGGCTCGGACCCACGCGGCGCCGCGGCC
CGCCTGGCCTGCAGCGCTCCCACCCCCGGCGGCGGCACGATGCCCTTTGA
CTTCAGGAGGTTTGACATCTACAGGAAGGTGCCCAAGGACCTTACGCAGC
CAACGTACACCGGGGCCATTATCTCCATCTGCTGCTGCCTCTTCATCCTC
TTCCTCTTCCTCTCGGAGCTCACCGGATTTATAACGACAGAAGTTGTGAA
CGAGCTCTATGTCGATGACCCAGACAAGGACAGCGGTGGCAAGATCGACG
TCAGTCTGAACATCAGTTTACCCAATCTGCACTGCGAGTTGGTTGGGCTT
GACATTCAGGATGAGATGGGCAGGCACGAAGTGGGCCACATCGACAACTC
CATGAAGATCCCGCTGAACAATGGGGCAGGCTGCCGCTTCGAGGGGCAGT
TCAGCATCKkCAAGGTCCCCGGCAACTTCCACGTGTCCACACACAGTGCC
ACAGCCCAGCCACAGAACCCAGACATGACGCATGTCATCCACAAGCTCTC
CTTTGGGGACACGCTACAGGTCCAGAACATCCACGGAGCTTTCAATGCTC
TCGGGGGAGCAGACAGACTCACCTCCAACCCCCTGGCCTCCCACGACTAC
ATCCTGAAGATTGTGCCCACGGTTTATGAGGACAAGAGTGGCAAGCAGCG
GTACTCCTACCAGTACACGGTGGCCAACAAGGAATACGTCGCCTACAGCC
ACACGGGCCGCATCATCCCTGCAATCTGGTTCCGCTACGACCTCAGCCCC
ATCACGGTCAAGTACACAGAGAGACGGCAGCCGCTGTACAGATTCATCAC
CACGATCTGTGCCATCATTGGCGGGACCTTCACCGTCGCCGGCATCCTGG
ACTCATGCATCTTCACAGCCTCTGAGGCCTGGAAGAAGATCCAGCTGGGC
AAGATGCATTGACGCCACACCCAGCCTAATGGCCGAGGACCCTGGGCATC
GCCAGCCTTGCCTCCAGTGCCCTGTCTCCTTTGGCCCTCAATCTGGTCCC
AAATCTGGCTGTGTCCCAAAGGGTGTGTGGGAAGTGGGGGGAAAGTAGAG
GATGGCTCGATGTTTTGCAGCTACCTCTTTTCCCCGTGTTTCTTTTTAGA
CAAATTACACTGCCTGAAGTTGCAGTTCCCCTTTCCCTGGGGAGCCCCAA
GAACAGAGTCAGGCAAGGGGTGGGGAGTCCAGGGATCTTGGGGACCCCTC
CTAGGAGAGCTGCAGTCTCTTCCCTCAGGGGAACATCCCAGAATGCATAT
CGATCAGCTCTCAGCCAGGCTTCGACAATCTCGCAGCCCCCACTAGGTGG
ACACATTAATGATTTGGTTTCTCCCCTGGGCAGCCAACCTGCCCCAGAGG
CACCAGACCTGGGCTTTCAGCTTTGGGACCAGGCTGCCCAAAGGTACTCC
TTTATACACCCGGCACCTTCCACGAAAGATGGTACTTCCCAAGCAAGCCC
CTATGATTTGTCACTATAGATGGAACCCTGACTTCTGCCCCATCCCTTCC
TGCCCAACCTAGAACCCAGGCCTCAAGTCTTTACCCCACCCCTTTCTTGT
TCTTCCAAGAAGCAGATGCCCAGTTGCTCAGCAGCAGCGGTAGAGACTTG
AATCTGCCCACCAGTCACAAGGCGGGTCACAGATTCCTCTTCCTCTCTTC
TCCTCGTTCCTCTGAACCCTCCACCAATGTGCCTCAGCCTGTGTGCTGTG
TGGCAACAGCATTCTGGTTCCCACTGCCAAGATCTCCCACCACTCTGCTG
GGATCTGCAGTGGCAGGGAGTGGGGGTTGTGTAAAGGGGAAGTCATCTTT
TGAGATCCAGATAGACATGGTTTGTGCACTTACGTCCAGATGGGAAGCAT
CCTTCCTGCAACCCTAAAATAATCATGCAGCCTCTCAGACGGACGCCATC
GGTCCCAAGGCCTTAGGTGGAGGAAGCAAAGCAGGCCAGGCCTGTCCTGT
CCGTGGACCTCTACCTTCTGGACTCCCTACGGGTGCAGAGCACTTGGGTT
TCTCTACAGCCATCGTGGCCCACTTGACACTGTGCTCCTCCATCAGCTGG
TCACATGCCAACACGTTCCCAGCCCCTGAGGCAGCTCCAGGGTGCCCCAC
CTGCTCCTGAGGTGGGTCCCTACCCTGCTGCTCCTCTTCATCCTTTCCCT
TTTGTCCTGAAAGGGAGGAGCAATGGTCCAGGCATTAATTCCACCCAGGG
AATTTTAGCTATGCCCTCATGTCCCAGGGAGAGAGCCACACGCCTGTTTT
CCATTTATAGCAAGATTGTTTGCATACTTTTGTAATGAAGGGGAGTGTCC
AGTGGAAGGATTTTTAAAATTATCTTATGGAT

### The Amino Acid Sequence of KIAA1181

(SEQ ID NO 29)

ASGEWRVSGGRPAGAGRPEEALAAGSDPRGAAARLACSAPTPGGGTMPFD
FRRFDIYRKVPKDLTQPTYTGAIISICCCLFILFLFLSELTGFITTEVVN
ELYVDDPDKDSGGKIDVSLNISLPNLHCELVGLDIQDEMGRHEVGHIDNS
MKIPLNNGAGCRFEGQFSINKVPGNFHVSTHSATAQPQNPDMTHVIHKLS
FGDTLQVQNIHGAFNALGGADRLTSNPLASHDYILKIVPTVYEDKSGKQR
YSYQYTVANKEYVAYSHTGRIIPAIWFRYDLSPITVKYTERRQPLYRFIT
TICAIIGGTGTVAGILDSCIFTASEAWKKIQLGKMH

This protein is predicted to have 2 TMs by SMART and 1 TM by SOSUI.

### AB033070/KIAA1244

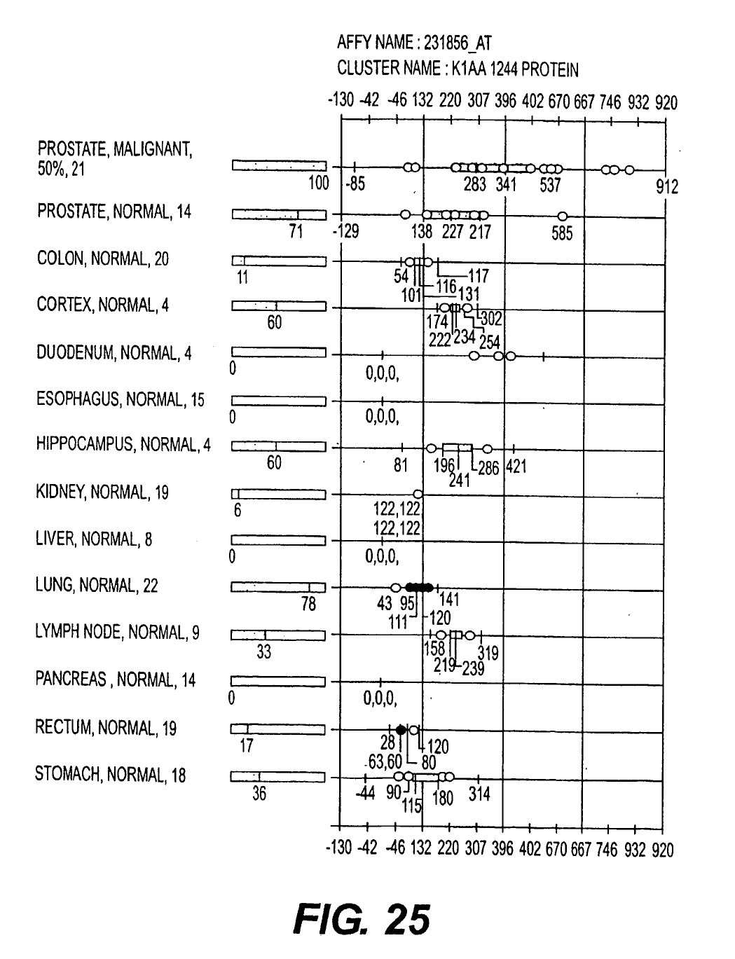

Using the GeneLogic database, we found fragment AB033070 was upregulated 20.47 fold in the malignant prostate samples compared to mixed normal tissue without normal prostate and female specific organs. Enorthern analysis of this fragment (FIG. 25) demonstrates that it is expressed in 100% of the prostate tumors with greater than 50% malignant cells with low expression in normal tissues other than the prostate.

### Nucleotide Sequence of AB033070:

(SEQ ID NO 30)

TGGGATATCAGTGAACTATGTTGTATACTTTTGAATTTTTACATTTTATA
AATGGAATTGAAAGTTGGATAACTGCTTTTTTTAAATTTTCCAACAGAAG
TAACACCACAGTTGCTTTGTTTCTTTTTATAGCTTACCTGAGGTTCAGTT
CTTCTTTGTGAACCTGTGAGTACTCCACAGTTTACTGGGGGAAAAGGCTT
CAGTAAAGCAGAGGCTAGAATTACAGTATTTATACATAGCAACTTTTCAT
AAAGTAGAAAAATTCAAAGGAAGCTGTCTCAATTTGAGAATACCAGCTGG
GCACGGTGGCTCACGCCTGTAATCCCAGCACTTACTTTGGGAGGCCAAGG
TGGGCAGATAACCTGCGGTCA

### This Corresponds to the Nucleic Acid Sequence of the KIAA1244 Gene Below:

(SEQ ID NO 31)

GGCTGCTCCTGCACTGCGCCGGCCCTGAGCGGACCTGTGGCTCGGACTAT
CTATTACATCGCAGCCGAGCTGGTCCGGCTGGTGGGGTCTGTGGACTCCA
TGAAGCCCGTGCTCCAGTCCCTCTACCACCGAGTGCTGCTCTACCCCCCA
CCCCAGCACCGGGTGGAAGCCATCAAAATAATGAAAGAGATACTTGGGAG
CCCACAGCGTCTCTGTGACTTGGCAGGACCCAGCTCCACTGAATCAGAGT
CCAGAAAAAGATCAATTTCAAAAAGAAAGTCTCATCTGGATCTCCTCAAA
CTCATCATGGATGGCATGACCGAAGCATGCATCAAGGGTGGCATCGAAGC
TTGCTATGCAGCCGTGTCCTGTGTCTGCACCTTGCTGGGTGCCCTGGATG
AGCTCAGCCAGGGGAAGGGCTTGAGCGAAGGTCAGGTGCAACTGCTGCTT
CTGCGCCTTGAGGAGCTGAAGGATGGGGCTGAGTGGAGCCGAGATTCCAT
GGAGATCAATGAGGCTGACTTCCGCTGGCAGCGGCGAGTGCTGTCCTCAG
AACACACGCCGTGGGAGTCAGGGAACGAGAGGAGCCTTGACATCAGCATC
AGTGTCACCACAGACACAGGCCAGACCACTCTCGAGGGAGAGTTGGGTCA
GACTACACCCGAGGACCATTCGGGAAACCACAAGAACAGTCTCAAGTCGC
CAGCCATCCCAGAGGGTAAGGAGACGCTGAGCAAAGTATTGGAAACAGAG
GCGGTAGACCAGCCAGATGTCGTGCAGAGAAGCCACACGGTCCCTTACCC
TGACATAACTAACTTCCTGTCAGTAGACTGCAGGACAAGGTCCTATGGAT
CTAGGTATAGTGAGAGCAATTTTAGCGTTGATGACCAAGACCTTTCTAGG
ACAGAGTTTGATTCCTGTGATCAGTACTCTATGGCAGCAGAAAAGGACTC
GGGCAGGTCCGACGTGTCAGACATTGGGTCGGACAACTGTTCACTAGCCG
ATGAAGAGCAGACACCCCGGGACTGCCTAGGCCACCGGTCCCTGCGAACT
GCCGCCCTGTCTCTAAAACTGCTGAAGAACCAGGAGGCGGATCAGCACAG
CGCCAGGCTGTTCATACAGTCCCTGGAAGGCCTCCTCCCTCGGCTCCTGT
CTCTCTCCAATGTAGAGGAGGTGGACACCGCTCTGCAGAACTTTGCCTCT
ACTTTCTGCTCAGGCATGATGCACTCTCCTGGCTTTGACGGGAATAGCAG
CCTCAGCTTCCAGATGCTGATGAACGCAGACAGCCTCTACACAGCTGCAC
ACTGCGCCCTGCTCCTCAACCTGAAGCTCTCCCACGGTGACTACTACAGG
AAGCGGCCGACCCTGGCGCCAGGCGTGATGAAGGACTTCATGAAGCAGGT
GCAGACCAGCGGCGTGCTGATGGTCTTCTCTCAGGCCTGGATTGAGGAGC
TCTACCATCAGGTGCTCGACAGGAACATGCTTGGAGAGGCTGGCTATTGG
GGCAGCCCAGAAGATAACAGCCTTCCCCTCATCACAATGCTGACCGATAT
TGACGGCTTAGAGAGCAGTGCCATTGGTGGCCAGCTGATGGCCTCGGCTG
CTACAGAGTCTCCTTTCGCCCAGAGCAGGAGAATTGATGACTCCACAGTG
GCAGGCGTGGCATTTGCTCGCTATATTCTGGTGGGCTGCTGGAAGAACTT
ATCGATACTTTATCAACCCCACTGACTGGTCGAATGGCGGGGAGCTCCAA
AGGGCTGGCCTTCATTCTGGGAGCTGAAGGCATCAAAGAGCAGAACCAGA
AGGAGCGGGACGCCATCTGCATGAGCCTCGACGGGCTGCGGAAAGCCGCA
CGGCTGAGCTGCGCTCTAGGCGTTGCTGCTAACTGCGCCTCAGCCCTTGC
CCAGATGGCAGCTGCCTCCTGTGTCCAAGAAGAAAAAGAAGAGAGGGAGG
CCCAAGAACCCAGTGATGCCATCACACAAGTGAAACTAAAAGTGGAGCAG
AAACTGGAGCAGATTGGGAAGGTGCAGGGGGTGTGGCTGCACACTGCCCA
CGTCTTGTGCATGGAGGCCATCCTCAGCGTAGGCCTGGAGATGGGAAGCC
ACAACCCGGACTGCTGGCCACACGTGTTCAGGGTGTGTGAATACGTGGGC
ACCCTGGAGCACAACCACTTCAGCGATGGTGCCTCGCAGCCCCCTCTGAC
CATCAGCCAGCCCCAGAAGGCCACTGGAAGCGCTGGCCTCCTTGGGGACC
CCGAGTGTGAGGGCTCGCCCCCCGAGCACAGCCCGGAGCAGGGGCGCTCC
CTGAGCACGGCCCCTGTCGTCCAGCCCCTGTCCATCCAGGACCTCGTCCG
GGAAGGCAGCCGGGGTCGGGCCTCCGACTTCCGCGGCGGGAGCCTCATGA
GCGGGAGCAGCGCGGCCAAGGTGGTGCTCACCCTCTCCACGCAAGCCGAC
AGGCTCTTTGAAGATGCTACGGATAAGTTGAACCTCATGGCCTTGGGAGG
TTTTCTTTACCAGCTGAAGAAAGCATCGCAGTCTCAGCTTTTCCATTCTG
TTACAGATACAGTTGATTACTCTCTGGCAATGCCAGGAGAAGTTAAATCC
ACTCAAGACCGAAAAAGCGCCCTCCACCTGTTCCGCCTGGGGAATGCCAT
GCTGAGGATTGTGCGGAGCAAAGCACGGCCCCTGCTCCACGTGATGCGCT
GCTGGAGCCTTGTGGCCCCACACCTGGTGGAGGCTGCTTGCCATAAGGAA
AGACATGTGTCTCAGAAGGCTGTTTCCTTCATCCATGACATACTGACAGA
AGTCCTCACTGACTGGAATGAGCCACCTCATTTTCACTTCAATGAAGCAC
TCTTCCGACCTTTCGAGCGCATTATGCAGCTGGAATTGTGTGATGAGGAC
GTCCAAGACCAGGTTGTCACATCCATTGGTGAGCTGGTTGAAGTGTGTTC
CACGCAGATCCAGTCGGGATGGAGACCCTTGTTCAGTGCCCTGGAAACAG
TGCATGGCGGGAACAAGTCAGAGATGAAGGAGTACCTGGTTGGTGACTAC
TCCATGGGAAAAGGCCAAGCTCCAGTGTTTGATGTATTTGAAGCTTTTCT
CAATACTGACAACATCCAGGTCTTTGCTAATGCAGCCACTAGCTACATCA
TGTGCCTTATGAAGTTTGTCAAAGGACTGGGGGAGGTGGACTGTAAAGAG
ATTGGAGACTGTGCCCCAGCACCCGGAGCCCCGTCCACAGACCTGTGCCT
CCCGGCCCTGGATTACCTCAGGCGCTGCTCTCAGTTATTGGCCAAAATCT
ACAAAATGCCCTTGAAGCCAATATTCCTTAGTGGGAGACTTGCCGGCTTG
CCTCGAAGACTTCAGGAACAGTCAGCCAGCAGTGAGGATGGAATTGAATC
AGTCCTGTCTGATTTTGATGATGACACCGGTCTGATAGAAGTCTGGATAA
TCCTGCTGGAGCAGCTGACAGCGGCTGTGTCCAATTGTCCACGGCAGCAC
CAACCACCAACTCTGGATTTACTCTTTGAGCTGTTGAGAGATGTGACGAA
AACACCAGGACCAGGGTTTGGTATCTATGCAGTGGTTCACCTCCTCCTTC
CTGTGATGTCCGTTTGGCTCCGCCGGAGCCATAAAGACCATTCCTACTGG
GATATGGCCTCTGCCAATTTCAAGCACGCTATTGGTCTGTCCTGTGAGCT
GGTGGTGGAGCACATTCAAAGCTTTCTACATTCAGATATCAGGTACGAGA
GCATGATCAATACCATGCTGAAGGACCTCTTTGAGTTGCTGGTCGCCTGT
GTGGCCAAGCCCACTGAAACCATCTCCAGAGTGGGCTGCTCCTGTATTAG
ATACGTCCTTGTGACAGCGGGCCCTGTGTTCACTGAGGAGATGTGGAGGC
TTGCCTGCTGTGCCCTGCAAGATGCGTTCTCTGCCACACTCAAGCCAGTG
AAGGACCTGCTGGGCTGCTTCCACAGCGGCACGGAGAGCTTCAGCGGGGA
AGGCTGCCAGGTGCGAGTGGCGGCCCCGTCCTCCTCCCCAAGTGCCGAGG
CCGAGTACTGGCGCATCCGAGCCATGGCCCAGCAGGTGTTTATGCTGGAC
ACCCAGTGCTCACCAAAGACACCAAACAACTTTGACCACGCTCAGTCCTG
CCAGCTCATTATTGAGCTGCCTCCTGATGAAAAACCAATGGACACACCAA
GAAAAGCGTGTCTTTCAGGGAAATTGTGGTGAGCCTGCTGTCTCATCAGG
TGTTACTCCAGAACTTATATGACATCTTGTTAGAAGAGTTTGTCAAAGGC
CCCTCTCCTGGAGAGGAAAAGACGATACAAGTGCCAGAAGCCAAGCTGGC
TGGCTTCCTCAGATACATCTCTATGCAGAACTTGGCAGTCATATTCGACC
TGCTGCTGGACTCTTATAGGACTGCCAGGGAGTTTGACACCAGCCCCGGG
CTGAAGTGCCTGCTGAAGAAAGTGTCTGGCATCGGGGGCGCCGCCAACCT
CTACCGCCAGTCTGCGATGAGCTTTAACATTTATTTCCACGCCCTGGTGT
GTGCTGTTCTCACCAATCAAGAAACCATCACGGCCGAGCAAGTGAAGAAG
GTCCTTTTTGAGGACGACGAGAGAAGCACGGATTCTTCCCAGCAGTGTTC
ATCTGAGGATGAAGACATCTTTGAGGAAACCGCCCAGGTCAGCCCCCCGA
GAGGCAAGGAGAAGAGACAGTGGCGGGCACGGATGCCCTTGCTCAGCGTC
CAGCCTGTCAGCAACGCAGATTGGGTGTGGCTGGTCAAGAGGCTGCACAA
GCTGTGCATGGAACTGTGCAACAACTACATCCAGATGCACTTGGACCTGG
AGAACTGTATGGAGGAGCCTCCCATCTTCAAGGGCGACCCGTTCTTCATC
CTGCCCTCCTTCCAGTCCGAGTCATCCACCCCATCCACCGGGGGCTTCTC
TGGGAAAGAAACCCCTTCCGAGGATGACAGAAGCCAGTCCCGGGAGCACA
TGGGCGAGTCCCTGAGCCTGAAGGCCGGTGGTGGGGACCTGCTGCTGCCC
CCCAGCCCCAAAGTGGAGAAGAAGGATCCCAGCCGGAAGAAGGAGTGGTG
GGAGAATGCGGGGAACAAAATCTACACCATGGCAGCCGACAAGACCATTT
CAAAGTTGATGACCGAATACAAAAAGAGGAAACAGCAGCACAACCTGTCC
GCGTTCCCCAAAGAGGTCAAAGTGGAGAAGAAAGGAGAGCCACTGGGTCC
CAGGGGCCAGGACTCCCCGCTGCTTCAGCGTCCCCAGCACTTGATGGACC
AAGGGCAAATGCGGCATTCCTTCAGCGCAGGCCCCGAGCTGCTGCGACAG
GACAAGAGGCCCCGCTCAGGCTCCACCGGGAGCTCCCTCAGTGTCTCGGT
GAGAGACGCAGAAGCACAGATCCAGGCATGGACCAACATGGTGCTAACAG
TTCTCAATCAGATTCAGATTCTCCCAGACCAGACCTTCACGGCCCTCCAG
CCCGCAGTGTTCCCGTGCATCAGTCAGCTGACCTGTCACGTGACCGACAT
CAGAGTTCGCCAGGCTGTGAGGGAGTGGCTGGGCAGGGTGGGCCGTGTCT
ATGACATCATTGTGTAGCCGACTCCTGTTCTACTCTCCCACCAAATAACA
GTAGTGAGGGTTAGAGTCCTGCCAATACAGCTGTTGCATTTTCCCCACCA
CTAGCCCCACTTAAACTACTACTACTGTCTCAGAGAACAGTGTTTCCTAA
TGTAAAAAGCCTTTCCAACCACTGATCAGCATTGGGGCCATACTAAGGTT
TGTATCTAGATGACACAAACGATATTCTGATTTTGCACATTATTATAGAA
GAATCTATAATCCTTGATATGTTTCTAACTCTTGAAGTATATTTCCCAGT
GCTTTTGCTTACAGTGTTGTCCCCAAATGGGTCATTTTCAAGGATTACTC
ATTTGAAAACACTATATTGATCCATTTGATCCATCATTTAAAAAATAAAT
ACAATTCCTAAGGCAATATCTGCTGGTAAGTCAAGCTGATAAACACTCAG
ACATCTAGTACCAGGGATTATTAATTGGAGGAAGATTTATGGTTATGGGT
CTGGCTGGGAAGAAGACAACTATAAATACATATTCTTGGGTGTCATAATC
AAGAAAGAGGTGACTTCTGTTGTAAAATAATCCAGAACACTTCAAAATTA
TTCCTAAATCATTAAGATTTTCAGGTATTCACCAATTTCCCCATGTAAGG
TACTGTGTTGTACCTTTATTTCTGTATTTCTAAAAGAAGAAAGTTCTTTC
CTAGCAGGGTTTGAAGTCTGTGGCTTATCAGCCTGTGACACAGAGTACCC
AGTGAAAGTGGCTGGTACGTAGATTGTCAAGAGACATAAGACCGACCAGC
CACCCTGGCTGTTCTTGTGGTGTTTGTTTCCATCCCCAAGGCAAACAAGG
AAAGGAAAGGAAAGAAGAAAAGGTGCCTTAGTCCTTTGTTGCACTTCCAT
TTCCATGCCCCACAATTGTCTGAACATAAGGTATAGCATTTGGTTTTTAA
GAAAACAAAACATTAAGACGCAACTCATTTTATATCAACACGCTTGGAGG
AAAGGGACTCAGGGAAGGGAGCAGGGAGTGTGGGGTGGGGATGGATTATG
ATGAAATCATTTTCAATCTTAAAATATAATACAACAATCTTGCAAAATTA
TGGTGTCAGTTACACAAGCTCTAGTCTCAAAATGAAAGTAATGGAGAAAG
ACACTGAAATTTAGAAAATTTTGTCGATTTAAAATATTTCTCCTATCTAC
CAAGTAAAGTTACCCTATGTTTGATGTCTTTGCATTCAGACCAATATTTC
AGGTGGATATTTCTAAGTATTACTAGAAAATACGTTTGAAAGCTTTATCT
TATTATTTACAGTATTTTTATATTTCTTACATTATCCTAATGATTGAAAA
CTCCTCAATCAAGCTTACTTACACACATTCTACAGAGTTATTTAAGGCAT
ACATTATAATCTCCCAGCCCCATTCATAATGAATAAGTCACCCTTTAAAT
ATAAGACACAAATTCTACAGTATTGAAATAAGGATTTAAAGGGGTATTTG
TAAACTTTGCCCTCCTTGAGAAATATGGAACTACCTTAGAGGTTAAGAGG
AAGGCAGTGTTCTGACTTCTTTAGGTGATCTGAAAAAAACACCCTTATCA
TCCAGTGTACCATCTAGAGATCACCACAGAATCCATTTTTTTCCCAGTTC
CACAAAACACTCTGTTTGCCTTCAGTTTTTACTCACTAGACAATAATTCA
AGTTTAGAAACAGGTAATCAGCTATTTGATCTTAAAAGGCAATGAATTGT
TGGGATATCAGTGAACTATGTTGTATACTTTTGAATTTTTACATTTTATA
AATGGAATTGAAAGTTGGATAACTGCTTTTTTTAAATTTTCCAACAGAAG
TAACACCACAGTTGCTTTGTTTCTTTTTATAGCTTACCTGAGGTTCAGTT
CTTCTTTGTGAACCTGTGAGTACTCCACAGTTTACTGGGGGAAAAGGCTT
CAGTAAAGCAGAGGCTAGAATTACAGTATTTATACATAGCAACTTTTCAT
AAAGTAGAAAAATTCAAAGGAAGCTGTCTCAATTTGAGAATACCAGCTGG
GCACGGTGGCTCACGCCTGTAATCCCAGCACTTACTTTGGGAGGCCAAGG
TGGGCAGATAACCTGCGGTCAGGAGTTTGAGACCAGGCTGGACAACATGG
TGAAACCTCGTCTCTACTAAAAATACAAAAATTAGCCAGGTGTGGTAGGA
TGCACCTGTAATCCCAGCTACTTAGGAGGCCGAGACAGGAGAATCGCTCG
AACCCAGGAGGCGGACGTTGCAGTGAGCCAAGATTGCACCATTGCACTCC
AGACTGGGTGACAAGAGTGAAACTCCATCT

### KIAA1244 Amino Acid Sequence:

(SEQ ID NO 32)

GCSCTAPALSGPVARTIYYIAAELVRLVGSVDSMKPVLQSLYHRVLLYPP
PQHRVEAIKIMKEILGSPQRLCDLAGPSSTESESRKRSISKRKSHLDLLK
LIMDGMTEACIKGGIEACYAAVSCVCTLLGALDELSQGKGLSEGQVQLLL
LRLEELKDGAEWSRDSMEINEADFRWQRRVLSSEHTPWESGNERSLDISI
SVTTDTGQTTLEGELGQTTPEDHSGNHKNSLKSPAIPEGKETLSKVLETE
AVDQPDVVQRSHTVPYPDITNFLSVDCRTRSYGSRYSESNFSVDDQDLSR
TEFDSCDQYSMAAEKDSGRSDVSDIGSDNCSLADEEQTPRDCLGHRSLRT
AALSLKLLKNQEADQHSARLFIQSLEGLLPRLLSLSNVEEVDTALQNFAS
TFCSGMMHSPGFDGNSSLSFQMLMNADSLYTAAHCALLLNLKLSHGDYYR
KRPTLAPGVMKDFMKQVQTSGVLMVFSQAWIEELYHQVLDRNMLGEAGYW
GSPEDNSLPLITMLTDIDGLESSAIGGQLMASAATESPFAQSRRIDDSTV
AGVAFARYILVGCWKNLIDTLSTPLTGRMAGSSKGLAFILGAEGIKEQNQ
KERDAICMSLDGLRKAARLSCALGVAANCASALAQMAAASCVQEEKEERE
AQEPSDAITQVKLKVEQKLEQIGKVQGVWLHTAHVLCMEAILSVGLEMGS
HNPDCWPHVFRVCEYVGTLEHNHFSDGASQPPLTISQPQKATGSAGLLGD
PECEGSPPEHSPEQGRSLSTAPVVQPLSIQDLVREGSRGRASDFRGGSLM
SGSSAAKVVLTLSTQADRLFEDATDKLNLMALGGFLYQLKKASQSQLFHS
VTDTVDYSLAMPGEVKSTQDRKSALHLFRLGNAMLRIVRSKARPLLHVMR
CWSLVAPHLVEAACHKERHVSQKAVSFIHDILTEVLTDWNEPPHFHFNEA
LFRPFERIMQLELCDEDVQDQVVTSIGELVEVCSTQIQSGWRPLFSALET
VHGGNKSEMKEYLVGDYSMGKGQAPVFDVFEAFLNTDNIQVFANAATSYI
MCLMKFVKGLGEVDCKEIGDCAPAPGAPSTDLCLPALDYLRRCSQLLAKI
YKMPLKPIFLSGRLAGLPRRLQEQSASSEDGIESVLSDFDDDTGLEIVWI
ILLEQLTAAVSNCPRQHQPPTLDLLFELLRDVTKTPGPGFGIYAVVHLLL
PVMSVWLRRSHKDHSYWDMASANFKHAIGLSCELVVEHIQSFLHSDIRYE
SMINTMLKDLFELLVACVAKPTETISRVGCSCIRYVLVTAGPVFTEEMAM
AQQVFMLDTQCSPKTPNNFDHAQSCQLIIELPPDEKPNGHTKKSVSFREI
VVSLLSHQVLLQNLYDILLEEFVKGPSPGEEKTIQVPEAKLAGFLRYISM
QNLAVIFDLLLDSYRTAREFDTSPGLKCLLKKVSGIGGAANLYRQSAMSF
NIYFHALVCAVLTNQETITAEQVKKVLFEDDERSTDSSQQCSSEDEDIFE
ETAQVSPPRGKEKRQWRARMPLLSVQPVSNADWVWLVKRLHKLVMELVNN
YIQMHLDLENCMEEPPIFKGDPFFILPSFQSESSTPSTGGFSGKETPSED
DRSQSREHMGESLSLKAGGGDLLLPPSPKVEKKDPSRKKEWWENAGNKIY
TMAADKTISKLMTEYKKRKQQHNLSAFPKEVKVEKKGEPLGPRGQDSPLL
QRPQHLMDQGQMRSHFSAGPELLRQDKRPRSGSTGSSLSVSVRDAEAQIQ
AWTNMVLTVLNQIQILPDQTFTALQPAVFPCISQLTCHVTDIRVRQAVRE
WLGRVGRVYDIIV

This sequence has no TMs by SMART, but appears to have 2 when analyzed by SOSUI and 4 by TmPred.

### AB037765/KIAA1344

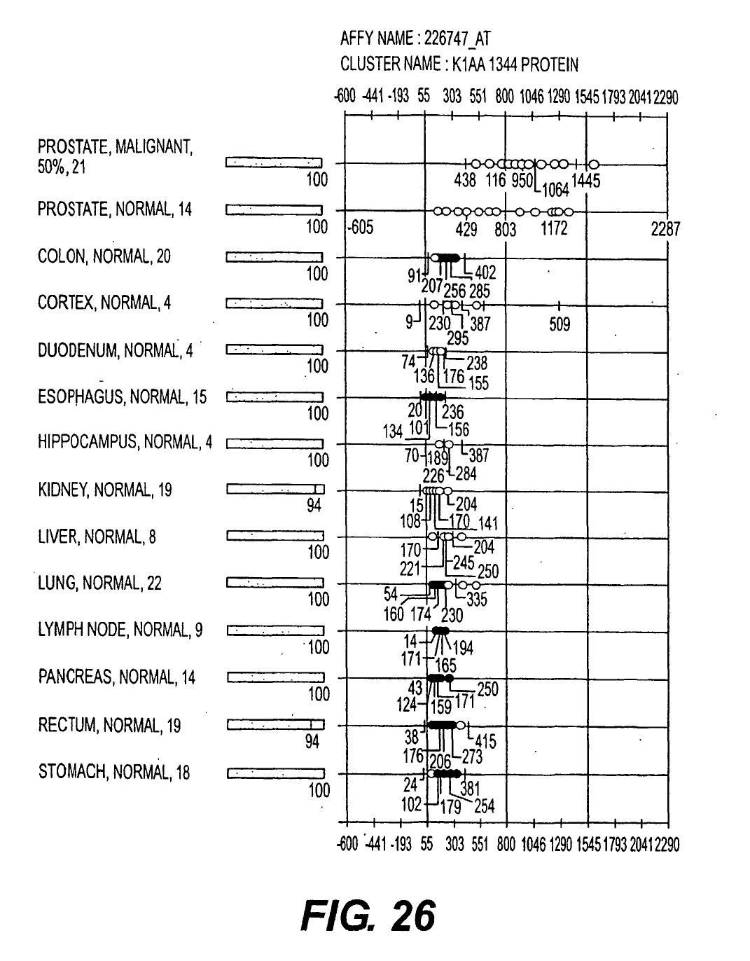

Using the GeneLogic database, we found fragment AB037765 was upregulated 5.15 fold in the malignant prostate samples compared to mixed normal tissue without normal prostate and female specific organs. Enorthern analysis of this fragment (FIG. 26) demonstrates that it is expressed in 100% of the prostate tumors with greater than 50% malignant cells with low expression in normal tissues other than the prostate.

### Sequence of AB037765:

(SEQ ID NO 33)

AATTTTCATTCCAAATCACTTAGCTGTTAGACTGATCTGTTTGTAGCAGT
TGTTTGTCTCATTTTTGCTCTGTGCATTTTTTGAGACATTTGTTGAGAAT
ATTCTATTTGGTGCTCTACTGTATTTTTCTTTTTAATATCTACTTGATAT
CTTGTTCTTTAAATTTTCTTCACATATGGTTTGCCTGATACAACTGATTT
TTATAACTGAAATTTAAGGAATCTAACAGCTAAAACTCAGTAAGTGCATN
TATTTCCTTATAACATAGACCCGTTGCTACTCTCAGCACCCTCTCCTCAA
TTTTTTTTCCTGTAGCATGTGATGCCTGATTAAACTCATTTTCATTTGCT
TTTATTTCTAATATGGGAACAATGAGAGTGAACTCTAAATATAGGTTGTA
GTAATAAAACATCATTAGCCTAATTATTAGAAAATGCTAATTAAGTACCA
GCACATAGAAACATGAAATTGCTTAGTCATTGTACCTTT

### This Corresponds to the Nucleic Acid Sequence of the KIAA1344 Gene:

(SEQ ID NO 34)

CGGCTGCAGGCTGGGAGGGAGAAGTGCTACGCCTTTGCAGGTTGGCGAAG
TGGTTCCAGGCTACCCGGCTAGTCTGGCACGGCCCCGTCTTCTGCCTCCT
CCTCCGTCGCGTGGCGGCGGGAACTGTTGGCCGCGCGGCCTCGGGAACGG
CCCAGGTCCCCGCCCGCAGGTCCCGGGCAGATAACATAGATCATCAGTAG
AAAACTTCTTGAAGTTGTTCAAGAAAAATTTGAAAGTAGCAAAATAGAAA
ATAAAGAATTAACAGCAGATACAGAGGACAGCATGGAAGTGTTGTCTTAG
GAAACAGAACACAGCAGTGAAAAAACAGACAAAATCCGCTCAGATACAAC
TGCAGCTGATAATGTTTTCCGGCTTCAATGTCTTTAGAGTTGGGATCTCT
TTTGTCATAATGTGCATTTTTTACATGCCAACAGTAAACTCTTTACCAGA
ACTGAGTCCTCAGAAATATTTTAGTACATTGCAACCAGGAAAAGCCTCTT
TAGCTTATTTTTGTCAAGCTGATTCCCCAAGAACATCTGTATTTCTTGAA
GAACTGAATGAGGCTGTTAGACCTCTGCAGGACTATGGAATTTCAGTTGC
CAAGGTTAATTGTGTCAAAGAAGAAATATCAAGATACTGTGGAAAAGAAA
AGGATTTGATGAAAGCATATTTATTCAAGGGCAACATATTGCTCAGAGAA
TTCCCTACTGACACCTTGTTTGATGTGAATGCCATTGTCGCCCATGTTCT
CTTTGCTCTTCTTTTTAGTGAAGTGAAATATATTACCAACCTGGAAGACC
TTCAGAACATAGAAAATGCTCTGAAAGGAAAAGCAAATATTATATTCTCA
TATGTAAGAGCCATTGGAATACCAGAGCACAGAGCAGTCATGGAAGCCGG
TTTTGTGTATGGGACTACATACCAATTTGTCTTAACCACAGAAATTGCCC
TTTTGGAAAGTATTGGCTCTGAGGATGTGGAATATGCACATCTCTACTTT
TTTCATTGTAAACTAGTCTTGGACTTGACCCAGCAATGTAGAAGAACACT
AATGGAACAGCCATTGACTACACTGAACATTCACCTGTTTATTAAGACAA
TGAAAGCACCTCTGTTGACTGAAGTTGCTGAAGATCCTCAACAAGTTTCA
ACTGTCCATCTCCAACTGGGCTTACCACTGGTTTTTATTGTTAGCCAACA
GGCTACTTATGAAGCTGATAGAAGAACTGCAGAATGGGTTGCTTGGCGTC
TTCTGGGAAAAGCAGGAGTTCTACTCTTGTTAAGGGACTCTTTGGAAGTG
AACATTCCTCAAGATGCTAATGTGGTCTTCAAAAGAGCAGAAGAGGGAGT
TCCAGTGGAATTTTTGGTATTACATGATGTTGATTTAATAATATCTCATG
TGGAAAATAATATGCACATTGAGGAAATACAAGAAGATGAAGACAATGAC
ATGGAAGGTCCAGATATAGATGTTCAGGATGATGAAGTGGCAGAAACTGT
TTTCAGAGATAGGAAGAGAAAATTACCTTTGGAACTTACAGTGGAACTAA
CAGAAGAAACATTTAATGCAACAGTGATGGCTTCTGACAGCATAGTACTC
TTCTATGCTGGTTGGCAAGCAGTATCCATGGCATTTTTGCAATCCTATAT
TGATGTGGCAGTTAAACTGAAAGGCACATCTACTATGCTTCTTACTAGAA
TAAACTGTGCAGATTGGTCTGATGTATGTACTAAGCAAAATGTTACTGAA
TTTCCTATCATAAAGATGTACAAGAAAGGCGAGAACCCAGTATCTTATGC
TGGAATGTTAGGAACCAAAGATCTCCTAAAATTTATCCAGCTCAACAGGA
TTTCATATCCAGTGAATATAACATCGATCCAAGAAGCAGAAGAATATTTA
AGTGGGGAATTATATAAAGACCTCATCTTGTATTCTAGTGTGTCAGTATT
GGGACTATTTAGTCCAACCATGAAAACAGCAAAAGAAGATTTTAGTGAAG
CAGGAAACTACCTAAAAGGATATGTTATCACTGGAATTTATTCTGAAGAA
GATGTTTTGCTACTGTCAACCAAATATGCTGCAAGTCTTCCAGCCCTGCT
GCTTGCCAGACACACAGAAGGCAAAATAGAGAGCATCCCACTAGCTAGCA
CACATGCACAAGACATAGTTCAAATAATAACAGATGCACTACTGGAAATG
TTTCCGGAAATCACTGTGGAAAATCTTCCCAGTTATTTCAGACTTCAGAA
ACCATTATTGATTTTGTTCAGTGATGGCACTGTAAATCCTCAATATAAAA
AAGCAATATTGACACTGGTAAAGCAGAAATACTTGGATTCATTTACTCCA
TGCTGGTTAAATCTAAAGAATACTCCAGTGGGGAGAGGAATCTTGCGGGC
ATATTTTGATCCTCTGCCTCCCCTTCCTCTTCTTGTTTTGGTGAATCTGC
ATTCAGGTGGCCAAGTATTTGCATTTCCTTCAGACCAGGCTATAATTGAA
GAAAACCTTGTATTGTGGCTGAAGAAATTAGAAGCAGGACTAGAAAATCA
TATCACAATTTTACCTGCTCAAGAATGGAAACCTCCTCTTCCAGCTTATG
ATTTTCTAAGTATGATAGATGCCGCAACATCTCAACGTGGCACTAGGAAA
GTTCCCAAGTGTATGAAAGAAACAGATGTGCAGGAGAATGATAAGGAACA
ACATGAAGATAAATCGGCAGTCAGAAAAGAACCGATTGAAACTCTGAGAA
TAAAGCATTGGAATAGAAGTAATTGGTTTAAAGAAGCAGAAAAATCATTT
AGACGTGATAAAGAGTTAGGATGCTCAAAAGTGAACTAATTTTATAGGGC
TGTGGTTTCCAAAATTTTTTTGGCATGATAGACTTAATTTATTTCCTTAA
AGAATAATATTAAATCATTTCAAGTTTGCAGACTAGTGCCATCCAATAGA
ATTATAATATAAGTCACATATTTTATTTAAAATTTTCTAGTAACTACATT
AAACAAAGTAAAAGTGAGCAGGGCAAAATAATTTTGATATTACTTTTCAC
CCAGTAGTATACCCAAAATAGCGAAATATAGAAATTATTAATGAGATATT
TTACATCCTTTTTTGTACCAAGTCTTCTAAATGCAGTACATATTTTATAC
TTACTGCATTTCTTACTTCCGAGTAGCCATATTTCAAGTGTTCATTGCCA
CATGTGGCCTGTGACTACTGTATTGGACAGTTCAGTACTAGACAAAAACT
AGCATAATTAACTTAGTTCTAGCCATGATTTCTATTTGGATTAAAATTAA
ACTCTAATCACAGTTAACTCCACAGTGCATTCATGCAGCTGACAGTTATA
TTTGTTTTATTGGAGTCATGATATTAAAATCAGCGTTTGTCAACCTCAGG
GGATATTTAGCAATTGTCGGGAGACATTTTTGATGTCATGACTAGGGCAG
TTATTGACATTTAGTGAGTAGAGGCCATGGATCCTGCTAAATAACCTGCA
TTGGACAGCGCCCCACAACAAAGAATTATCCTGCCCGAAATGGTAGTCGT
GCCAAGGCTGAGTAACCTTGTGTTAAAAGTAACCTGTGGCAGACTAGGTT
TCCAGAATTTCCTGGTTCTGCTCACGTATCATGTTTGAAAAAATTTTGGC
TATTAAAGATATGTATTAGATGGTCTTATCCTGATTATTACCTGGATACA
ACTTGATCTTTTCTAATATTTTCAGAAAGTGATGGGATAACCCTAGAAGA
GGACTCAGAATGATATTTATATTTTAAGTGAGTCTTAAAACCTCCTCTTA
TTTCTACAAGTTATATGGCTAAATTTCAGATTGAACAGGGATTCAGCATT
CTGCCATCTCCTCATGGAAAGAGAGGCTCCCTCATCTGAAGCGTCTCTGA
AATCTACCCTTGCAAGCTTCAGACAAATCAGTTGATCTCCCTGAGCCACA
CGGCCTCATTCTGTGAGGGAGGGAAAGATTAGCCAAAGAGTTAATTTTCA
TTCCAAATCACTTAGCTGTTAGACTGATCTGTTTGTAGCAGTTGTTTGTC
TCATTTTTGCTCTGTGCATTTTTTGAGACATTTGTTGAGAATATTCTATT
TGGTGCTCTACTGTATTTTTCTTTTTAATATCTACTTGATATCTTGTTCT
TTAAATTTTCTTCACATATGGTTTGCCTGATACAACTGATTTTTATAACT
GAAATTTAAGGAATCTAACAGCTAAAACTCAGTAAGTGCATCTATTTCCT
TATAACATAGACCCGTTGCTACTCTCAGCACCCTCTCCTCAATTTTTTTT
CCTGTAGCATGTGATGCCTGATTAAACTCATTTTCATTTGCTTTTATTTC
TAATATGGGAACAATGAGAGTGAACTCTAAATATAGGTTGTAGTAATAAA
ACATCATTAGCCTAATTATTAGAAAATGCTAATTAAGTACCAGCACATAG
AAACATGAAATTGCTTAGTCATTGTACCTTTGTCAGCAATTTTGACAGTC
ATTAATGTTTGTCATAATTTTAAATAAAGTGTCTGGGTTTCAGAATACCT
TC

### Amino Acid Sequence of KIAA1344

(SEQ ID NO 35)

QQIQRTAWKCCLRKQNTAVKKQTKSAQIQLQLIMFSGFNVFRVGISFVIM
CIFYMPTVNSLPELSPQKYFSTLQPGKASLAYFCQADSPRTSVFLEELNE
AVRPLQDYGISVAKVNCVKEEISRYCGKEKDLMKAYLFKGNILLREFPTD
TLFDVNAIVAHVLFALLFSEVKYITNLEDLQNIENALKGKANIIFSYVRA
IGIPEHRAVMEAGFVYGTTYQFVLTTEIALLESIGSEDVEYAHLYFFHCK
LVLDLTQQCRRTLMEQPLTTLNIHLFIKTMKAPLLTEVAEDPQQVSTVHL
QLGLPLVFIVSQQATYEADRRTAEWVAWRLLGKAGVLLLLRDSLEVNIPQ
DANVVFKRAEEGVPVEFLVLHDVDLIISHVENNMHIEEIQEDEDNDMEGP
DIDVQDDEVAETVFRDRKRKLPLELTVELTEETFNATVMASDSIVLFYAG
WQAVSMAFLQSYIDVAVKLKGTSTMLLTRINCADWSDVCTKQNVTEFPII
KMYKKGENPVSYAGMLGTKDLLFKIQLNRISYPVNITSIQEAEEYLSGEL
YKDLILYSSVSVLGLFSPTMKTAKEDFSEAGNYLKGYVITGIYSEEDVLL
LSTKYAASLPALLLARHTEGKIESIPLASTHAQDIVQIITDALLEMFPEI
TVENLPSYFRLQKPLLILFSDGTVNPQYKKAILTLVKQKYLDSFTPCWLN
LKNTPVGRGILRAYFDPLPPLPLLVLVNLHSGGQVFAFPSDQAIIEENLV
LWLKKLEAGLENHITILPAQEWKPPLPAYDFLSMIDAATSQRGTRKVPKC
MKETDVQENDKEQHEDKSAVRKEPIETLRIKHWNRSNWFKEAEKSFRRDK
ELGCSKVN

SOSUI™ predicts 2 TM domains and SMART™ predicts 1 TM domain.

### AI742872/Hs6_{—}25897_{—}28_{—}16_{—}1426.a

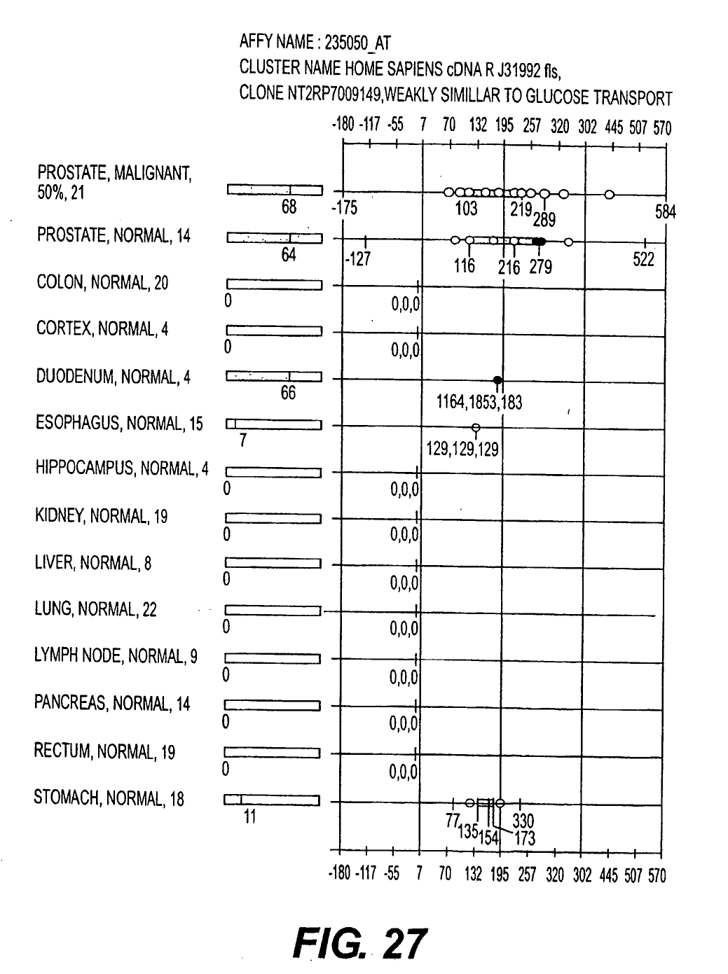

Using GeneLogic database, we found fragment AI742872 was upregulated 10.10 fold in the malignant prostate samples compared to mixed normal tissue without normal prostate and female specific organs. Enorthern analysis of this fragment (FIG. 27) demonstrates that it is expressed in 85% of the prostate tumors with greater than 50% malignant cells with low expression in normal tissues other than the prostate and dudodenum.

### Sequence of AI742872

(SEQ ID NO 36)

GTCAGGCCATTAGGTTATTTATCCAAATCTCTAAGCAATTAGGTTGAAGT
TATTAAGTCAAGCCTAGAAAAGCTGCCTCCTTGTAAGGCTTTCATGACAA
TGTATAGTAATCCACAGTGTCCAATTCTTCACACTCCTCAGGAATATCAC
TACCTCAGGTTACGGTACACAGGCTATAATTGATGATGATGTTCAGATAA
CTGAAGACACAATAAATGACATTCAGACATCANNANAANNNCCTCATGTT
CTTTTCTATGATGGCCACCTGTACCAGCAACGTGGGTTTCACCCACACAA
CGATGAACT

This Corresponds to the Hypothetical Gene Hs6_{—}25897_{—}28_{—}16_{—}1426. There are Predicted to be Alternatively Spliced Forms of this Gene, the Longest is the Form Shown Below:

(SEQ ID NO 37)

ACGGTTCTTATAGTGGGACGCATTGCCATAGGGGTCTCCATCTCCCTCTC
TTCCATTGCCACTTGTGTTTACATCGCAGAGATTGCTCCTCAACACAGAA
GAGGCCTTCTTGTGTCACTGAATGAGCTGATGATTGTCATCGGCATTCTT
TCTGCCTATATTTCAAATTACGCATTTGCCAATGTTTTCCATGGCTGGAA
GTACATGTTTGGTCTTGTGATTCCCTTGGGAGTTTTGCAAGCAATTGCAA
TGTATTTTCTTCCTCCAAGCCCTCGGTTTCTGGTGATGAAAGGACAAGAG
GGAGCTGCTAGCAAGGTTCTTGGAAGGTTAAGAGCACTCTCAGATACAAC
TGAGGAACTCACTGTGATCAAATCCTCCCTGAAAGATGAATATCAGTACA
GTTTTTGGGATCTGTTTCGTTCAAAAGACAACATGCGGACCCGAATAATG
ATAGGACTAACACTAGTATTTTTTGTACAAATCACTGGCCAACCAAACAT
ATTGTTCTATGCATCAACTGTTTTGAAGTCAGTTGGATTTCAAAGCAATG
AGGCAGCTAGCCTCGCCTCCACTGGGGTTGGAGTCGTCAAGGTCATTAGC
ACCATCCCTGCCACTCTTCTTGTAGACCATGTCGGCAGCAAAACATTCCT
CTGCATTGGCTCCTCTGTGATGGCAGCTTCGTTGGTGACCATGGGCATCG
TAAATCTCAACATCCACATGAACTTCACCCATATCTGCAGAAGCCACAAT
TCTATCAACCAGTCCTTGGATGAGTCTGTGATTTATGGACCAGGAAACCT
GTCAACCAACAACAATACTCTCAGAGACCACTTCAAAGGGATTTCTTCCC
ATAGCAGAAGCTCACTCATGCCCCTGAGAAATGATGTGGATAAGAGAGGG
GAGACGACCTCAGCATCCTTGCTAAATGCTGGATTAAGCCACACTGAATA
CCAGATAGTCACAGACCCTGGGGACGTCCCAGCTTTTTTGAAATGGCTGT
CCTTAGCCAGCTTGCTTGTTTATGTTGCTGCTTTTTCAATTGGTCTAGGA
CCAATGCCCTGGCTGGTGCTCAGCGAGATCTTTCCTGGTGGGATCAGAGG
ACGAGCCATGGCTTTAACTTCTAGCATGAACTGGGGCATCAATCTCCTCA
TCTCGCTGACATTTTTGACTGTAACTGATCTTATTGGCCTGCCATGGGTG
TGCTTTATATATACAATCATGAGTCTAGCATCCCTGCTTTTTGTTGTTAT
GTTTATACCTGAGACAAAGGGATGCTCTTTGGAACAAATATCAATGGAGC
TAGCCAAAGGTGAACTATGTGAAAAACAACATTTGTTTTATGAGTCATCA
CCAAGAAGAATTAGTGCCAAAACAGCCTCAAAAAAGAAAACCCCAGGAGC
AGCTCTTGGAGTGTAAcaagctgtgtggtaggggccaatccaggcagctt
tctccagagacctaatggcctcaacaccttctgaacgtggatagtgccag
aacacttaggagggtgtctttggaccaatgcatagttgcgactcctgtgc
tctcttttcagtgtcatggaactggttttgaagagacactctgaaatgat
aaagacagcctttaatccccctcctccccagaaggaacctcaaaaggtag
atgaggtacaaggtcctaagtgatctctttttctgagcaggatatcaggt
taaaaaaaaaaagttactggctggtttaatactttctaccttcttcacag
agcagcctttgaatagactatgtcctagtgaagacatcaacctccgcctt
aagctatgtatgtatggaggccagtcgcagctttattatgcagacacaca
agtggtctggacatgagggtacagtttctgcctaccaagacactacttgc
actggatcttacgcaaaaaagaaccagaacacacagtgtggacaactgcc
catatattctatctagattaggagagggtcctggctaggattttagtggt
aattcctagttacattcaacaagtataaagattatagagcttattttatg
aactataaactataatttaatgcaaaatatccttttatgaatttcatgtt
aatattgtgaaatattaaaataattccacaatagttgagaaaaatgagca
tttttttccatttttaaaaaatgcatagaaaagacaattttaaaatcctg
ggaccatatttatttagaagtagctgttagtaaaacattagaaaaggagt
caggccattaggttatttatccaaatctctaagcaattaggttgaagtta
ttaagtcaagcctagaaaagctgcctccttgtaaggctttcatgacaatg
tatagtaatccacagtgtccaattcttcacactcctcaggaatatcacta
cctcaggttacggtacacaggctataattgatgatgatgttcagataact
gaagacacaataaatgacattcagacatcaggacaattccctcatgttct
tttctatgatggccacctgtaccagcaacgtgggtttcacccacacaacg
atgaactgttctcttacttctccagttgattttaaagacttgttaagagg
tcttactaataaaatttgggtatgatagaaaatccacaatcaaatcttga
accaaataacatattaaattactaatatttaagtgatggaagacacacaa
aaaacttaaaagcacgaacaacctaacttgaaaaagaattttaaaatatg
attaacctgaagaaaagagaatcctaagagccaaagctcctttttattta
gcttggaattttcctattggttcctaacaaactgtcccaatgtcatataa
ggaaacatgatctattacattcctttataacaatgtggagagactataaa
cctatgtaagtagtaaaactatatcagagactcaggagactgactaaaag
gcctggatctgcagtgtattatctgtataaaaattggcagggggaagcta
aaaggaaaggagattggagatctcaattctatcatggtgtatttcatacg
caaatcagagcatgcattgttttttgtttttggaaagagaagggaagtgt
gttctgccccatgtttccttccgtgtttatagttcaaactctatatatac
ttcaggtattttttgtttagcccttcattataaatgggcaggaaattgtt
tatcaacctagccagtttattactagtgaccttgacttcagtatcttgag
cattcttttatatttttcttttattatcctgagtctgtaactaaacaatt
ttgtcttcaaatttttatccaatatccattgcaccacaccaaatcaagct
tcttgattttcaaaaataaaaagggggaaatacttacaacttgtacatat
atattcacagtttttatttataaaaaaaatttacagtacttatggagagc
cagcagaagacatcagagcactcacttcttcccatctttgttaaggttag
cgaattacccatggacactgttaggtgaggctcattcggcagccctgaaa
acaaacctggtcacactgtctttaccctctcccttcagataaagcacttc
gattatctattgatctgcccagttttcaagtcatgcgaatactaaaaagg
ttacatcatctggatctgtaccttggctatataagcatgttttcccccta
ttctatgtttctttttttggtgaacattgaaaaacaggaggtgacttatt
actgttaattaaaactaaatgaaaaatgtcaagtctttaaaacagtgagc
ttgtaactctttcatgtaattttattctctatgaatttggctatcctact
gaatcttaaaataaaggaaataaacactttttttttaaaaaaaa

### The Amino Acid Sequence of Hs6_{—}25897_{—}28_{—}16_{—}1426.a:

(SEQ ID NO 38)

TVLIVGRIAIGVSISLSSIATCVYIAEIAPQHRRGLLVSLNELMIVIGIL
SAYISNYAFANVFHGWKYMFGLVIPLGVLQAIAMYFLPPSPRFLVMKGQE
GAASKVLGRLRALSDTTEELTVIKSSLKDEYQYSFWDLFRSKDNMRTRIM
IGLTLVFFVQITGQPNILFYASTVLKSVGFQSNEAASLASTGVGVVKVIS
TIPATLLVDHVFSKTFLCIGSSVMAASLVTMGIVNLNIHMNFTHICRSHN
SINQSLDESVIYGPGNLSTNNNTLRDHFKGISSHSRSSLMPLRNDVDKRG
ETTSASLLNAGLSHTEYQIVTDPGDVPAFLKWLSLASLLVYVAAFSIGLG
PMPWLVLSEIFPGGIRGRAMALTSSMNWGINLLISLTFLTVTDLIGLPWV
CFIYTIMSLASLLFVVMFIPETKGCSLEQISMELAKGELCEKQHLFYESS
PRRISAKTASKKKTPGAALGV

SMART™ and SOSUI™ predict 9 TM domains.

### AW023227/Hs10_{—}8766_{—}28_{—}5_{—}2415

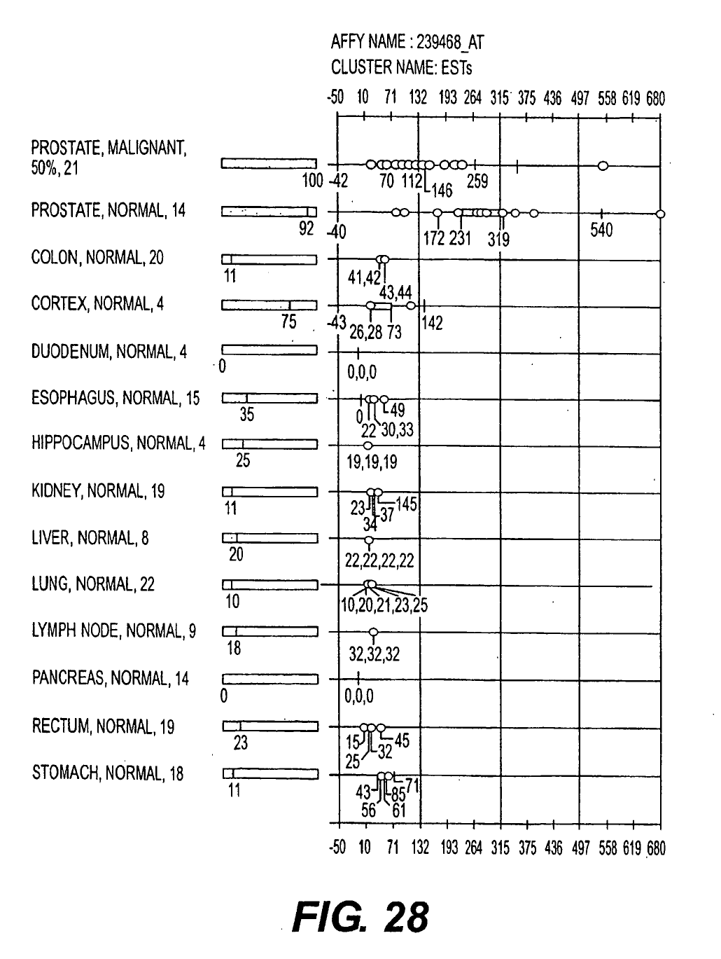

Using the GeneLogic database, we found fragment AW023227 was upregulated 9.82 fold in the malignant prostate samples compared to mixed normal tissue without normal prostate and female specific organs. Enorthern analysis of this fragment (FIG. 28) demonstrates that it is expressed in 85% of the prostate tumors with greater than 50% malignant cells with low expression in normal tissues other than the prostate.

### AW023227 Sequence

(SEQ ID NO 39)

TTCACTCTTTTTCATACTATTATAAGTTATTCTGGTATTAAATATGTTAA
NTAAAAGTGTTTTTGTTTTGACATATTTCAGTTAAATGAATGAATGCTGG
TTGTATTTTATTTGAATGAGTCATGATTCATGNTTGCCATCTTTTTAAAA
AAATCAGCAAATTTCTTCTATGTTATAAATTATAGATGACAAGGCAATAT
AGGACAACTATTCACATGATTTTTTTTAATACCAAAGGNTTGGAAGATTT
TATAATTAACATGTCNNNNNNNCTTTATAGTAAGCACATCCTTGGTAATA
TCTCCAATTGCAATGACTTTTTAATTTATTTTTTCTTTTGCTGCTTTAAC
ATTTTCTGGATATTAAAATCCCCCCAGTCCTTTAAAAGAATCTTGAACAA
TGCTGAGCCGGCAGCTGAAAATCTAACTCATAATTTATGTTGTAGAGAAA
TAGAATTACCTCTATTCTTTGTTTTGCCATATGTAATCATTTTAATAAAA
TTAATAACTGCCAGGAGTTCTTGACAGATTTAA

This corresponds to Nucleic Acid Sequence of Hs10_{—}8766_{—}28_{—}5_{—}2415 Shown Below:

(SEQ ID NO 40)

ttgaaagaaaacattttgtttctaaattagtctaccattgagtgagaata
atcaatatcaagaaagaagactatctttctcaactaaacaataatattcc
aatcagcttgggaagacctgaaacttgaataagcagtggaaatgccaaat
ataacagagggtatgtgctacagagaagtaaaaagggtttgactttttat
gatgggattttttttttctgggtatgtaatctattttttttttaaactgg
aaagcatttttgtcagtgtgaatgagggtcaatagtgcagccagtggtga
catttttctttattttgcaaaatgcttttaaaaccaaaggctgctctagt
tgatggacagtatcagtcttgatctaaattgtaggacactttttcatgta
acataacatttggggattgggtttatttagtgtaatgaagataatttgat
ataaaaatgcaaaatatataagttatgactgtatgatcagatgaagtatg
agttcttttggtttgcatccttaaatagttagagatctctgataaaaact
ttggaatctttgcaaaacaatacaaaaatgccaaaatgtgagcatgtcaa
tgaaaactaaagacaaatacttcactctttttcatactattataagttat
tctggtattaaatatgttaataaaagtgtttttgttttgacatatttcag
ttaaatgaatgaatgctggttgtattttatttgaatgagtcatgattcat
gtttgccatctttttaaaaaaatcagcaaatttcttctatgttataaatt
atagatgacaaggcaatataggacaactattcacatgattttttttaata
ccaaaggttggaagattttataattaacatgtcaagaagactttatagta
agcacatccttggtaatatctccaattgcaatgactttttaatttatttt
ttcttttgctgctttaacattttctggatattaaaatccccccagtcctt
taaaagaatcttgaacaatgctgagccggcagctgaaaatctaactcata
atttatgttgtagagaaatagaattacctctattctttgttttgccatat
gtaatcattttaataaaattaataactgccaggagttcttgacagattta
aaataaaagttaatttctagacctcga

### Encoding the Protein Hs10_{—}8766_{—}28_{—}5_{—}2415

(SEQ ID NO 41)

MSRRLYSKHILGNISNCNDFLIYFFFCCFNIFWILKSPQSFKRILNNAEP
AAENLTHNLCCREIELPLFFVLPYVIILIKLITARSS

SOSUI™ and SMART™ predict 2 TM domains.

### BC005335/DKFZP564G2022

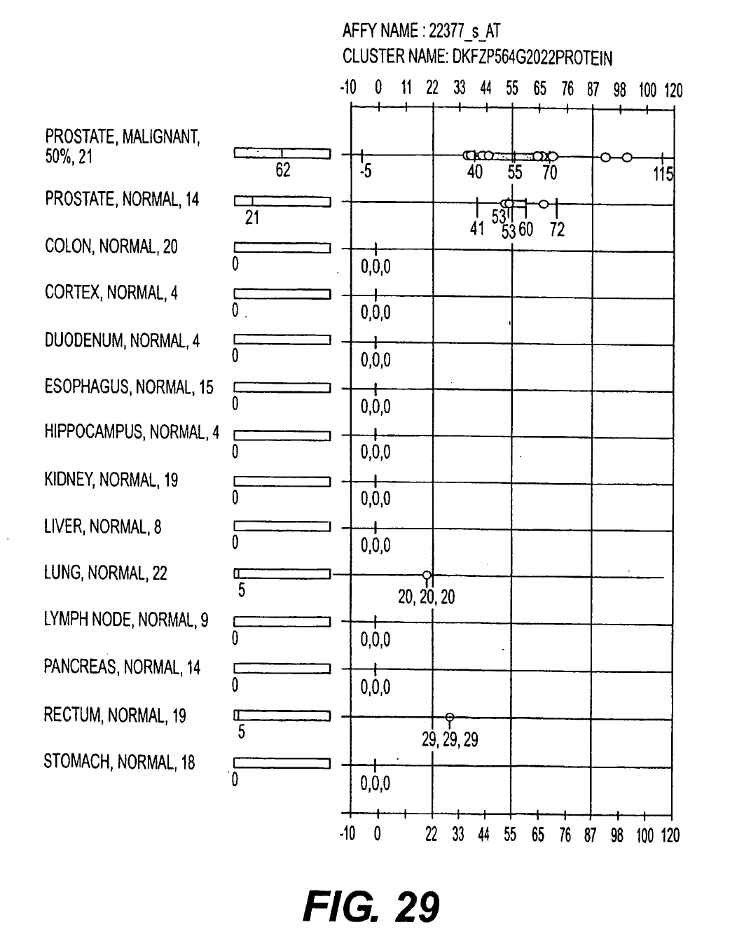

Using the GeneLogic database, we found fragment BC005335 was upregulated 5.28 fold in the malignant prostate samples compared to mixed normal tissue without normal prostate and female specific organs. Enorthern analysis of this fragment (FIG. 29) demonstrates that it is expressed in 52% of the prostate tumors with greater than 50% malignant cells and almost no expression in normal tissues other than the prostate.

### Sequence of BC005335

(SEQ ID NO 42)

GATATTCATTGGATTTTCTCTTACTAATAGGTATATATTCACTGTGAAAA
TGGAGACGATATACATAAATGAAAAGAAGAAAATAGTAATCTATAATACC
ATGCAGTGATATATTTATCTTCCTATTCTTTTGTATATGGGCATGTTTAT
ATTATTTTAAAAAGGGAATCTTAGAGTATGTATTATATGACTTTTTTTTG
TAGCTTAGCAATATAACATGGACATGTCGTCAGTTTGGTAAATATTGTAT
TGCATCGTTACTTAAATGCTTGTATAGGGTCTTATTGTATGAGTACATTG
CAATTTGTTCAATTCCCTGTTCTTGAACTTTTATGAGTTTCATTATCTTG
GAATTTTATGCAGTGTTGTGATTAATATTTTAACTACATTTGCTTTTAAG
TCTTTATTTTCTGATCTCAG

### This Corresponds to a Nucleic Acid Encoding Hypothetical Protein DKFZp564G2022

(SEQ ID NO 43)

GGTGAAATGCTTTCGGTAGGCACTCCACGGCTGTGAAGATGGCGGCGGCT
GCGTGGCTTCAGGTGTTGCCTGTCATTCTTCTGCTTCTGGGAGCTCACCC
GTCACCACTGTCGTTTTTCAGTGCGGGACCGGCAACCGTAGCTGCTGCCG
ACCGGTCCAAATGGCACATTCCGATACCGTCGGGGAAAAATTATTTTAGT
TTTGGAAAGATCCTCTTCAGAAATACCACTATCTTCCTGAAGTTTGATGG
AGAACCTTGTGACCTGTCTTTGAATATAACCTGGTATCTGAAAAGCGCTG
ATTGTTACAATGAAATCTATAACTTCAAGGCAGAAGAAGTAGAGTTGTAT
TTGGAAAAACTTAAGGAAAAAAGAGGCTTGTCTGGGAAATATCAAACATC
ATCAAAATTGTTCCAGAACTGCAGTGAACTCTTTAAAACACAGACCTTTT
CTGGAGATTTTATGCATCGACTGCCTCTTTTAGGAGAAAAACAGGAGGCT
AAGGAGAATGGAACAAACCTTACCTTTATTGGAGACAAAACCATTCAGAT
GCCTTTCTTGAAGAAACATTTCTTGGATTGTTGAAAGACTTTAATAATTT
CCAAAGTTCCAAAAGTTGATTTTGATAGTTTTTGCCAGTGTTTTCGTTGC
TTTTATGGATGAGTAGATTTTCAGAGTTTCTTATTCTGCCATTCTGAAAG
TGTTCTCACTACCTAAACCCCAGTTTTATTTGTACAGAATTTTAACTGAA
TGTAAGTTAGGCATGACAGTCTTTGTTAATTTTTTTAAACAAAAGATAGC
CATTAGGACTGGGTACAGTGGCTCACGCCTGTAATGCCAACACTTTGGGA
GGCCAAGGTGGGCAGATGACTTGAGGTTGGGAGTTCGAGACCAGCTTGGC
CAATGTGGTGAAACTTTGTCTTTACTAAAAATACAAAAATTAGTTGCTCA
TGGTGGCAGGCACCTGTAATCCAAGCTACTCAGGAGGCTGAGGCAGGAGA
ATCGCGTGAACTTGGGAGGTGGAGGCTGCAGTGAGCTGAGATCACGCTAC
TTCACTCCAGCCTGGGCAGCCAGTGAGATTCCATCTCAAAAAAAAAAGAA
AAAAGATATTCATTGGATTTTCTCTTACTAATAGGTATATATTCACTGTG
AAAATGGAGACGATATACATAAATGAAAAGAAGAAAATAGTAATCTATAA
TACCATGCAGTGATATATTTATCTTCCTATTCTTTTGTATATGGGCATGT
TTATATTATTTTAAAAAGGGAATCTTAGAGTATGTATTATATGACTTTTT
TTTGTAGCTTAGCAATATAACATGGACATGTCGTCAGTTTGGTAAATATT
GTATTGCATCGTTACTTAAATGCTTGTATAGGGTCTTATTGTATGAGTAC
ATTGCAATTTGTTCAATTCCCTGTTCTTGAACTTTTATGAGTTTCATTAT
CTTGGAATTTTATGCAGTGTTGTGATTAATATTTTAACTACATTTGCTTT
TAAGTCTTTATTTTCTGATCTCAGAAGAATTGTATATTGGGATAAGTTTT
TAATTCTATAACTTAAAAGTAAAAATCCTTTGTAATTTTATGTTCGAAAA
AAAAAAAAAAAAAAAAAAAAAAAAAAA

### Amino Acid Sequence of DKFZp564G2022

(SEQ ID NO 44)

KGFRIVTCQSDWRELWVDDAIWRLLFSMILFVIMVLWRPSANNQRFAFSP
LSEEEEEDEQKVPMLKESFEGMKMRSTKQEPNGNSKVNKAQEDDLKWVEE
NVPSSVTDVALPALLDSDEERMITHFERSKME

SOSUI™ and SMART™ predict 1 TM.
BF055352/Hs18_{—}11087_{—}28_{—}3_t18_Hs18_{—}11087_{—}28_{—}4_{—}3064.a

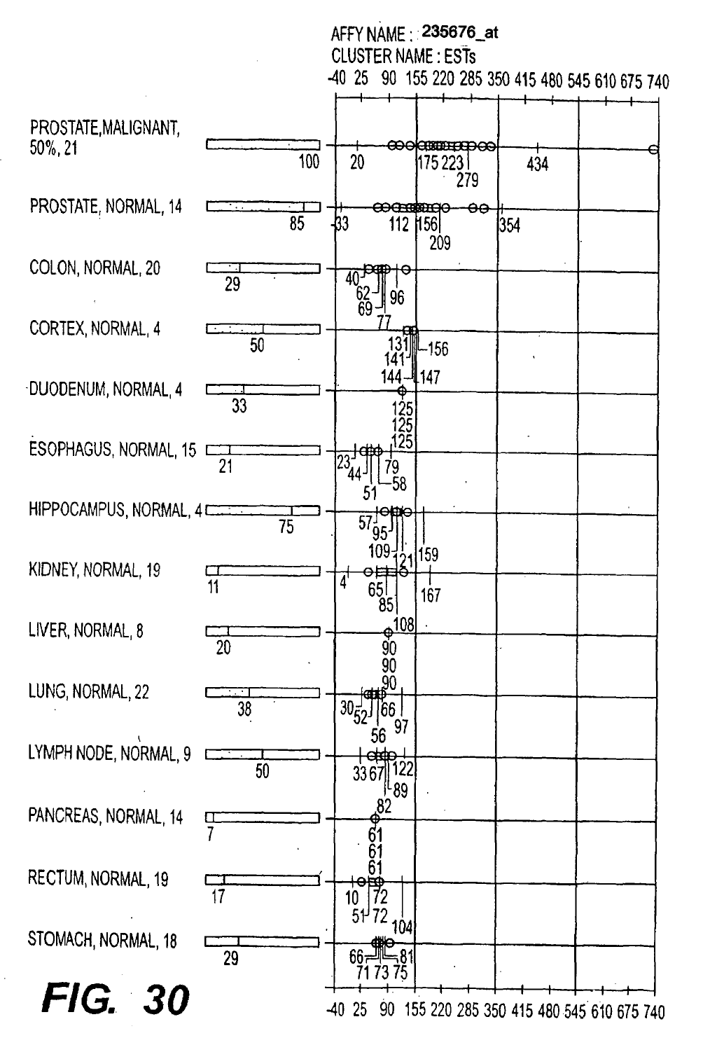

Using the GeneLogic database, we found fragment BF055352 was upregulated 3.59 fold in the malignant prostate samples compared to mixed normal tissue without normal prostate and female specific organs. Enorthern analysis of this fragment (FIG. 30) demonstrates that it is expressed in 100% of the prostate tumors with greater than 50% malignant cells and almost no expression in normal tissues other than the prostate.

### BF055352

(SEQ ID NO 45)

GTTTCCCATGAGCAGAGATGATTGAGACCTGGGTCCATCTGATTACATAT
TGCTGTTGATTTTGTGAGCATAATCGTTGGCTGGTTTATGCACTGAACCT
CCTTGCTCTGGGATCATAATCATATTTGAGTATAAGTTATGGTATTCACA
TTTGTATTTGCTACCCAATACATTTATTTGTTATATCTGACAAGCACTGG
GAAATGAAAATAATTATTTGCATTACAAACTCATTATTCATGTACTTTGA
AAGCTTTATCTAACAGCAGTTTTTATATGGGCTATCTGAATCTTATCTTC
TAAATAAAAACTAGATTTGTGAAANNNNNNTATTCTTTTTGTACNAGCGG
CNTNNCTATTTTAATTGTAGCNAGTGNAGACNACCAGCATCACTATCTCN
ANCCNAGTGCCTACTTNNGNNNACTTGTCCTGGCTGCCNGTGCTGATGCT
CCTTACTAATAAAAGCTGTTGAGACAGGGCTGAATACATCCTTACAGCCC
TGGTCAGTGGCATTCCCTCGTACAATTCATTTCTTA

This Corresponds to Hs18_{—}11087_{—}28_{—}3_t18_Hs18_{—}11087_{—}28_{—}4_{—}3064.a

(SEQ ID NO 46)

gcgggggccggcaggtgctccgcagccgtctgtgccacccagagccggcg
ggccgctaggtccccggagaccctgctatggtgcgtgcgggcgccgtggg
ggctcatctccccgcgtccggcttggatatcttcggggacctgaagaaga
tgaacaagcgccagctctattaccaggttttaaacttcgccatgatcgtg
tcttctgcactcatgatatggaaaggcttgatcgtgctcacaggcagtga
gagccccatcgtggtggtgctgagtggcagtatggagccggcctttcaca
gaggagacctcctgttcctcacaaatttccgggaagacccaatcagagct
ggtgaaatagttgtttttaaagttgaaggacgagacattccaatagttca
cagagtaatcaaagttcatgaaaaagataatggagacatcaaatttctga
ctaaaggagataataatgaagttgatgatagaggcttgtacaaagaaggc
cagaactggctggaaaagaaggacgtggtgggaagagcaagagggtgagg
attcacctttaagttatatagaaggttatgaaaaacacttagaaatgaag
aaattaaatcaataggctaatgagtcgttaattacaaatatgacatatca
ggagagttttaagcagttctagtttatcctgtgaagactaaatacaactt
agaaattcctaaagacctaaaatctaaaactgaacccaattatattatct
atatgatgggttcaaatctgtttcaaaataaatccagccaggcgcagtgg
ctcacacctgtaatcccagcacctttgggaggctgaggcaggaggatcac
ttgagcccaggagttccagaccagcctgagtaacatagggataccccatc
tctattaataaaaattttaaaaaatttgttctaaaaaaagaagaaatata
aatcctcactgagagattagttatttgtggattttaaataaccattacaa
gaaagtctcccagagataaccactgtttaacatttcagggaatgctgtag
gtactctctgggctggtacagatgtgtgttatgcctatatttattt

Amino Acid Sequence of Hs18_{—}11087_{—}28_{—}3_t18_Hs18_{—}11087_{—}28_{—}4_{—}3064.a

(SEQ ID NO 47)

MVRAGAVGAHLPASGLDIFGDLKKMNKRQLYYQVLNFAMIVSSALMIWKG
LIVLTGSESPIVVVLSGSMEPAFHRGDLLFLTNFREDPIRAGEIVVFKVE
GRDIPIVHRVIKVHEKDNGDIKFLTKGDNNEVDDRGLYKEGQNWLEKKDV
VGRARG

SOSUI™ and SMART™ predict 1 TM.

### N62096/Hs2_{—}5396_{—}28_{—}4_{—}677

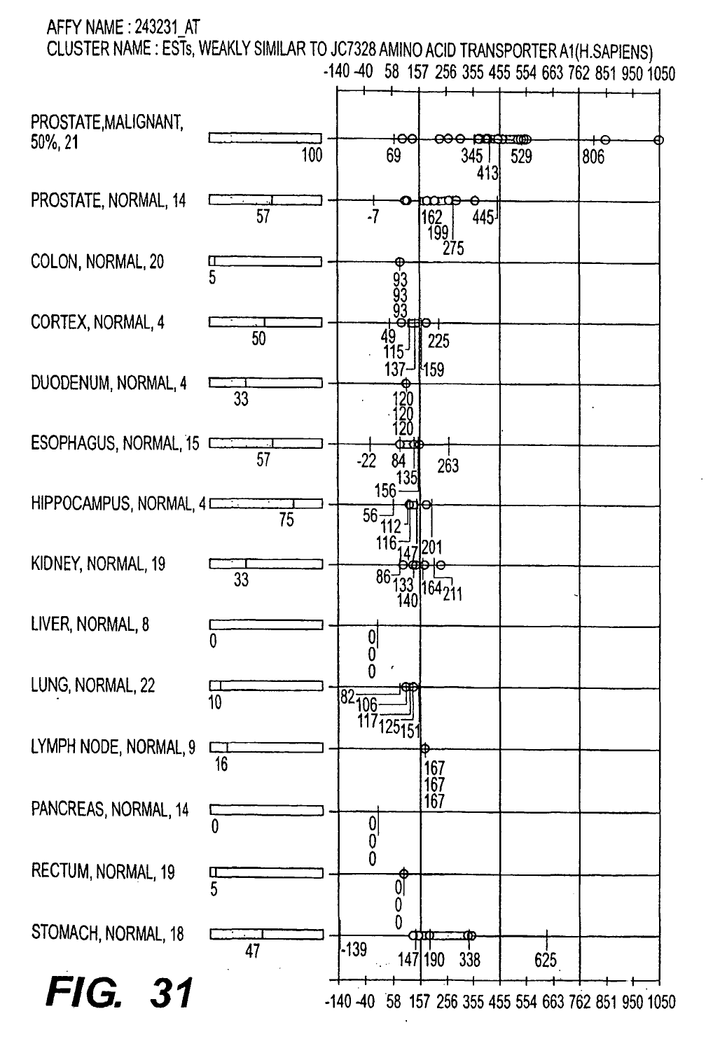

Using the GeneLogic database, we found fragment BF055352 was upregulated 3.73 fold in the malignant prostate samples compared to mixed normal tissue without normal prostate and female specific organs. Enorthern analysis of this fragment (FIG. 31) demonstrates that it is expressed in 100% of the prostate tumors with greater than 50% malignant cells and low expression in normal tissues other than the prostate.

### Sequence of N62096

(SEQ ID NO 48)

TGGTGGGAATCTTTCATCGGTTTTCCACATTGTTGTAACAGTGATGGTCA
TCACTGTAGCCACGCTTGTGTCATTGCTGATTGATTGCCTCGGGATAGTT
CTAGAACTCAATGGTGTGCTCTGTGCAACTCCCCTCATTTTTATCATTCC
ATCAGCCTGTTATCTGAAACTGTCTGAAGAACCAAGGACACACTCCGATA
AGATTATGTCTTGTGTCATGCTTCCCATTGGTGCTGTGGTGATGGTTTTT
GGATTCGTCATGGCTATTACAAATACTCAAGACTGCACCCATGGGCAGGA
AATGTTCTACTGCTTTCCTGACAATTTCTCTCTCACAAATACCTCAGAGT
CTCATGTTCAGCA

### This Corresponds to Hs2_{—}5396_{—}28_{—}4_{—}677

(SEQ ID NO 49)

gctgaagaatttagggagttgattctgatgtaagaagacaatggataaag
tatttttcagaagtcagtacaaattggcagcaaatctaccaaaaacaaat
aataagagaaaaactatcagtgatggatttatcttcacatgtagcatgta
ctggtttaaatcagtgaataactacatagttattgaattcaaaaactttt
atttagacctggtcatctattctcttaattaaatgaaatgaagtttatgg
agattcacttataagtcatgtgttgcttaatgacagggaaacattctgag
aaatgcattgttaggtgatttcctcattgtgcaaacatcacagagtatac
gtacacaaatctagatggtagcacctattacacacctaggctatatgcta
tagcttattgctcctaggctataaacctctacagcatgtttctgtactga
attctgtaggcaactgtagcagaatggaaagtatttatgtatctaaacat
agaaaaatatatagtaaaaatacagcattgtaatcatatatgtgggccat
taggtgatgcataactgtaatatctaatatttaatttattagatagttat
ctcaaacatttagtatctagtaaataaacttattttatattactatctag
gggacttatttgaaaattactgcagaaatgatgacctggtaacatttgga
agattttgttatggtgtcactgtcattttgacataccctATGGAATGCTT
TGTGACAAGAGAGGTAATTGCCAATGTGTTTTTTGGTGGGAATCTTTCAT
CGGTTTTCCACATTGTTGTAACAGTGATGGTCATCACTGTAGCCACGCTT
GTGTCATTGCTGATTGATTGCCTCGGGATAGTTCTAGAACTCAATGGTGT
GCTCTGTGCAACTCCCCTCATTTTTATCATTCCATCAGCCTGTTATCTGA
AACTGTCTGAAGAACCAAGGACACACTCCGATAAGATTATGTCTTGTGTC
ATGCTTCCCATTGGTGCTGTGGTGATGGTTTTTGGATTCGTCATGGCTAT
TACAAATACTCAAGACTGCACCCATGGGCAGGAAATGTTCTACTGCTTTC
CTGACAATTTCTCTCTCACAAATACCTCAGAGTCTCATGTTCAGCAGACA
ACACAACTTTCTACTTTAAATATTAGTATCTTTCAATGAgttgactgctt
taaaaatatgtatgttttcatagactttaaaacacataacatttacgctt
gctttagtctgtatttatgttatataaaattattattttggctttta

### Amino Acid Sequence of Hs2_{—}5396_{—}28_{—}4_{—}677

(SEQ ID NO 50)

MECFVTREVIANVFFGGNLSSVFHIVVTVMVITVATLVSLLIDCLGIVLE
LNGVLCATPLIFIIPSACYLKLSEEPRTHSDKIMSCVMLPIGAVVMVFGF
VMAITNTQDCTHGQEMFYCFPDNFSLTNTSESHVQQTTQLSTLNISIFQ

SMART™ predicts 2 TM and a signal sequence, SOSUI™ predicts 3 TM domains.

### NM_{—}018542/PRO2834

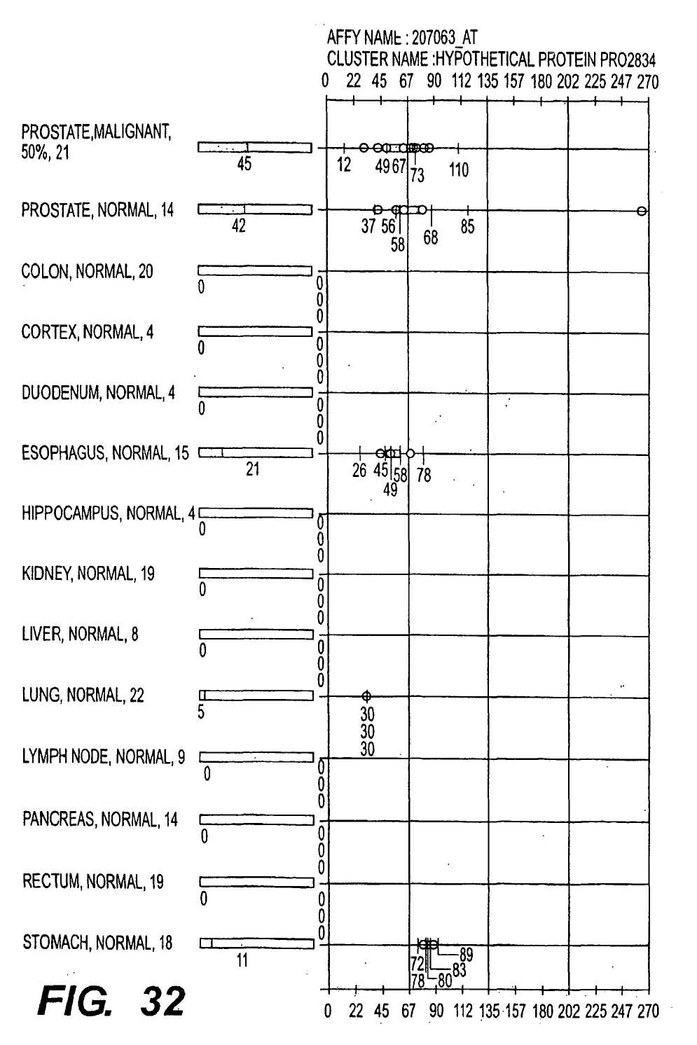

Using the GeneLogic database, we found fragment NM_{—}018542 was upregulated 4.52 fold in the malignant prostate samples compared to mixed normal tissue without normal prostate and female specific organs. Enorthern analysis of this fragment (FIG. 32) demonstrates that it is expressed in 45% of the prostate tumors with greater than 50% malignant cells and low expression in normal tissues other than the prostate.

### Sequence of NM_{—}018542

(SEQ ID NO 51)

TGTTGGGAATTGGTACTGGCTAGAAATTTCTGTTGAGTATTTATTACCCC
ATGGTAATAATGGTAAACCACAGTTTAGAAAGATTTTTTTTGACAGCCAC
AGCATGTTCCGAAGAGATGATTGGAAGATGGAAGTGGAGGGTTAAATAAT
GAAATGCAGCTAACATTTCGGAAAGTTTCTAAAAGTTGTACAACATGCCC
TACAGCTACTCTTTAAATCTCCAAATCAAATGAGTTTCAGGTGGAGCCTC
TGGGAGGTGATGAGGTCATGAGAGTGGAGCCTCATGAATGGGATGAGCAC
TCCTACAAAAAGGATTCCAGAGAGCTCGCTTGCTCCTTCCACAGTGTGAG
GACACAGAGGGAAGGCTCTGTCTATGAATGAGAAAGTGGGTCCCCACCAG
ACATTGAATCTGCCGCATCTTGATACTGGACTTCCAGTCTCCAGAACTGT
GGGCAATAAATGTCTGTTGTTTATTACCTGTCCAGTATCTTTGGTATTTT
GCTATAGCAACCCAAATGGACTAAGAAAACACCAGAGGCCATACCTAAT

### Nucleic Acid Sequence of PO2834

(SEQ ID NO 52)

CAAAAGCAACCCTTCTTGCTCCAGGCATGTGCAGGAGGTTTTTTGGTTTC
AGCATTTTGTTGCATGCTGACTATGTCCTTTACCTTCTCTTAAATTATGT
ATCAATTCATGCTGGTTTATTCACTTCCTGATGTCTATATGAAGAGGCTG
TCTGCCAACATCTTTCATCACTCTGCCTGCAACTATGAAAAATTTAGTTC
TAAAAAATGCAACCTTGCTAAATTGAGTACTAATAGGATTGGTTCAATTA
TGTTCTATGTCTGTTCCATATTGACATTGTGTGCATCTTTGCCATGCAGG
CTTTTTAGGAATTATCGCATCTCTAACTTCCCACGAGTGTTTATGAAAAT
GTTTAGATTTAAAGAACTTTATTGCTTTAGACAGAATAAGGCATGCAGTT
CTAACAGAAAGATCCATGAATTCCAGAAATATCACTGAAAATTATTGACA
TTTAAGATTATTTTCTGTTTGTTACTATGGTTCACAATTCAAGAATAACT
CTGGCCAGGTGCAGTAGCTCACACCCTGTAATCCCAGCACTTTGGGAGGC
TGAGGTAGGCAGATCACTTGAGCTCAAGAGTTCAAGACCAGCCTGGGAAA
CATGGCAAACTCCCACCATTACAAAAAAATACAAAAATTAGTTGGTCATG
GTGGTGTTCACCTATAGTCCCAGTGACTTGGGAGGCTGGGATGGGAGGAT
CTCTTGAGCCCAGGAGATGCAGGCTTGCAGTGAGCCATGATCATGCCACT
GTACTGCAGACTGAGTGAAACAGCAAGATCTTGTCTGAAAAGAAAAAAAA
AGTAAAAGAAAAAGAAAAGAAAATAACTCCCATTGCTAAAGACATATATG
CTTATCAGGTTAAGATAAAGTGAATTTTGTTCTTCCCAATGACATTTCAG
GATATTTGTTCACAGGAAAGAACATGTTGGGAATTGGTACTGGCTAGAAA
TTTCTGTTGAGTATTTATTACCCCATGGTAATAATGGTAAACCACAGTTT
AGAAAGATTTTTTTTGACAGCCACAGCATGTTCCGAAGAGATGATTGGAA
GATGGAAGTGGAGGGTTAAATAATGAAATGCAGCTAACATTTCGGAAAGT
TTCTAAAAGTTGTACAACATGCCCTACAGCTACTCTTTAAATCTCCAAAT
CAAATGAGTTTCAGGTGGAGCCTCTGGGAGGTGATGAGGTCATGAGAGTG
GAGCCTCATGAATGGGATGAGCACTCCTACAAAAAGGATTCCAGAGAGCT
CGCTTGCTCCTTCCACAGTGTGAGGACACAGAGGGAAGGCTCTGTCTATG
AATGAGAAAGTGGGTCCCCACCAGACATTGAATCTGCCGCATCTTGATAC
TGGACTTCCAGTCTCCAGAACTGTGGGCAATAAATGTCTGTTGTTTATTA
CCTGTCCAGTATCTTTGGTATTTTGCTATAGCAACCCAAATGGACTAAGA
AAACACCAGAGGCCATACCTAATAAAAATATTGACATCACAAAAAAAAAA
AAAAA

### Amino Acid Sequence of PRO2834

(SEQ ID NO 53)

MYQFMLVYSLPDVYMKRLSANIFHHSACNYEKFSSKKCNLAKLSTNRIGS
IMFYVCSILTLCASLPCRLFRNYRISNFPRVFMKMFRFKELYCFRQNKAC
SSNRKIHEFQKYH

SOSUI and SMART predict 1 TM.

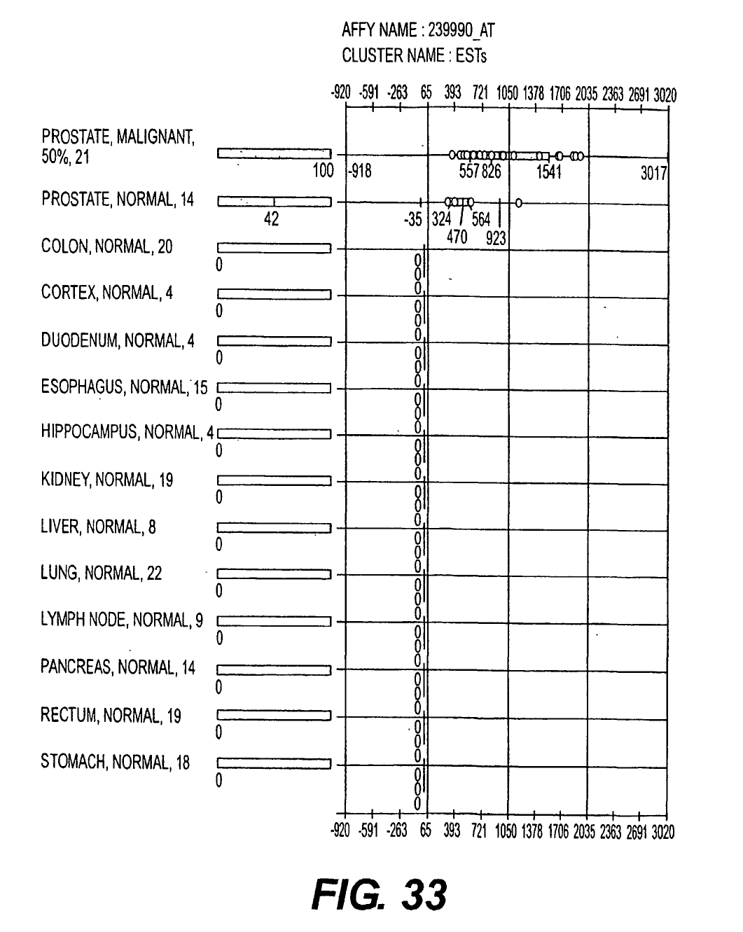

### AI821426 (FIG. 33)

(SEQ ID NO 54)

TAAAGAGCGCCCGAAGCACTAGCAGAGTCAACCCCCCGGGGACCCATAAG
ACAGGGCTTCTAGTATAAGGATTGGAGTTTGACCCACCCCCAAAAAATGC
CCTGGGGATATTGGTTTTCTCAGGTGGCATATGACTCTCCGGCTTGGATT
GCCTCGCTNCGGANAGGGGACAAAAGGTTTTGCCCTGAGCATCTGGTGNT
GTCTTCCAGTGCCTGGTTAGGTTGCTCCGNGGCTGGACAGTCTGACTACT
CTCAAAACTCCTCGTGACAGGCCTTTCTGGGGTCTGATCGCCCTTTGTTT
CCTTACACTTGGGCCTGTTATCAGAAGAACTCTGAATCCGGAAATACCTT
GTTTAAATTTGGGCTACAGTTTTCAAGATCCAGGCATTTGGGTGAATCAC
TTAACCCGAGTATTAGGATCTGGAAAATGGGGCTAGTAATTGTTGTAAAT
GTGAGGTGTTTAAAAGTGTCTGGCATTTTAGTGCGTAGATAAATGCTACT
TCCTGTGCCCATTCTCTTGGGAGTTCTC

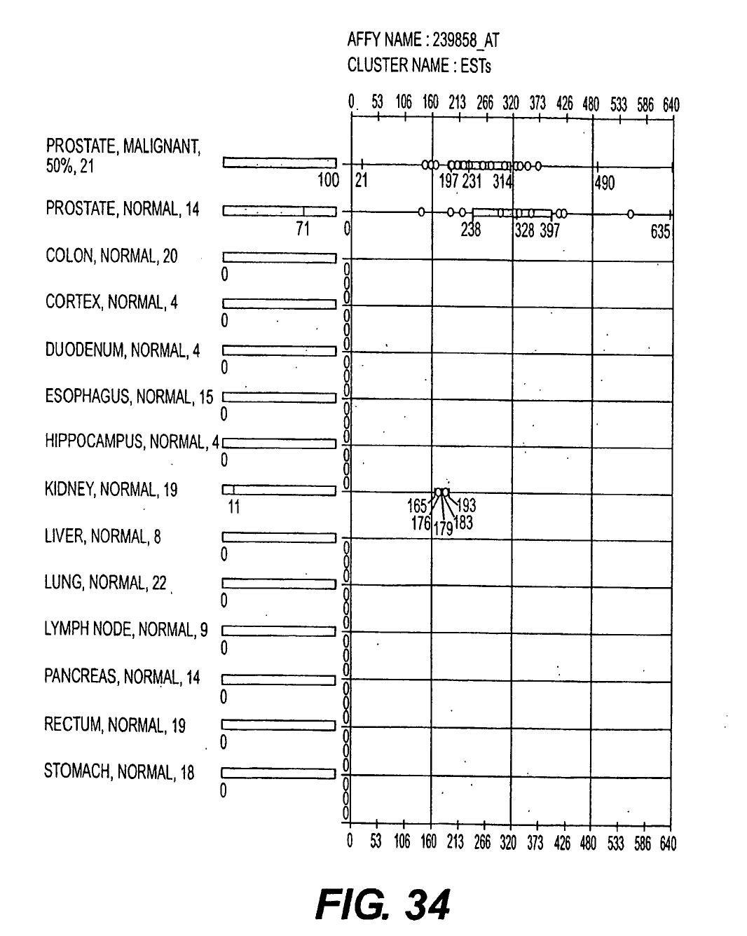

### AI973051 (FIG. 34)

(SEQ ID NO 55)

TAGAATGCCCTAGGTGAATCCCTCCAGTCTTCCAGTACCATCCNTGACTC
CTCTCTCTGATGACACATGAACTTTATGCTTTTGCACACTTCAGGCAACN
CNAAAAGAAAGGAAAAGAACAGCTTAGCTTCTTAATGTGTGTAAGAAACC
ACAGTGAAAAAAAATCAGGTGTGTTGTTGAGGCTGCTAAAAGCTTTCCTT
TTTTTTCTGTGCCAGTTCTCGCTGCCTCATTGGTTGAGATGGGATGTCTT
TTTTGATGTCCTCTTTAGAGAGTGTTATCCTCACCTTTTTGCATAGTCCT
ACCAAAAGACACCTCACATGCAAAGTGTAACAGAAAATTACAGTCATGAC
TTTAGTTTTAAAAACAGGACGTATATTCATGAAGAATGTTTGCTGTTTTC
CCAGTGGGTTAATC

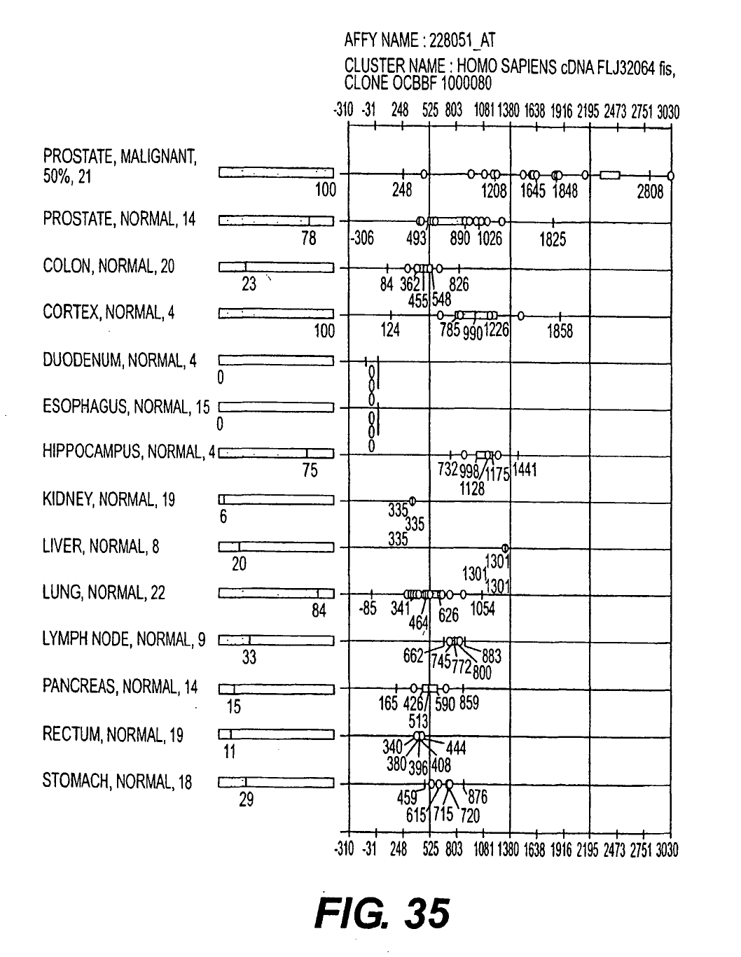

### AI979261/AW953116 (FIG. 35)

### AI979261

(SEQ ID NO 56)

TATTCAATATGCTTTTCCCGCTTTTCTAAGAGGAATAAACTTAGACAAAT
TACATTATAAACAGTTCCCCTACTACTATCTCCCACTCTAGATAAAGCCN
GTGGGTGGTANNNGNNCTTTTATTCCTTATAGTATTATGCCAAAGAATCA
ACTTATTTTCATTGAAGATTATAAATAAATGAAGCTTGTTATAGCCATAA
TGATTTGAGTCAGTATACCATTTTACCTATAAAATGCAAAATTCATCCTT
GCAACCCCATTCACCAGGAGCCTTGAAGCATTTTGTTTACTCCAAAGGCC
TTGTCAAGGAAGCATAATTTTTTGTTTTGCCTTCTTATTTAGTCAGTTTG
GTCATATTTACTTAAAAAAACAAACTGAAAATCACACTCCTTTATATGTT
GATATAACTGATTTTATAGAATCTGTCTGTTCTTTGTTTAACAGGTCTCT
GTAAGCAAGCTTGCA

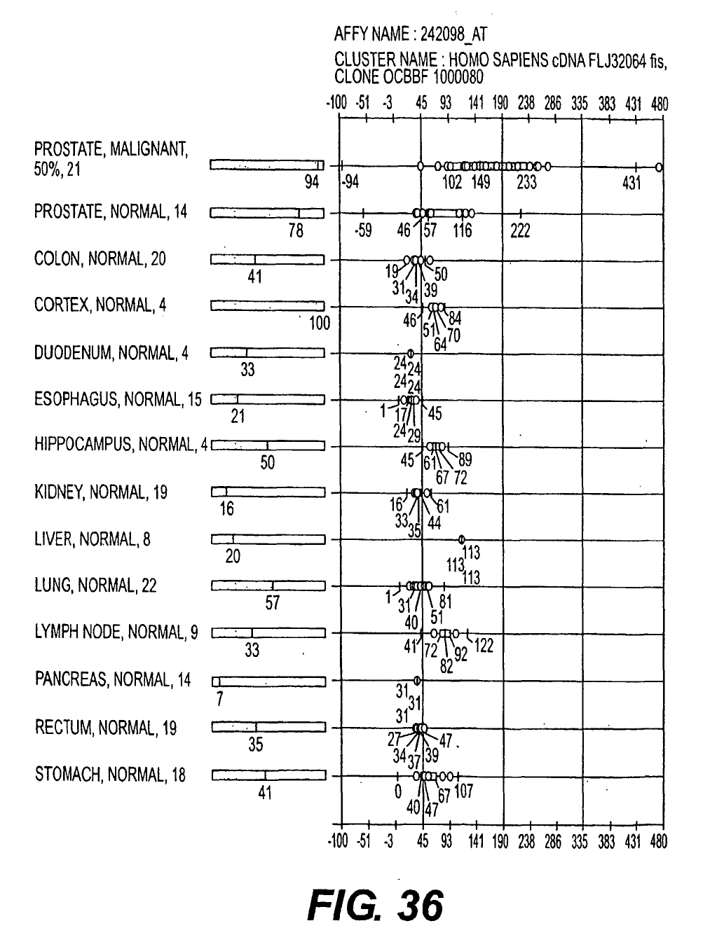

### AW953116 (FIG. 36)

(SEQ ID NO 57)

GTTGTTTGTGCACATATCTACATGGTGGAGACCATATTCATTATTTCATC
TTCCAAATAATGGGAAAAATATAAAAGNGANTCAGTGTGCTTTGGGAATT
CAGTGAAATCATGTTAACTCATATAGAGGGGGCCTTAGTTTATCTCTNCT
TTACTGAATTAATTAGTTTTGGAAATTCTTTTACCATTAAAAAAAATTAA
GGACCATACAGAGAATGATTTAAGAAAAAACAAGTCACTTAAAAATCATC
ACCTATTTATAAACTGTATTAATTACACATAATGCTTATTGATTCAATGA
GGTTTCTCTAAAGACTTCTGCTTAATAAATATGCTGACTTCATTTAAATT
AGTTTAGACTATTGTAGGAATGGAAGGAAATGATTATATTTACTAGAATT
AGTGAGATCAGAAAGCATATCAGAATGTTGATGATATCAAGGAGACAATC
TACAGAGTTTTTGCCT

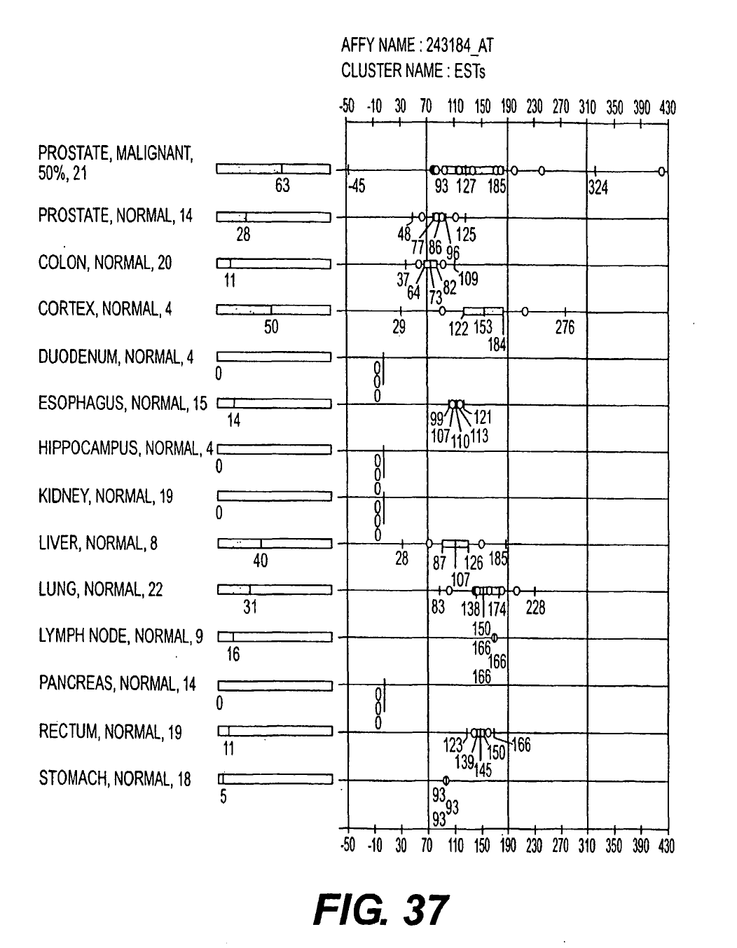

### AW173166 (FIG. 37)

(SEQ ID NO 58)

GAAACCATTGAAACCCTATTCATTCTTAAAGACTAAGTAATTTTTTAGTG
TTCTACTGTATGCCAAGCACTGTTGTACTCTTGTGGGCCCTGGAATTANA
TCAGAAAAAAACAGGCAGAATTTGCCTCCTCATGGATTCTGATCNCNNCT
ACTGGNCCTCAGTGACAGTTGAATATGTACATCAGATAGTTGTTTNCCCC
ANTCTCCTANCTACATTATAACTTTCACAAGGGTTGGAAATCTTAAGTCC
GTTTTCTATCTCCTTAGTGCTTGGTACCTAGTTCTGCCCCAAAAAACTTA
ATTCCCTAGGACACTAACCATGTCGAATAAAGTCACTCTTGGGAGGTCTA
CANCAGCACCGCCCAGTAGCAGTATAATA

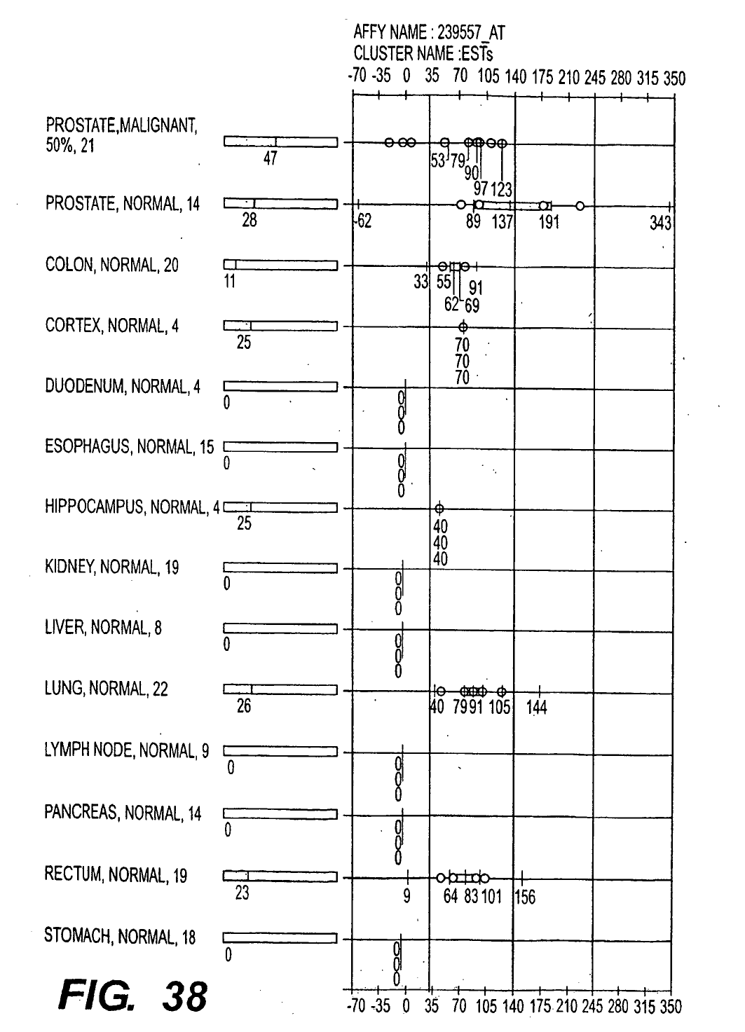

### AW474960 (FIG. 38)

(SEQ ID NO 59)

CATTAATAATTTGCCTTTTTACATCTCTTAGGAGTGAATCATTATTTGAA
AAGTTTTCACTTTTTCTTCTTTGTTGCTGTTTTATGCACATACATGTGTG
TGCAGTTCACCAAAGACAAATTTCTTCAGCAAAATTAATGTTTCCATATT
GTATAAAACTCATAACTATGGATTACAAATCATGTTACCATTAATTGCTT
TCTATATTGTTGTATTTAGATTTAACCAGTGTTTATCCACCTGTTAAGAC
CTGTAATCCAGTCAGGGTGGCTCATG

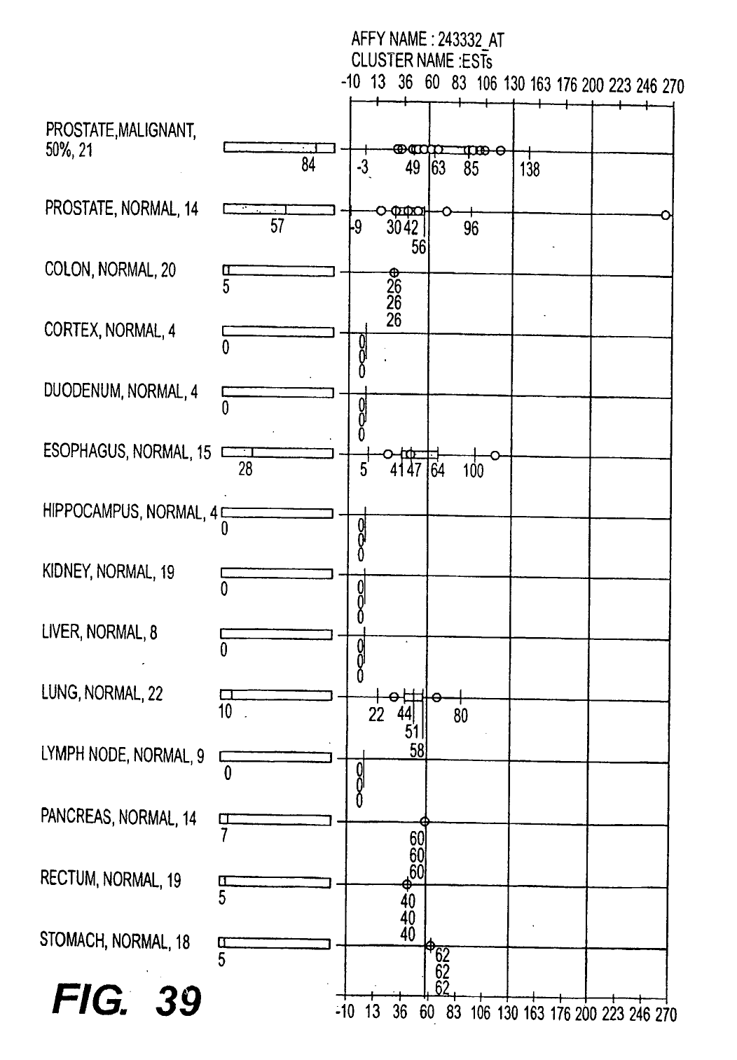

### BE972639 (FIG. 39)

(SEQ ID NO 60)

TTTACTAAACGATGATTACTCCTTCNATATTCATATTCCTAAACACATAC
AGTTTCTTANTGTAATTAAGTTTTTANNNAAAAAAANNGGGAAATGCATT
ATTGAGGCGATAGGATTACTGGGTGGCTATAAACACATCTGCTGCACAGC
TGACATTTATCTTCTACAATGAGCANTGACAATTTTATTTTTTAATAATC
AGTATGGACTAATCCTGATGATTTTTTTTNAACATTTTCAAATAGGGCTG
CATATGGCTTAAAATTAATATATACATGTGTACCTATATAATATTCTTAT
TTATTAATGGACTTCCTACATAGCTCATATTGACGTTAGATTTAAATGAA
ATTCCAGAAGGGTTTTCTATAGGTAAGTCATACATTGGATTTCCATATTA
CCTATGATTATTGAAGTATTTATTTCTGTTTTTAAGACTTCAGAGCAATT
TTGCTGGTCATTTGTTTTCTGTGTTTTTATTTTGAAATNGTTCTTTGAGG
CATTGTCCTATTAC

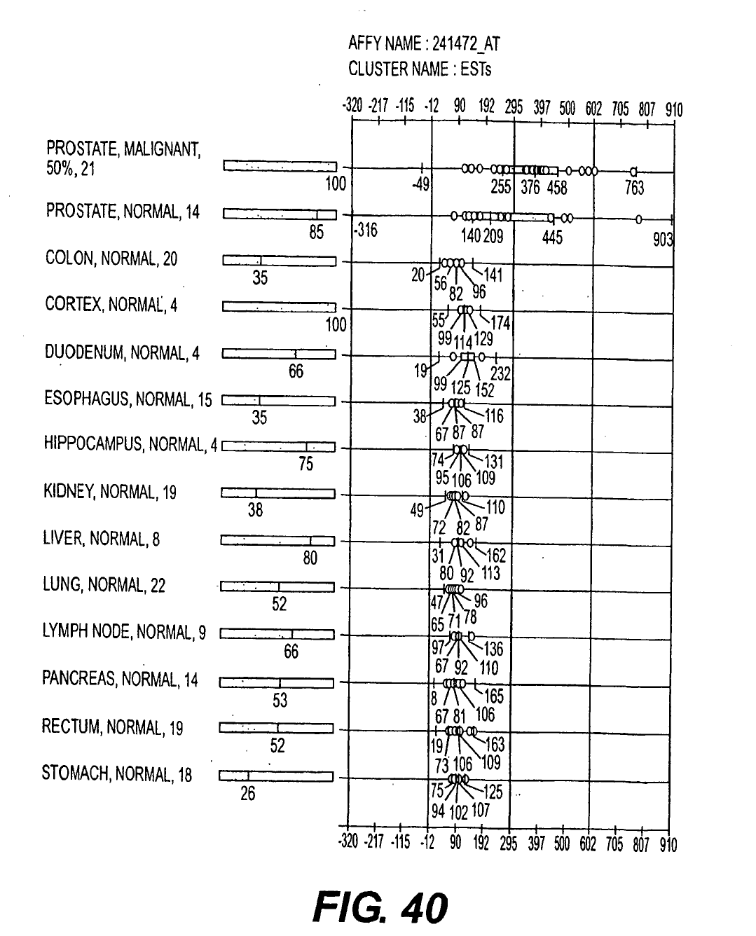

### N74444 (FIG. 40)

(SEQ ID NO 61)

TTATCATTCAGCTTGCTTTGTGTTGTTTTGAGGGGTTGGGGTACAGTGGG
ACAGTTTTATTTTGTTTGGCATTTATAGAAAATTGAGAAGTTTCCTTTGA
TCAAGCCATATTTTTGATTTAAAACAATGATTAGCAGTTTAGAAAACTAT
CTCTGCTATTTTATTCTGCTTTTAAATTCTTTGTTTTTTATATTTCTGTC
CCTTAGACTTTAACATTTTAAAGTGTGTAAAAATAAAACACTGTCAGTGC
TAATCATAGAAAATCAGACTATGGCTTGAAATGACTAGAAAAACATTTCA
AATTAGGCTGCTTTATGATTTGCATATTATGATTCCGGCCATTGGAGTTT
TTGGATTTCTAAGTGTTCATAATACCATGAAAAGTAAATATTTTAAACAA
TTGTATCCCCGTTTAAAAACTTTCTAATGTTAAAACTGTATTTTTTTCAT
GTATTAGCCCATGTGTGATAATCTTAGTTTTCCAATTATGGAGGGCATGA
GGAGTAGCTTTATT

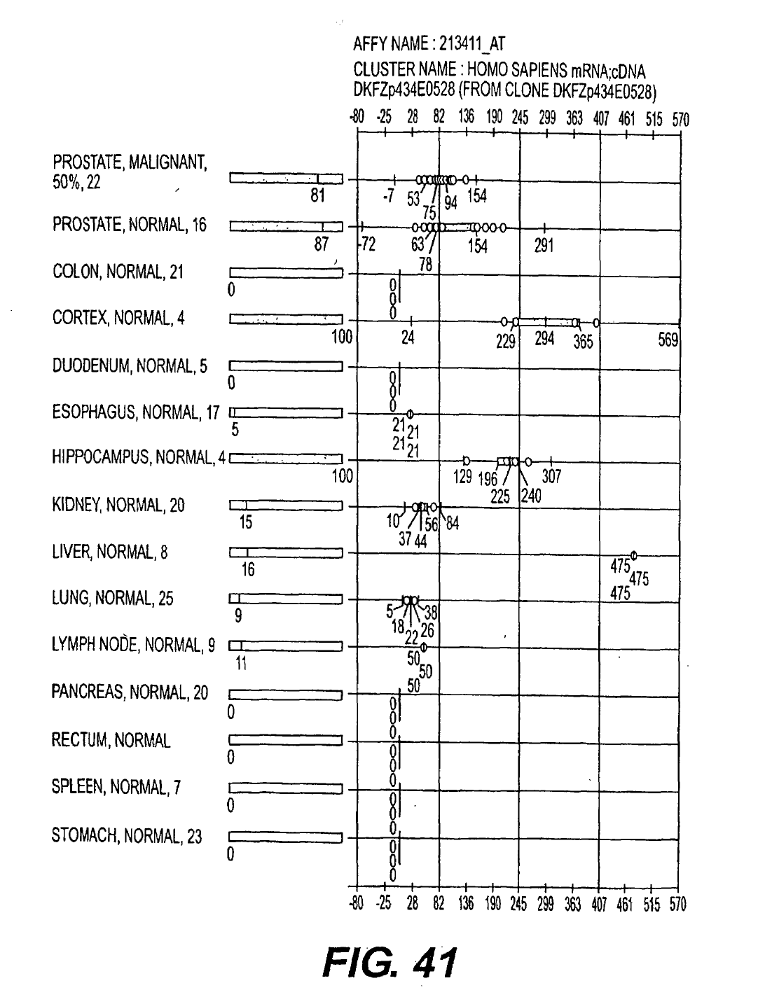

### AW242701/ADAM22 (FIG. 41)

### The DNA AW242701:

(SEQ ID NO: 62)

TAGCACCCCCAAAAGACAACTTCTTTCAGAAACGGGGTGTTTTACCTAAA
CATAGTAGCTTACATGTTAGCCAGCAGTAGGTCGGCACTAGTGTTTTCCA
CGGTTATCACCTTTGACAGGTGATGTGCATCTATAGATAGTGGAAGCCAC
CCCATGAGGAGGTGTTAATAGCAGCATGGTTTCACTTTTGGTAATCAGGT
AATCATGTGTATATACTTAGATTCGCATTATTTTAACATTTCTCTGCTAC
TCTGCACTTCAGGTTCGTTAAGCTATTTTAATAATTACTGGGGTTATGGC
AAACACCAATGGAAATGTATATGGCAACTGCTTTCCTGAGCAAGTGTGAT
TTGTTTTATGGCTGTTCAAGTTATAAAATTGTTCTTACATTGTAGGTAAA
CAAAATCTTGATGTTTTTAAAGGTCACTGTAACTTAAGGTTCAAATTTCT
GGCACAGTTTTATTAGTATTCACTTCGGAAGCTAATAAGATACCATGGTT
TTCTATGTTACTCCCATTGTA

Searching the BLAT database indicates that this sequence codes for an alternative 3′UTR of the gene ADAM22, a gene with a number of alternative splices. The longest version of ADAM22 is below.

### Nucleotide Sequence of ADAM22:

(SEQ ID NO: 63)

catgaggagctgagcgtctcgggcgaggcgggctgacggcagcaccatgc
aggcggcagtggctgtgtccgtgcccttcttgctgctctgtgtcctgggg
acctgccctccggcgcgctgcggccaggcaggagacgcctcattgatgga
gctagagaagaggaaggaaaaccgcttcgtggagcgccagagcatcgtgc
cactgcgcctcatctaccgctcgggcggcgaagacgaaagtcggcacgac
gcgctcgacacgcgggtgcggggcgacctcggtggcccgcagttgactca
tgttgaccaagcaagcttccaggttgatgcctttggaacgtcattcattc
tcgatgtcgtgctaaatcatgatttgctgtcctctgaatacatagagaga
cacattgaacatggaggcaagactgtggaagttaaaggaggagagcactg
ttactaccagggccatatccgaggaaaccctgactcatttgttgcattgt
caacatgccacggacttcatgggatgttctatgacgggaaccacacatat
ctcattgagccagaagaaaatgacactactcaagaggatttccattttca
ttcagtttacaaatccagactgtttgaattttccttggatgatcttccat
ctgaatttcagcaagtaaacattactccatcaaaatttattttgaagcca
agaccaaaaaggagtaaacggcagcttcgtcgatatcctcgtaatgtaga
agaagaaaccaaatacattgaactgatgattgtgaatgatcaccttatgt
ttaaaaaacatcggctttccgttgtacataccaatacctatgcgaaatct
gtggtgaacatggcagatttaatatataaagaccaacttaagaccaggat
agtattggttgctatggaaacctgggcgactgacaacaagtttgccatat
ctgaaaatccattgatcaccctacgtgagtttatgaaatacaggagggat
tttatcaaagagaaaagtgatgcagttcaccttttttcgggaagtcaatt
tgagagtagccggagcggggcagcttatattggtgggatttgctcgttgc
tgaaaggaggaggcgtgaatgaatttgggaaaactgatttaatggctgtt
acacttgcccagtcattagcccataatattggtattatctcagacaaaag
aaagttagcaagtggtgaatgtaaatgcgaggacacgtggtccgggtgca
taatgggagacactggctattatcttcctaaaaagttcacccagtgtaat
attgaagagtatcatgacttcctgaatagtggaggtggtgcctgcctttt
caacaaaccttctaagcttcttgatcctcctgagtgtggcaatggcttca
ttgaaactggagaggagtgtgattgtggaaccccggccgaatgtgtcctt
gaaggagcagagtgttgtaagaaatgcaccttgactcaagactctcaatg
cagtgacggtctttgctgtaaaaagtgcaagtttcagcctatgggcactg
tgtgccgagaagcagtaaatgattgtgatattcgtgaaacgtgctcagga
aattcaagccagtgtgcccctaatattcataaaatggatggatattcatg
tgatggtgttcagggaatttgctttggaggaagatgcaaaaccagagata
gacaatgcaaatacatttgggggcaaaaggtgacagcatcagacaaatat
tgctatgagaaactgaatattgaagggacggagaagggtaactgtgggaa
agacaaagacacatggatacagtgcaacaaacgggatgtgctttgtggtt
accttttgtgtaccaatattggcaatatcccaaggcttggagaactcgat
ggtgaaatcacatctactttagttgtgcagcaaggaagaacattaaactg
cagtggtgggcatgttaagcttgaagaagatgtagatcttggctatgtgg
aagatgggacaccttgtggtccccaaatgatgtgcttagaacacaggtgt
cttcctgtggcttctttcaactttagtacttgcttgagcagtaaagaagg
cactatttgctcaggaaatggagtttgcagtaatgagctgaagtgtgtgt
gtaacagacactggataggttctgattgcaacacttacttccctcacaat
gatgatgcaaagactggtatcactctgtctggcaatggtgttgctggcac
caatatcataataggcataattgctggcaccattttagtgctggccctca
tattaggaataactgcgtggggttataaaaactatcgagaacagaggtca
aatgggctctctcattcttggagtgaaaggattccagacacaaaacatat
ttcagacatctgtgaaaatgggcgacctcgaagtaactcttggcaaggta
acctgggaggcaacaaaaagaaaatcagaggcaaaagatttagacctcgg
tctaattcaactgagtatttaaacccatggttcaaaagagactataatgt
agctaagtgggtagaagatgtgaataaaaacactgaagaaccatacttta
ggactttatctcctgccaagtctccttcttcatcaactgggtctattgcc
tccagcagaaaatacccttacccaatgcctccacttcctgatgaggacaa
gaaagtgaaccgacaaagtgccaggctatgggagacatccatttaagatc
aactgtttacatgtgatacatcgaaaactgtttacttcaacttttacttc
agacaatacgaagaccctctgagatgctacagaggagaggaagcggagtt
tcacnnnnnntnaccattttctttttgtcattggcttaggatttaactaa
ccatgaaaagaactactgaaatattacactataacatggaacaataaagg
tactggtatgttaatggataatccgcatgacagataatatgtagaaatat
tcataaagttaactcacatgacccaaatgtagcaagtttcctaaggtaca
atagtggattcagaacttgacgttctgaggcacatcctcactgtaaacag
taatgctatatgcatgaagcttctgtttattgttttccatatttaaggaa
acaacatcccataatagaaatgagcatgcagggctaaggcatataggatt
tttctgcaggactttaaagctttgaaaggccaatatcccataggctaact
ttaaacatgtatttttatttttgttttgttttttacttttcatatttata
ttagcatacaaggacaattgtatatatgtaacatttttaaaattttaaaa
aaaaaaaaaa

### Protein Sequence of ADAM22:

(SEQ ID NO: 64)

MQAAVAVSVPFLLLCVLGTCPPARCGQAGDASLMELEKRKENRFVERQSI
VPLRLIYRSGGEDESRHDALDTRVRGDLGGPQLTHVDQASFQVDAFGTSF
ILDVVLNHDLLSSEYIERHIEHGGKTVEVKGGEHCYYQGHIRGNPDSFVA
LSTCHGLHGMFYDGNHTYLIEPEENDTTQEDFHFHSVYKSRLFEFSLDDL
PSEFQQVNITPSKFILKPRPKRSKRQLRRYPRNVEEETKYIELMIVNDHL
MFKKHRLSVVHTNTYAKSVVNMADLIYKDQLKTRIVLVAMETWATDNKFA
ISENPLITLREFMKYRRDFIKEKSDAVHLFSGSQFESSRSGAAYIGGICS
LLKGGGVNEFGKTDLMAVTLAQSLAHNIGIISDKRKLASGECKCEDTWSG
CIMGDTGYYLPKKFTQCNIEEYHDFLNSGGGACLFNKPSKLLDPPECGNG
FIETGEECDCGTPAECVLEGAECCKKCTLTQDSQCSDGLCCKKCKFQPMG
TVCREAVNDCDIRETCSGNSSQCAPNIHKMDGYSCDGVQGICFGGRCKTR
DRQCKYIWGQKVTASDKYCYEKLNIEGTEKGNCGKDKDTWIQCNKRDVLC
GYLLCTNIGNIPRLGELDGEITSTLVVQQGRTLNCSGGHVKLEEDVDLGY
VEDGTPCGPQMMCLEHRCLPVASFNFSTCLSSKEGTICSGNGVCSNELKC
VCNRHWIGSDCNTYFPHNDDAKTGITLSGNGVAGTNIIIGIIAGTILVLA
LILGITAWGYKNYREQRSNGLSHSWSERIPDTKHISDICENGRPRSNSWQ
GNLGGNKKKIRGKRFRPRSNSTEYLNPWFKRDYNVAKWVEDVNKNTEEPY
FRTLSPAKSPSSSTGSIASSRKYPYPMPPLPDEDKKVNRQSARLWETSI

This protein contains one TM, a signal sequence, a disintegrin motif, and an ADAM cysteine rich repeat by SMART, and two TMs by SOSUI and TmPred prediction programs. This protein has been previously purported to have use in treating neurological disorders and to have activity as an anti-angiogenic factor.

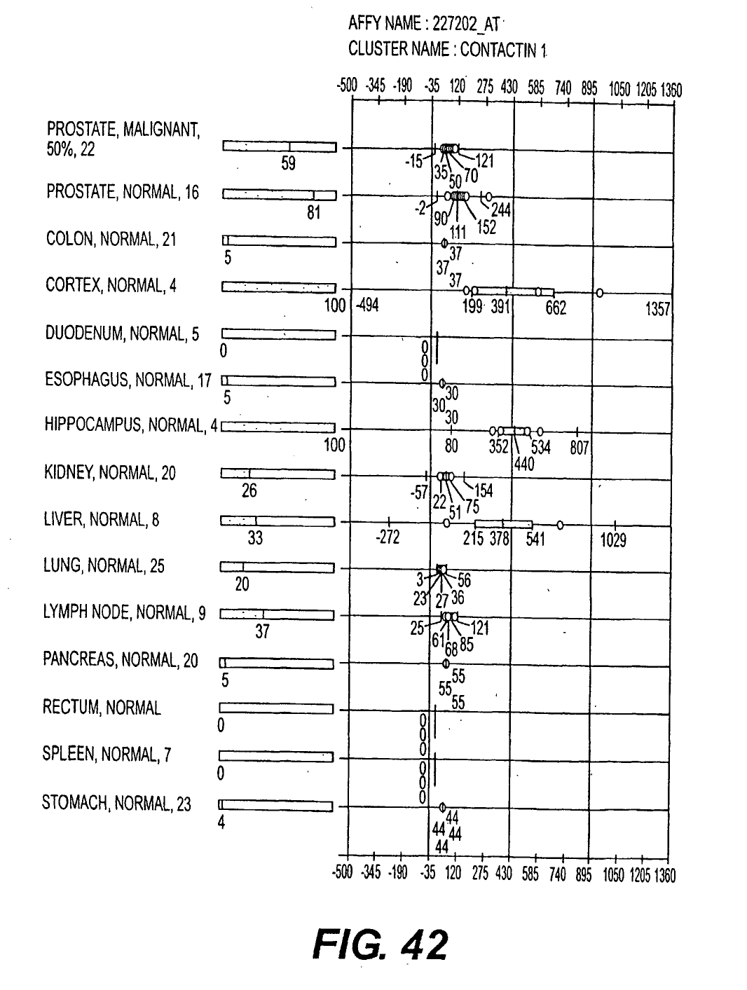

### AW072790/Contactin (FIG. 42)

Using the GeneLogic database, we found fragment AW072790 was upregulated 3.42 fold in the all prostate samples compared to mixed normal tissue without normal prostate, brain, and female specific organs. Enorthern analysis of this fragment in FIG. 42 demonstrates that it is expressed in 87% of the prostate tumors with greater than 50% malignant cells with low expression in normal tissues other than the prostate and the brain.

### The Nucleotide Sequence of AW072790

(SEQ ID NO: 65)

TTTTGCAATGTGACCCATGTTGGGCATTTTTATATAATCAACAACTAAAT
CTTTTGCCAAANGCANNNNNNNNNNNNATNNNCTAANANANGNNAATAAC
GAGCAAAACTGGTTAGATTTNGCATGAAATGGTTCTGAAAGGTAAGAGGA
AAACAGACTTTGGAGGNNGTTTAGTTTTGAATTTCTGACAGAGATAAAGT
AGTTTAAAATCTCTCGTACACTGATAACTCAAGCTTTTCATTTTCTCATA
CAGTTGTACAGATTTAACTGGGACCATCAGTTTTAAACTGTTGTCAAGCT
AACTAATAATCATCTGCTTTAAGACGCAAGATTCTGAATTAAACTTTATA
TAGGTATAGATACATCTGTTGTTTCTTTGTATTTCAGGAAAGGTGATAGT
AGTTTTATTTGATACTGATAAATATTGAATTGATTTTTTAGTTATTTTTT
ATCATTTTTTCAATGGAGTAGTATAGGACTGTGCTTTGTCCTTTT

This sequence corresponds to contactin.

### Nucleotide Sequence of Contactin:

(SEQ ID NO: 66)

gaattccggctgtgccgcaccgaggcgagcaggagcagggaacaggtgtt
taaaattatccaactgccatagagctaaattcttttttggaaaattgaac
cgaacttctactgaatacaagatgaaaatgtggttgctggtcagtcatct
tgtgataatatctattactacctgtttagcagagtttacatggtatagaa
gatatggtcatggagtttctgaggaagacaaaggatttggaccaattttt
gaagagcagccaatcaataccatttatccagaggaatcactggaaggaaa
agtctcactcaactgtagggcacgagccagccctttcccggtttacaaat
ggagaatgaataatggggacgttgatctcacaagtgatcgatacagtatg
gtaggaggaaaccttgttatcaacaaccctgacaaacagaaagatgctgg
aatatactactgtttagcatctaataactacgggatggtcagaagcactg
aagcaaccctgagctttggatatcttgatcctttcccacctgaggaacgt
cctgaggtcagagtaaaagaagggaaaggaatggtgcttctctgtgaccc
cccataccattttccagatgatcttagctatcgctggcttctaaatgaat
ttcctgtatttatcacaatggataaacggcgatttgtgtctcagacaaat
ggcaatctctacattgcaaatgttgaggcttccgacaaaggcaattattc
ctgctttgtttccagtccttctattacaaagagcgtgttcagcaaattca
tcccactcattccaatacctgaacgaacaacaaaaccatatcctgctgat
attgtagttcagttcaaggatgtatatgcattgatgggccaaaatgtgac
cttagaatgttttgcacttggaaatcctgttccggatatccgatggcgga
aggttctagaaccaatgccaagcactgctgagattagcacctctggggct
gttcttaagatcttcaatattcagctagaagatgaaggcatctatgaatg
tgaggctgagaacattagaggaaaggataaacatcaagcaagaatttatg
ttcaagcattccctgagtgggtagaacacatcaatgacacagaggtggac
ataggcagtgatctctactggccttgtgtggccacaggaaagcccatccc
tacaatccgatggttgaaaaatggatatgcgtatcataaaggggaattaa
gactgtatgatgtgacttttgaaaatgccggaatgtatcagtgcatagct
gaaaacacatatggagccatttatgcaaatgctgagttgaagatcttggc
gttggctccaacttttgaaatgaatcctatgaagaaaaagatcctggctg
ctaaaggtggaagggtgataattgaatgcaaacctaaagctgcaccgaaa
ccaaagttttcatggagtaaagggacagagtggcttgtcaatagcagcag
aatactcatttgggaagatggtagcttggaaatcaacaacattacaagga
atgatggaggtatctatacatgctttgcagaaaataacagagggaaagct
aatagcactggaacccttgttatcacagatcctacgcgaattatattggc
cccaattaatgccgatatcacagttggagaaaacgccaccatgcagtgtg
ctgcgtcctttgatcctgccttggatctcacatttgtttggtccttcaat
ggctatgtgatcgattttaacaaagagaatattcactaccagaggaattt
tatgctggattccaatggggaattactaatccgaaatgcgcagctgaaac
atgctggaagatacacatgcactgcccagacaattgtggacaattcttca
gcttcagctgaccttgtagtgagaggccctccaggccctccaggtggtct
gagaatagaagacattagagccacttctgtggcacttacttggagccgtg
gttcagacaatcatagtcctatttctaaatacactatccagaccaagact
attctttcagatgactggaaagatgcaaagacagatcccccaattattga
aggaaatatggaggcagcaagagcagtggacttaatcccatggatggagt
atgaattccgcgtggtagcaaccaatacactgggtagaggagagcccagt
ataccatctaacagaattaaaacagacggtgctgcaccaaatgtggctcc
ttcagatgtaggaggtggaggtggaagaaacagagagctgaccataacat
gggcgcctttgtcaagagaataccactatggcaacaattttggttacata
gtggcatttaagccatttgatggagaagaatggaaaaaagtcacagttac
taatcctgatactggccgatatgtccataaagatgaaaccatgagccctt
ccactgcatttcaagttaaagtcaaggccttcaacaacaaaggagatgga
ccttacagcctactagcagtcattaattcagcacaagacgctcccagtga
agccccaacagaagtaggtgtaaaagtcttatcatcttctgagatatctg
ttcattgggaacatgttttagaaaaaatagtggaaagctatcagattcgg
tattgggctgcccatgacaaagaagaagctgcaaacagagttcaagtcac
cagccaagagtactcggccaggctcgagaaccttctgccagacacccagt
attttatagaagtcggggcctgcaatagtgcagggtgtggacctccaagt
gacatgattgaggctttcaccaagaaagcacctcctagccagcctccaag
gatcatcagttcagtaaggtctggttcacgctatataatcacctgggatc
atgtcgttgcactatcaaatgaatctacagtgacgggatataaggtactc
tacagacctgatggccagcatgatggcaagctgtattcaactcacaaaca
ctccatagaagtcccaatccccagagatggagaatacgttgtggaggttc
gcgcgcacagtgatggaggagatggagtggtgtctcaagtcaaaatttca
ggtgcacccaccctatccccaagtcttctcggcttactgctgcctgcctt
tggcatccttgtctacttggaattctgaatgtgttgtgacagctgctgtt
cccatcccagctcagaagacacccttcaaccctgggatgaccacaattcc
ttccaatttctgcggctccatcctaagccaaataaattatactttaacaa
actattcaactgatttacaacacacatgatgactgaggcattcaggaacc
ccttcatcca

### Amino Acid Sequence

(SEQ ID NO: 67)

MKMWLLVSHLVIISITTCLAEFTWYRRYGHGVSEEDKGFGPIFEEQPINT
IYPEESLEGKVSLNCRARASPFPVYKWRMNNGDVDLTSDRYSMVGGNLVI
NNPDKQKDAGIYYCLASNNYGMVRSTEATLSFGYLDPFPPEERPEVRVKE
GKGMVLLCDPPYHFPDDLSYRWLLNEFPVFITMDKRRFVSQTNGNLYIAN
VEASDKGNYSCFVSSPSITKSVFSKFIPLIPIPERTTKPYPADIVVQFKD
VYALMGQNVTLECFALGNPVPDIRWRKVLEPMPSTAEISTSGAVLKIFNI
QLEDEGIYECRAENIRGKDKHQARIYVQAFPEWVEHINDTEVDIGSDLYW
PCVATGKPIPTIRWLKNGYAYHKGELRLYDVTFENAGMYQCIAENTYGAI
YANAELKILALAPTFEMNPMKKKILAAKGGRVIIECKPKAAPKPKFSWSK
GTEWLVNSSRILIWEDGSLEINNITRNDGGITYCFAENNRGKANSTGTLV
ITDPTRIILAPINADITVGENATMQCAASFDPALDLTFVWSFNGYVIDFN
KENIHYQRNFMLDSNGELLIRNAQLKHAGRYTCTAQTIVDNSSASADLVV
RGPPGPPGGLRIEDIRATSVALTWSRGSDNHSPISKYTIQTKTILSDDWK
DAKTDPPIIEGNMEAARAVDLIPWMEYEFRVVATNTLGRGEPSIPSNRIK
TDGAAPNVAPSDVGGGGGRNRELTITWAPLSREYHYGNNFGYIVAFKPFD
GEEWKKVTVTNPDTGRYVHKDETMSPSTAFQVKVKAFNNKGDGPYSLLAV
INSAQDAPSEAPTEVGVKVLSSSEISVHWEHVLEKIVESYQIRYWAAHDK
EEAANRVQVTSQEYSARLENLLPDTQYFIEVGACNSAGCGPPSDMIEAFT
KKAPPSQPPRIISSVRSGSRYIITWDHVVALSNESTVTGYKVLYRPDGQH
DGKLYSTHKHSIEVPIPRDGEYVVEVRAHSDGGDGVVSQVKISGAPTLSP
SLLGLLLPAFGILVYLEF

This protein is reported to attach to the cell surface by a GPI anchor, so there are no TM domains. The coding sequence of contactin has been earlier reported in an early application WO01 94629 by Avalon, and U.S. Pat. No. 5,739,289.

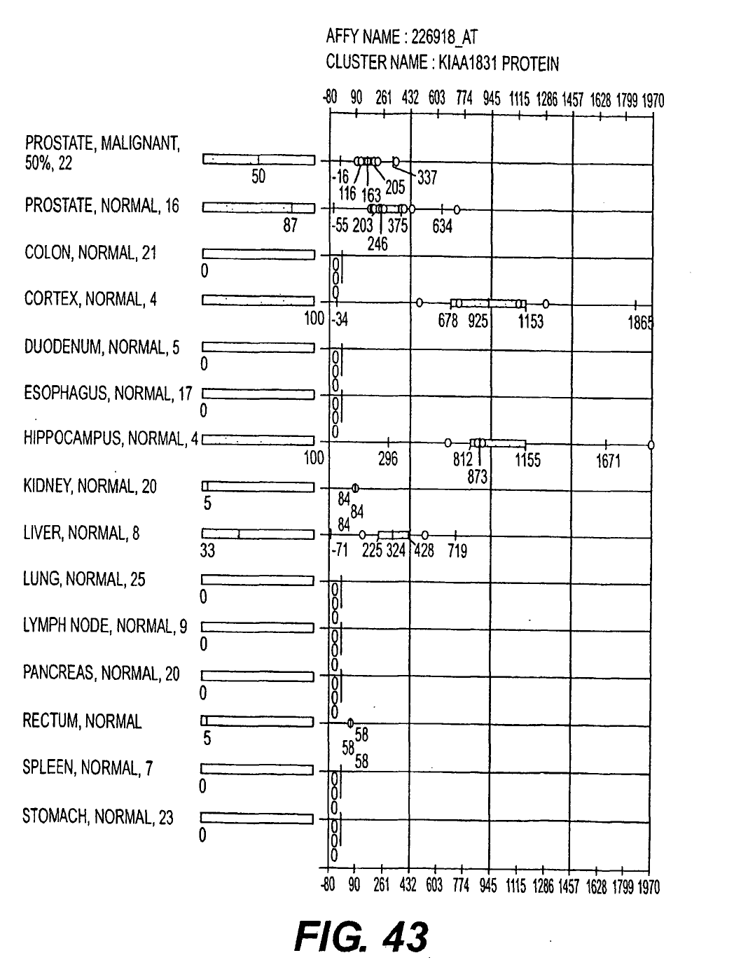

### BF513474/KIAA1831 (FIG. 43)

Using the GeneLogic database, we found fragment BF513474 was upregulated 3.62 fold in the all prostate samples from shown in FIG. 43 compared to mixed normal tissue without normal prostate, brain and female specific organs. Enorthern analysis of this fragment demonstrates that it is expressed in 50% of the prostate tumors with greater than 50% malignant cells with low expression in normal tissues other than the prostate and brain.

### Sequence of BF513474:

(SEQ ID NO: 68)

AAGCAGAAGCTGTGACAAGTTTAGTAGTCCCAAAATGGGTTATATCCCTT
CCCCCTTNACATCAGAATCTTGTGAAATGGGAAAACAACAGAAGGAGGGG
ATCAAAGATAGCTGATCTCACATGCTTCCCAGGCAGGGCAGAGGTGGGAG
TCAAACCCGGGTGACAGGTGGGTGGAGAGCCCTGTTTGAGGTTGTGGCTG
ATCCCTCTCTGGTATTAGTTTTTCCCCTGGGAGCAGGAAGCCCTAGGAAG
AGGGGACTGCAGGGTCCCCAGGGGATCTTTCCTCCCTCCCCTGCATGAGG
CAGAGGCAAGCTGCCTGCCAACCCCCTCCCTCAAGGAATGGCCTTGCCCA
GGAATGCCCACCACACATACCCTCTTCTTTTTTTCTAGTCAAACTCTTGT
TTATTCCTTGGCTTGCCTCCCTCCTTCCTCCCCTCTCAACCTTTACTTCT
GATTTCTATTTCATGGAATTTGGGATTGAGTTAAACTACAACAGTGCCGC
CAACACCAAGTCTTGCAGGAA

### This Sequence Corresponds to the Hypothetical Gene KIAA1831 Show Below:

(SEQ ID NO: 69)

TGGGGGTCTCAGTGCATCTCCTTCTCCTCTCTGCCTGCCTCCTCCCTCAC
CGAAGGGTTAGCGGACACCCATCCTTTTCTGCTTGGGGACCCCACCACCA
CCCGCAACACTGCCGCTGTCTCTTCTTCACCGTATCCTTCTCTACCCACC
CTCTTCTCTCTTCTCTTCTCCCTGCCCCTTTAAATCTGCCTGGCCCAGCC
TCCCCCGTGATGCTGGGATGGAGCAAACATTGATTTGTGCTGGGATGGAA
TCGGAATTTTGATTTATTTTTCCTCTCCCAACCATAAGAAGAAAAAAATA
ATAAAAACACCCCCTCTTGAGAGCCCCCTCCCCCTTTGCATCCAGCTCCC
AGCTCTTCTTCCCTATCTCCATCCAAGGCAGATTTTTTCCCCTACACTAT
TCTCATCTTCCCCCACCCTTGCCACTACCTCGCCCCCCCACCCAGCCTGC
TCCTCCAGCTGGGGAGAGAGGGGACTCTCCGGACTCCCCCACCTTTCCTC
TCTGGGTTGGAGCAGTCTCTCCGGAAGGGGAGGGGGCTTGGCTTGTCCGG
GCGAGGTGGGAGTGGAGGTATCCTGCCATGGATGCTGTGCCGGGGAGGCA
GCCTGAGCCCCAGCCCACATGCCACTCAGGATGAGGGTCCGGCCCTGCCT
GCCCTCGCTGGGGCCCCCCCGCCCGGCCCCGGTCTAACTGCCCCCGCCCC
GAGGCCTCGCCCGGCTCCAAGGCCCCCAGCAGGCTCTCCAGTCCCAGGAT
GCGCTGAGCCGCCGGGGGGCTGAGGCCGCGCCAACTACATGCATGTCCCC
CGGGGGCAAGTTCGACTTTGACGACGGGGGCTGCTACGTGGGGGGCTGGG
AGGCGGGGCGGGCACATGGCTACGGCGTGTGCACGGGCCCCGGCGCCCAG
GGCGAGTACAGCGGCTGCTGGGCACACGGCTTCGAGTCACTGGGCGTCTT
CACGGGGCCCGGCGGACACAGCTACCAGGGCCACTGGCAGCAGGGCAAGC
GCGAAGGGCTGGGCGTGGAGCGCAAGAGCCGCTGGACGTACCGCGGCGAG
TGGCTGGGCGGGCTGAAGGGGCGCAGCGGCGTGTGGGAAAGCGTGTCCGG
CCTGCGCTACGCCGGGCTCTGGAAGGACGGTTTCCAGGACGGCTACGGCA
CTGAGACCTACTCCGACGGAGGCACCTACCAGGGCCAGTGGCAGGCCGGG
AAGCGCCACGGCTACGGGGTACGCCAGAGTGTGCCCTACCATCAGGCGGC
GCTGCTGCGCTCGCCCCGCCGCACCTCCCTGGATTCCGGCCACAGCGACC
CCCCGACGCCACCCCCGCCCCTGCCCTTGCCGGGCGACGAGGGAGGCAGC
CCCGCCTCGGGCTCCCGGGGCGGCTTCGTGCTGGCCGGGCCCGGGGACGC
CGACGGCGCGTCGTCCCGAAAGCGCACTCCGGCGGCCGGCGGATTCTTTC
GCCGTTCGCTGCTGCTCAGCGGGCTCCGAGCGGGCGGACGTCGCAGCTCC
CTGGGCAGCAAGCGAGGCTCCCTGCGCAGCGAGGTGAGCAGCGAGGTGGG
CAGCACCGGACCGCCCGGCTCGGAGGCCAGCGGGCCCCCGGCCGCAGCGC
CGCCCGCCCTCATCGAGGGCTCGGCCACAGAGGTGTACGCGGGCGAGTGG
CGCGCAGATCGGCGCAGCGGCTTCGGCGTCAGCCAGCGCTCCAACGGGCT
GCGCTACGAGGGCGAGTGGCTGGGCAACCGGCGGCACGGCTACGGGCGCA
CCACCCGCCCCGACGGCTCCCGCGAGGAGGGCAAGTACAAGCGCAACCGG
CTGGTGCACGGCGGGCGCGTCCGCAGTCTCCTGCCTCTGGCCCTTCGGCG
GGGCAAGGTTAAGGAGAAGGTGGACAGGGCTGTCGAGGGCGCCCGTCGAG
CCGTGAGTGCTGCCCGTCAGCGCCAGGAGATCGCCGCTGCCAGGGCAGCA
GACGCCCTCCTAAAGGCAGTGGCAGCCAGCAGTGTCGCTGAGAAGGCCGT
GGAGGCAGCTCGAATGGCCAAACTGATAGCCCAGGACCTGCAGCCCATGC
TAGAGGCCCCAGGCCGCAGACCCAGGCAGGACTCAGAAGGTTCCGACACG
GAGCCCCTGGATGAGGACAGCCCTGGGGTATATGAGAACGGACTGACCCC
CTCAGAGGGATCCCCTGAACTGCCCAGCAGTCCTGCCTCCTCCCGCCAAC
CCTGGCGACCCCCTGCCTGCCGGAGCCCACTGCCTCCTGGAGGGGACCAG
GGTCCCTTCTCCAGCCCCAAAGCTTGGCCTGAGGAGTGGGGGGGGGCAGG
CGCACAGGCAGAGGAACTAGCTGGCTATGAGGCTGAGGATGAGGCTGGGA
TGCAAGGGCCAGGGCCCAGAGACGGTTCCCCACTCCTCGGAGGCTGCAGC
GACAGTTCAGGAAGTCTTCGAGAGGAGGAGGGGGAGGATGAAGAGCCCCT
GCCCCCGCTGAGGGCCCCAGCAGGCACGGAGCCTGAGCCCATCGCCATGC
TGGTCCTGAGGGGCTCGTCCTCGAGGGGTCCTGATGCTGGGTGCCTGACA
GAAGAGCTCGGGGAGCCCGCTGCAACCGAGAGGCCTGCCCAGCCGGGAGC
TGCCAACCCCCTGGTGGTGGGAGCCGTGGCCCTCCTGGACCTCAGCCTGG
CATTCCTGTTCTCCCAGCTCCTCACCTGAGGCTACTTCCTGGCCTGGTTC
TGGCTTTGGTTGCGTGCCTCTTCACCCCTTTGACCTGCCTTTTTTCTCTT
CTCCTCTTCCTGGCTGTGTTTTCTCCTATCTTTCTTTCTCTTCTTCCTTT
CTTTTCTGTGCTCCTTTGTTTTTTTCTCTCGCTTTTTCTTTCCCTGTCTT
CTTTCAGATTATCTCATTTCTTCTGGATCTGTCTCTGTATTCCTCACTCC
CTTCCCCATCCCAACCCCTTCTTTCTCTAGATTGTTTACATATGAAGGGC
TTTTCTCTCTCAGAGTTGCTGTCTTCTCTGAGACACACAAATCTAAGTCA
GACCATTGCTCCACGCCCTCCCACCTTTTCTTTAGACCTCAACTTCGCTG
CGGGTGGGGGTTTGGTGTCCTAAGGAGACTCCTGGAAGCTGAATGGAGAG
GAGGAAGAAAATGAAGAAGGAGTGATTGAATGTCGGGCAAGGCACTGGCT
GAGCTGCTGTGGCTCCCTAGCCTAAGGGGCCTGCTGTCCCTCTGAGGCCT
AGTGAAAAAGCTGCAGGAGGTGCATCCTCCACCTCTAATCTTGGAGGCTA
TTATCTTACCTCCAAGCACTGAGCTGGGTTACTGCCCAATTCCATCCTTC
CCTGAAGGAGAGAAGGGAAGTGAAAAGTAGAGTAACTCCCCAGCATTTCC
CTCTTTTTCTCCTCATCGGCCAGCCCCTCCTCCAGCCCCCTCTGGTGGCA
TGCCATGCCAAGAGCAACGTGTAAAGGAACAGAGAATATCCAATGCAGTC
AAGTCCACCCTGCCCAGACTTTGCCACTGACTTCTCCCACCCTTCTGTCT
CCCCCATAATAGTTTATTTGGTTGGTCTGGACTCACTTGTGGCCTTTGAT
TAAATTCCTAAGGGGCCTGAAGAAGACATTTCTACTGCAGAGGGTTAGAG
GCACTTGAGCAAGGCCCCCACATCCCAACTCTGGGAGTTGTGGTGGGAGG
AGGCACTTCTGGGGGATAGGACCAGACAAGATAACAGGAGCTCACATGGA
AGCAGAAGCTGTGACAAGTTTAGTAGTCCCAAAATGGGTTATATCCCTTC
CCCCTTTACATCAGAATCTTGTGAAATGGGAAAACAACAGAAGGAGGGGA
TCAAAGATAGCTGATCTCACATGCTTCCCAGGCAGGGCAGAGGTGGGAGT
CAAACCCGGGTGACAGGTGGGTGGAGAGCCCTGTTTGAGGTTGTGGCTGA
TCCCTCTCTGGTATTAGTTTTTCCCCTGGGAGCAGGAAGCCCTAGGAAGA
GGGGACTGCAGGGTCCCCAGGGGATCTTTCCTCCCTCCCCTGCATGAGGC
AGAGGCAAGCTGCCTGCCAACCCCCTCCCTCAAGGAATGGCCTTGCCCAG
GAATGCCCACCACACATACCCTCTTCTTTTTTTCTAGTCAAACTCTTGTT
TATTCCTTGGCTTGCCTCCCTCCTTCCTCCCCTCTCAACCTTTACTTCTG
ATTTCTATTTCATGGAATTTGGGATTGAAGTTAAACTACAACAGTGCCGC
CAACACCAAGTCTTGCAGGAAAAAAATACAAAGAAATTTAACAAAAAAAA
TATATTAATAAAAAAGTTCA AAAAAGGG

### The Amino Acid Sequence of KIAA1831

(SEQ ID NO: 70)

LPPPRGLARLQGPQQALQSQDALSRRGAEAAPTTCMSPGGKRDFDDGGCY
VGGWEAGRAHGYGVCTGPGAQGEYSGCWAHGFESLGVFTGPGGHSYQGHW
QQGKREGLGVERKSRWTYRGEWLGGLKRRSGVWESVSGLRYAGLWKDGFQ
DGYGTETYSDGGTYQGQWQAGHRHGYGVRQSVPYHQAALLRSPRRTSLDS
GHSDPPTPPPPLPLPGDEGGSPASGSRGGFVLAGPGDADGASSRKRTPAA
GGFFRRSLLLSGLRAGGRRSSLGSKRGSLRSEVSSEVGSTGPPGSEASGP
PAAAPPALIEGSATEVYAGEWRADRRSGFGVSQRSNGLRYEGEWLGNRRH
GYGRTTRPPGSREEGKYKRNRLVHGGRVRSLLPLALRRGKVKEKVDRAVE
GARRAVSAARQRQEIAAARAADALLKAVAASSVAEKAVEAARMAKLIAQD
LQPMLEAPGRRPRQDSEGSDTEPLDEDSPGVYENGLTPSEGSPELPSSPA
SSRQPWRPPACRSPLPPGGDQGPFSSPKAWPEEWGGAGAQAEELAGYEAE
DEAGMQGPGPRDGSPLLGGCSDSSGSLREEEGEDEEPLPPLRAPAGTEPE
PIAMLVLRGSSSRGPDAGCLTEELGEPAATERPAQPGAANPLVVGAVALL
DLSLAFLFSQLLT

This protein is predicted to have no TMs by SMART and 1 TM by SOSUI and TmPred.
BF969986/hs_{—}9_{—}17724_{—}29_{—}5_{—}665

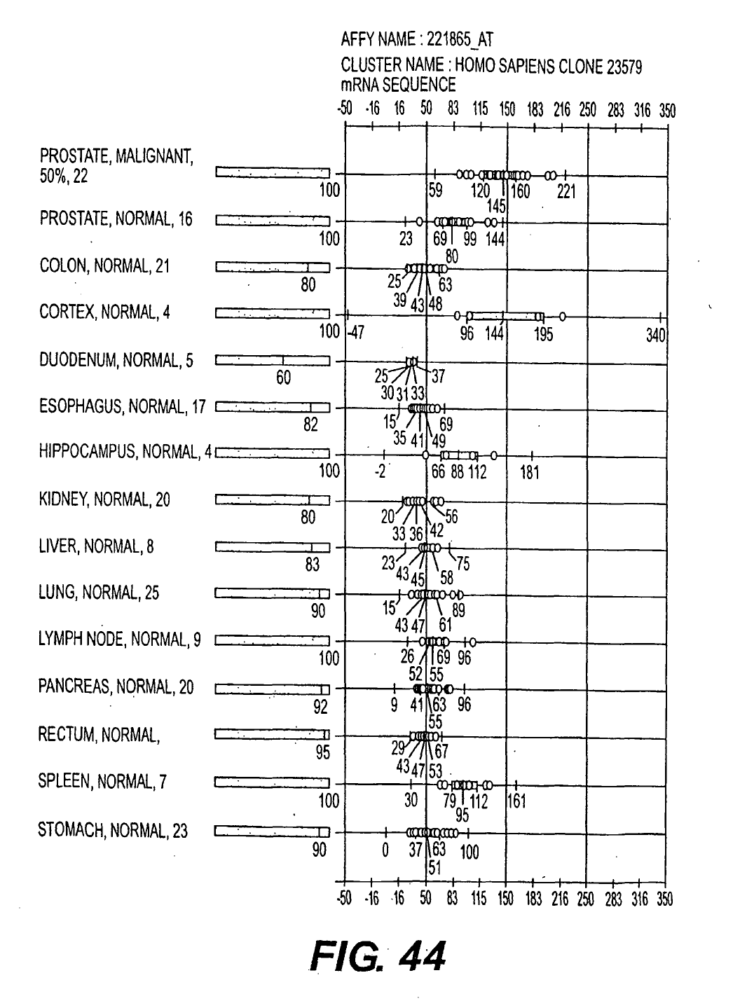

Using the GeneLogic database, we found fragment BF969986 was upregulated 3.02 fold in the malignant prostate samples compared to mixed normal tissue without normal prostate, brain and female specific organs. Enorthern analysis of this fragment shown in FIG. 44 demonstrates that it is expressed in 100% of the prostate tumors with greater than 50% malignant cells with low expression in normal tissues other than the prostate and brain.

### Nucleotide Sequence of BF969986:

(SEQ ID NO: 71)

TAAAATCCCTATGATCTCTGTCTCACCTACTTNACAGGGTTGCTGTGAAG
ATCGCATACTACACACAGGAATGCTCATCAGTTTTTAAATTTTATTTAAT
TTTTATTTATTTTTTTTTAAATGTAATTTTTTCAGAGAGATAAGGTCTTG
CTATGTTACCCAGCCTAGTCTTGAACTCCTGGCCTCAAGTGATCCTCCTG
CCTTGGCCTCCCATGCTGCTGGGATTACAGGTGTGAACTACCATGCCCAG
CCAGCTCCTAAGTCTTAAGGCTCTGTGTTAGTGATAGATGTGGCCATGGT
GTAGGCAGTGCAATGTCTTCGAGTGAGAGTGAAGGTGGTAACTCATTGCA
TGGATTCTAGAGTTCTGTTTATTCTAATCCAAGTTCTTCCACTTAAAAAC
AATGTTCTTCCTCTCATTGAGTCTCATTCCTCATCTATAGGATGGGAATA
AGAGCATGTACCTGGCAGGTTGTTGTAAGGATTAAATGGTGTAAAAAAAT
GTCAAGTGCTTGCAACTTTGAATACCAAA

### This Corresponds to the Hypothetical Gene Hs9_{—}17724_{—}29_{—}5_{—}665; the Longest of Possible Alternative Splices is Shown Below:

(SEQ ID NO: 72)

gcgcgttccctcttggccccaaagcgagtccggcgggcggctcctcgggg
ttgggcgaccgagcggggccggccgggcggggggcgggcccgtgaaggcg
gcgcagcgcggcgcgggaggcgtgctgggcgcggggctgcggtgcccaga
ggctgcggcattaggggctcggcgcccccgaccttccgcgtcccggggtg
gcggcggcggcggcggcggcggcgcgggcggcatatgatgctgagctggc
tgctccagaatgaaccacagctctgagaaggggaagtagaaacagctggc
gccctgccatggcctgtgaaccacaggtggacccgggggccactggccca
ttgcccccctcctcccctggctggagtgccctgcctggagggagccctcc
tggctgggggcaagagctccacaatggccaggtcctcactgttctccgga
ttgacaatacctgtgcacccatctccttcgacctgggagccgcagaagag
caactgcaaacttggggcatccaggtcccggctgaccagtacaggagctt
ggctgagagtgccctcttggagccccaagtgagaagatatatcatctaca
actcgaggcctatgcggctggcctttgctgtggttttctatgtggtggtg
tgggccaatatctactctaccagtcagatgtttgccttggggaaccactg
ggctggcatgctgctcgtgaccctggccgcggtgagcctgaccttgactc
ttgtgctggtctttgaaagacaccagaagaaggccaacaccaacacggac
ctgaggctggcagctgccaatggagccctcctgagacaccgggtgctgct
gggggtgacagacacagtggaaggatgccagagtgtgattcagctttggt
ttgtctacttcgacctggagaactgtgtgcagtttttgtctgatcatgtt
caagaaatgaagactagccaagaggtattgctgagaagcagattgagcca
gttgtgtgttgtcatggagactggggtgagccctgcaacagcggaggggc
ctgagaacttggaggatgctcctctcctgcccggcaattcttgtcctaac
gagaggccactcatgcagactgagcttcatcagcttgttcctgaggctga
gccggaggaaatggcccgccagctgctggcagtgtttggcggctactaca
tccggcttctagtgacctcccagctccctcaggcaatggggacacgacac
acgaactctccgagaattccatgcccctgccagctcatagaagcctacat
cctaggcacagggtgctgcccgttcctggcgaggtgacctagggatgaag
gtactcatcttccttcaagactgagcagtcaggaaggcttcaggagccca
agatggccaatggggagccccaggtgaggagagaagcatctgggggcact
ccaaaaggggcctgtgatgtcagccactggggtgttgtgctcacttcagg
gcccagcacaaaaatccttgtttgacatctcatgctgaccccctggcctt
tgcagaagctgatggttacagagctagtcccaccaaagctactctctctg
ctgcttagaactgtggacacgtatggaaagactggacccccattgctttc
attgttcagagaacccaggagacatgaagatgaccagactgggcaaatta
tgtgtccaaaacttggcctcagatgatgtttccatctccaaccccttcat
gccagatggggaaactgaggctcagagaggatactgctctatgtggcatt
gccttgaacccctaaaattatcagacttcctttttccaatataaagaaaa
aaagtaagttttcagaattctctcaatttttaagtttttctcccccatat
tttgtgaaaagcagtggtatgtgtacgtgttgtctaccagtacacaggct
gcagaagacagagacagaagaaagagatcaagggcagataactgttgata
ggaatatttgagaaagattgatcctgtttgacttgaggacttattttgtt
cacaggcatgcacgcttgtggttgtggttttatattacagatgtagaaca
atggttatgtttcccgacatgaacattgtcctggaatgaagtgtgatcag
ccacttgtggaattctttgaagagctcagaggcttccaagtgatctgctc
ctgaacaagtttgaagacctattgtttcatagacccaagaccaaacgcat
ctaaaggatccccagcccccaagacctagcctttgtctgcgattttggct
tcatctcccacaaaacccctttatgagttcacgctctttcctggactgac
atacctattcctttccatttgttggactcctattcatgcttcaaagtcca
gctttcttaagcccttctttaggaagccttcccacacagccaaccctgct
gctctctgcctcctttaaattcttgatacagctgctgcttgttctgatgt
tttatggtattgattctgttttcctgtgtatatgccagtttttctagcta
gactgtaaactccttaaggacagagactacaccttgtactttttgtgcat
gacctggacctgctaaggaaaaaaaaatcttgtggattgattgctttgcc
atccccacagcagcttttgcaaattgctttccaaactcacttgaatgatg
acattgctgtggacctgggttctggacctgatctgccacttcaagctgtg
taatttttggcaagttgctttctttgcctggtcctcagtttgcccatcaa
tataatgggtggattggatgatttttttttttttaattgagatggagtct
tgcactgtcacccaggctggagtgcagtggcgcgatcttggctcactgca
acccccgccacctaggttcaagtgattctcatgcctcagcctcccaagta
gctgggactacaggtgtgcaccactactcctggatatttttttgtgtttt
tagtagagatggggtttcgccatgttggccaagctggtcttgaactcctg
acctcaggtgatccacccgcctcgggctcccaaagtgctgggattacaga
cgtgaggcaccacaaccagcctggatgattcttaagggcccttctaggac
caaagttctgggaatttctagcttattctgccccctcatagcccttggcc
tatctatctttatccacatgcagaaacatctggcaaccccacatggctga
gatgacctggtcctaggacacccttggacagaagactggcctacctagca
gacctggatttttcttcctgatctgctgcttccaagttgtgtgaccttgg
ctaagtcacttaacctttctgattgtcatttcgctttttaataaagtggg
tctggtgaacaagaaatgtaataaacacgtggcttgccattcaagagatg
agtctgaccattcactttctgtgtgccagagaagagagatcatgggtata
gaccagcccctggaaaggctgctttggtcaaggctgagagcagctttgct
caaggaaattattcacgaaggtgaccactgtctttctgacctggcacaga
ggaaatgttggctgtgaatgtgaccaatagaaagaagcccgtatttctca
gtcagtcctagaaccccggtaagtaattaacagagaataaaaatgtgttt
gttaaatgacaaagcagcagtttttcaattgtaaggtctgcttgagagcc
tttgatgtgtgtttcttttcctgacttttcctttctttagaatttttgat
ggtctcacctggtgggtggggctttcagggtatgcccacaatgtacattt
ctcggcatctgtgcctcagtttcctcatttataaaatccctatgatctct
gtctcacctactttacagggttgctgtgaagatcgcatactacacacagg
aatgtaattttttcagagagataaggtcttgctatgttacccagcctagt
cttgaactcctggcctcaagtgatcctcctgccttggcctcccatgctgc
tgggattacaggtgtgaactaccatgcccagccagctcctaagtcttaag
gctctgtgttagtgatagatgtggccatggtgtaggcagtgcaatgtctt
cgagtgagagtgaaggtggtaactcattgcatggattctagagttctgtt
tattctaatccaagttcttccacttaaaaacaatgttcttcctctcattg
agtctcattcctcatctataggatgggaataagagcatgtacctggcagg
ttgttgtaaggattaaatggtgtaaaaaaatgtcaagtgcttgcaacttt
gaataccaaacttgagtgaaagctcaataaattgttacttaaaaaa

### Hs9_{—}17724_{—}29_{—}5_{—}665 Amino Acid Sequence

(SEQ ID NO: 73)

MACEPQVDPGATGPLPPSSPGWSALPGGSPPGWGQELHNGQVLTVLRIDN
TCAPISFDLGAAEEQLQTWGIQVPADQYRSLAESALLEPQVRRYIIYNSR
PMRLAFAVVFYVVVWANIYSTSQMFALGNHWAGMLLVTLAAVSLTLTLVL
VFERHQKKANTNTDLRLAAANGALLRHRVLLGVTDTVEGCQSVIQLWFVY
FDLENCVQFLSDHVQEMKTSQEVLLRSRLSQLCVVMETGVSPATAEGPEN
LEDAPLLPGNSCPNERPLMQTELHQLVPEAEPEEMARQLLAVFGGYYIRL
LVTSQLPQAMGTRHTNSPRIPCPCQLIEAYILGTGCCPFLAR

This sequence has 2 TMs by SMART™, SOSUI™ and TmPred.

### NM_{—}020372

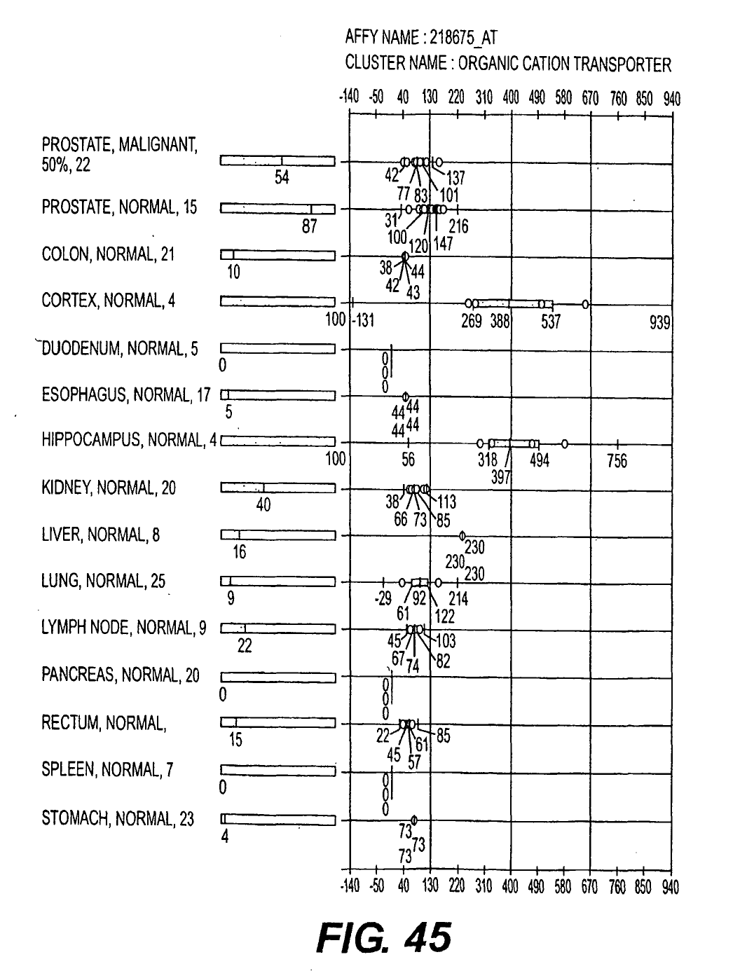

Using the GeneLogic database, we found fragment NM_{—}020372 was upregulated 3.14 fold in the all prostate samples compared to mixed normal tissue without normal prostate, brain, and female specific organs. Enorthern analysis of this fragment shown in FIG. 45 demonstrates that it is expressed in 54% of the prostate tumors with greater than 50% malignant cells with low expression in normal tissues other than the prostate and brain.

### Sequence of NM_{—}020372:

(SEQ ID NO: 74)

CTTCCTGCAGCACGTGGTGCTGGCGGCCTGCGCCCTCCTCTGCATTCTCA
GCATTATGCTGCTGCCGGAGACCAAGCGCAAGCTCCTGCCCGAGGTGCTC
CGGGACGGGGAGCTGTGTCGCCGGCCTTCCCTGCTGCGGCAGCCACCCCC
TACCCGCTGTGACCACGTCCCGCTGCTTGCCACCCCCAACCCTGCCCTCT
GAGCGGCCTCTGAGTACCCTGGCGGGAGGCTGGCCCACACAGAAAGGTGG
CAAGAAGATCGGGAAGACTGAGTAGGGAAGGCAGGGCTGCCCAGAAGTCT
CAGAGGCACCTCACGCCAGCCATCGCGGAGAGCTCAGAGGGCCGTCCCCA
CCCTGCCTCCTCCCTGCTGCTTTGCATTCACTTCCTTGGCCAGAGTCAGG
GGACAGGGAGGGAGCTCCACACTGTAACCACTGGGTCTGGGCTCCATCCT
GCGCCCAAAGACATCCACCCAGACCTCATTATTTCTTGCTCTATCATT

### This Corresponds to the LOC57100 Gene:

(SEQ ID NO: 75)

cctccacaggcgtcatggccctccgattcctcttgggctttctgcttgcc
ggtgttgacctgggtgtctacctgatgcgcctggagctgtgcgacccaac
ccagaggcttcgggtggccctggcaggggagttggtgggggtgggagggc
acttcctgttcctgggcctggcccttgtctctaaggattggcgattccta
cagcgaatgatcaccgctccctgcatcctcttcctgttttatggctggcc
tggtttgttcctggagtccgcacggtggctgatagtgaagcggcagattg
aggaggctcagtctgtgctgaggatcctggctgagcgaaaccggccccat
gggcagatgctgggggaggaggcccaggaggccctgcaggacctggagaa
tacctgccctctccctgcaacatcctcctcttcctttgcttccctcctca
actaccgcaacatctggaaaaatctgcttatcctgggcttcaccaacttc
attgcccatgccattcgccactgctaccagcctgtgggaggaggagggag
cccatcggacttctacctgtgctctctgctggccagcggcaccgcagccc
tggcctgtgtcttcctgggggtcaccgtggaccgatttggccgccggggc
atccttcttctctccatgacccttaccggcattgcttccctggtcctgct
gggcctgtgggattatctgaacgaggctgccatcaccactttctctgtcc
ttgggctcttctcctcccaagctgccgccatcctcagcaccctccttgct
gctgaggtcatccccaccactgtccggggccgtggcctgggcctgatcat
ggctctaggggcgcttggaggactgagcggcccggcccagcgcctccaca
tgggccatggagccttcctgcagcacgtggtgctggcggcctgcgccctc
ctctgcattctcagcattatgctgctgccggagaccaagcgcaagctcct
gcccgaggtgctccgggacggggagctgtgtcgccggccttccctgctgc
ggcagccaccccctacccgctgtgaccacgtcccgctgcttgccaccccc
aaccctgccctctgagcggcctctgagtaccctggcgggaggctggccca
cacagaaaggtggcaagaagatcgggaagactgagtagggaaggcagggc
tgcccagaagtctcagaggcacctcacgccagccatcgcggagagctcag
agggccgtccccaccctgcctcctccctgctgctttgcattcacttcctt
ggccagagtcaggggacagggagggagctccacactgtaaccactgggtc
tgggctccatcctgcgcccaaagacatccacccagacctcattatttctt
gctctatcattctgtttcaataaagacatttggaataaacgagcatatca
tagcctggac

### Amino Acid Sequence of LOC57100

(SEQ ID NO: 76)

MALRFLLGFLLAGVDLGVYLMRLELCDPTQRLRVALAGELVGVGGHFLFL
GLALVSKDWRFLQRMITAPCILFLFYGWPGLFLESARWLIVKRQIEEAQS
VLRILAERNRPHGQMLGEEAQEALQDLENTCPLPATSSSSFASLLNYRNI
WKNLLILGFTNFIAHAIRHCYQPVGGGGSPSDFYLCSLLASGTAALACVF
LGVTVDRFGRRGILLLSMTLTGIASLVLLGLWDYLNEAAITTFSVLGLFS
SQAAAILSTLLAAEVIPTTVRGRGLGLIMALGALGGLSGPAQRLHMGHGA
FLQHVVLAACALLCILSIMLLPETKRKLLPEVLRDGELCRRPSLLRQPPP
TRCDHVPLLATPNPAL

SOSUI and TmPred predict 9 TM domains and SMART predicts 8 TM domains and a signal peptide. This gene was previously reported to be involved in atherosclerosis and to function as an amino acid transporter. (See WO/0104264 and U.S. Pat. No. 6,313,271).

### GLUT12

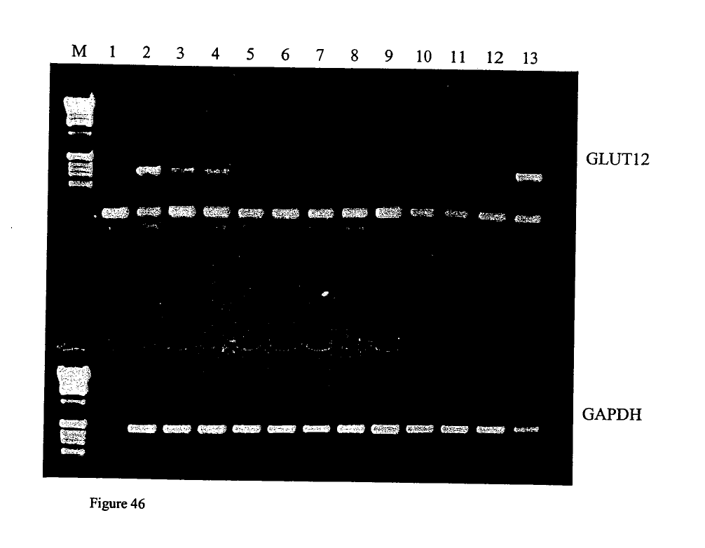

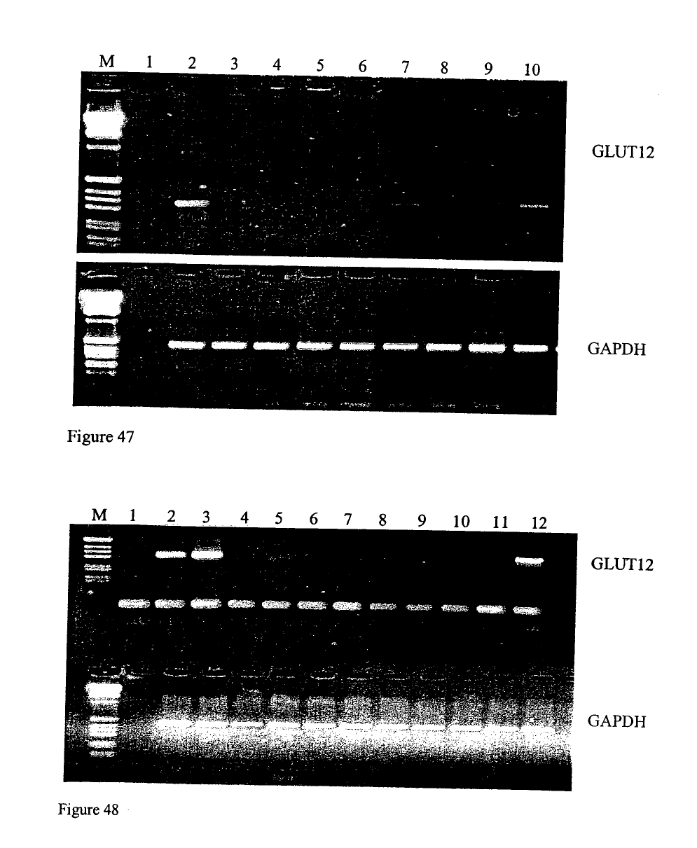

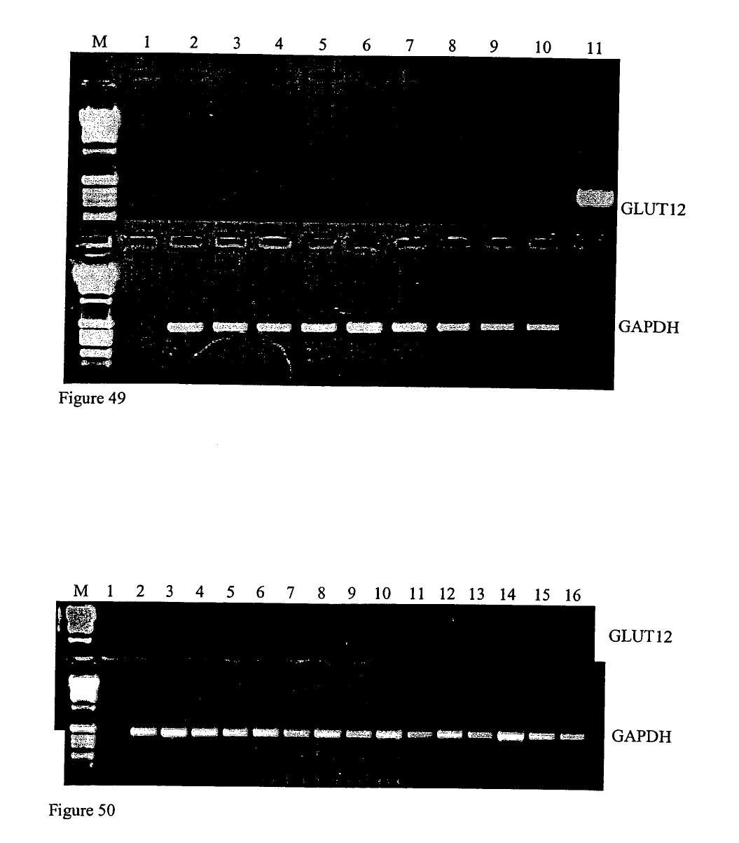

Using the GeneLogic database, we found that fragment A1742872 corresponded to the hypothetical protein Hs6_{—}25897_{—}28_{—}16_{—}1426.a in the BLAT database. This gene has been named GLUT12 Rogers et al. Am J Physiol Endorcrinol Metab, 2002, 283, E788-E738) and SLC2A12 (June 2002 update of BLAT). We refer to the gene as GLUT12. The Roger's manuscript confirms that this is a glucose transporter. However, the Roger's manuscript also suggests that the gene is expressed in heart and skeletal muscle in addition to prostate, this is not consistent with our GeneLogic data. We had previously begun PCR panels for this gene. The data is contained in FIGS. 46-50.

### N62096/Hs2_{—}5396_{—}28_{—}4_{—}677/PSAT

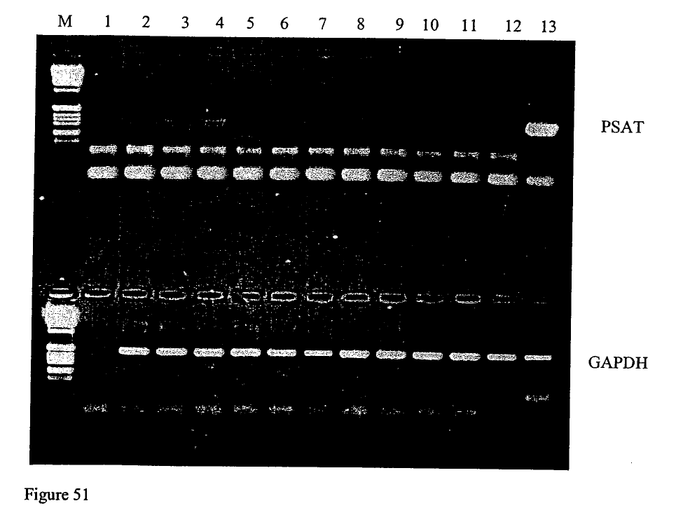

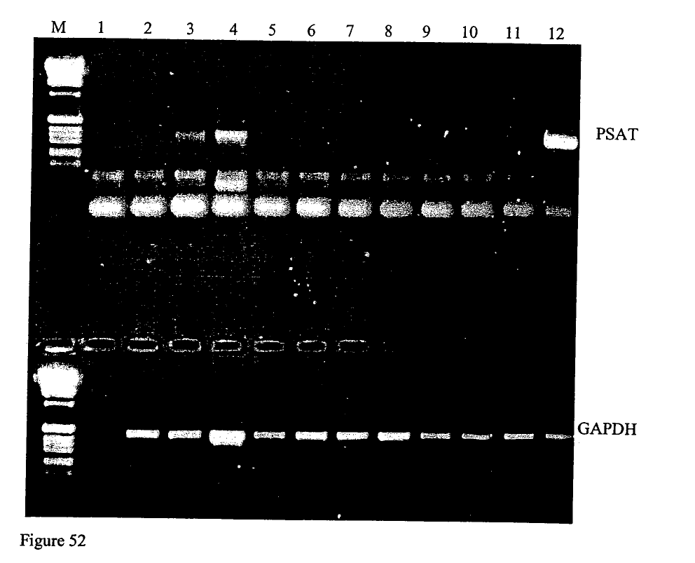

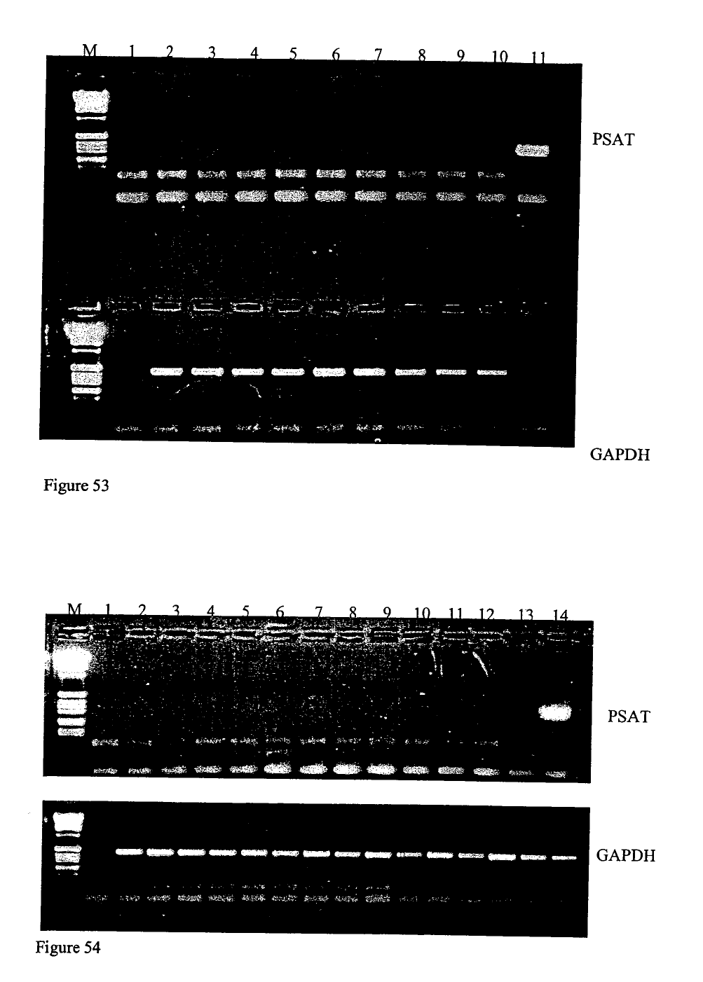

The April 2002 BLAT database predicted the protein Hs2_{—}5396_{—}28_{—}4_{—}677. We used this sequence to perform the PCR panels shown in FIGS. 51-54. This gene has homology to amino acid transporters, we have been calling this gene PSAT (Prostate Specific Amino acid Transporter).

### Possible Alternative Splices of PSAT

We purchased EST N62096 and sequenced the insert of the plasmid. The sequence is below and matches (with a few minor sequencing errors) bases 287-1297 of Hs2_{—}5396_{—}28_{—}4_{—}677a, indicating that this message including the predicted 5′UTR (bases 1-739, so the least bases 287-739 are present).

Hs2_{—}5396_{—}28_{—}4_{—}677a (a.k.a. PSAT Short)

(SEQ ID NO: 77)

gctgaagaatttagggagttgattctgatgtaagaagacaatggataaag
tatttttcagaagtcagtacaaattggcagcaaatctaccaaaaacaaat
aataagagaaaaactatcagtgatggatttatcttcacatgtagcatgta
ctggtttaaatcagtgaataactacatagttattgaattcaaaaactttt
atttagacctggtcatctattctcttaattaaatgaaatgaagtttatgg
agattcacttataagtcatgtgttgcttaatgacagggaaacattctgag
aaatgcattgttaggtgatttcctcattgtgcaaacatcacagagtatac
gtacacaaatctagatggtagcacctattacacacctaggctatatgcta
tagcttattgctcctaggctataaacctctacagcatgtttctgtactga
attctgtaggcaactgtagcagaatggaaagtatttatgtatctaaacat
agaaaaatatatagtaaaaatacagcattgtaatcatatatgtgggccat
taggtgatgcataactgtaatatctaatatttaatttattagatagttat
ctcaaacatttagtatctagtaaataaacttattttatattactatctag
gggacttatttgaaaattactgcagaaatgatgacctggtaacatttgga
agattttgttatggtgtcactgtcattttgacataccctATGGAATGCTT
TGTGACAAGAGAGGTAATTGCCAATGTGTTTTTTGGTGGGAATCTTTCAT
CGGTTTTCCACATTGTTGTAACAGTGATGGTCATCACTGTAGCCACGCTT
GTGTCATTGCTGATTGATTGCCTCGGGATAGTTCTAGAACTCAATGGTGT
GCTCTGTGCAACTCCCCTCATTTTTATCATTCCATCAGCCTGTTATCTGA
AACTGTCTGAAGAACCAAGGACACACTCCGATAAGATTATGTCTTGTGTC
ATGCTTCCCATTGGTGCTGTGGTGATGGTTTTTGGATTCGTCATGGCTAT
TACAAATACTCAAGACTGCACCCATGGGCAGGAAATGTTCTACTGCTTTC
CTGACAATTTCTCTCTCACAAATACCTCAGAGTCTCATGTTCAGCAGACA
ACACAACTTTCTACTTTAAATATTAGTATCTTTCAATGAgttgactgctt
taaaaatatgtatgttttcatagactttaaaacacataacatttacgctt
gctttagtctgtatttatgttatataaaattattattttggctttta

### PSAT Short Protein

(SEQ ID NO: 78)

MECFVTREVIANVFFGGNLSSVFHIVVTVMVITVATLVSLLIDCLGIVLE
LNGVLCATPLIFIIPSACYLKLSEEPRTHSDKIMSCVMLPIGAVVMVFGF
VMAITNTQDCTHGQEMFYCFPDNFSLTNTSESHVQQTTQLSTLNISIFQ

SMART analysis suggests that this protein has three TM domains. However, this protein has homology to amino acid transporter. These proteins have 10-12 membrane spanning segments, PSAT-short has only 3. Continued searching of the databases indicates that there are four possible alternatively spliced genes in this region, three from the June 2002 update of BLAT and one from the BLAST database. The BLAT predictions are shown below:

The first BLAT prediction is from GENESCAN:

### >NT_{—}022154.57

(SEQ ID NO: 79)

ATGACTTTTGGACAAAGGACTGGTTTTAGGAATCCTGAAAGTTTCTGGGA
GACTTTACCAGTCTTATTTCTGCAAGTCATGATTACCACATATTTTGTAG
CTAAACAATTGCTGTTCCTACACAGTAAGATCATCATCTTGCCCTCGCGG
CCTGCCGAGGGAGCAGGGGGCGCCCGTGGAACTGGCTCCCTGCAGCTCTG
CGGCTACACGCGGACCTCGGCTGTGTGCGAGGTGGCGGAGGAGGCTGGCC
GGGTGCGAATCCGTACCCAGCCCCAGCATCTTCCACCTGCTGAGGACCAC
CGCTCAGCCATGGGCTACCAGAGGCAGGAGCCTGTCATCCCGCCGCAGAG
AGATTTAGATGACAGAGAAACCCTTGTTTCTGAACATGAGTATAAAGAGA
AAACCTGTCAGTCTGCTGCTCTTTTTAATGTTGTCAACTCGATTATAGGA
TCTGGTATAATAGAAAGTAGTAGATGGGGAAGTCATTTTAAAGCTTCATT
AAGGCTAAGAGACGACTGTGCTCTGAAAGTGCAGATAGCAGGGCTTCGTG
GGCAGGTGCGTGTGAATGAGCAACCTTATTCAGCTGTTGTTTGTGGAGAC
TTTTCCCTTGTTTTATTGATAAAAGGAGGGGCCCTCTCTGGAACAGATAC
CTACCAGTCTTTGGTCAATAAAACTTTCGGCTTTCCAGGGTATCTGCTCC
TCTCTGTTCTTCAGTTTTTGTATCCTTTTATAGTTGATCCTGAAAACGTG
TTTATTGGTCGCCACTTCATTATTGGACTTTCCACAGTTACCTTTACTCT
GCCTTTATCCTTGTACCGAAATATAGCAAAGCTTGGAAAGGTCTCCCTCA
TCTCTACAGGTTTAACAACTCTGATTCTTGGAATTGTAATGGCAAGGGCA
ATTTCACTGGGTCCACACATACCAAAAACAGAAGACGCTTGGGTATTTGC
AAAGCCCAATGCCATTCAAGCGGTCGGGGTTATGTCTTTTGCATTTATTT
GCCACCATAACTCCTTCTTAGTTTACAGTTCTCTAGAAGAACCCACAGTA
GCTAAGTGGTCCCGCCTTATCCATATGTCCATCGTGATTTCTGTATTTAT
CTGTATATTCTTTGCTACATGTGGATACTTGACATTTACTGGCTTCACCC
AAGGGGACTTATTTGAAAATTACTGCAGAAATGATGACCTGGTAACATTT
GGAAGATTTTGTTATGGTGTCACTGTCATTTTGACATACCCTATGGAATG
CTTTGTGACAAGAGAGGTAATTGCCAATGTGTTTTTTGGTGGGAATCTTT
CATCGGTTTTCCACATTGTTGTAACAGTGATGGTCATCACTGTAGCCACG
CTTGTGTCATTGCTGATTGATTGCCTCGGGATAGTTCTAGAACTCAATAT
AGGCACATCTTCCATACAAGCTCAGATTCCAGGAAAGAATCAGATGACAG
CCTTGTCCTCAAATGAAAGAACTATCCTGAGTTGTACAAAGACTACAGAC
AGCCTTGACTTCTGTACTGATAGCCAAACAAAAGTGAAGCAAACTCACTG
CCCTGTTGGCGCACCAGCCTTCCCGAAGCGCAGCCTAGCGGTGGGAATGG
GAACACCTCGTCTGGGAGCTTTCTTTCGGTTCAGCTTCCCCAGCCGGACC
CCAAAGACCCGAAGCCCTGGGGGAAGGAAATTCCAACTTGCTCCCGGCCC
ACCCCCGCCCCGTTCCTCTCTCCGGCTCGCTGCTTCCCTCGCTCCAATGC
CGCCGAGCTGGTCCCCACTTATGTGCGGCCGTGCTGCAGAGGCGGCGGCG
AGCTCCCGGACTCCGGGCAGGGAAATGGGGCAGGGACGCCCCAGCCAGGT
AAGCCCAGAGCGCCGCGCCGCCTCTCACCGGGGAGGGCGAGGCCGGCGAG
GACAGCGAGGCCTCGGCCGTTTCACCTGGCTGGCAACTCGCTGCCCTGCC
GGCGGCCTGACTCACTGA

### Encoding Protein

### >NT 022154.57

(SEQ ID NO: 80)

MTFGQRTGFRNPESFWETLPVLFLQVMITTYFVAKQLLFLHSKIIILPSR
PAEGAGGARGTGSLQLCGYTRTSAVCEVAEEAGRVRIRTQPQHLPPAEDH
RSAMGYQRQEPVIPPQRDLDDRETLVSEHEYKEKTCQSAALFNVVNSIIG
SGIIESSRWGSHFKASLRLRDDCALKVQIAGLRGQVRVNEQPYSAVVCGD
FSLVLLIKGGALSGTDTYQSLVNKTFGFPGYLLLSVLQFLYPFIVDPENV
FIGRHFIIGLSTVTFTLPLSLYRNIAKLGKVSLISTGLTTLILGIVMARA
ISLGPHIPKTEDAWVFAKPNAIQAVGVMSFAFICHHNSFLVYSSLEEPTV
AKWSRLIHMSIVISVFICIFFATCGYLTFTGFTQGDLFENYCRNDDLVTF
GRFCYGVTVILTYPMECFVTREVIANVFFGGNLSSVFHIVVTVMVITVAT
LVSLLIDCLGIVLELNIGTSSIQAQIPGKNQMTALSSNERTILSCTKTTD
SLDFCTDSQTKVKQTHCPVGAPAFPKRSLAVGMGTPRLGAFFRFSFPSRT
PKTRSPGGRKFQLAPGPPPPRSSLRLAASLAPMPPSWSPLMCGRAAEAAA
SSRTPGREMGQGRPSQVSPERRAASHRGGRGRRGQRGLGRFTWLATRCPA
GGLTH

ESTs from the region do not back up this prediction.
Second BLAT prediction is from Fgenesh++

### >C2001829

(SEQ ID NO: 81)

AGAGATTTAGATGACAGAGAACCCTTGTTTCTGAACATGAGTATAAAGAG
AAAACCTGTCAGTCTGCTGCTCTTTTTAATGTTGTCAACTCGATTATAGG
ATCTGGTATAATAGGATTGCCTTATTCAATGAAGCAAGCTGGGTTTCCTT
TGGGAATATTGCTTTTATTCTGGGTTTCATATGTTACAGACTTTTCCCTT
GTTTTATTGATAAAAGGAGGGGCCCTCTCTGGAACAGATACCTACCAGTC
TTTGGTCAATAAAACTTTCGGCTTTCCAGGGTATCTGCTCCTCTCTGTTC
TTCAGTTTTTGTATCCTTTTATAGCAATGATAAGTTACAATATAATAGCT
GGAGATACTTTGAGCAAAGTTTTTCAAAGAATCCCAGGAGCATTTATTTG
CCACCATAACTCCTTCTTAGTTTACAGTTCTCTAGAAGAACCCACAGTAG
CTAAGTGGTCCCGCCTTATCCATATGTCCATCGTGATTTCTGTATTTATC
TGTATATTCTTTGCTACATGTGGATACTTGACATTTACTGGCTTCACCCA
AGGGGACTTATTTGAAAATTACTGCAGAAATGATGACCTGGTAACATTTG
GAAGATTTTGTTATGGTGTCACTGTCATTTTGACATACCCTATGGAATGC
TTTGTGACAAGAGAGGTAATTGCCAATGTGTTTTTTGGTGGGAATCTTTC
ATCGGTTTTCCACATTGTTGTAACAGTGATGGTCATCACTGTAGCCACGC
TTGTGTCATTGCTGATTGATTGCCTCGGGATAGTTCTAGAACTCAATGGT
GTGCTCTGTGCAACTCCCCTCATTTTTATCATTCCATCAGCCTGTTATCT
GAAACTGTCTGAAGAACCAAGGACACACTCCGATAAGATTATGTCTTGTG
TCATGCTTCCCATTGGTGCTGTGGTGATGGTTTTTGGATTCGTCATGGCT
ATTACAAATACTCAAGACTGCACCCATGGGCAGGAAATGTTCTACTGCTT
TCCTGACAATTTCTCTCTCACAAATACCTCAGAGTCTCATGTTCAGCAGA
CAACACAACTTTCTACTTTAAATATTAGTATCTTTCAATGA

### Encoding Protein

### >C2001829

(SEQ ID NO: 82)

RDLDDRETLVSEHEYKEKTCQSAALFNVVNSIIGSGIIGLPYSMKQAGFP
LGILLLFWVSYVTDFSLVLLIKGGALSGTDTYQSLVNKTFGFPGYLLLSV
LQFLYPFIAMISYNIIAGDTLSKVFQRIPGAFICHHNSFLVYSSLEEPTV
AKWSRLIHMSIVISVFICIFFATCGYLTFTGFTQGDLFENYCRNDDLVTF
GRFCYGVTVILTYPMECFVTREVIANVFFGGNLSSVFHIVVTVMVITVAT
LVSLLIDCLGIVLELNGVLCATPLIFIIPSACYLKLSEEPRTHSDKIMSC
VMLPIGAVVMVFGFVMAITNTQDCTHGQEMFYCFPDNFSLTNTSESHVQQ
TTQLSTLNISIFQ

The EST database backs up this prediction, however, the start codon is not an ATG.
The third BLAT prediction is from Twinscan:
>chr2.164.004.a

(SEQ ID NO: 83)

ATGAAGTTTCCAACAGGTGGTTGCTTCAGGGAAAAGCTCCAGCTTCAGCC
ATCATGTCTCTGCATTCTGGCCAGTGAGAAGGAGCAAAAGAAAGCATCTC
CGTCTCCGGAGGAAAAATACATTTGTCTGGGCGAACTCCGGTGGAAAAGC
GCCCCAGGCTGCCACAGCCTAGAGATCTTGGGGCTGCAGCCCTCGCGGCC
TGCCGAGGGAGCAGGGGGCGCCCGTGGAACTGGCTCCCTGCAGCTCTGCG
GCTACACGCGGACCTCGGCTGTGTGCGAGGTGGCGGAGGAGGCTGGCCGG
GTGCGAATCCGTACCCAGCCCCAGCATCTTCCACCTGCTGAGGACCACCG
CTCAGCCATGGGCTACCAGAGGCAGGAGCCTGTCATCCCGCCGCAGAGAG
ATTTAGATGACAGAGAAACCCTTGTTTCTGAACATGAGTATAAAGAGAAA
ACCTGTCAGTCTGCTGCTCTTTTTAATGTTGTCAACTCGATTATAGGATC
TGGTATAATAGACTTTTCCCTTGTTTTATTGATAAAAGGAGGGGCCCTCT
CTGGAACAGATACCTACCAGTCTTTGGTCAATAAAACTTTCGGCTTTCCA
GGGTATCTGCTCCTCTCTGTTCTTCAGTTTTTGTATCCTTTTATAGCAAT
GATAAGTTACAATATAATAGCTGGAGATACTTTGAGCAAAGTTTTTCAAA
GAATCCCAGGAGTTGATCCTGAAAACGTGTTTATTGGTCGCCACTTCATT
ATTGGACTTTCCACAGTTACCTTTACTCTGCCTTTATCCTTGTACCGAAA
TATAGCAAAGCTTGGAAAGGTCTCCCTCATCTCTACAGGTTTAACAACTC
TGATTCTTGGAATTGTAATGGCAAGGGCAATTTCACTGGGTCCACACATA
CCAAAAACAGAAGACGCTTGGGTATTTGCAAAGCCCAATGCCATTCAAGC
GGTCGGGGTTATGTCTTTTGCATTTATTTGCCACCATAACTCCTTCTTAG
TTTACAGTTCTCTAGAAGAACCCACAGTAGCTAAGTGGTCCCGCCTTATC
CATATGTCCATCGTGATTTCTGTATTTATCTGTATATTCTTTGCTACATG
TGGATACTTGACATTTACTGGCTTCACCCAAGGGGACTTATTTGAAAATT
ACTGCAGAAATGATGACCTGGTAACATTTGGAAGATTTTGTTATGGTGTC
ACTGTCATTTTGACATACCCTATGGAATGCTTTGTGACAAGAGAGGTAAT
TGCCAATGTGTTTTTTGGTGGGAATCTTTCATCGGTTTTCCACATTGTTG
TAACAGTGATGGTCATCACTGTAGCCACGCTTGTGTCATTGCTGATTGAT
TGCCTCGGGATAGTTCTAGAACTCAATGGTGTGCTCTGTGCAACTCCCCT
CATTTTTATCATTCCATCAGCCTGTTATCTGAAACTGTCTGAAGAACCAA
GGACACACTCCGATAAGATTATGTCTTGTGTCATGCTTCCCATTGGTGCT
GTGGTGATGGTTTTTGGATTCGTCATGGCTATTACAAATACTCAAGACTG
CACCCATGGGCAGGAAATGTTCTACTGCTTTCCTGACAATTTCTCTCTCA
CAAATACCTCAGAGTCTCATGTTCAGCAGACAACACAACTTTCTACTTTA
AATATTAGTATCTTTCAA

### Encoding Protein

>chr2.164.004.a

(SEQ ID NO: 84)

MKFPTGGCFREKLQLQPSCLCILASEKEQKKASPSPEEKYICLGELRWKS
APGCHSLEILGLQPSRPAEGAGGARGTGSLQLCGYTRTSAVCEVAEEAGR
VRIRTQPQHLPPAEDHRSAMGYQRQEPVIPPQRDLDDRETLVSEHEYKEK
TCQSAALFNVVNSIIGSGIIDFSLVLLIKGGALSGTDTYQSLVNKTFGFP
GYLLLSVLQFLYPFIAMISYNIIAGDTLSKVFQRIPGVDPENVFIGRHFI
IGLSTVTFTLPLSLYRNIAKLGKVSLISTGLTTLILGIVMARAISLGPHI
PKTEDAWVFAKPNAIQAVGVMSFAFICHHNSFLVYSSLEEPTVAKWSRLI
HMSIVISVFICIFFATCGYLTFTGFTQGDLFENYCRNDDLVTFGRGCYGV
TVILTYPMECFVTREVIANVFFGGNLSSVFHIVVTVMVITVATLVSLLID
CLGIVLELGGVLCATPLIFIIPSACYLKLSEEPRTHSDKIMSCVMLPIGA
VVMVFGFVMAITNTQDCTHGQEMFYCFPDNFSLTNTSESHVQQTTQLSTL
NISIFQ

EST data backs-up this prediction and it has an ATG start.
The final prediction came from the BLAST database.
>

### AX480878

(SEQ ID NO: 85)

agcatccccgtcccggaggaaaaaacatttgtctggcgaactccgggtgg
aaagcgccccaggctgccacagcctagagatcttggggcttcagcccctc
gcggcctgccgagggagcagggggcgcccgtggaactggctccctgcagc
tctgcggctacacgcggacctcggctgtgtgcgaggtggcggaggaggct
ggccgggtgcgaatccgtacccagccccagcatcttccacctgctgagga
ccaccgctcagccatgggctaccagaggcaggagcctgtcatcccgccgc
agagagatttagatgacagagaaacccttgtttctgaacatgagtataaa
gagaaaacctgtcagtctgctgctctttttaatgttgtcaactcgattat
aggatctggtataataggattgccttattcaatgaagcaagctgggtttc
ctttgggaatattgcttttattctgggtttcatatgttacagacttttcc
cttgttttattgataaaaggaggggccctctctggaacagatacctacca
gtctttggtcaataaaactttcggctttccagggtatctgctcctctctg
ttcttcagtttttgtatccttttatagcaatgataagttacaatataata
gctggagatactttgagcaaagtttttcaaagaatcccaggagttgatcc
tgaaaacgtgtttattggtcgccacttcattattggactttccacagtta
cctttactctgcctttatccttgtaccgaaatatagcaaagcttggaaag
gtctccctcatctctacaggtttaacaactctgattcttggaattgtaat
ggcaagggcaatttcactgggtccacacataccaaaaacagaagacgctt
gggtatttgcaaagcccaatgccattcaagcggtcggggttatgtctttt
gcatttatttgccaccataactccttcttagtttacagttctctagaaga
acccacagtagctaagtggtcccgccttatccatatgtccatcgtgattt
ctgtatttatctgtatattctttgctacatgtggatacttgacatttact
ggcttcacccaaggggacttatttgaaaattactgcagaaatgatgacct
ggtaacatttggaagattttgttatggtgtcactgtcattttgacatacc
ctatggaatgctttgtgacaagagaggtaattgccaatgtgttttttggt
gggaatctttcatcggttttccacattgttgtaacagtgatggtcatcac
tgtagccacgcttgtgtcattgctgattgattgcctcgggatagttctag
aactcaatggtgtgctctgtgcaactcccctcatttttatcattccatca
gcctgttatctgaaactgtctgaagaaccaaggacacactccgataagat
tatgtcttgtgtcatgcttcccattggtgctgtggtgatggtttttggat
tcgtcatggctattacaaatactcaagactgcacccatgggcaggaaatg
ttctactgctttcctgacaatttctctctcacaaatacctcagagtctca
tgttcagcagacaacacaactttctactttaaatattagtatctttcaat
gagttgactgctttaaaaatatgtatgttttcatagactttaaaacacat
aacatttacgcttgctttagtctgtatttatgttatataaaattattatt
ttggcttttatcaagacttggcttttatgagtagtgcaatataaaaa

### Encoding Protein

### >AX480878

(SEQ ID NO: 86)

vcevaeeagrvrirtqpqhlppaedhrsamgyqrqepvippqrdlddret
lvseheykektcqsaalfnvvnsiigsgiiglpysmkqagfplgilllfw
vsyvtdfslvllikggalsgtdtyqslvnktfgfpgylllsvlqflypfi
amisyniiqgdtlskvfqripgvdpenvfigrhfiiglstvtftlplsly
rniaklgkvslistglttlilgivmaraislgphipktedawvfakpnai
qavgvmsfafichhnsflvyssleeptvakwsrlihmsivisvficiffa
tcgyltftgftqgdlfenycrnddlvtfgrfcygvtviltypmecfvtre
vianvffggnlssvfhivvtvmvitvatlvsllidclgivlelngvlcat
plifiipsacylklseeprthsdkimscvmlpigavvmvfgfvmaitntq
dcthgqemfycfpdnfsltntseshvqqttqlstlnisifq

The EST database backs up this prediction, however, the start coding is not an ATG.

We are assembling PCR data to determine which of these predictions is correct. Preliminary data suggests that a combination of the AX480878 and C2001829 is correct, giving the following sequence:

### >PSAT-Long

(SEQ ID NO: 87)

atgaagtttccaacaggtggttgcttcagggaaaagctccagcttcagcc
atcatgtctctgcattctggccagtgagaaggagcaaaagaaagcatctc
cgtctccggaggaaaaatacatttgtctgggcgaactccggtggaaaagc
gccccaggctgccacagcctagagatcttggggctgcagccctcgcggcc
tgccgagggagcagggggcgcccgtggaactggctccctgcagctctgcg
gctacacgcggacctcggctgtgtgcgaggtggcggaggaggctggccgg
gtgcgaatccgtacccagccccagcatcttccacctgctgaggaccaccg
ctcagccatgggctaccagaggcaggagcctgtcatcccgccgcagagag
atttagatgacagagaaacccttgtttctgaacatgagtataaagagaaa
acctgtcagtctgctgctctttttaatgttgtcaactcgattataggatc
tggtataataggattgccttattcaatgaagcaagctgggtttcctttgg
gaatattgcttttattctgggtttcatatgttacagacttttcccttgtt
ttattgataaaaggaggggccctctctggaacagatacctaccagtcttt
ggtcaataaaactttcggctttccagggtatctgctcctctctgttcttc
agtttttgtatccttttatagcaatgataagttacaatataatagctgga
gatactttgagcaaagtttttcaaagaatcccaggagttgatcctgaaaa
cgtgtttattggtcgccacttcattattggactttccacagttaccttta
ctctgcctttatccttgtaccgaaatatagcaaagcttggaaaggtctcc
ctcatctctacaggtttaacaactctgattcttggaattgtaatggcaag
ggcaatttcactgggtccacacataccaaaaacagaagacgcttgggtat
ttgcaaagcccaatgccattcaagcggtcggggttatgtcttttgcattt
atttgccaccataactccttcttagtttacagttctctagaagaacccac
agtagctaagtggtcccgccttatccatatgtccatcgtgatttctgtat
ttatctgtatattctttgctacatgtggatacttgacatttactggcttc
acccaaggggacttatttgaaaattactgcagaaatgatgacctggtaac
atttggaagattttgttatggtgtcactgtcattttgacataccctatgg
aatgctttgtgacaagagaggtaattgccaatgtgttttttggtgggaat
ctttcatcggttttccacattgttgtaacagtgatggtcatcactgtagc
cacgcttgtgtcattgctgattgattgcctcgggatagttctagaactca
atggtgtgctctgtgcaactcccctcatttttatcattccatcagcctgt
tatctgaaactgtctgaagaaccaaggacacactccgataagattatgtc
ttgtgtcatgcttcccattggtgctgtggtgatggtttttggattcgtca
tggctattacaaatactcaagactgcacccatgggcaggaaatgttctac
tgctttcctgacaatttctctctcacaaatacctcagagtctcatgttca
gcagacaacacaactttctactttaaatattagtatctttcaa

### Encoding Protein

### >PSAT-Long

(SEQ ID NO: 88)

mkfptggcfreklqlqpsclcilasekeqkkaspspeekyiclgelrwks
apgchsleilglqpsrpaegaggargtgslqlcgytrtsavcevaeeagr
vrirtqpqhlppaedhrsamgyqrqepvippqrdlddretlvseheykek
tcqsaalfnvvnsiigsgiiglpysmkqagfplgilllfwvsyvtdfslv
llikggalsgtdtyqslvnktfgfpgylllsvlqflypfiamisyniiag
dtlskvfqripgvdpenvfigrhfiiglstvtftlplslyrniaklgkvs
listglttlilgivmaraislgphipktedawvfakpnaiqavgvmsfaf
ichhnsflvyssleeptvakwsrlihmsivisvficiffatcgyltftgf
tqgdlfenycrnddlvtfgrfcygvtviltypmecfvtrevianvffggn
lssvfhivvtvmvitvatlvsllidclgivlelngvlcatplifiipsac
ylklseeprthsdkimscvmlpigavvmvfgfvmaitntqdcthgqemfy
cfpdnfsltntseshvqqttqlstlnisifq

We have also performed TaqMan analysis using primers that would detect both the long form and the short form of PSAT and confirmed that the message is malignant prostate specific. We have attempted PCR to determine if the short form exists by trying to PCR from the 5′UTR of the short form into the coding sequence. If the message is spliced correctly, we should only get a band if the short form exists in the cell. Using this method, we demonstrated that the short form is in the cell (or at least an unspliced form of the longer message). We have also tried to amplify the area around the Twinscan prediction start codon, however, to date have been unsuccessful. Our current thinking is that the real start codon is at bases 358-360 of the PSAT-long message (as opposed to bases 1-3). This would give the following protein:

(SEQ ID NO: 89)

Mgyqrqepvippqrdlddretlvseheykektcqsaalfnvvnsiigsgi
iglpysmkqagfplgilllfwvsyvtdfslvllikggalsgtdtyqslvn
ktfgfpgylllsvlqflypfiamisyniiagdtlskvfqripgvdpenvf
igrhfiiglstvtftlplslyrniaklgkvslistglttlilgivmarai
slgphipktedawvfakpnaiqavgvmsfafichhnsflvyssleeptva
kwsrlihmsivisvficiffatcgyltftgftqgdlfenycrnddlvtfg
rfcygvtviltypmecfvtrevianvffggnlssvfhivvtvmvitvatl
vsllidclgivlelngvlcatplifiipsacylklseeprthsdkimscv
mlpigavvmvfgfvmaitntqdcthgqemfycfpdnfsltntseshvqqt
tqlstlnisifq

### REFERENCES

Alcaraz et al., Cancer Res., 55:3998-4002, 1994.
Alihoff et al., World J. Urol., 7:12-16, 1989.
An et al., Proc. Amer. Assn. Canc. Res., 36:82, 1995.
An et al., Molec. Urol., 2: 305-309, 1998.
Antibodies: A Laboratory Manual, Cold Spring Harbor Laboratory, Cold Spring Harbor Press, Cold Spring Harbor, N.Y., 1988.
Babian et al., J. Urol., 156:432-437, 1996.
Badalament et al., J. Urol., 156:1375-1380, 1996.
Baichwal and Sugden, In: Gene Transfer, Kucherlapati (Ed.), Plenum Press, New York, pp 117-148, 1986.
Bangharn et al., J. Mol. Biol. 13: 238-252, 1965.
Barinaga, Science, 271: 1233, 1996.
Bedzyk et al., J. Biol. Chem., 265:18615, 1990
Bell et al., “Gynecological and Genitourinary Tumors,” In: Diagnostic Immunopathology, Colvin, Bhan and McCluskey (Eds.), 2nd edition, Ch. 31, Raven Press, New York, pp 579-597, 1995.
Bellus, J. Macromol. Sci. Pure Appl. Chem., A31(1):1355-1376, 1994.
Benvenisty and Neshif, Proc. Nat. Acad. Sci. USA, 83:9551-9555, 1986.
Bittner et al., Methods in Enzymol, 153:516-544, 1987.
Bookstein et al., Science, 247:712-715, 1990a.
Bookstein et al., Proc. Nat'l Acad. Sci. USA, 87:7762-7767, 1990b.
Bova et al., Cancer Res., 53:3869-3873, 1993
Brawn et al., The Prostate, 28:295-299, 1996.
Campbell, In: Monoclonal Antibody Technology, Laboratory Techniques in Biochemistry and Molecular Biology, Burden and Von Knippenberg (Eds.), Vol. 13:75-83, Elsevier, Amsterdam, 1984.
Capaldi et al., Biochem. Biophys. Res. Comm, 76:425, 1977.
Carter and Coffey, In: Prostate Cancer: The Second Tokyo Symposium, J. P.
Karr and H. Yamanak (Eds.), Elsevier, New York, pp 19-27, 1989.
Carter and Coffey, Prostate, 16:3948, 1990.
Carter et al., Proc. Nat'l Acad Sci. USA, 87:8751-8755, 1990.
Carter et al., Proc. Nat'l Acad Sci. USA 93: 749-753, 1996.
Carter et al., J. Urol., 157:2206-2209, 1997.
Cech et al., Cell, 27:487-496, 1981.
Chang et al., Hepatology, 14: 124A, 1991.
Chaudhary et al., Proc. Nat'l Acad. Sci., 87:9491, 1990
Chen and Okayama, Mol. Cell Biol., 7:2745-2752, 1987.
Chen et al., Clin. Chem., 41:273-282, 1995a.
Chen et al., Proc. Am. Urol. Assn, 153:267 A, 1995.
Chinault and Carbon, “Overlap Hybridization Screening: Isolation and Characterization of Overlapping DNA Fragments Surrounding the LEU2 Gene on Yeast Chromosome III,” Gene, 5:111-126, 1979.
Chomczynski and Sacchi, Anal. Biochem., 162:156-159, 1987.
Christensson et al., J. Urol., 150:100-105, 1993.
Coffin, In: Virology, Fields et al. (Eds.), Raven Press, New York, pp 1437-1500, 1990.
Colberre-Garapin et al., J. Mol. Biol., 150:1, 1981.
Colvin et al., Diagnostic Immunopathology, 2nd edition, Raven Press, New York, 1995.
Cooner et al., J. Urol., 143:1146-1154, 1990.
Couch et al., Am. Rev. Resp. Dis., 88:394-403, 1963.
Coupar et al., Gene, 68:1-10, 1988.
Culver et al., Science, 256:1550-1552, 1992.
Davey et al., EPO No. 329 822.
Deamer and Uster, “Liposome Preparation: Methods and Mechanisms,” In: Liposomes, M. Ostro (Ed.), 1983.
Diamond et al., J. Urol., 128:729-734, 1982.
Donahue et al., J. Biol. Chem., 269:8604-8609, 1994.
Dong et al., Science, 268:884-886, 1995.
Dubensky et al., Proc. Nat. Acad. Sci. USA, 81:7529-7533, 1984.
Dumont et al., J. Immunol., 152:992-1003, 1994.
Elledge et al., Cancer Res. 54:3752-3757, 1994
European Patent Application EPO No. 320 308
Fearon et al., Science, 247:47-56, 1990.
Fechheimer et al., Proc. Natl. Acad. Sci. USA, 84:8463-8467, 1987.
Forster and Symons, Cell, 49:211-220, 1987.
Fraley et al., Proc. Natl. Acad. Sci USA, 76:3348-3352, 1979.
Friedmann, Science, 244:1275-1281, 1989.
Freifelder, In: Physical Biochemistry Applications to Biochemistry and Molecular Biology, 2nd ed., Wm. Freeman and Co., New York, N.Y., 1982.
Frohman, In: PCR Protocols: A Guide to Methods and Applications, Academic Press, New York, 1990.
Gefter et al., Somatic Cell Genet., 3:231-236, 1977.
Gerlach et al., Nature (London), 328:802-805, 1987.
Ghosh-Choudhury et al., EMBO J., 6:1733-1739, 1987.
Gingeras et al., PCT Application WO 88/10315.
Ghosh and Bachhawat, In: Liver Diseases, Targeted Diagnosis and Therapy Using Specific Receptors and Ligands, Wu et al. (Eds.), Marcel Dekker, New York, pp 87-104, 1991.
Goding, In: Monoclonal Antibodies: Principles and Practice, 2nd ed., Academic Press, Orlando, Fla., pp 60-61, 65-66, 71-74, 1986.
Gomez-Foix et al., J. Biol. Chem., 267:25129-25134, 1992.
Gopal, Mol. Cell Biol., 5:1188-1190, 1985.
Graham et al., J. Gen. Virol., 36:59-72, 1977.
Graham and van der Eb, Virology, 52:456-467, 1973.
Graham and Prevec, In: Methods in Molecular Biology: Gene Transfer and Expression Protocols 7, E. J. Murray (Ed.), Humana Press, Clifton, N.J., pp 205-225, 1991.
Gregoriadis (ed.), In: Drug Carriers in Biology and Medicine, pp 287-341, 1979.
Grunhaus and Horwitz, Sem. Virol., 3:237-252, 1992.
Harland and Weintraub, J. Cell Biol., 101:1094-1099, 1985.
Harris et al., J. Urol., 157:1740-1743, 1997.
Heng et al., Proc. Nat. Acad. Sci. USA, 89: 9509-9513, 1992.
Hermonat and Muzycska, Proc. Nat. Acad. Sci USA, 81:6466-6470, 1984.
Hersdorffer et al., DNA Cell Biol., 9:713-723, 1990.
Herz and Gerard, Proc. Natl. Acad Sci. USA, 90:2812-2816, 1993.
Hess et al., J. Adv. Enzyme Reg., 7:149, 1968.
Hitzeman et al., J. Biol. Chem., 255:2073, 1980.
Holland et al., Biochemistry, 17:4900, 1978.
Horoszewicz, Kawinski and Murphy, Anticancer Res., 7:927-936, 1987.
Horwich, et al., J. Virol., 64:642-650, 1990.
Huang et al., Prostate, 23: 201-212, 1993.
Innis et al., In: PCR Protocols, Academic Press, Inc., San Diego Calif., 1990.
Inouye et al., Nucl. Acids Res., 13:3101-3109, 1985.
Isaacs et al., Cancer Res., 51:4716-4720, 1991.
Isaacs et al., Sem. Oncol., 21:1-18, 1994.
Israeli et al., Cancer Res., 54:1807-1811, 1994.
Jacobson et al., JAMA, 274:1445-1449, 1995.
Johnson et al., In: Biotechnology and Pharmacy, Pezzuto et al., (Eds.), Chapman and Hall, New York, 1993.
Jones, Genetics, 85:12, 1977.
Jones and Shenk, Cell, 13:181-188, 1978.
Joyce, Nature, 338:217-244, 1989.
Kaneda et al., Science, 243:375-378, 1989.
Kato et al., J. Biol. Chem., 266:3361-3364, 1991.
Kim and Cech, Proc. Natl. Acad. Sci. USA, 84:8788-8792, 1987.
Kingsman et al., Gene, 7:141, 1979.
Klein et al., Nature, 327:70-73, 1987.
Kohler and Milstein, Nature, 256:495-497, 1975.
Kohler and Milstein, Eur. J. Immunol., 6:511-519, 1976.
Kwoh et al., Proc. Nat. Acad. Sci. USA, 86:1173, 1989.
Landis et al., CA Cancer J. Clin., 48: 6-29, 1998.
Le Gal La Salle et al., Science, 259:988-990, 1993.
Levrero et al., Gene, 10 1: 195-202, 1991.
Liang and Pardee, Science, 257:967-971, 1992.
Liang and Pardee, U.S. Pat. No. 5,262,311, 1993.
Liang et al., Cancer Res., 52:6966-6968, 1992.
Lifton, Science, 272:676, 1996.
Lilja et al., Clin. Chem., 37:1618-1625, 1991.
Lithrup et al., Cancer, 74:3146-3150, 1994.
Lowy et al., Cell, 22:817, 1980.
Macoska et al., Cancer Res., 54:3824-3830, 1994.
Mann et al., Cell, 33:153-159, 1983.
Markowitz et al., J. Virol., 62:1120-1124, 1988.
Marley et al., Urology, 48(6A): 16-22, 1996.
McCormack et al., Urology, 45:729-744, 1995.
Michel and Westhof, J. Mol. Biol. 216:585-610, 1990.
Miki et al., Science, 266:66-71, 1994.
Miller et al., PCT Application, WO 89/06700.
Mok et al., Gynecol. Oncol., 52:247-252, 1994.
Morahan et al., Science 272:1811, 1996.
Mulligan et al., Proc. Nat'l Acad. Sci. USA, 78:2072, 1981.
Mulligan, Science, 260:926-932, 1993.
Murphy et al., Cancer, 78: 809-818, 1996.
Murphy et al., Prostate, 26:164-168, 1995.
Nakamura et al., In: Handbook of Experimental Immunology, (4th Ed.), Weir, E., Herzenberg, L. A., Blackwell, C., Herzenberg, L. (Eds.), Vol. 1, Chapter 27, Blackwell Scientific Publ., Oxford, 1987.
Nicolas and Rubinstein, In: Vectors: A Survey of Molecular Cloning Vectors and Their Uses, Rodriguez and Denhardt (Eds.), Butterworth, Stoneham, p 494-513, 1988.
Nicolau and Sene, Biochim. Biophys. Acta, 721:185-190, 1982.
O'Dowd et al., J. Urol., 158:687-698, 1997.
O'Hare et al., Proc. Nat'l Acad Sci. USA, 78:1527, 1981.
Oesterling et al., J. Urol., 154:1090-1095, 1995.
Ohara et al., Proc. Nat'l Acad. Sci. USA, 86:5673-5677, 1989.
Orozco et al., Urology, 51:186-195, 1998.
Parker et al., CA Cancer J. Clin., 65:5-27, 1996.
Partin and Oesterling, Urology, 48 (6A):1-3, 1996.
Partin and Oesterling, J. Urol., 152:1358-1368, 1994.
Partin and Oesterling (Eds.), Urology, 48(6A) Supplement: 1-87, 1996.
Paskind et al., Virology, 67:242-248, 1975.
PCT Application No. PCT/US87/00880
Pettersson et al., Clin. Chem., 41(10):1480-1488, 1995.
Piironen et al., Clin. Chem. 42:1034-1041, 1996.
Potter et al., Proc. Nat. Acad. Sci. USA, 81:7161-7165, 1984.
Racher et al., Biotechnology Techniques, 9:169-174, 1995.
Ragot et al., Nature, 361:647-650, 1993.
Ralph and Veltri, Advanced Laboratory, 6:51-56, 1997.
Ralph et al., Proc. Natl. Acad. Sci. USA, 90(22):10710-10714, 1993.
Reinhold-Hurek and Shub, Nature, 357:173-176, 1992.
Renan, Radiother. Oncol., 19:197-218, 1990.
Ribas de Pouplana and Fothergill-Gilmore, Biochemistry, 33:7047-7055, 1994.
Rich et al., Hum. Gene Ther., 4:461-476, 1993.
Ridgeway, In: Vectors: A Survey of Molecular Cloning Vectors and Their Uses, Rodriguez R L, Denhardt D T (Eds.), Butterworth, Stoneham, pp 467-492, 1988.
Rippe et al., Mol. Cell Biol., 10:689-695, 1990.
Rosenfeld et al., Science, 252:431-434, 1991.
Rosenfeld et al., Cell, 68:143-155, 1992.
Roux et al., Proc. Nat'l Acad. Sci. USA, 86:9079-9083, 1989.
Sager et al., FASEB J., 7:964-970, 1993.
Sambrook et al., (ed.), In: Molecular Cloning, Cold Spring Harbor Laboratory Press, Cold Spring Harbor, N.Y., 1989.
Santerre et al., Gene, 30: 147-156, 1984.
Sarver, et al., Science, 247:1222-1225, 1990.
Scanlon et al., Proc Natl Acad Sci USA, 88:10591-10595, 1991.
Sidransky et al., Science, 252:706-709, 1991.
Sidransky et al., Cancer Res., 52:2984-2986, 1992.
Silver et al., Clin. Cancer Res., 3:81-85, 1997.
Slamon et al., Science, 224:256-262, 1984.
Slamon et al., Science, 235:177-182, 1987.
Slamon et al., Science, 244:707-712, 1989.
Smith, U.S. Pat. No. 4,215,051.
Soh et al., J. Urol., 157:2212-2218, 1997.
Stenman et al., Cancer Res., 51:222-226, 1991.
Stinchcomb et al., Nature, 282:39, 1979.
Strafford-Perricaudet and Perricaudet, In: Human Gene Transfer, O. Cohen-Haguenauer et al., (Eds.), John Libbey Eurotext, France, pp 51-61, 1991.
Strafford-Perricaudet et al., Hum. Gene. Ther., 1:241-256, 1990.
Sun and Cohen, Gene, 137:127-132, 1993.
Szoka and Papahadjopoulos, Proc. Nat'l. Acad. Sci. USA, 75: 4194-4198, 1978.
Szybalska et al., Proc. Nat'l Acad. Sci. USA, 48:2026, 1962.
Takahashi et al., Cancer Res., 54:3574-3579, 1994.
Taparowsky et al., Nature, 300:762-764, 1982.
Temin, In: Gene Transfer, Kucherlapati R. (Ed.), Plenum Press, New York, pp 149-188, 1986.
Tooze, In: Molecular Biology of DNA Tumor Viruses, 2nd ed., Cold Spring Harbor Laboratory, Cold Spring Harbor, N.Y., 1991.
Top et al., J. Infect. Dis., 124:155-160, 1971.
Tschemper et al., Gene, 10:1 57, 1980.
Tur-Kaspa et al., Mol. Cell Biol., 6:716-718, 1986.
U.S. patent application Ser. No. 08/692,787
U.S. Pat. No. 4,196,265
U.S. Pat. No. 4,215,051
U.S. Pat. No. 4,683,195
U.S. Pat. No. 4,683,202
U.S. Pat. No. 4,800,159
U.S. Pat. No. 4,883,750
U.S. Pat. No. 5,354,855
U.S. Pat. No. 5,359,046
Varmus et al., Cell, 25:23-36, 1981.
Veltri et al., J. Cell Biochem., 19(suppl):249-258, 1994.
Veltri et al., Urology, 48: 685-691, 1996.
Veltri et al., Sem. Urol. Oncol., 16:106-117, 1998.
Veltri et al., Urology, 53:139-147, 1999.
Visakorpi et al., Am. J. Pathol., 145:1-7, 1994.
Wagner et al., Science, 260:1510-1513, 1993.
Walker et al., Proc. Nat'l Acad. Sci. USA, 89:392-396, 1992.
Watson et al., Cancer Res., 54:4598-4602, 1994.
Welsh et al., Nucl. Acids Res., 20:4965-4970, 1992.
Wigler et al, Cell, 11:223, 1977.
Wigler et al., Proc. Nat'l Acad. Sci. USA, 77:3567, 1980.
Wingo et al., CA Cancer J. Clin., 47: 239-242, 1997.
WO 90/07641, filed Dec. 21, 1990.
Wong et al., Int. J. Oncol., 3:13-17, 1993.
Wu and Wu, J. Biol. Chem., 262: 4429-4432, 1987.
Wu and Wu, Biochemistry, 27: 887-892, 1988.
Wu and Wu, Adv. Drug Delivery Rev., 12: 159-167, 1993.
Wu et al., Genomics, 4:560, 1989.
Yang et al., Proc. Natl. Acad. Sci. USA, 87:9568-9572, 1990.
Yokoda et al., Cancer Res. 52, 3402-3408, 1992.
Zlotta et al, J. Urol., 157:1315-1321, 1997.

1.-39. (canceled)

40. An isolated monoclonal antibody or antigen-binding fragment thereof that specifically binds the extracellular domain of the Kv3.2a (SEQ ID NO:92) antigen, wherein said antibody or antigen-binding fragment thereof is produced from a hybridoma selected from the group consisting of: 1B8, 5C9, 5E1, 9B9, 16E6, 17C1, 21E10, 21G6, 23D8, 24E6, 25C6, 34B5, 37E12, 42B9, 42G4, and 43D3.

41. An isolated monoclonal antibody or antigen-binding fragment thereof that specifically binds to the same epitope of the Kv3.2a (SEQ ID NO:92) antigen, as an antibody or antigen-binding fragment thereof produced from a hybridoma selected from the group consisting of: 1B8, 5C9, 5E1, 9B9, 16E6, 17C1, 21E10, 21G6, 23D8, 24E6, 25C6, 34B5, 37E12, 42B9, 42G4, and 43D3.

42. The antibody of claim 40, wherein said antibody is produced from the 37E12 hybridoma.

43. The antibody of claim 40, wherein said antibody is produced from the 5C9 hybridoma.

44. The antibody of claim 40 which is attached directly or indirectly to a detectable label.

45. The antibody of claim 40 which is a human, humanized, chimeric, or bispecific antibody.

46. The antibody of claim 45 which is a human or humanized antibody.

47. The antibody of claim 45 which is a domain-deleted antibody.

48. A diagnostic kit for detection of prostate cancer which comprises a monoclonal antibody according to claim 40 and a detectable label.

49. A method of treating prostate cancer comprising administering a monoclonal antibody according to claim 40.

50. The method of claim 49 wherein said antibody is attached to an effector.

51. The method of claim 50 wherein said effector is a radionuclide, enzyme, cytotoxin, hormone, or hormone antagonist.

52. The antibody of claim 41, wherein said antibody is produced from the 37E12 hybridoma.

53. The antibody of claim 41, wherein said antibody is produced from the 5C9 hybridoma.

54. The antibody of claim 41 which is attached directly or indirectly to a detectable label.

55. The antibody of claim 41 which is a human, humanized, chimeric, or bispecific antibody.

56. The antibody of claim 55 which is a human or humanized antibody.

57. The antibody of claim 55 which is a domain-deleted antibody.

58. A diagnostic kit for detection of prostate cancer which comprises a monoclonal antibody according to claim 41 and a detectable label.

59. A method of treating prostate cancer comprising administering a monoclonal antibody according to claim 41.

60. The method of claim 59 wherein said antibody is attached to an effector.

61. The method of claim 60 wherein said effector is a radionuclide, enzyme, cytotoxin, hormone, or hormone antagonist.


SEQUENCE LISTING (93 sequences)

SEQ ID NO: 1 - DNA, 716 bp, Homo sapiens
agttactcatttttcaggcctgagttgatcgttaatcatcttaattatgttcattctgaagccaacaggagaaccaagaccaaaactttattgtctctgctttcatttcttgatgaaacctctggactaagcacacatcttccttgtttatctctctcaaaggagtgtggagtgcttcatctggacatccacgggaagaaggaagacatgagggaatgctggaagaggagacaggccccagatttgggcaggaagtaaacagttttcaggctgaggccaatctgagcaggaacattccaatatttcttcagctacgttgtcccagcacttcactggttaaccttttatgtccaccatttgtggatttcacagctacttgtcaatggtgaatattgatcatcatcattatctactgagctgctaccatatcccagctactccttgcatgttgttcattattttctcaacactcagcatatttgcaatatgttatgtaatatcacagacaaggaaactgaacgcagaaatgttttatttcttgccaaacatcacatgaggatgaacaatgaaaccgatttgaaaccaggattgtctgattccaacatctctgggtcctttttcactctgatatgctgcaattaaaaagccatttctaagactgtaaaaaaaaaaaaaaaaaacacctgcggccgcaagcttattcccttaggaggtat

SEQ ID NO: 2 - protein, 69 aa, Homo sapiens
MSTICGFHSYLSMVNIDHHHYLLSCYHIPATPCMLFIIFSTLSIFAICYVISQTRKLNAEMFYFLPNIT

SEQ ID NO: 3 - DNA, 29 bp, Artificial Sequence
  Description of Artificial Sequence Primer
gaagctttcaatattgttaaaaacaagac

SEQ ID NO: 4 - DNA, 29 bp, Artificial Sequence
  Description of Artificial Sequence Primer
atgtgtcactctgtgtactattgcaggcc

SEQ ID NO: 5 - DNA, 34 bp, Artificial Sequence
  Description of Artificial Sequence Primer
gcggcgaagctttcaatattgttaaaaacaagac

SEQ ID NO: 6 - DNA, 29 bp, Artificial Sequence
  Description of Artificial Sequence Primer
atgtgtcactctgtgtactattgcaggcc

SEQ ID NO: 7 - DNA, 980 bp, Homo sapiens
  modified_base (33): a, t, c, g, other or unknown
ggaaagcgaagagcgcccaatacgcaaaccgcntctccccgcgngtgggcgattcattatgcagctggcacgacagggtttcccgactggaaagcngggcagtgagnggcaacgcaattaatgtgagttagctcactcattaggcccccccaggctttacactttatgcttcccggctcgtatgttgtgtggaattgtgagcggataacaatttcacacaggaaacagctatgacatgattacgaatttaatacgactcactatagggaatttggccctcgaggccaagaattcggcacgaggtgctttcatggtgaccaaactaatgagcagcacccttctgcagaggtaaactttgccttgctgagaaaccaattgttggcgtgtttatttcatttatgactttgagctttatttctaacatggcccaaagtaatcctcttttcttgaacacatggtagaatgccctaggtgaatccctccagtcttccagtaccatccttgactcctctctctgatgacacatgaactttatgcttttgcacacttcaggcaacaccaaaagaaaggaaaagaacagcttagcttcttaatgtgtgtaagaaaccacagtgaaaaaaaatcaggtgtgttgttgaggctgctaaaagctttccttttttttctgtgccagttctcgctgcctcattggttgagatgggatgtcttttttgatgtcctctttagagagtgttatcctcacctttttgcatagtcctaccaaaagacacctcacatgcaaagtgtaacagaaaattacagtcatgactttagttttaaaaacaggacgtatattcatgaagaatgtttgctgttttcccagtgggttaatcatatgaatataaaacagactaaaaatatcaagttgtttttgcatttatttattgtagaaataaaatggattgctacctctgagcttctgaaaaaaaaaaaaaaaaaaa

SEQ ID NO: 8 - DNA, 108 bp, Homo sapiens
tgggggacagctgaggatgggcctagcagatgaagcttgccagcaaggccaaagcaaacggtttctcctgtggatagtggacagagacctttgtaaccaatggaatta

SEQ ID NO: 9 - DNA, 509 bp, Homo sapiens
  modified_base (54): a, t, c, g, other or unknown
attctacggcgactggagagggtgagtagccactgctccagcctcctgcggagngcctacatccananccgngtgnaancagtgccntatcttttctgccncancnanganggtccggcctgcanggcatggtgtggtatagcatcctcaaggncaccaaaatcacgtgtgaggagaagatggtgtcaatggcccgaaacacatattatttgactctatcaaaagtctctcctttttaaaccttttcttatggatggctgtcaatcccgaggcagaagttttcaggtggagaccaagcggcctttgctcttcttccttcttcctgccacactctgctttcttcctgccatggacccctggaggagacctatggagggacagttttgacctgacccctagaggagacagttttgacctcttcagcaccaggaaggaagctctgaggatggttgcagtgaggaagcatgggtctttaaggacttctctctcttttttgctggacattattg

SEQ ID NO: 10 - DNA, 3837 bp, Homo sapiens
ctgtacgcccagcgacgttggcagaagcgtcgccgcatcccccagaagagcgcaagcacagaagccactcatgagatccactacatcccatctgtgctgctgggtccccaggcgcgggagagcttccgttcatcccggctgcaaacccacaattccgtcattggcgtgcccatccgggagactcccatcctggatgactatgactgtgaggaggatgaggagccacctaggcgggccaaccatgtctcccgcgaggacgagtttggcagccaggtgacccacactctggacagtctgggacatccaggggaagagaaggtggactttgagaagaaaggaggaatcagctttgggagagccaaggggacgtcgggctcagaggcagacgatgaaactcagctgacattctacacggagcagtaccgcagtcgccgccgcagcaaaggtttgctgaaaagcccagtgaacaagacagccctgacactgattgctgtgagttcctgcatcctggccatggtgtgtggcagccagatgtcttgtccactcactgtgaaggtgactctgcatgtgcccgagcacttcatagcagatggaagcagcttcgtggtgagtgaagggagctacctggacatctccgactggttaaacccagccaagctttccctgtattaccagatcaatgccacctcgccatgggtgagggacctctgtggacaaaggacgacagatgcctgtgagcagctctgcgacccagaaaccggagagtgcagctgtcatgaaggctatgcccctgaccctgttcacagacacctgtgtgtgcgcagtgactggggacagagtgaaggaccttggccctacacgacacttgagaggggctatgatctggtgacaggggagcaagcccctgaaaagattctcaggtctactttcagcttgggccaaggcctctggcttcctgtcagcaaaagctttgtggttccgcctgtggagctgtccatcaaccccctggccagctgcaagaccgatgtgctcgtcacggaagaccctgcagatgtcagggaagaagcgatgctgtccacatactttgaaaccatcaatgacctgctgtcttccttcgggccagttcgtgactgctctcggaacaatgggggctgcactcgcaacttcaagtgtgtgtctgaccggcaggtggattcctcgggatgtgtgtgccctgaggagctgaaacccatgaaggatggctctggctgctacgaccactccaaaggcattgactgctctgatggctttaatggcggctgtgagcagctgtgcctgcagcagacgctgcccctgccctacgatgccacttcgagcaccatcttcatgttctgcggttgcgtggaggagtacaaactggctcctgatggaaaatcctgcttaatgctctcagatgtctgcgagggccccaagtgcctcaaacctgactccaaattcaatgataccctctttggagagatgctacatggttacaacaaccggacccagcatgtgaaccaaggccaagtcttccagatgacctttagggagaacaacttcatcaaggactttccccagctggccgatgggctgttggtgatcccgctgccggtggaggagcagtgccggggggtcctctccgagccccttccggacctccaactgctcactggagatatcaggtatgatgaggccatgggttaccccatggtgcagcagtggcgggtccggagcaacctctaccgtgtgaagctcagcaccatcaccctcgcagcaggcttcactaatgttctcaagatcctgaccaaggagagcagtcgggaggagctgctgtccttcatccagcactatggctcccactacatcgcagaggccctctatggctcagagctcacctgcatcatccactttcccagcaagaaggtccagcagcagctgtggctccagtatcagaaagagaccacagagctgggcagcaagaaggagctcaagtccatgcccttcatcacctacctctcaggtttgctgacagcccagatgctgtcagatgaccagctcatttcaggtgtggagattcgctgtgaggagaaggggcgctgtccatctacctgtcacctttgccgccggccaggcaaggagcagctgagccccacaccagtgctgctggaaatcaaccgtgtggtgccactttataccctcatccaagacaatggcacaaaggaggccttcaagagtgcactgatgagttcctactggtgctcagggaaaggggatgtgatcgatgactggtgcaggtgtgacctcagcgcctttgatgccaatgggctccccaactgcagcccccttctgcagccggtgctgcggctgtccccaacagtggagccctccagtactgtggtctccttggagtgggtggatgttcagccagctattgggaccaaggtctccgactatattctgcagcataagaaagtggatgaatacacagacactgacctgtacacaggagaattcctgagttttgctgatgacttactctctggcctgggcacatcttgtgtagcagctggtcgaagccatggagaggtccctgaagtcagtatctactcagtcatcttcaagtgtctggagcccgacggtctctacaagttcactctgtatgctgtggatacacgagggaggcactcagagctaagcacggtgaccctgaggacggcctgtccactggtagatgacaacaaggcagaagaaatagctgacaagatctacaatctgtacaatgggtacacaagtggaaaggagcagcagatggcctacaacacactgatggaggtctcagcctcgatgctgttccgagtccagcaccactacaactctcactatgaaaagtttggcgacttcgtctggagaagtgaggatgagctggggcccaggaaggcccacctgattctacggcgactggagagggtgagtagccactgctccagcctcctgcggagtgcctacatccagagccgcgtggaaacagtgccctatcttttctgccgcagcgaggaggtccggcctgcaggcatggtgtggtatagcatcctcaaggacaccaaaatcacgtgtgaggagaagatggtgtcaatggcccgaaacacgtacggggagtccaagggccggtgagggagggtattgccctccgtgagcacagagactctccatgggagggggagcagtattctcctggatcctggggcctgggtgggctgggggacagctgaggatgggcctagcagatgaagcttgccagcaaggccaaagcaaacggtttctcctgtggatagtggacagagacctttgtaaccaatggaattattcatttttctctatcttttattttttcaaagatattatttgactctatcaaaagtctctcctttttaaaccttttcttatggatggctgtcaatcccgaggcagaagttttcaggtggagaccaagcggcctttgctcttcttccttcttcctgccacactctgctttcttcctgccatggacccctggaggagacctatggagggacagttttgacctgacccctagaggagacagttttgacctcttcagcaccaggaaggaagctctgaggatggttgcagtgaggaagcatgggtctttaaggacttctctctcttttttgctggacattattgagtttgtggaaccctgcctcttcctgctacctgtgggtctgcccagagtccctgcaggcctgtccatgcattaaaaattcctattgtctctcaaaaaaaaaaaaaaaaaaaaaaaaa

SEQ ID NO: 11 - protein, 1321 aa, Homo sapiens
FASASAVSAAASSSSFATAATAAAARSTAAPPAMAAAGARLSPGPGSGLRGRPRLCFHPGPPPLLPLLLLFLLLLPPPPLLAGATAAASREPDSPCRLKTVTVSTLPALRESDIGWSGARAGAGAGTGAGAAAAAASPGSPGSAGTAAESRLLLFVRNELPGRIAVQDDLDNTELPFFTLEMSGTAADISLVHWRQQWLENGTLYFHVSMSSSGQLAQATAPTLQEPSEIVEEQMHILHISVMGGLIALLLLLLVFTVALYAQRRWQKRRRIPQKSASTEATHEIHYIPSVLLGPQARESFRSSRLQTHNSVIGVPIRETPILDDYDCEEDEEPPRRANHVSREDEFGSQVTHTLDSLGHPGEEKVDFEKKGGISFGRAKGTSGSEADDETQLTFYTEQYRSRRRSKGLLKSPVNKTALTLIAVSSCILAMVCGSQMSCPLTVKVTLHVPEHFIADGSSFVVSEGSYLDISDWLNPAKLSLYYQINATSPWVRDLCGQRTTDACEQLCDPETGECSCHEGYAPDPVHRHLCVRSDWGQSEGPWPYTTLERGYDLVTGEQAPEKILRSTFSLGQGLWLPVSKSFVVPPVELSINPLASCKTDVLVTEDPADVREEAMLSTYFETINDLLSSFGPVRDCSRNNGGCTRNFKCVSDRQVDSSGCVCPEELKPMKDGSGCYDHSKGIDCSDGFNGGCEQLCLQQTLPLPYDATSSTIFMFCGCVEEYKLAPDGKSCLMLSDVCEGPKCLKPDSKFNDTLFGEMLHGYNNRTQHVNQGQVFQMTFRENNFIKDFPQLADGLLVIPLPVEEQCRGVLSEPLPDLQLLTGDIRYDEAMGYPMVQQWRVRSNLYRVKLSTITLAAGFTNVLKILTKESSREELLSFIQHYGSHYIAEALYGSELTCIIHFPSKKVQQQLWLQYQKETTELGSKKELKSMPFITYLSGLLTAQMLSDDQLISGVEIRCEEKGRCPSTCHLCRRPGKEQLSPTPVLLEINRVVPLYTLIQDNGTKEAFKSALMSSYWCSGKGDVIDDWCRCDLSAFDANGLPNCSPLLQPVLRLSPTVEPSSTVVSLEWVDVQPAIGTKVSDYILQHKKVDEYTDTDLYTGEFLSFADDLLSGLGTSCVAAGRSHGEVPEVSIYSVIFKCLEPDGLYKFTLYAVDTRGRHSELSTVTLRTACPLVDDNKAEEIADKIYNLYNGYTSGKEQQMAYNTLMEVSASMLFRVQHHYNSHYEKFGDFVWRSEDELGPRKAHLILRRLERVSSHCSSLLRSAYIQSRVETVPYLFCRSEEVRPAGMVWYSILKDTKITCEEKMVSMARNTYGESKGR

SEQ ID NO: 12 - DNA, 476 bp, Homo sapiens
aacttcattatcttggccatccagttagtcatgtgtaactgagtattagatttcggatggagtcatcatggccaattataggacctaattgctctcagcaggcctgagaaatgagttgaaatgtgcagaactgtagaaactttagaggcaacagattttgcctccccgatcagtgtgtgcctgtttacagcactatctatctttctctctccaaatgtcactgagccctttagatgtttatattcaccacgagaagccagtcataaagataaaggaaatttgtgcattataaatgcaatatcactgttttaaacttgactgttttatattatttttgtgtgatcaagtgttccgcaagctattccaactttacaagagaaattgtgattatgttcttttcacctgtgggttataaaaaatgttgtattctgaagacccacaaaatatcaaagacattctgtagtttatacaccgtg

SEQ ID NO: 13 - DNA, 3935 bp, Homo sapiens
caggctcagaggctgaagcaggaggaaggaaggactggaaggaaaaagagacaggttagagggaaagaggcttgggaagaaaacagcagaaaagaaactgctcattacacttacagagaggcaagtaacggtggagatgaggacagagggaaccaagactctgaaagacaaaaaatacaaatagagcgaaagaggaaaaaaatgtcaagaagaacatccatccggagaaatgaagagaatgaaagttttaaactgcagagccgttctgtgcttttccggcacaaaattatatcgctgattttaagcccttttgcatttgccagccgttgacattaagaggcatgtttaacggtgccaacagcatctccttttccttctcctcttcctcttcttcttcttcctcctcctcctcctctttttcctcctcctcgttctcctcccatcagcaagaagacaaaccgaggacagtcttgaaatatcgaaatttcctctttgggatttgccagcgccaagactgtcggaataaaggacgctgactattgtattattgttattttattaattagtcagtggaaagattacagatgaggaaaggggacgcctgtcacccttcctgtgctaagatttaaaaaaaaatgaggctggattgcgggaagctctaaaatgaagcaaaaggagtaagatttttaaagacagaaagccacaggagcccccacgtagcgcacttttatttgtattttttcagatttttttttgtttcgtggtggtgggggaggtgattgggtggctgactggctgcgggaagctacttcctttccttttggagatgattgtgctattattgtttgccttgctctggatggtggaaggagtcttttcccagcttcactacacggtacaggaggagcaggaacatggcactttcgtggggaatatcgctgaagatctgggtctggacattacaaaactttcggctcgcgggtttcagacggtgcccaactcaaggaccccttacttagacctcaacctggagacaggggtgctgtacgtgaacgagaaaatagaccgcgaacaaatctgcaaacagagcccctcctgtgtcctgcacctggaggtctttctggagaaccccctggagctgttccaggtggagatcgaggtgctggacattaatgacaaccccccctctttcccggagccagacctgacggtggaaatctctgagagcgccacgccaggcactcgcttccccttggagagcgcattcgacccagacgtgggcaccaactccttgcgcgactacgagatcacccccaacagctacttctccctggacgtgcagacccagggggatggcaaccgattcgctgagctggtgctggagaagccactggaccgagagcagcaagcggtgcaccgctacgtgctgaccgcggtggacggaggaggtgggggaggagtaggagaaggagggggaggtggcgggggagcaggcctgcccccccagcagcagcgcaccggcacggccctactcaccatccgagtgctggactccaatgacaatgtgcccgctttcgaccaacccgtctacactgtgtccctaccagagaactctcccccaggcactctcgtgatccagctcaacgccaccgacccggacgagggccagaacggtgaggtcgtgtactccttcagcagccacatttcgccccgggcgcgggagcttttcggactctcgccgcgcactggcagactggaggtaagcggcgagttggactatgaagagagcccagtgtaccaagtgtacgtgcaagccaaggacctgggccccaacgccgtgcctgcgcactgcaaggtgctagtgcgagtactggatgctaatgacaacgcgccagagatcagcttcagcaccgtgaaggaagcggtgagtgagggcgcggcgcccggcactgtggtggcccttttcagcgtgactgaccgcgactcagaggagaatgggcaggtgcagtgcgagctactgggagacgtgcctttccgcctcaagtcttcctttaagaattactacaccatcgttaccgaagcccccctggaccgagaggcgggggactcctacaccctgactgtagtggctcgggaccggggcgagcctgcgctctccaccagtaagtcgatccaggtacaagtgtcggatgtgaacgacaacgcgccgcgtttcagccagccggtctacgacgtgtatgtgactgaaaacaacgtgcctggcgcctacatctacgcggtgagcgccaccgaccgggatgagggcgccaacgcccagcttgcctactctatcctcgagtgccagatccagggcatgagcgtcttcacctacgtttctatcaactctgagaacggctacttgtacgccctgcgctccttcgactatgagcagctgaaggacttcagttttcaggtggaagcccgggacgctggcagcccccaggcgctggctggtaacgccactgtcaacatcctcatagtggatcaaaatgacaacgcccctgccatcgtggcgcctctaccagggcgcaacgggactccagcgcgtgaggtgctgccccgctcggcggagccgggttacctgctcacccgcgtggccgccgtggacgcggacgacggcgagaacgcccggctcacttacagcatcgtgcgtggcaacgaaatgaacctctttcgcatggactggcgcaccggggagctgcgcacagcacgccgagtcccggccaagcgcgacccccagcggccttatgagctggtgatcgaggtgcgcgaccatgggcagccgcccctttcctccaccgccaccctggtggttcagctggtggatggcgccgtggagccccagggcgggggcgggagcggaggcggagggtcaggagagcaccagcgccccagtcgctctggcggcggggaaacctcgctagacctcaccctcatcctcatcatcgcgttgggctcggtgtccttcatcttcctgctggccatgatcgtgctggccgtgcgttgccaaaaagagaagaagctcaacatctatacttgtctggccagcgattgctgcctctgctgctgctgctgcggtggcggaggttcgacctgctgtggccgccaagcccgggcgcgcaagaagaaactcagcaagtcagacatcatgctggtgcagagctccaatgtacccagtaacccggcccaggtgccgatagaggagtccgggggctttggctcccaccaccacaaccagaattactgctatcaggtatgcctgacccctgagtccgccaagaccgacctgatgtttcttaagccctgcagcccttcgcggagtacggacactgagcacaacccctgcggggccatcgtcaccggttacaccgaccagcagcctgatatcatctccaacggaagcattttgtccaacgaggtaaggctgaagcgaaaggaccaccatctctcatctcctccatcagaaagcctcctctagcccggcccttgtatctctggtgcactgtatctatttttaggatattagcttatgtgtatcgttgtgggagcagagatgggcggtcaccttctcccactccttcgtgtgtaacctaactttcgcgttgttccaccctttcacatttattttcattccgtccccttggtactttgccaccttggagctccctcctttgctcttccatcctgtcagtcctttcccttctcagtaacctgggcatgaagggaaactgcgtgaagggagagggaaatgtggaggagggacttactttctagcactggcaaaggtcttttttctttgcgtctgtcccaggcattaataaagttggctctattttgctttgtttaacgatgcttttagtcgcgtgtacaagtaagctatagattgtttaacttta

SEQ ID NO: 14 - protein, 896 aa, Homo sapiens
MIVLLLFALLWMVEGVFSQLHYTVQEEQEHGTFVGNIAEDLGLDITKLSARGFQTVPNSRTPYLDLNLETGVLYVNEKIDREQICKQSPSCVLHLEVFLENPLELFQVEIEVLDINDNPPSFPEPDLTVEISESATPGTRFPLESAFDPDVGTNSLRDYEITPNSYFSLDVQTQGDGNRFAELVLEKPLDREQQAVHRYVLTAVDGGGGGGVGEGGGGGGGAGLPPQQQRTGTALLTIRVLDSNDNVPAFDQPVYTVSLPENSPPGTLVIQLNATDPDEGQNGEVVYSFSSHISPRARELFGLSPRTGRLEVSGELDYEESPVYQVYVQAKDLGPNAVPAHCKVLVRVLDANDNAPEISFSTVKEAVSEGAAPGTVVALFSVTDRDSEENGQVQCELLGDVPFRLKSSFKNYYTIVTEAPLDREAGDSYTLTVVARDRGEPALSTSKSIQVQVSDVNDNAPRFSQPVYDVYVTENNVPGAYIYAVSATDRDEGANAQLAYSILECQIQGMSVFTYVSINSENGYLYALRSFDYEQLKDFSFQVEARDAGSPQALAGNATVNILIVDQNDNAPAIVAPLPGRNGTPAREVLPRSAEPGYLLTRVAAVDADDGENARLTYSIVRGNEMNLFRMDWRTGELRTARRVPAKRDPQRPYELVIEVRDHGQPPLSSTATLVVQLVDGAVEPQGGGGSGGGGSGEHQRPSRSGGGETSLDLTLILIIALGSVSFIFLLAMIVLAVRCQKEKKLNIYTCLASDCCLCCCCCGGGGSTCCGRQARARKKKLSKSDIMLVQSSNVPSNPAQVPIEESGGFGSHHHNQNYCYQVCLTPESAKTDLMFLKPCSPSRSTDTEHNPCGAIVTGYTDQQPDIISNGSILSNEVRLKRKDHHLSSPPSESLL

SEQ ID NO: 15 - DNA, 537 bp, Homo sapiens
  modified_base (74): a, t, c, g, other or unknown
acagctgtgggacttgaacatgcaagtgttcaggttgtgtcaagaagcttttctttccttctatgatggaatcngttcttttcnatcnnncttttttctntctncntntcctcnccncattataccnngntcttacgcagtaaacgttttaatggccngtttatgtctcatgcctccaancaacactgaatttgaaaccccccattttttcttttcaccaccctgttgagcaattttcccaaaaaaagggcagcaattattaaattnnnntcaagtnnnnnnnnnnnnnnttcntagattttactaagttttattttgtcnaggttttttaaattttttcagtgagcgtggtgactgcagaggttagtgctgtgaaaagctgggctaaatattctttctgtaaagtcaaacaggattccatcccctgtgaaataacacaaaatttcactctctaaaagcaacagcatgtaaactagaatgaaagaaggaaattatgtacgtatgcctaatattctttgtgaatgtctttcatttaac

SEQ ID NO: 16 - DNA, 8107 bp, Homo sapiens
tgagggaagaagaggaagcgggaggagcttggcttcctcgcgtatttgaggacagcccatctcccttcaagaaccctacggagagtcggactgcatctccgcagcgagctcttggagcgccgccggccgggaggcgaaggatgcaggcggctccgcgcgccggctgcggggcagcgctcctgctgtggattgtcagcagctgcctctgcagagcctggacggctccctccacgtcccaaaaatgtgatgagccacttgtctctggactcccccatgtggctttcagcagctcctcctccatctctggtagctattctcccggctatgccaagataaacaagagaggaggtgctgggggatggtctccatcagacagcgaccattatcaatggcttcaggttgactttggcaatcggaagcagatcagtgccattgcaacccaaggaaggtatagcagctcagattgggtgacccaataccggatgctctacagcgacacagggagaaactggaaaccctatcatcaagatgggaatatctgggcatttcccggaaacattaactctgacggtgtggtccggcacgaattacagcatccgattattgcccgctatgtgcgcatagtgcctctggattggaatggagaaggtcgcattggactcagaattgaagtttatggctgttcttactgggctgatgttatcaactttgatggccatgttgtattaccatatagattcagaaacaagaagatgaaaacactgaaagatgtcattgccttgaactttaagacgtctgaaagtgaaggagtaatcctgcacggagaaggacagcaaggagattacattaccttggaactgaaaaaagccaagctggtcctcagtttaaacttaggaagcaaccagcttggccccatatatggccacacatcagtgatgacaggaagtttgctggatgaccaccactggcactctgtggtcattgagcgccaggggcggagcattaacctcactctggacaggagcatgcagcacttccgtaccaatggagagtttgactacctggacttggactatgagataacctttggaggcatccctttctctggcaagcccagctccagcagtagaaagaatttcaaaggctgcatggaaagcatcaactacaatggcgtcaacattactgatcttgccagaaggaagaaattagagccctcaaatgtgggaaatttgagcttttcttgtgtggaaccctatacggtgcctgtctttttcaacgctacaagttacctggaggtgcccggacggcttaaccaggacctgttctcagtcagtttccagtttaggacatggaaccccaatggtctcctggtcttcagtcactttgcggataatttgggcaatgtggagattgacctcactgaaagcaaagtgggtgttcacatcaacatcacacagaccaagatgagccaaatcgatatttcctcaggttctgggttgaatgatggacagtggcacgaggttcgcttcctagccaaggaaaattttgctattctcaccatcgatggagatgaagcatcagcagttcgaactaatagtccccttcaagttaaaactggcgagaagtacttttttggaggttttctgaaccagatgaataactcaagtcactctgtccttcagccttcattccaaggatgcatgcagctcattcaagtggacgatcaacttgtaaatttatacgaagtggcacaaaggaagccgggaagtttcgcgaatgtcagcattgacatgtgtgcgatcatagacagatgtgtgcccaatcactgtgagcatggtggaaagtgctcgcaaacatgggacagcttcaaatgcacttgtgatgagacaggatacagtggggccacctgccacaactctatctacgagccttcctgtgaagcctacaaacacctaggacagacatcaaattattactggatagatcctgatggcagcggacctctggggcctctgaaagtttactgcaacatgacagaggacaaagtgtggaccatagtgtctcatgacttgcagatgcagacgcctgtggtcggctacaacccagaaaaatactcagtgacacagctcgtttacagcgcctccatggaccagataagtgccatcactgacagtgccgagtactgcgagcagtatgtctcctatttctgcaagatgtcaagattgttgaacaccccagatggaagcccttacacttggtgggttggcaaagccaacgagaagcactactactggggaggctctgggcctggaatccagaaatgtgcctgcggcatcgaacgcaactgcacagatcccaagtactactgtaactgcgacgcggactacaagcaatggaggaaggatgctggtttcttatcatacaaagatcacctgccagtgagccaagtggtggttggagatactgaccgtcaaggctcagaagccaaattgagcgtaggtcctctgcgctgccaaggagacaggaattattggaatgccgcctctttcccaaacccatcctcctacctgcacttctctactttccaaggggaaactagcgctgacatttctttctacttcaaaacattaaccccctggggagtgtttcttgaaaatatgggaaaggaagatttcatcaagctggagctgaagtctgccacagaagtgtccttttcatttgatgtgggaaatgggccagtagagattgtagtgaggtcaccaacccctctcaacgatgaccagtggcaccgggtcactgcagagaggaatgtcaagcaggccagcctacaggtggaccggctaccgcagcagatccgcaaggccccaacagaaggccacacccgcctggagctctacagccagttatttgtgggtggtgctgggggccagcagggcttcctgggctgcatccgctccttgaggatgaatggggtgacacttgacctggaggaaagagcaaaggtcacatctgggttcatatccggatgctcgggccattgcaccagctatggaacaaactgtgaaaatggaggcaaatgcctagagagataccacggttactcctgcgattgctctaatactgcatatgatggaacattttgcaacaaagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggaagcttacagattcgatacaacctgggtggcacccgagagccatacaatattgacgtagaccacaggaacatggccaatggacagccccacagtgtcaacatcacccgccacgagaagaccatctttctcaagctcgatcattatccttctgtgagttaccatctgccaagttcatccgacaccctcttcaattctcccaagtcgctctttctgggaaaagttatagaaacagggaaaattgaccaagagattcacaaatacaacaccccaggattcactggttgcctctccagagtccagttcaaccagatcgcccctctcaaggccgccttgaggcagacaaacgcctcggctcacgtccacatccagggcgagctggtggagtccaactgcggggcctcgccgctgaccctctcccccatgtcgtccgccaccgacccctggcacctggatcacctggattcagccagtgcagattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatttgaggggtggctacttggctatgggatagggaggagggaattactagggaggagagaaagggacaaaagcaccctgcttcatactcttgagcacatccttaaaatatcagcacaagttgggggaggcaggcaatggaatataatggaatattcttgagactgatcacaaaaaaaaaaaaaacctttttaatatttctttatagctgagttttcccttctgtatcaaaacaaaataatacaaaaaatgcttttagagtttaagcaatggttgaaatttgtaggtactatctgtcttattttgtgtgtgtttagaggtgttctaaagacccgtggtaacagggcaagttttctacgtttttaagagcccttagaacgtgggtattttttttcttgagaaaagctaatgcacctacagatggcccccaacattctcttccttttgcttctagtcaaccttaatgggctgttacagaaactagttcgtgtttatatactatttcctttgatgtcctataagtcggaaaagaaaggggcaaagagaacctattatttgccagtttttaagcagagctcaatctatgccagctctctggcatctggggttcctgactgataccagcagttgaaggaagagagtgcatggcacctggtgtgtaacgacacaatcagcacaactggagagaggcattaaagaaccagggaaggtagtttgatttttcattgaattctacaagctaatattgttccacgtatgtagtcttagaccaatagctgtaactatcagctgcaataccatggtgaccagctgttacaaaagattttttcctgttttatctgaaacatactggatttatatatgtataagcgcctcaatggggaattagagccagatgttatgatttgtttgctctttttcttttatagttatagcaaaaatatggataatttctagtgaatgcataaattaggttgcgtttcttattttgctttaaatctctggtagtttttccacccctgtgacacaatcctaatagacagtgtcctgtaaatggacacaacacaataaagtcaagttattattgctgttactctggatgatatggaaaacactgccatattttaaatcaactactccacgtgtttttccatccaatcacactgctgtgattcagggatctttcttctaaaacggacacatttgaacctcaggttcatcacaaacctggtacctgttgcttcccagaggatggagaagtgtagttaatcacacctcttagtttaatctgaaatcttgacccagttatttaacaaataaatacctcattgattatatttaaaagtaatacacttcctgtaaacaaatggggacaatgcatccaaaaaatctttttaaacagattacacaaaaattatttccagaaaggctaccatttatcatcattatatttcaagcctcttatacttaataagcactttctaaaaagtcttgagatcccaccattctgaggaattcaatatgatcactttttccttctttgcctgggagaggttaagaggcggtttcgaaggtatagatgctattgttctgatggcccggctgaataaaatggaaattctagtttgttagaattatgcattctttttcaagattctcagtgtgcctaacttattggagcacatcagtttcttgggtaatggaaaacattacctagagttgccagtggcacattacaccagtacagagcacattccaaaggagacattggaccagttaattcccatacaagtcaaggtaacagaacaaaagggaatcctgatgcccttttaccattgctggttgagctcaggcactgtcatggacacccttaattttaaaaggttttaatcattcttctataaaatacatttaaaatggaaaaatacttaatatcactaaatatcagaacaatgtaacatttacaaatgacatattgaaagcaaaggctgttttatttagccaagatgattaccattaggagttactttatgtattgttgaaagcaaattttaaacatgatgttttagaagtgtttctgatttttaaacctggtttacaggtattacttctgcacttaccaaataatgccagatggaaatttattatttcttgcaattcccgtgatagctctgttctttatgcattgtctcaacactttcccttttttcccaaaatgagtagagaattaaagccacccaaaacagcttctgctactaaaatgttctcatcctttctcctccctctccttttcctgccacaaaaggtgaaaaatgagatccaatcctctcaccaaaatttcaaacctaggacactggaatgactgcagggatcagtggttctcccatatcaccatcaattaagacatataggacactgtcttccttcaagagggttacaatgtggccatcagacaggaaaccaaacggtggataaagtattaagtaactaagtgccaaataaatgctggaaatcttgacctctccttgggattatgggtgtaacaaaaatccctacatctgtttatgaaggccatattcagtacattttaaatggtaaataatctgtttatgtgaagaaaaagaattaagtctttcttccaactctctccttggatagcctagcacagtgcagcctccataaccatgacattcccgcccaagctctcagtgcctaatcctgctttgtcattcacatctcacaaaatcttgacatcttacattccaatacattatcaagcaagcacaagtatgctggtagtagcctctttaaataatatgtatagacaacaacaacgacaaaaaatagactgttttaaagtttcagggaaagttggtggctgatttaaagttgtgcaggaaacatcttctgtgtatgaagcaaatgtcgatgttttgaaaagctaggagatgactttgaatgaatgcaaggttagtgagatcctaagctctcaaaatagcatattccctagagctcaagaaagctggtccaggaggttgaaaaagctattttgttgttaaattattttctggcccttcttaatatttaaaaatgtatttccccttgtggctttcaaccacctgctcaaaaaaagagacttgttacatgaaagttttcattaaagagctgaaaacaagaatttagagagccattcctagaaaatgtcctactgccctgcatttgacaaacaagcatcctttactaacaagagcaggaattcagaggcacaagaaaaagcattggcatgagccaaagagtctgtcttaatgttacttttgaaaatctgctgagcggccaccatatgcaggctgagagctgggcacaggcgaagccattggaagcacttcaggaacaagcacacagctgtgggacttgaacatgcaagtgttcaggttgtgtcaagaagcttttctttccttctatgatggaatctgttcttttctatcctacttttttctctcttcctctcctcaccacattataccctgctcttacgcagtaaacgttttaatggcccgtttatgtctcatgcctccaaacaacactgaatttgaaaccccccattttttcttttcaccaccctgttgagcaattttcccaaaaaaagggcagcaattattaaattgaattcaagtttctagattttactaagttttattttgtcaggttttttaaattttttcagtgagcgtggtgactgcagaggttagtgctgtgaaaagctgggctaaatattctttctgtaaagtcaaacaggattccatcccctgtgaaataacacaaaatttcactctctaaaagcaacagcatgtaaactagaatgaaagaaggaaattatgtacgtatgcctaatattctttgtgaatgtctttcatttaactaaaattatattagaaaccagattgataaataaaaaattcaaagtagttttaattatcct

SEQ ID NO: 17 - protein, 1331 aa, Homo sapiens
MQAAPRAGCGAALLLWIVSSCLCRAWTAPSTSQKCDEPLVSGLPHVAFSSSSSISGSYSPGYAKINKRGGAGGWSPSDSDHYQWLQVDFGNRKQISAIATQGRYSSSDWVTQYRMLYSDTGRNWKPYHQDGNIWAFPGNINSDGVVRHELQHPIIARYVRIVPLDWNGEGRIGLRIEVYGCSYWADVINFDGHVVLPYRFRNKKMKTLKDVIALNFKTSESEGVILHGEGQQGDYITLELKKAKLVLSLNLGSNQLGPIYGHTSVMTGSLLDDHHWHSVVIERQGRSINLTLDRSMQHFRTNGEFDYLDLDYEITFGGIPFSGKPSSSSRKNFKGCMESINYNGVNITDLARRKKLEPSNVGNLSFSCVEPYTVPVFFNATSYLEVPGRLNQDLFSVSFQFRTWNPNGLLVFSHFADNLGNVEIDLTESKVGVHINITQTKMSQIDISSGSGLNDGQWHEVRFLAKENFAILTIDGDEASAVRTNSPLQVKTGEKYFFGGFLNQMNNSSHSVLQPSFQGCMQLIQVDDQLVNLYEVAQRKPGSFANVSIDMCAIIDRCVPNHCEHGGKCSQTWDSFKCTCDETGYSGATCHNSIYEPSCEAYKHLGQTSNYYWIDPDGSGPLGPLKVYCNMTEDKVWTIVSHDLQMQTPVVGYNPEKYSVTQLVYSASMDQISAITDSAEYCEQYVSYFCKMSRLLNTPDGSPYTWWVGKANEKHYYWGGSGPGIQKCACGIERNCTDPKYYCNCDADYKQWRKDAGFLSYKDHLPVSQVVVGDTDRQGSEAKLSVGPLRCQGDRNYWNAASFPNPSSYLHFSTFQGETSADISFYFKTLTPWGVFLENMGKEDFIKLELKSATEVSFSFDVGNGPVEIVVRSPTPLNDDQWHRVTAERNVKQASLQVDRLPQQIRKAPTEGHTRLELYSQLFVGGAGGQQGFLGCIRSLRMNGVTLDLEERAKVTSGFISGCSGHCTSYGTNCENGGKCLERYHGYSCDCSNTAYDGTFCNKDVGAFFEEGMWLRYNFQAPATNARDSSSRVDNAPDQQNSHPDLAQEEIRFSFSTTKAPCILLYISSFTTDFLAVLVKPTGSLQIRYNLGGTREPYNIDVDHRNMANGQPHSVNITRHEKTIFLKLDHYPSVSYHLPSSSDTLFNSPKSLFLGKVIETGKIDQEIHKYNTPGFTGCLSRVQFNQIAPLKAALRQTNASAHVHIQGELVESNCGASPLTLSPMSSATDPWHLDHLDSASADFPYNPGQGQAIRNGVNRNSAIIGGVIAVVIFTILCTLVFLIRYMFRHKGTYHTNEAKGAESAESADAAIMNNDPNFTETIDESKKEWLI

SEQ ID NO: 18 - DNA, 411 bp, Homo sapiens
gctaccactacgaggtgtgtttgaccggagactcaggggccggcgagttcaagttcctgaagccgattattcctaaccttttgccccagggcgctggtgaagaaatagggaaaactgctgccttccggaatagctttggattaaattagagatctcgtgatgacgcgttgttttctgccatttatcccaaactttttcagatctagaattcgagagtgtcatggacaaaaatttcaccttgagattgagcttttatttccctttttaatggatttgtctgttgaacttcatgctgtccaagtgttgaaaagtcaattttatttcattgcatttatttacatagtgtcattccaaatccatgcatgctgttgattttcctgagatttttttctcttcttgttggtatttgtt

SEQ ID NO: 19 - DNA, 2915 bp, Homo sapiens
gcggataactcagacgccattaagctggggaatccaaactctaaaagaaggacgcattttaggtaagatctagtggctagatcttcagggtgggcttcgttcttgtggaaatcagtcaagaaagatcggattcgcggttatttatgcaaatcatctgggtggattgtgtacggagttaaactgcgccttctggaccgggtctgaacaatggagactgcgctagcaaaaacgccacagaaaaggcaagttatgtttcttgctatattgttgcttttgtgggaggctggctctgaggcagttaggtattccataccagaagaaacagaaagtggctattctgtggccaacctggcaaaagacctgggtcttggggtgggggaactggccactcggggcgcgcgaatgcattacaaaggaaacaaagagctcttgcagcttgatataaagaccggcaatttgcttctatatgaaaaactagaccgggaggtgatgtgcggggcgacagaaccctgtatattgcatttccagctcttactagaaaatccagtgcagttttttcaaactgatctgcagctcacagatataaatgaccatgccccagagttcccagagaaggaaatgctcctaaaaatcccagagagcacccagccagggactgtgtttcccttaaaaatagcccaggactttgacataggtagcaacactgttcagaactacacaatcagcccaaattcacactttcatgttgctacgcataatcgcggagatggcagaaaatacccagagctggtgctggacaaagcgctggaccgggaggagcggcctgagctcagcttaacactcactgcactggacggtggggctccgcccaggtccgggaccaccacaattcgcattgtcgtcttggataataatgacaacgcccccgaatttttacaatcattctatgaggtacaggtgcccgagaacagcccccttaactccttagttgtcgttgtctccgctcgagatttagatgcaggagcatatgggagtgtagcctatgctctattccaaggcgatgaagttactcaaccatttgtaatagacgagaaaacagcagaaattcgcctgaaaagggcattggatttcgaggcaactccatattataacgtggaaattgtagccacagatggtgggggcctttcaggaaaatgcactgtggctatagaagtggtggatgtgaatgacaacgcccctgaactcaccatgtctacgctctccagccctaccccagaaaatgccccggaaactgtagttgccgttttcagtgtttctgatccagactccggggacaacggtaggatgatttgctccatccagaatgatctcccctttcttttgaagcccacattaaaaaacttttacaccctagtgacacagagaacactggacagagagagccaagccgagtacaacatcaccatcactgtcaccgacatggggacacccaggctgaaaaccgagcacaacataacggtcctggtctccgacgtcaatgacaacgcccccgccttcacccaaacctcctacaccctgttcgtccgagagaacaacagccccgccctgcacatcggcagtgtcagcgccacagacagagactcaggcaccaacgcccaggtcacctactcgctgctgccgccccagaatccacacctgcgcctcgcctccctggtctccatcaacgcggacaacggccacctgtttgccctcaggtcgctggactacgaggccctgcaggcgttcgagttccgcgtgggagccacagaccgcggctccccggcgctgagcagcgaggcgctggtgcgcgtgctggtgctggacgccaacgacaactcgcccttcgtgctgtatccgctgcagaacggctcggcgccttgcaccgagctggtgccccgggcggccgagccgggctacctggtgaccaaggtggtggcggtggacggtgactcgggccagaacgcctggctgtcgtaccagctgctcaaggccacggagcccgggctgttcagcatgtgggcgcacaatggcgaggtgcgcaccgccaggctgctgagcgagcgcgacgcggccaagcacaggctggtggtgctggtcaaggacaatggcgagcctccgcgctcggccaccgccacgctgcacgtgctcctggtggacggcttctcccagccctacctgccgctgccggaggcggccccggcccaggcccaggccgactcgctcactgtctacctggtggtggcattggcctcggtgtcgtcgctcttcctcttttcggtgctcctgttcgtggcagtgcggctgtgcaggaggagcagggcggccccggtcggtcgctgctcggtgcccgagggcccctttccagggcatctggtggacgtgagcggcaccgggaccctatcccagagctaccactacgaggtgtgtttgaccggagactcaggggccggcgagttcaagttcctgaagccgattattcctaaccttttgccccagggcgctggtgaagaaatagggaaaactgctgccttccggaatagctttggattaaattagagatctcgtgatgacgcgttgttttctgccatttatcccaaactttttcagatctagaattcgagagtgtcatggacaaaaatttcaccttgagattgagcttttatttccctttttaatggatttgtctgttgaacttcatgctgtccaagtgttgaaaagtcaattttatttcattgcatttatttacatagtgtcattccaaatccatgcatgctgttgattttcctgagatttttttctcttcttgttggtatttgttgtgataaaccaccttaataaaatcaagtattaattttaaaaaaaaaaaaaaaaaaaaa

SEQ ID NO: 20 - protein, 701 aa, Homo sapiens
MCGATEPCILHFQLLLENPVQFFQTDLQLTDINDHAPEFPEKEMLLKIPESTQPGTVFPLKIAQDFDIGSNTVQNYTISPNSHFHVATHNRGDGRKYPELVLDKALDREERPELSLTLTALDGGAPPRSGTTTIRIVVLDNNDNAPEFLQSFYEVQVPENSPLNSLVVVVSARDLDAGAYGSVAYALFQGDEVTQPFVIDEKTAEIRLKRALDFEATPYYNVEIVATDGGGLSGKCTVAIEVVDVNDNAPELTMSTLSSPTPENAPETVVAVFSVSDPDSGDNGRMICSIQNDLPFLLKPTLKNFYTLVTQRTLDRESQAEYNITITVTDMGTPRLKTEHNITVLVSDVNDNAPAFTQTSYTLFVRENNSPALHIGSVSATDRDSGTNAQVTYSLLPPQNPHLRLASLVSINADNGHLFALRSLDYEALQAFEFRVGATDRGSPALSSEALVRVLVLDANDNSPFVLYPLQNGSAPCTELVPRAAEPGYLVTKVVAVDGDSGQNAWLSYQLLKATEPGLFSMWAHNGEVRTARLLSERDAAKHRLVVLVKDNGEPPRSATATLHVLLVDGFSQPYLPLPEAAPAQAQADSLTVYLVVALASVSSLFLFSVLLFVAVRLCRRSRAAPVGRCSVPEGPFPGHLVDVSGTGTLSQSYHYEVCLTGDSGAGEFKFLKPIIPNLLPQGAGEEIGKTAAFRNSFGLN

SEQ ID NO: 21 - DNA, 521 bp, Homo sapiens
ggcacagagcgcggagatgtaccactaccagcaccaacggcaacagatgctgtgcctggagcggcataaagagccacccaaggagctggacacggcctcctcggatgaggagaatgaggacggagacttcacggtgtacgagtgcccgggcctggccccgaccggggaaatggaggtgcgcaaccctctgttcgaccacgccgcactgtccgcgcccctgccggcccccagctcaccgcctgcactgccatgacctggaggcagacagacgcccacctgctccccgacctcgaggcccccggggaggggcagggcctggagcttcccactaaaaacatgttttgatgctgtgtgcttttggctgggcctcgggctccaggccctgggaccccttgccagggagacccccgaacctttgtgccaggacacctcctggtcccctgcacctctcctgttcggtttagacccccaaactggagggggcatggagaaccgtagagcgcaggaacgggtgggtaatt

SEQ ID NO: 22 - DNA, 1441 bp, Homo sapiens
ggcacgagggcctcttcttcctcctgcgtcctcccccgctgcctccgctgctcccgacgcggagcccggagcccgcgccgagcccctggcctcgcggtgccatgctgccccggcggcggcgctgaaggatggcgacgccgctgcctccgccctccccgcggcacctgcggctgctgcggctgctgctctccggcctcgtcctcggcgccgccctgcgtggagccgccgccggccacccggatgtagccgcctgtcccgggagcctggactgtgccctgaagaggcgggcaaggtgtcctcctggtgcacatgcctgtgggccctgccttcagcccttccaggaggaccagcaagggctctgtgtgcccaggatgcgccggcctccaggcgggggccggccccagcccagactggaagatgagattgacttcctggcccaggagcttgcccggaaggagtctggacactcaactccgcccctacccaaggaccgacagcggctcccggagcctgccaccctgggcttctcggcacgggggcaggggctggagctgggcctcccctccactccaggaacccccacgcccacgccccacacctccctgggctcccctgtgtcatccgacccggtgcacatgtcgcccctggagccccggggagggcaaggcgacggcctcgcccttgtgctgatcctggcgttctgtgtggccggtgcagccgccctctccgtagcctccctctgctggtgcaggctgcagcgtgagatccgcctgactcagaaggccgactacgccactgcgaaggcccctggctcacctgcagctccccggatctcgcctggggaccagcggctggcacagagcgcggagatgtaccactaccagcaccaacggcaacagatgctgtgcctggagcggcataaagagccacccaaggagctggacacggcctcctcggatgaggagaatgaggacggagacttcacggtgtacgagtgcccgggcctggccccgaccggggaaatggaggtgcgcaaccctctgttcgaccacgccgcactgtccgcgcccctgccggcccccagctcaccgcctgcactgccatgacctggaggcagacagacgcccacctgctccccgacctcgaggcccccggggaggggcagggcctggagcttcccactaaaaacatgttttgatgctgtgtgcttttggctgggcctcgggctccaggccctgggaccccttgccagggagacccccgaacctttgtgccaggacacctcctggtcccctgcacctctcctgttcggtttagacccccaaactggagggggcatggagaaccgtagagcgcaggaacgggtgggtaattctagagacaaaagccaattaaagtccatttcagacctgcggcttctgaaaaaaaaaaaaaaaaaaaa

SEQ ID NO: 23 - protein, 325 aa, Homo sapiens
MATPLPPPSPRHLRLLRLLLSGLVLGAALRGAAAGHPDVAACPGSLDCALKRRARCPPGAHACGPCLQPFQEDQQGLCVPRMRRPPGGGRPQPRLEDEIDFLAQELARKESGHSTPPLPKDRQRLPEPATLGFSARGQGLELGLPSTPGTPTPTPHTSMGSPVSSDPVHMSPLEPRGGQGDGLALVLILAFCVAGAAALSVASLCWCRLHREIRLTQKADYATAKAPGSPAAPRISPGDQRLAQSAEMYHYQHQRQQMLCLERHKEPPKELDTASSDEENEDGDFTVYECPGLAPTGEMEVRNPLFDHAALSAPLPAPSSPPALP

SEQ ID NO: 24 - DNA, 578 bp, Homo sapiens
  modified_base (138): a, t, c, g, other or unknown
gaaatccttcctgctcaggctttcattctaaaactacagtcttcattaaagctgaactttctgggtagctgagcttatatgcccggcatctgaatgagagctctctttgtaactgtgtgacttgagatctagtttgcnagntccnggnaaacaatacatgtgttnttnnntttgtgtttgctcagcaagcagatgtctgagatgtaagaagcttttcttttcctgtggcattgattctgacttagagctgaagtaaagatcactgaaacatcacgtcaagttgaagtcactcataggtctttgtcctttaggcaggacaggagagtcattaagaagcatttcactgtagcattctatcacaatatcatctggaattnttttctttgcccagaaagccttaacttgcctctagagaatccctggnnnnnnnnnnnnnnnnnnnnnnnnnnnntncaactcttctgctgtggaagtttgaagcgacngncnaggcananccagagaatttcctcaagtngcctntaggtnccntgttatcttatgcccccacccctccctcaacaatatgagtgatccag

SEQ ID NO: 25 - DNA, 1788 bp, Homo sapiens
gaattcgggcggggagctgcaggaaccagactgggggcgagctgagcacctgtagtcaatcacacgcagcttttaggtttgtttgaataagagatctgacctgaccggcccaactgtacaactcttcaaggaaaattcgtatttgcagtgggaagaataagtaacattgatcaagatgaatgccatgctggagactcccgaactcccagccgtgtttgatggagtgaagctggctgcagtggctgctgtgctgtacgtgatcgtccggtgtttgaacctgaagagccccacagccccacctgacctctacttccaggactcggggctctcacgctttctgctcaagtcctgtcctcttctgaccaaagaatacattccaccgttgatctgggggaaaagtggacacatccagacagccttgtatgggaagatgggaagggtgaggtcgccacatccttatgggcaccggaagttcatcactatgtctgatggagccacttctacattcgacctcttcgagcccttggctgagcactgtgttggagatgatatcaccatggtcatctgccctggaattgccaatcacagcgagaagcaatacatccgcactttcgttgactacgcccagaaaaatggctatcggtgcgccgtgctgaaccacctgggtgccctgcccaacattgaattgacctcgccacgcatgttcacctatggctgcacgtgggaatttggagccatggtgaactacatcaagaagacatatcccctgacccagctggtcgtcgtgggcttcagcctgggtggtaacattgtgtgcaaatacttgggggagactcaggcaaaccaagagaaggtcctgtgctgcgtcagcgtgtgccaggggtacagtgcactgagggcccaggaaaccttcatgcaatgggatcagtgccggcggttctacaacttcctcatggctgacaacatgaagaagatcatcctctcgcacaggcaagctctttttggagaccatgttaagaaaccccagagcctggaagacacggacttgagccggctctacacagcaacatccctgatgcagattgatgacaatgtgatgaggaagtttcacggctataactccctgaaggaatactatgaggaagaaagttgcatgcggtacctgcacaggatttatgttcctctcatgctggttaatgcagctgacgatccgttggtgcatgaaagtcttctaaccattccaaaatctctttcagagaaacgagagaacgtcatgtttgtgctgcctctgcatgggggccacttgggcttctttgagggctctgtgctgttccccgagcccctgacatggatggataagctggtggtggagtacgccaacgccatttgccaatgggagcgtaacaagttgcagtgctctgacacggagcaggtggaggccgacctggagtgaggcctccggactctggcacgctccagcagccctcctctggaagctgcgtcccctcaccccctgtttcaggtctcccatctccctcagtgacctggatctgacctcacaccatcagcagggggcacccaccatgcacacctgtctcggagtaggcagctcttcctgggagctccaggctatttttgtgcttagttactggttttctccattgcattgttaggcatggtgacaagtgacagagttcttgccctctgtccagtttcagcatctggttgcttttaagccaagtacatctagtttccctattaaaaatgtgtctgaatccccccgaattc

SEQ ID NO: 26 - protein, 425 aa, Homo sapiens
MNAMLETPELPAVFDGVKLAAVAAVLYVIVRCLNLKSPTAPPDLYFQDSGLSRFLLKSCPLLTKEYIPPLIWGKSGHIQTALYGKMGRVRSPHPYGHRKFITMSDGATSTFDLFEPLAEHCVGDDITMVICPGIANHSEKQYIRTFVDYAQKNGYRCAVLNHLGALPNIELTSPRMFTYGCTWEFGAMVNYIKKTYPLTQLVVVGFSLGGNIVCKYLGETQANQEKVLCCVSVCQGYSALRAQETFMQWDQCRRFYNFLMADNMKKIILSHRQALFGDHVKKPQSLEDTDLSRLYTATSLMQIDDNVMRKFHGYNSLKEYYEEESCMRYLHRIYVPLMLVNAADDPLVHESLLTIPKSLSEKRENVMFVLPLHGGHLGFFEGSVLFPEPLTWMDKLVVEYANAICQWERNKLQCSDTEQVEADLE

SEQ ID NO: 27 - DNA, 436 bp, Homo sapiens
ggaagtcatcttttgagatccagatagacatggtttgtgcacttacgtccagatgggaagcatccttcctgcaaccctaaaataatcatgcagcctctcagacggacgccatcggtcccaaggccttaggtggaggaagcaaagcaggccaggcctgtcctgtccgtggacctctaccttctggactccctacgggtgcagagcacttgggtttctctacagccatcgtggcccacttgacactgtgctcctccatcagctggtcacatgccaacacgttcccagcccctgaggcagctccagggtgccccacctgctcctgaggtgggtccctaccctgctgctcctcttcatcctttcccttttgtcctgaaagggaggagcaatggtccaggcattaattccacccagggaattttagctatgccctcatgtc

SEQ ID NO: 28 - DNA, 2432 bp, Homo sapiens
ggcgagtggcgagtggcgagtgtcaggggggcggccggcgggggcggggcggccggaggaggcgttggcagcgggctcggacccacgcggcgccgcggcccgcctggcctgcagcgctcccacccccggcggcggcacgatgccctttgacttcaggaggtttgacatctacaggaaggtgcccaaggaccttacgcagccaacgtacaccggggccattatctccatctgctgctgcctcttcatcctcttcctcttcctctcggagctcaccggatttataacgacagaagttgtgaacgagctctatgtcgatgacccagacaaggacagcggtggcaagatcgacgtcagtctgaacatcagtttacccaatctgcactgcgagttggttgggcttgacattcaggatgagatgggcaggcacgaagtgggccacatcgacaactccatgaagatcccgctgaacaatggggcaggctgccgcttcgaggggcagttcagcatcaacaaggtccccggcaacttccacgtgtccacacacagtgccacagcccagccacagaacccagacatgacgcatgtcatccacaagctctcctttggggacacgctacaggtccagaacatccacggagctttcaatgctctcgggggagcagacagactcacctccaaccccctggcctcccacgactacatcctgaagattgtgcccacggtttatgaggacaagagtggcaagcagcggtactcctaccagtacacggtggccaacaaggaatacgtcgcctacagccacacgggccgcatcatccctgcaatctggttccgctacgacctcagccccatcacggtcaagtacacagagagacggcagccgctgtacagattcatcaccacgatctgtgccatcattggcgggaccttcaccgtcgccggcatcctggactcatgcatcttcacagcctctgaggcctggaagaagatccagctgggcaagatgcattgacgccacacccagcctaatggccgaggaccctgggcatcgccagccttgcctccagtgccctgtctcctttggccctcaatctggtcccaaatctggctgtgtcccaaagggtgtgtgggaagtggggggaaagtagaggatggctcgatgttttgcagctacctcttttccccgtgtttctttttagacaaattacactgcctgaagttgcagttcccctttccctggggagccccaagaacagagtcaggcaaggggtggggagtccagggatcttggggacccctcctaggagagctgcagtctcttccctcaggggaacatcccagaatgcatatcgatcagctctcagccaggcttcgacaatctcgcagcccccactaggtggacacattaatgatttggtttctcccctgggcagccaacctgccccagaggcaccagacctgggctttcagctttgggaccaggctgcccaaaggtactcctttatacacccggcaccttccacgaaagatggtacttcccaagcaagcccctatgatttgtcactatagatggaaccctgacttctgccccatcccttcctgcccaacctagaacccaggcctcaagtctttaccccacccctttcttgttcttccaagaagcagatgcccagttgctcagcagcagcggtagagacttgaatctgcccaccagtcacaaggcgggtcacagattcctcttcctctcttctcctcgttcctctgaaccctccaccaatgtgcctcagcctgtgtgctgtgtggcaacagcattctggttcccactgccaagatctcccaccactctgctgggatctgcagtggcagggagtgggggttgtgtaaaggggaagtcatcttttgagatccagatagacatggtttgtgcacttacgtccagatgggaagcatccttcctgcaaccctaaaataatcatgcagcctctcagacggacgccatcggtcccaaggccttaggtggaggaagcaaagcaggccaggcctgtcctgtccgtggacctctaccttctggactccctacgggtgcagagcacttgggtttctctacagccatcgtggcccacttgacactgtgctcctccatcagctggtcacatgccaacacgttcccagcccctgaggcagctccagggtgccccacctgctcctgaggtgggtccctaccctgctgctcctcttcatcctttcccttttgtcctgaaagggaggagcaatggtccaggcattaattccacccagggaattttagctatgccctcatgtcccagggagagagccacacgcctgttttccatttatagcaagattgtttgcatacttttgtaatgaaggggagtgtccagtggaaggatttttaaaattatcttatggat

SEQ ID NO: 29 - protein, 336 aa, Homo sapiens
ASGEWRVSGGRPAGAGRPEEALAAGSDPRGAAARLACSAPTPGGGTMPFDFRRFDIYRKVPKDLTQPTYTGAIISICCCLFILFLFLSELTGFITTEVVNELYVDDPDKDSGGKIDVSLNISLPNLHCELVGLDIQDEMGRHEVGHIDNSMKIPLNNGAGCRFEGQFSINKVPGNFHVSTHSATAQPQNPDMTHVIHKLSFGDTLQVQNIHGAFNALGGADRLTSNPLASHDYILKIVPTVYEDKSGKQRYSYQYTVANKEYVAYSHTGRIIPAIWFRYDLSPITVKYTERRQPLYRFITTICAIIGGTFTVAGILDSCIFTASEAWKKIQLGKMH

SEQ ID NO: 30 - DNA, 371 bp, Homo sapiens
tgggatatcagtgaactatgttgtatacttttgaatttttacattttataaatggaattgaaagttggataactgctttttttaaattttccaacagaagtaacaccacagttgctttgtttctttttatagcttacctgaggttcagttcttctttgtgaacctgtgagtactccacagtttactgggggaaaaggcttcagtaaagcagaggctagaattacagtatttatacatagcaacttttcataaagtagaaaaattcaaaggaagctgtctcaatttgagaataccagctgggcacggtggctcacgcctgtaatcccagcacttactttgggaggccaaggtgggcagataacctgcggtca

SEQ ID NO: 31 - DNA, 7931 bp, Homo sapiens
ggctgctcctgcactgcgccggccctgagcggacctgtggctcggactatctattacatcgcagccgagctggtccggctggtggggtctgtggactccatgaagcccgtgctccagtccctctaccaccgagtgctgctctaccccccaccccagcaccgggtggaagccatcaaaataatgaaagagatacttgggagcccacagcgtctctgtgacttggcaggacccagctccactgaatcagagtccagaaaaagatcaatttcaaaaagaaagtctcatctggatctcctcaaactcatcatggatggcatgaccgaagcatgcatcaagggtggcatcgaagcttgctatgcagccgtgtcctgtgtctgcaccttgctgggtgccctggatgagctcagccaggggaagggcttgagcgaaggtcaggtgcaactgctgcttctgcgccttgaggagctgaaggatggggctgagtggagccgagattccatggagatcaatgaggctgacttccgctggcagcggcgagtgctgtcctcagaacacacgccgtgggagtcagggaacgagaggagccttgacatcagcatcagtgtcaccacagacacaggccagaccactctcgagggagagttgggtcagactacacccgaggaccattcgggaaaccacaagaacagtctcaagtcgccagccatcccagagggtaaggagacgctgagcaaagtattggaaacagaggcggtagaccagccagatgtcgtgcagagaagccacacggtcccttaccctgacataactaacttcctgtcagtagactgcaggacaaggtcctatggatctaggtatagtgagagcaattttagcgttgatgaccaagacctttctaggacagagtttgattcctgtgatcagtactctatggcagcagaaaaggactcgggcaggtccgacgtgtcagacattgggtcggacaactgttcactagccgatgaagagcagacaccccgggactgcctaggccaccggtccctgcgaactgccgccctgtctctaaaactgctgaagaaccaggaggcggatcagcacagcgccaggctgttcatacagtccctggaaggcctcctccctcggctcctgtctctctccaatgtagaggaggtggacaccgctctgcagaactttgcctctactttctgctcaggcatgatgcactctcctggctttgacgggaatagcagcctcagcttccagatgctgatgaacgcagacagcctctacacagctgcacactgcgccctgctcctcaacctgaagctctcccacggtgactactacaggaagcggccgaccctggcgccaggcgtgatgaaggacttcatgaagcaggtgcagaccagcggcgtgctgatggtcttctctcaggcctggattgaggagctctaccatcaggtgctcgacaggaacatgcttggagaggctggctattggggcagcccagaagataacagccttcccctcatcacaatgctgaccgatattgacggcttagagagcagtgccattggtggccagctgatggcctcggctgctacagagtctcctttcgcccagagcaggagaattgatgactccacagtggcaggcgtggcatttgctcgctatattctggtgggctgctggaagaacttgatcgatactttatcaaccccactgactggtcgaatggcggggagctccaaagggctggccttcattctgggagctgaaggcatcaaagagcagaaccagaaggagcgggacgccatctgcatgagcctcgacgggctgcggaaagccgcacggctgagctgcgctctaggcgttgctgctaactgcgcctcagcccttgcccagatggcagctgcctcctgtgtccaagaagaaaaagaagagagggaggcccaagaacccagtgatgccatcacacaagtgaaactaaaagtggagcagaaactggagcagattgggaaggtgcagggggtgtggctgcacactgcccacgtcttgtgcatggaggccatcctcagcgtaggcctggagatgggaagccacaacccggactgctggccacacgtgttcagggtgtgtgaatacgtgggcaccctggagcacaaccacttcagcgatggtgcctcgcagccccctctgaccatcagccagccccagaaggccactggaagcgctggcctccttggggaccccgagtgtgagggctcgccccccgagcacagcccggagcaggggcgctccctgagcacggcccctgtcgtccagcccctgtccatccaggacctcgtccgggaaggcagccggggtcgggcctccgacttccgcggcgggagcctcatgagcgggagcagcgcggccaaggtggtgctcaccctctccacgcaagccgacaggctctttgaagatgctacggataagttgaacctcatggccttgggaggttttctttaccagctgaagaaagcatcgcagtctcagcttttccattctgttacagatacagttgattactctctggcaatgccaggagaagttaaatccactcaagaccgaaaaagcgccctccacctgttccgcctggggaatgccatgctgaggattgtgcggagcaaagcacggcccctgctccacgtgatgcgctgctggagccttgtggccccacacctggtggaggctgcttgccataaggaaagacatgtgtctcagaaggctgtttccttcatccatgacatactgacagaagtcctcactgactggaatgagccacctcattttcacttcaatgaagcactcttccgacctttcgagcgcattatgcagctggaattgtgtgatgaggacgtccaagaccaggttgtcacatccattggtgagctggttgaagtgtgttccacgcagatccagtcgggatggagacccttgttcagtgccctggaaacagtgcatggcgggaacaagtcagagatgaaggagtacctggttggtgactactccatgggaaaaggccaagctccagtgtttgatgtatttgaagcttttctcaatactgacaacatccaggtctttgctaatgcagccactagctacatcatgtgccttatgaagtttgtcaaaggactgggggaggtggactgtaaagagattggagactgtgccccagcacccggagccccgtccacagacctgtgcctcccggccctggattacctcaggcgctgctctcagttattggccaaaatctacaaaatgcccttgaagccaatattccttagtgggagacttgccggcttgcctcgaagacttcaggaacagtcagccagcagtgaggatggaattgaatcagtcctgtctgattttgatgatgacaccggtctgatagaagtctggataatcctgctggagcagctgacagcggctgtgtccaattgtccacggcagcaccaaccaccaactctggatttactctttgagctgttgagagatgtgacgaaaacaccaggaccagggtttggtatctatgcagtggttcacctcctccttcctgtgatgtccgtttggctccgccggagccataaagaccattcctactgggatatggcctctgccaatttcaagcacgctattggtctgtcctgtgagctggtggtggagcacattcaaagctttctacattcagatatcaggtacgagagcatgatcaataccatgctgaaggacctctttgagttgctggtcgcctgtgtggccaagcccactgaaaccatctccagagtgggctgctcctgtattagatacgtccttgtgacagcgggccctgtgttcactgaggagatgtggaggcttgcctgctgtgccctgcaagatgcgttctctgccacactcaagccagtgaaggacctgctgggctgcttccacagcggcacggagagcttcagcggggaaggctgccaggtgcgagtggcggccccgtcctcctccccaagtgccgaggccgagtactggcgcatccgagccatggcccagcaggtgtttatgctggacacccagtgctcaccaaagacaccaaacaactttgaccacgctcagtcctgccagctcattattgagctgcctcctgatgaaaaaccaatggacacaccaagaaaagcgtgtctttcagggaaattgtggtgagcctgctgtctcatcaggtgttactccagaacttatatgacatcttgttagaagagtttgtcaaaggcccctctcctggagaggaaaagacgatacaagtgccagaagccaagctggctggcttcctcagatacatctctatgcagaacttggcagtcatattcgacctgctgctggactcttataggactgccagggagtttgacaccagccccgggctgaagtgcctgctgaagaaagtgtctggcatcgggggcgccgccaacctctaccgccagtctgcgatgagctttaacatttatttccacgccctggtgtgtgctgttctcaccaatcaagaaaccatcacggccgagcaagtgaagaaggtcctttttgaggacgacgagagaagcacggattcttcccagcagtgttcatctgaggatgaagacatctttgaggaaaccgcccaggtcagccccccgagaggcaaggagaagagacagtggcgggcacggatgcccttgctcagcgtccagcctgtcagcaacgcagattgggtgtggctggtcaagaggctgcacaagctgtgcatggaactgtgcaacaactacatccagatgcacttggacctggagaactgtatggaggagcctcccatcttcaagggcgacccgttcttcatcctgccctccttccagtccgagtcatccaccccatccaccgggggcttctctgggaaagaaaccccttccgaggatgacagaagccagtcccgggagcacatgggcgagtccctgagcctgaaggccggtggtggggacctgctgctgccccccagccccaaagtggagaagaaggatcccagccggaagaaggagtggtgggagaatgcggggaacaaaatctacaccatggcagccgacaagaccatttcaaagttgatgaccgaatacaaaaagaggaaacagcagcacaacctgtccgcgttccccaaagaggtcaaagtggagaagaaaggagagccactgggtcccaggggccaggactccccgctgcttcagcgtccccagcacttgatggaccaagggcaaatgcggcattccttcagcgcaggccccgagctgctgcgacaggacaagaggccccgctcaggctccaccgggagctccctcagtgtctcggtgagagacgcagaagcacagatccaggcatggaccaacatggtgctaacagttctcaatcagattcagattctcccagaccagaccttcacggccctccagcccgcagtgttcccgtgcatcagtcagctgacctgtcacgtgaccgacatcagagttcgccaggctgtgagggagtggctgggcagggtgggccgtgtctatgacatcattgtgtagccgactcctgttctactctcccaccaaataacagtagtgagggttagagtcctgccaatacagctgttgcattttccccaccactagccccacttaaactactactactgtctcagagaacagtgtttcctaatgtaaaaagcctttccaaccactgatcagcattggggccatactaaggtttgtatctagatgacacaaacgatattctgattttgcacattattatagaagaatctataatccttgatatgtttctaactcttgaagtatatttcccagtgcttttgcttacagtgttgtccccaaatgggtcattttcaaggattactcatttgaaaacactatattgatccatttgatccatcatttaaaaaataaatacaattcctaaggcaatatctgctggtaagtcaagctgataaacactcagacatctagtaccagggattattaattggaggaagatttatggttatgggtctggctgggaagaagacaactataaatacatattcttgggtgtcataatcaagaaagaggtgacttctgttgtaaaataatccagaacacttcaaaattattcctaaatcattaagattttcaggtattcaccaatttccccatgtaaggtactgtgttgtacctttatttctgtatttctaaaagaagaaagttctttcctagcagggtttgaagtctgtggcttatcagcctgtgacacagagtacccagtgaaagtggctggtacgtagattgtcaagagacataagaccgaccagccaccctggctgttcttgtggtgtttgtttccatccccaaggcaaacaaggaaaggaaaggaaagaagaaaaggtgccttagtcctttgttgcacttccatttccatgccccacaattgtctgaacataaggtatagcatttggtttttaagaaaacaaaacattaagacgcaactcattttatatcaacacgcttggaggaaagggactcagggaagggagcagggagtgtggggtggggatggattatgatgaaatcattttcaatcttaaaatataatacaacaatcttgcaaaattatggtgtcagttacacaagctctagtctcaaaatgaaagtaatggagaaagacactgaaatttagaaaattttgtcgatttaaaatatttctcctatctaccaagtaaagttaccctatgtttgatgtctttgcattcagaccaatatttcaggtggatatttctaagtattactagaaaatacgtttgaaagctttatcttattatttacagtatttttatatttcttacattatcctaatgattgaaaactcctcaatcaagcttacttacacacattctacagagttatttaaggcatacattataatctcccagccccattcataatgaataagtcaccctttaaatataagacacaaattctacagtattgaaataaggatttaaaggggtatttgtaaactttgccctccttgagaaatatggaactaccttagaggttaagaggaaggcagtgttctgacttctttaggtgatctgaaaaaaacacccttatcatccagtgtaccatctagagatcaccacagaatccatttttttcccagttccacaaaacactctgtttgccttcagtttttactcactagacaataattcaagtttagaaacaggtaatcagctatttgatcttaaaaggcaatgaattgttgggatatcagtgaactatgttgtatacttttgaatttttacattttataaatggaattgaaagttggataactgctttttttaaattttccaacagaagtaacaccacagttgctttgtttctttttatagcttacctgaggttcagttcttctttgtgaacctgtgagtactccacagtttactgggggaaaaggcttcagtaaagcagaggctagaattacagtatttatacatagcaacttttcataaagtagaaaaattcaaaggaagctgtctcaatttgagaataccagctgggcacggtggctcacgcctgtaatcccagcacttactttgggaggccaaggtgggcagataacctgcggtcaggagtttgagaccaggctggacaacatggtgaaacctcgtctctactaaaaatacaaaaattagccaggtgtggtaggatgcacctgtaatcccagctacttaggaggccgagacaggagaatcgctcgaacccaggaggcggacgttgcagtgagccaagattgcaccattgcactccagactgggtgacaagagtgaaactccatct

SEQ ID NO: 32 - protein, 1872 aa, Homo sapiens
GCSCTAPALSGPVARTIYYIAAELVRLVGSVDSMKPVLQSLYHRVLLYPPPQHRVEAIKIMKEILGSPQRLCDLAGPSSTESESRKRSISKRKSHLDLLKLIMDGMTEACIKGGIEACYAAVSCVCTLLGALDELSQGKGLSEGQVQLLLLRLEELKDGAEWSRDSMEINEADFRWQRRVLSSEHTPWESGNERSLDISISVTTDTGQTTLEGELGQTTPEDHSGNHKNSLKSPAIPEGKETLSKVLETEAVDQPDVVQRSHTVPYPDITNFLSVDCRTRSYGSRYSESNFSVDDQDLSRTEFDSCDQYSMAAEKDSGRSDVSDIGSDNCSLADEEQTPRDCLGHRSLRTAALSLKLLKNQEADQHSARLFIQSLEGLLPRLLSLSNVEEVDTALQNFASTFCSGMMHSPGFDGNSSLSFQMLMNADSLYTAAHCALLLNLKLSHGDYYRKRPTLAPGVMKDFMKQVQTSGVLMVFSQAWIEELYHQVLDRNMLGEAGYWGSPEDNSLPLITMLTDIDGLESSAIGGQLMASAATESPFAQSRRIDDSTVAGVAFARYILVGCWKNLIDTLSTPLTGRMAGSSKGLAFILGAEGIKEQNQKERDAICMSLDGLRKAARLSCALGVAANCASALAQMAAASCVQEEKEEREAQEPSDAITQVKLKVEQKLEQIGKVQGVWLHTAHVLCMEAILSVGLEMGSHNPDCWPHVFRVCEYVGTLEHNHFSDGASQPPLTISQPQKATGSAGLLGDPECEGSPPEHSPEQGRSLSTAPVVQPLSIQDLVREGSRGRASDFRGGSLMSGSSAAKVVLTLSTQADRLFEDATDKLNLMALGGFLYQLKKASQSQLFHSVTDTVDYSLAMPGEVKSTQDRKSALHLFRLGNAMLRIVRSKARPLLHVMRCWSLVAPHLVEAACHKERHVSQKAVSFIHDILTEVLTDWNEPPHFHFNEALFRPFERIMQLELCDEDVQDQVVTSIGELVEVCSTQIQSGWRPLFSALETVHGGNKSEMKEYLVGDYSMGKGQAPVFDVFEAFLNTDNIQVFANAATSYIMCLMKFVKGLGEVDCKEIGDCAPAPGAPSTDLCLPALDYLRRCSQLLAKIYKMPLKPIFLSGRLAGLPRRLQEQSASSEDGIESVLSDFDDDTGLIEVWIILLEQLTAAVSNCPRQHQPPTLDLLFELLRDVTKTPGPGFGIYAVVHLLLPVMSVWLRRSHKDHSYWDMASANFKHAIGLSCELVVEHIQSFLHSDIRYESMINTMLKDLFELLVACVAKPTETISRVGCSCIRYVLVTAGPVFTEEMWRLACCALQDAFSATLKPVKDLLGCFHSGTESFSGEGCQVRVAAPSSSPSAEAEYWRIRAMAQQVFMLDTQCSPKTPNNFDHAQSCQLIIELPPDEKPNGHTKKSVSFREIVVSLLSHQVLLQNLYDILLEEFVKGPSPGEEKTIQVPEAKLAGFLRYISMQNLAVIFDLLLDSYRTAREFDTSPGLKCLLKKVSGIGGAANLYRQSAMSFNIYFHALVCAVLTNQETITAEQVKKVLFEDDERSTDSSQQCSSEDEDIFEETAQVSPPRGKEKRQWRARMPLLSVQPVSNADWVWLVKRLHKLCMELCNNYIQMHLDLENCMEEPPIFKGDPFFILPSFQSESSTPSTGGFSGKETPSEDDRSQSREHMGESLSLKAGGGDLLLPPSPKVEKKDPSRKKEWWENAGNKIYTMAADKTISKLMTEYKKRKQQHNLSAFPKEVKVEKKGEPLGPRGQDSPLLQRPQHLMDQGQMRHSFSAGPELLRQDKRPRSGSTGSSLSVSVRDAEAQIQAWTNMVLTVLNQIQILPDQTFTALQPAVFPCISQLTCHVTDIRVRQAVREWLGRVGRVYDIIV

SEQ ID NO: 33 - DNA, 489 bp, Homo sapiens
  modified_base (250): a, t, c, g, other or unknown
aattttcattccaaatcacttagctgttagactgatctgtttgtagcagttgtttgtctcatttttgctctgtgcattttttgagacatttgttgagaatattctatttggtgctctactgtatttttctttttaatatctacttgatatcttgttctttaaattttcttcacatatggtttgcctgatacaactgatttttataactgaaatttaaggaatctaacagctaaaactcagtaagtgcatntatttccttataacatagacccgttgctactctcagcaccctctcctcaattttttttcctgtagcatgtgatgcctgattaaactcattttcatttgcttttatttctaatatgggaacaatgagagtgaactctaaatataggttgtagtaataaaacatcattagcctaattattagaaaatgctaattaagtaccagcacatagaaacatgaaattgcttagtcattgtaccttt

SEQ ID NO: 34 - DNA, 4552 bp, Homo sapiens
cggctgcaggctgggagggagaagtgctacgcctttgcaggttggcgaagtggttccaggctacccggctagtctggcacggccccgtcttctgcctcctcctccgtcgcgtggcggcgggaactgttggccgcgcggcctcgggaacggcccaggtccccgcccgcaggtcccgggcagataacatagatcatcagtagaaaacttcttgaagttgttcaagaaaaatttgaaagtagcaaaatagaaaataaagaattaacagcagatacagaggacagcatggaagtgttgtcttaggaaacagaacacagcagtgaaaaaacagacaaaatccgctcagatacaactgcagctgataatgttttccggcttcaatgtctttagagttgggatctcttttgtcataatgtgcattttttacatgccaacagtaaactctttaccagaactgagtcctcagaaatattttagtacattgcaaccaggaaaagcctctttagcttatttttgtcaagctgattccccaagaacatctgtatttcttgaagaactgaatgaggctgttagacctctgcaggactatggaatttcagttgccaaggttaattgtgtcaaagaagaaatatcaagatactgtggaaaagaaaaggatttgatgaaagcatatttattcaagggcaacatattgctcagagaattccctactgacaccttgtttgatgtgaatgccattgtcgcccatgttctctttgctcttctttttagtgaagtgaaatatattaccaacctggaagaccttcagaacatagaaaatgctctgaaaggaaaagcaaatattatattctcatatgtaagagccattggaataccagagcacagagcagtcatggaagccggttttgtgtatgggactacataccaatttgtcttaaccacagaaattgcccttttggaaagtattggctctgaggatgtggaatatgcacatctctacttttttcattgtaaactagtcttggacttgacccagcaatgtagaagaacactaatggaacagccattgactacactgaacattcacctgtttattaagacaatgaaagcacctctgttgactgaagttgctgaagatcctcaacaagtttcaactgtccatctccaactgggcttaccactggtttttattgttagccaacaggctacttatgaagctgatagaagaactgcagaatgggttgcttggcgtcttctgggaaaagcaggagttctactcttgttaagggactctttggaagtgaacattcctcaagatgctaatgtggtcttcaaaagagcagaagagggagttccagtggaatttttggtattacatgatgttgatttaataatatctcatgtggaaaataatatgcacattgaggaaatacaagaagatgaagacaatgacatggaaggtccagatatagatgttcaggatgatgaagtggcagaaactgttttcagagataggaagagaaaattacctttggaacttacagtggaactaacagaagaaacatttaatgcaacagtgatggcttctgacagcatagtactcttctatgctggttggcaagcagtatccatggcatttttgcaatcctatattgatgtggcagttaaactgaaaggcacatctactatgcttcttactagaataaactgtgcagattggtctgatgtatgtactaagcaaaatgttactgaatttcctatcataaagatgtacaagaaaggcgagaacccagtatcttatgctggaatgttaggaaccaaagatctcctaaaatttatccagctcaacaggatttcatatccagtgaatataacatcgatccaagaagcagaagaatatttaagtggggaattatataaagacctcatcttgtattctagtgtgtcagtattgggactatttagtccaaccatgaaaacagcaaaagaagattttagtgaagcaggaaactacctaaaaggatatgttatcactggaatttattctgaagaagatgttttgctactgtcaaccaaatatgctgcaagtcttccagccctgctgcttgccagacacacagaaggcaaaatagagagcatcccactagctagcacacatgcacaagacatagttcaaataataacagatgcactactggaaatgtttccggaaatcactgtggaaaatcttcccagttatttcagacttcagaaaccattattgattttgttcagtgatggcactgtaaatcctcaatataaaaaagcaatattgacactggtaaagcagaaatacttggattcatttactccatgctggttaaatctaaagaatactccagtggggagaggaatcttgcgggcatattttgatcctctgcctccccttcctcttcttgttttggtgaatctgcattcaggtggccaagtatttgcatttccttcagaccaggctataattgaagaaaaccttgtattgtggctgaagaaattagaagcaggactagaaaatcatatcacaattttacctgctcaagaatggaaacctcctcttccagcttatgattttctaagtatgatagatgccgcaacatctcaacgtggcactaggaaagttcccaagtgtatgaaagaaacagatgtgcaggagaatgataaggaacaacatgaagataaatcggcagtcagaaaagaaccgattgaaactctgagaataaagcattggaatagaagtaattggtttaaagaagcagaaaaatcatttagacgtgataaagagttaggatgctcaaaagtgaactaattttatagggctgtggtttccaaaatttttttggcatgatagacttaatttatttccttaaagaataatattaaatcatttcaagtttgcagactagtgccatccaatagaattataatataagtcacatattttatttaaaattttctagtaactacattaaacaaagtaaaagtgagcagggcaaaataattttgatattacttttcacccagtagtatacccaaaatagcgaaatatagaaattattaatgagatattttacatccttttttgtaccaagtcttctaaatgcagtacatattttatacttactgcatttcttacttccgagtagccatatttcaagtgttcattgccacatgtggcctgtgactactgtattggacagttcagtactagacaaaaactagcataattaacttagttctagccatgatttctatttggattaaaattaaactctaatcacagttaactccacagtgcattcatgcagctgacagttatatttgttttattggagtcatgatattaaaatcagcgtttgtcaacctcaggggatatttagcaattgtcgggagacatttttgatgtcatgactagggcagttattgacatttagtgagtagaggccatggatcctgctaaataacctgcattggacagcgccccacaacaaagaattatcctgcccgaaatggtagtcgtgccaaggctgagtaaccttgtgttaaaagtaacctgtggcagactaggtttccagaatttcctggttctgctcacgtatcatgtttgaaaaaattttggctattaaagatatgtattagatggtcttatcctgattattacctggatacaacttgatcttttctaatattttcagaaagtgatgggataaccctagaagaggactcagaatgatatttatattttaagtgagtcttaaaacctcctcttatttctacaagttatatggctaaatttcagattgaacagggattcagcattctgccatctcctcatggaaagagaggctccctcatctgaagcgtctctgaaatctacccttgcaagcttcagacaaatcagttgatctccctgagccacacggcctcattctgtgagggagggaaagattagccaaagagttaattttcattccaaatcacttagctgttagactgatctgtttgtagcagttgtttgtctcatttttgctctgtgcattttttgagacatttgttgagaatattctatttggtgctctactgtatttttctttttaatatctacttgatatcttgttctttaaattttcttcacatatggtttgcctgatacaactgatttttataactgaaatttaaggaatctaacagctaaaactcagtaagtgcatctatttccttataacatagacccgttgctactctcagcaccctctcctcaattttttttcctgtagcatgtgatgcctgattaaactcattttcatttgcttttatttctaatatgggaacaatgagagtgaactctaaatataggttgtagtaataaaacatcattagcctaattattagaaaatgctaattaagtaccagcacatagaaacatgaaattgcttagtcattgtacctttgtcagcaattttgacagtcattaatgtttgtcataattttaaataaagtgtctgggtttcagaataccttc

SEQ ID NO: 35 - protein, 858 aa, Homo sapiens
QQIQRTAWKCCLRKQNTAVKKQTKSAQIQLQLIMFSGFNVFRVGISFVIMCIFYMPTVNSLPELSPQKYFSTLQPGKASLAYFCQADSPRTSVFLEELNEAVRPLQDYGISVAKVNCVKEEISRYCGKEKDLMKAYLFKGNILLREFPTDTLFDVNAIVAHVLFALLFSEVKYITNLEDLQNIENALKGKANIIFSYVRAIGIPEHRAVMEAGFVYGTTYQFVLTTEIALLESIGSEDVEYAHLYFFHCKLVLDLTQQCRRTLMEQPLTTLNIHLFIKTMKAPLLTEVAEDPQQVSTVHLQLGLPLVFIVSQQATYEADRRTAEWVAWRLLGKAGVLLLLRDSLEVNIPQDANVVFKRAEEGVPVEFLVLHDVDLIISHVENNMHIEEIQEDEDNDMEGPDIDVQDDEVAETVFRDRKRKLPLELTVELTEETFNATVMASDSIVLFYAGWQAVSMAFLQSYIDVAVKLKGTSTMLLTRINCADWSDVCTKQNVTEFPIIKMYKKGENPVSYAGMLGTKDLLKFIQLNRISYPVNITSIQEAEEYLSGELYKDLILYSSVSVLGLFSPTMKTAKEDFSEAGNYLKGYVITGIYSEEDVLLLSTKYAASLPALLLARHTEGKIESIPLASTHAQDIVQIITDALLEMFPEITVENLPSYFRLQKPLLILFSDGTVNPQYKKAILTLVKQKYLDSFTPCWLNLKNTPVGRGILRAYFDPLPPLPLLVLVNLHSGGQVFAFPSDQAIIEENLVLWLKKLEAGLENHITILPAQEWKPPLPAYDFLSMIDAATSQRGTRKVPKCMKETDVQENDKEQHEDKSAVRKEPIETLRIKHWNRSNWFKEAEKSFRRDKELGCSKVN

SEQ ID NO: 36 - DNA, 309 bp, Homo sapiens
  modified_base (233)..(234): a, t, c, g, other or unknown
gtcaggccattaggttatttatccaaatctctaagcaattaggttgaagttattaagtcaagcctagaaaagctgcctccttgtaaggctttcatgacaatgtatagtaatccacagtgtccaattcttcacactcctcaggaatatcactacctcaggttacggtacacaggctataattgatgatgatgttcagataactgaagacacaataaatgacattcagacatcannanaannncctcatgttcttttctatgatggccacctgtaccagcaacgtgggtttcacccacacaacgatgaact

SEQ ID NO: 37 - DNA, 3894 bp, Homo sapiens
acggttcttatagtgggacgcattgccataggggtctccatctccctctcttccattgccacttgtgtttacatcgcagagattgctcctcaacacagaagaggccttcttgtgtcactgaatgagctgatgattgtcatcggcattctttctgcctatatttcaaattacgcatttgccaatgttttccatggctggaagtacatgtttggtcttgtgattcccttgggagttttgcaagcaattgcaatgtattttcttcctccaagccctcggtttctggtgatgaaaggacaagagggagctgctagcaaggttcttggaaggttaagagcactctcagatacaactgaggaactcactgtgatcaaatcctccctgaaagatgaatatcagtacagtttttgggatctgtttcgttcaaaagacaacatgcggacccgaataatgataggactaacactagtattttttgtacaaatcactggccaaccaaacatattgttctatgcatcaactgttttgaagtcagttggatttcaaagcaatgaggcagctagcctcgcctccactggggttggagtcgtcaaggtcattagcaccatccctgccactcttcttgtagaccatgtcggcagcaaaacattcctctgcattggctcctctgtgatggcagcttcgttggtgaccatgggcatcgtaaatctcaacatccacatgaacttcacccatatctgcagaagccacaattctatcaaccagtccttggatgagtctgtgatttatggaccaggaaacctgtcaaccaacaacaatactctcagagaccacttcaaagggatttcttcccatagcagaagctcactcatgcccctgagaaatgatgtggataagagaggggagacgacctcagcatccttgctaaatgctggattaagccacactgaataccagatagtcacagaccctggggacgtcccagcttttttgaaatggctgtccttagccagcttgcttgtttatgttgctgctttttcaattggtctaggaccaatgccctggctggtgctcagcgagatctttcctggtgggatcagaggacgagccatggctttaacttctagcatgaactggggcatcaatctcctcatctcgctgacatttttgactgtaactgatcttattggcctgccatgggtgtgctttatatatacaatcatgagtctagcatccctgctttttgttgttatgtttatacctgagacaaagggatgctctttggaacaaatatcaatggagctagccaaaggtgaactatgtgaaaaacaacatttgttttatgagtcatcaccaagaagaattagtgccaaaacagcctcaaaaaagaaaaccccaggagcagctcttggagtgtaacaagctgtgtggtaggggccaatccaggcagctttctccagagacctaatggcctcaacaccttctgaacgtggatagtgccagaacacttaggagggtgtctttggaccaatgcatagttgcgactcctgtgctctcttttcagtgtcatggaactggttttgaagagacactctgaaatgataaagacagcctttaatccccctcctccccagaaggaacctcaaaaggtagatgaggtacaaggtcctaagtgatctctttttctgagcaggatatcaggttaaaaaaaaaaagttactggctggtttaatactttctaccttcttcacagagcagcctttgaatagactatgtcctagtgaagacatcaacctccgccttaagctatgtatgtatggaggccagtcgcagctttattatgcagacacacaagtggtctggacatgagggtacagtttctgcctaccaagacactacttgcactggatcttacgcaaaaaagaaccagaacacacagtgtggacaactgcccatatattctatctagattaggagagggtcctggctaggattttagtggtaattcctagttacattcaacaagtataaagattatagagcttattttatgaactataaactataatttaatgcaaaatatccttttatgaatttcatgttaatattgtgaaatattaaaataattccacaatagttgagaaaaatgagcatttttttccatttttaaaaaatgcatagaaaagacaattttaaaatcctgggaccatatttatttagaagtagctgttagtaaaacattagaaaaggagtcaggccattaggttatttatccaaatctctaagcaattaggttgaagttattaagtcaagcctagaaaagctgcctccttgtaaggctttcatgacaatgtatagtaatccacagtgtccaattcttcacactcctcaggaatatcactacctcaggttacggtacacaggctataattgatgatgatgttcagataactgaagacacaataaatgacattcagacatcaggacaattccctcatgttcttttctatgatggccacctgtaccagcaacgtgggtttcacccacacaacgatgaactgttctcttacttctccagttgattttaaagacttgttaagaggtcttactaataaaatttgggtatgatagaaaatccacaatcaaatcttgaaccaaataacatattaaattactaatatttaagtgatggaagacacacaaaaaacttaaaagcacgaacaacctaacttgaaaaagaattttaaaatatgattaacctgaagaaaagagaatcctaagagccaaagctcctttttatttagcttggaattttcctattggttcctaacaaactgtcccaatgtcatataaggaaacatgatctattacattcctttataacaatgtggagagactataaacctatgtaagtagtaaaactatatcagagactcaggagactgactaaaaggcctggatctgcagtgtattatctgtataaaaattggcagggggaagctaaaaggaaaggagattggagatctcaattctatcatggtgtatttcatacgcaaatcagagcatgcattgttttttgtttttggaaagagaagggaagtgtgttctgccccatgtttccttccgtgtttatagttcaaactctatatatacttcaggtattttttgtttagcccttcattataaatgggcaggaaattgtttatcaacctagccagtttattactagtgaccttgacttcagtatcttgagcattcttttatatttttcttttattatcctgagtctgtaactaaacaattttgtcttcaaatttttatccaatatccattgcaccacaccaaatcaagcttcttgattttcaaaaataaaaagggggaaatacttacaacttgtacatatatattcacagtttttatttataaaaaaaatttacagtacttatggagagccagcagaagacatcagagcactcacttcttcccatctttgttaaggttagcgaattacccatggacactgttaggtgaggctcattcggcagccctgaaaacaaacctggtcacactgtctttaccctctcccttcagataaagcacttcgattatctattgatctgcccagttttcaagtcatgcgaatactaaaaaggttacatcatctggatctgtaccttggctatataagcatgttttccccctattctatgtttctttttttggtgaacattgaaaaacaggaggtgacttattactgttaattaaaactaaatgaaaaatgtcaagtctttaaaacagtgagcttgtaactctttcatgtaattttattctctatgaatttggctatcctactgaatcttaaaataaaggaaataaacactttttttttaaaaaaaa

SEQ ID NO: 38 - protein, 471 aa, Homo sapiens
TVLIVGRIAIGVSISLSSIATCVYIAEIAPQHRRGLLVSLNELMIVIGILSAYISNYAFANVFHGWKYMFGLVIPLGVLQAIAMYFLPPSPRFLVMKGQEGAASKVLGRLRALSDTTEELTVIKSSLKDEYQYSFWDLFRSKDNMRTRIMIGLTLVFFVQITGQPNILFYASTVLKSVGFQSNEAASLASTGVGVVKVISTIPATLLVDHVGSKTFLCIGSSVMAASLVTMGIVNLNIHMNFTHICRSHNSINQSLDESVIYGPGNLSTNNNTLRDHFKGISSHSRSSLMPLRNDVDKRGETTSASLLNAGLSHTEYQIVTDPGDVPAFLKWLSLASLLVYVAAFSIGLGPMPWLVLSEIFPGGIRGRAMALTSSMNWGINLLISLTFLTVTDLIGLPWVCFIYTIMSLASLLFVVMFIPETKGCSLEQISMELAKGELCEKQHLFYESSPRRISAKTASKKKTPGAALGV

SEQ ID NO: 39 - DNA, 533 bp, Homo sapiens
  modified_base (51): a, t, c, g, other or unknown
ttcactctttttcatactattataagttattctggtattaaatatgttaantaaaagtgtttttgttttgacatatttcagttaaatgaatgaatgctggttgtattttatttgaatgagtcatgattcatgnttgccatctttttaaaaaaatcagcaaatttcttctatgttataaattatagatgacaaggcaatataggacaactattcacatgattttttttaataccaaaggnttggaagattttataattaacatgtcnnnnnnnctttatagtaagcacatccttggtaatatctccaattgcaatgactttttaatttattttttcttttgctgctttaacattttctggatattaaaatccccccagtcctttaaaagaatcttgaacaatgctgagccggcagctgaaaatctaactcataatttatgttgtagagaaatagaattacctctattctttgttttgccatatgtaatcattttaataaaattaataactgccaggagttcttgacagatttaa

SEQ ID NO: 40 - DNA, 1177 bp, Homo sapiens
ttgaaagaaaacattttgtttctaaattagtctaccattgagtgagaataatcaatatcaagaaagaagactatctttctcaactaaacaataatattccaatcagcttgggaagacctgaaacttgaataagcagtggaaatgccaaatataacagagggtatgtgctacagagaagtaaaaagggtttgactttttatgatgggattttttttttctgggtatgtaatctattttttttttaaactggaaagcatttttgtcagtgtgaatgagggtcaatagtgcagccagtggtgacatttttctttattttgcaaaatgcttttaaaaccaaaggctgctctagttgatggacagtatcagtcttgatctaaattgtaggacactttttcatgtaacataacatttggggattgggtttatttagtgtaatgaagataatttgatataaaaatgcaaaatatataagttatgactgtatgatcagatgaagtatgagttcttttggtttgcatccttaaatagttagagatctctgataaaaactttggaatctttgcaaaacaatacaaaaatgccaaaatgtgagcatgtcaatgaaaactaaagacaaatacttcactctttttcatactattataagttattctggtattaaatatgttaataaaagtgtttttgttttgacatatttcagttaaatgaatgaatgctggttgtattttatttgaatgagtcatgattcatgtttgccatctttttaaaaaaatcagcaaatttcttctatgttataaattatagatgacaaggcaatataggacaactattcacatgattttttttaataccaaaggttggaagattttataattaacatgtcaagaagactttatagtaagcacatccttggtaatatctccaattgcaatgactttttaatttattttttcttttgctgctttaacattttctggatattaaaatccccccagtcctttaaaagaatcttgaacaatgctgagccggcagctgaaaatctaactcataatttatgttgtagagaaatagaattacctctattctttgttttgccatatgtaatcattttaataaaattaataactgccaggagttcttgacagatttaaaataaaagttaatttctagacctcga

SEQ ID NO: 41 - protein, 87 aa, Homo sapiens
MSRRLYSKHILGNISNCNDFLIYFFFCCFNIFWILKSPQSFKRILNNAEPAAENLTHNLCCREIELPLFFVLPYVIILIKLITARSS

SEQ ID NO: 42 - DNA, 420 bp, Homo sapiens
gatattcattggattttctcttactaataggtatatattcactgtgaaaatggagacgatatacataaatgaaaagaagaaaatagtaatctataataccatgcagtgatatatttatcttcctattcttttgtatatgggcatgtttatattattttaaaaagggaatcttagagtatgtattatatgacttttttttgtagcttagcaatataacatggacatgtcgtcagtttggtaaatattgtattgcatcgttacttaaatgcttgtatagggtcttattgtatgagtacattgcaatttgttcaattccctgttcttgaacttttatgagtttcattatcttggaattttatgcagtgttgtgattaatattttaactacatttgcttttaagtctttattttctgatctcag

SEQ ID NO: 43 - DNA, 1627 bp, Homo sapiens
ggtgaaatgctttcggtaggcactccacggctgtgaagatggcggcggctgcgtggcttcaggtgttgcctgtcattcttctgcttctgggagctcacccgtcaccactgtcgtttttcagtgcgggaccggcaaccgtagctgctgccgaccggtccaaatggcacattccgataccgtcggggaaaaattattttagttttggaaagatcctcttcagaaataccactatcttcctgaagtttgatggagaaccttgtgacctgtctttgaatataacctggtatctgaaaagcgctgattgttacaatgaaatctataacttcaaggcagaagaagtagagttgtatttggaaaaacttaaggaaaaaagaggcttgtctgggaaatatcaaacatcatcaaaattgttccagaactgcagtgaactctttaaaacacagaccttttctggagattttatgcatcgactgcctcttttaggagaaaaacaggaggctaaggagaatggaacaaaccttacctttattggagacaaaaccattcagatgcctttcttgaagaaacatttcttggattgttgaaagactttaataatttccaaagttccaaaagttgattttgatagtttttgccagtgttttcgttgcttttatggatgagtagattttcagagtttcttattctgccattctgaaagtgttctcactacctaaaccccagttttatttgtacagaattttaactgaatgtaagttaggcatgacagtctttgttaatttttttaaacaaaagatagccattaggactgggtacagtggctcacgcctgtaatgccaacactttgggaggccaaggtgggcagatgacttgaggttgggagttcgagaccagcttggccaatgtggtgaaactttgtctttactaaaaatacaaaaattagttgctcatggtggcaggcacctgtaatccaagctactcaggaggctgaggcaggagaatcgcgtgaacttgggaggtggaggctgcagtgagctgagatcacgctacttcactccagcctgggcagccagtgagattccatctcaaaaaaaaaagaaaaaagatattcattggattttctcttactaataggtatatattcactgtgaaaatggagacgatatacataaatgaaaagaagaaaatagtaatctataataccatgcagtgatatatttatcttcctattcttttgtatatgggcatgtttatattattttaaaaagggaatcttagagtatgtattatatgacttttttttgtagcttagcaatataacatggacatgtcgtcagtttggtaaatattgtattgcatcgttacttaaatgcttgtatagggtcttattgtatgagtacattgcaatttgttcaattccctgttcttgaacttttatgagtttcattatcttggaattttatgcagtgttgtgattaatattttaactacatttgcttttaagtctttattttctgatctcagaagaattgtatattgggataagtttttaattctataacttaaaagtaaaaatcctttgtaattttatgttcgaaaaaaaaaaaaaaaaaaaaaaaaaaaaaaa

SEQ ID NO: 44 - protein, 132 aa, Homo sapiens
KGFRIVTCQSDWRELWVDDAIWRLLFSMILFVIMVLWRPSANNQRFAFSPLSEEEEEDEQKVPMLKESFEGMKMRSTKQEPNGNSKVNKAQEDDLKWVEENVPSSVTDVALPALLDSDEERMITHFERSKME

SEQ ID NO: 45 - DNA, 536 bp, Homo sapiens
  modified_base (325)..(330): a, t, c, g, other or unknown
gtttcccatgagcagagatgattgagacctgggtccatctgattacatattgctgttgattttgtgagcataatcgttggctggtttatgcactgaacctccttgctctgggatcataatcatatttgagtataagttatggtattcacatttgtatttgctacccaatacatttatttgttatatctgacaagcactgggaaatgaaaataattatttgcattacaaactcattattcatgtactttgaaagctttatctaacagcagtttttatatgggctatctgaatcttatcttctaaataaaaactagatttgtgaaannnnnntattctttttgtacnagcggcntnnctattttaattgtagcnagtgnagacnaccagcatcactatctcnanccnagtgcctacttnngnnnacttgtcctggctgccngtgctgatgctccttactaataaaagctgttgagacagggctgaatacatccttacagccctggtcagtggcattccctcgtacaattcatttctta

SEQ ID NO: 46 - DNA, 1096 bp, Homo sapiens
gcgggggccggcaggtgctccgcagccgtctgtgccacccagagccggcgggccgctaggtccccggagaccctgctatggtgcgtgcgggcgccgtgggggctcatctccccgcgtccggcttggatatcttcggggacctgaagaagatgaacaagcgccagctctattaccaggttttaaacttcgccatgatcgtgtcttctgcactcatgatatggaaaggcttgatcgtgctcacaggcagtgagagccccatcgtggtggtgctgagtggcagtatggagccggcctttcacagaggagacctcctgttcctcacaaatttccgggaagacccaatcagagctggtgaaatagttgtttttaaagttgaaggacgagacattccaatagttcacagagtaatcaaagttcatgaaaaagataatggagacatcaaatttctgactaaaggagataataatgaagttgatgatagaggcttgtacaaagaaggccagaactggctggaaaagaaggacgtggtgggaagagcaagagggtgaggattcacctttaagttatatagaaggttatgaaaaacacttagaaatgaagaaattaaatcaataggctaatgagtcgttaattacaaatatgacatatcaggagagttttaagcagttctagtttatcctgtgaagactaaatacaacttagaaattcctaaagacctaaaatctaaaactgaacccaattatattatctatatgatgggttcaaatctgtttcaaaataaatccagccaggcgcagtggctcacacctgtaatcccagcacctttgggaggctgaggcaggaggatcacttgagcccaggagttccagaccagcctgagtaacatagggataccccatctctattaataaaaattttaaaaaatttgttctaaaaaaagaagaaatataaatcctcactgagagattagttatttgtggattttaaataaccattacaagaaagtctcccagagataaccactgtttaacatttcagggaatgctgtaggtactctctgggctggtacagatgtgtgttatgcctatatttattt

SEQ ID NO: 47 - protein, 156 aa, Homo sapiens
MVRAGAVGAHLPASGLDIFGDLKKMNKRQLYYQVLNFAMIVSSALMIWKGLIVLTGSESPIVVVLSGSMEPAFHRGDLLFLTNFREDPIRAGEIVVFKVEGRDIPIVHRVIKVHEKDNGDIKFLTKGDNNEVDDRGLYKEGQNWLEKKDVVGRARG

SEQ ID NO: 48 - DNA, 363 bp, Homo sapiens
tggtgggaatctttcatcggttttccacattgttgtaacagtgatggtcatcactgtagccacgcttgtgtcattgctgattgattgcctcgggatagttctagaactcaatggtgtgctctgtgcaactcccctcatttttatcattccatcagcctgttatctgaaactgtctgaagaaccaaggacacactccgataagattatgtcttgtgtcatgcttcccattggtgctgtggtgatggtttttggattcgtcatggctattacaaatactcaagactgcacccatgggcaggaaatgttctactgctttcctgacaatttctctctcacaaatacctcagagtctcatgttcagca

SEQ ID NO: 49 - DNA, 1297 bp, Homo sapiens
gctgaagaatttagggagttgattctgatgtaagaagacaatggataaagtatttttcagaagtcagtacaaattggcagcaaatctaccaaaaacaaataataagagaaaaactatcagtgatggatttatcttcacatgtagcatgtactggtttaaatcagtgaataactacatagttattgaattcaaaaacttttatttagacctggtcatctattctcttaattaaatgaaatgaagtttatggagattcacttataagtcatgtgttgcttaatgacagggaaacattctgagaaatgcattgttaggtgatttcctcattgtgcaaacatcacagagtatacgtacacaaatctagatggtagcacctattacacacctaggctatatgctatagcttattgctcctaggctataaacctctacagcatgtttctgtactgaattctgtaggcaactgtagcagaatggaaagtatttatgtatctaaacatagaaaaatatatagtaaaaatacagcattgtaatcatatatgtgggccattaggtgatgcataactgtaatatctaatatttaatttattagatagttatctcaaacatttagtatctagtaaataaacttattttatattactatctaggggacttatttgaaaattactgcagaaatgatgacctggtaacatttggaagattttgttatggtgtcactgtcattttgacataccctatggaatgctttgtgacaagagaggtaattgccaatgtgttttttggtgggaatctttcatcggttttccacattgttgtaacagtgatggtcatcactgtagccacgcttgtgtcattgctgattgattgcctcgggatagttctagaactcaatggtgtgctctgtgcaactcccctcatttttatcattccatcagcctgttatctgaaactgtctgaagaaccaaggacacactccgataagattatgtcttgtgtcatgcttcccattggtgctgtggtgatggtttttggattcgtcatggctattacaaatactcaagactgcacccatgggcaggaaatgttctactgctttcctgacaatttctctctcacaaatacctcagagtctcatgttcagcagacaacacaactttctactttaaatattagtatctttcaatgagttgactgctttaaaaatatgtatgttttcatagactttaaaacacataacatttacgcttgctttagtctgtatttatgttatataaaattattattttggctttta

SEQ ID NO: 50 - protein, 149 aa, Homo sapiens [sequence omitted]

SEQ ID NO: 51 - DNA, 549 bp, Homo sapiens
tgttgggaattggtactggctagaaatttctgttgagtatttattaccccatggtaataatggtaaaccacagtttagaaagattttttttgacagccacagcatgttccgaagagatgattggaagatggaagtggagggttaaataatgaaatgcagctaacatttcggaaagtttctaaaagttgtacaacatgccctacagctactctttaaatctccaaatcaaatgagtttcaggtggagcctctgggaggtgatgaggtcatgagagtggagcctcatgaatgggatgagcactcctacaaaaaggattccagagagctcgcttgctccttccacagtgtgaggacacagagggaaggctctgtctatgaatgagaaagtgggtccccaccagacattgaatctgccgcatcttgatactggacttccagtctccagaactgtgggcaataaatgtctgttgtttattacctgtccagtatctttggtattttgctatagcaacccaaatggactaagaaaacaccagaggccatacctaat

SEQ ID NO: 52 - DNA, 1505 bp, Homo sapiens
caaaagcaacccttcttgctccaggcatgtgcaggaggttttttggtttcagcattttgttgcatgctgactatgtcctttaccttctcttaaattatgtatcaattcatgctggtttattcacttcctgatgtctatatgaagaggctgtctgccaacatctttcatcactctgcctgcaactatgaaaaatttagttctaaaaaatgcaaccttgctaaattgagtactaataggattggttcaattatgttctatgtctgttccatattgacattgtgtgcatctttgccatgcaggctttttaggaattatcgcatctctaacttcccacgagtgtttatgaaaatgtttagatttaaagaactttattgctttagacagaataaggcatgcagttctaacagaaagatccatgaattccagaaatatcactgaaaattattgacatttaagattattttctgtttgttactatggttcacaattcaagaataactctggccaggtgcagtagctcacaccctgtaatcccagcactttgggaggctgaggtaggcagatcacttgagctcaagagttcaagaccagcctgggaaacatggcaaactcccaccattacaaaaaaatacaaaaattagttggtcatggtggtgttcacctatagtcccagtgacttgggaggctgggatgggaggatctcttgagcccaggagatgcaggcttgcagtgagccatgatcatgccactgtactgcagactgagtgaaacagcaagatcttgtctgaaaagaaaaaaaaagtaaaagaaaaagaaaagaaaataactcccattgctaaagacatatatgcttatcaggttaagataaagtgaattttgttcttcccaatgacatttcaggatatttgttcacaggaaagaacatgttgggaattggtactggctagaaatttctgttgagtatttattaccccatggtaataatggtaaaccacagtttagaaagattttttttgacagccacagcatgttccgaagagatgattggaagatggaagtggagggttaaataatgaaatgcagctaacatttcggaaagtttctaaaagttgtacaacatgccctacagctactctttaaatctccaaatcaaatgagtttcaggtggagcctctgggaggtgatgaggtcatgagagtggagcctcatgaatgggatgagcactcctacaaaaaggattccagagagctcgcttgctccttccacagtgtgaggacacagagggaaggctctgtctatgaatgagaaagtgggtccccaccagacattgaatctgccgcatcttgatactggacttccagtctccagaactgtgggcaataaatgtctgttgtttattacctgtccagtatctttggtattttgctatagcaacccaaatggactaagaaaacaccagaggccatacctaataaaaatattgacatcacaaaaaaaaaaaaaaa

SEQ ID NO: 53 - protein, 113 aa, Homo sapiens
MYQFMLVYSLPDVYMKRLSANIFHHSACNYEKFSSKKCNLAKLSTNRIGSIMFYVCSILTLCASLPCRLFRNYRISNFPRVFMKMFRFKELYCFRQNKACSSNRKIHEFQKYH

SEQ ID NO: 54 - DNA, 528 bp, Homo sapiens
  modified_base (159): a, t, c, g, other or unknown
taaagagcgcccgaagcactagcagagtcaaccccccggggacccataagacagggcttctagtataaggattggagtttgacccacccccaaaaaatgccctggggatattggttttctcaggtggcatatgactctccggcttggattgcctcgctncgganaggggacaaaaggttttgccctgagcatctggtgntgtcttccagtgcctggttaggttgctccgnggctggacagtctgactactctcaaaactcctcgtgacaggcctttctggggtctgatcgccctttgtttccttacacttgggcctgttatcagaagaactctgaatccggaaataccttgtttaaatttgggctacagttttcaagatccaggcatttgggtgaatcacttaacccgagtattaggatctggaaaatggggctagtaattgttgtaaatgtgaggtgtttaaaagtgtctggcattttagtgcgtagataaatgctacttcctgtgcccattctcttgggagttctc

SEQ ID NO: 55 - DNA, 414 bp, Homo sapiens
  modified_base (44): a, t, c, g, other or unknown
tagaatgccctaggtgaatccctccagtcttccagtaccatccntgactcctctctctgatgacacatgaactttatgcttttgcacacttcaggcaacncnaaaagaaaggaaaagaacagcttagcttcttaatgtgtgtaagaaaccacagtgaaaaaaaatcaggtgtgttgttgaggctgctaaaagctttccttttttttctgtgccagttctcgctgcctcattggttgagatgggatgtcttttttgatgtcctctttagagagtgttatcctcacctttttgcatagtcctaccaaaagacacctcacatgcaaagtgtaacagaaaattacagtcatgactttagttttaaaaacaggacgtatattcatgaagaatgtttgctgttttcccagtgggttaatc

SEQ ID NO: 56 - DNA, 465 bp, Homo sapiens
  modified_base (100): a, t, c, g, other or unknown
tattcaatatgcttttcccgcttttctaagaggaataaacttagacaaattacattataaacagttcccctactactatctcccactctagataaagccngtgggtggtannngnncttttattccttatagtattatgccaaagaatcaacttattttcattgaagattataaataaatgaagcttgttatagccataatgatttgagtcagtataccattttacctataaaatgcaaaattcatccttgcaaccccattcaccaggagccttgaagcattttgtttactccaaaggccttgtcaaggaagcataattttttgttttgccttcttatttagtcagtttggtcatatttacttaaaaaaacaaactgaaaatcacactcctttatatgttgatataactgattttatagaatctgtctgttctttgtttaacaggtctctgtaagcaagcttgca

SEQ ID NO: 57 - DNA, 466 bp, Homo sapiens
  modified_base (78): a, t, c, g, other or unknown
gttgtttgtgcacatatctacatggtggagaccatattcattatttcatcttccaaataatgggaaaaatataaaagngantcagtgtgctttgggaattcagtgaaatcatgttaactcatatagagggggccttagtttatctctnctttactgaattaattagttttggaaattcttttaccattaaaaaaaattaaggaccatacagagaatgatttaagaaaaaacaagtcacttaaaaatcatcacctatttataaactgtattaattacacataatgcttattgattcaatgaggtttctctaaagacttctgcttaataaatatgctgacttcatttaaattagtttagactattgtaggaatggaaggaaatgattatatttactagaattagtgagatcagaaagcatatcagaatgttgatgatatcaaggagacaatctacagagtttttgcct

SEQ ID NO: 58 - DNA, 379 bp, Homo sapiens
  modified_base (99): a, t, c, g, other or unknown
gaaaccattgaaaccctattcattcttaaagactaagtaattttttagtgttctactgtatgccaagcactgttgtactcttgtgggccctggaattanatcagaaaaaaacaggcagaatttgcctcctcatggattctgatcncnnctactggncctcagtgacagttgaatatgtacatcagatagttgtttnccccantctcctanctacattataactttcacaagggttggaaatcttaagtccgttttctatctccttagtgcttggtacctagttctgccccaaaaaacttaattccctaggacactaaccatgtcgaataaagtcactcttgggaggtctacancagcaccgcccagtagcagtataata

SEQ ID NO: 59 - DNA, 276 bp, Homo sapiens
cattaataatttgcctttttacatctcttaggagtgaatcattatttgaaaagttttcactttttcttctttgttgctgttttatgcacatacatgtgtgtgcagttcaccaaagacaaatttcttcagcaaaattaatgtttccatattgtataaaactcataactatggattacaaatcatgttaccattaattgctttctatattgttgtatttagatttaaccagtgtttatccacctgttaagacctgtaatccagtcagggtggctcatg

SEQ ID NO: 60 - DNA, 514 bp, Homo sapiens
  modified_base (26): a, t, c, g, other or unknown
tttactaaacgatgattactccttcnatattcatattcctaaacacatacagtttcttantgtaattaagtttttannnaaaaaaanngggaaatgcattattgaggcgataggattactgggtggctataaacacatctgctgcacagctgacatttatcttctacaatgagcantgacaattttattttttaataatcagtatggactaatcctgatgattttttttnaacattttcaaatagggctgcatatggcttaaaattaatatatacatgtgtacctatataatattcttatttattaatggacttcctacatagctcatattgacgttagatttaaatgaaattccagaagggttttctataggtaagtcatacattggatttccatattacctatgattattgaagtatttatttctgtttttaagacttcagagcaattttgctggtcatttgttttctgtgtttttattttgaaatngttctttgaggcattgtcctattac

SEQ ID NO: 61 - DNA, 514 bp, Homo sapiens
ttatcattcagcttgctttgtgttgttttgaggggttggggtacagtgggacagttttattttgtttggcatttatagaaaattgagaagtttcctttgatcaagccatatttttgatttaaaacaatgattagcagtttagaaaactatctctgctattttattctgcttttaaattctttgttttttatatttctgtcccttagactttaacattttaaagtgtgtaaaaataaaacactgtcagtgctaatcatagaaaatcagactatggcttgaaatgactagaaaaacatttcaaattaggctgctttatgatttgcatattatgattccggccattggagtttttggatttctaagtgttcataataccatgaaaagtaaatattttaaacaattgtatccccgtttaaaaactttctaatgttaaaactgtatttttttcatgtattagcccatgtgtgataatcttagttttccaattatggagggcatgaggagtagctttatt

SEQ ID NO: 62 - DNA, 521 bp, Homo sapiens
tagcacccccaaaagacaacttctttcagaaacggggtgttttacctaaacatagtagcttacatgttagccagcagtaggtcggcactagtgttttccacggttatcacctttgacaggtgatgtgcatctatagatagtggaagccaccccatgaggaggtgttaatagcagcatggtttcacttttggtaatcaggtaatcatgtgtatatacttagattcgcattattttaacatttctctgctactctgcacttcaggttcgttaagctattttaataattactggggttatggcaaacaccaatggaaatgtatatggcaactgctttcctgagcaagtgtgatttgttttatggctgttcaagttataaaattgttcttacattgtaggtaaacaaaatcttgatgtttttaaaggtcactgtaacttaaggttcaaatttctggcacagttttattagtattcacttcggaagctaataagataccatggttttctatgttactcccattgta

SEQ ID NO: 63 - DNA, 3360 bp, Homo sapiens
  modified_base (2855)..(2860): a, t, c, g, other or unknown
catgaggagctgagcgtctcgggcgaggcgggctgacggcagcaccatgcaggcggcagtggctgtgtccgtgcccttcttgctgctctgtgtcctggggacctgccctccggcgcgctgcggccaggcaggagacgcctcattgatggagctagagaagaggaaggaaaaccgcttcgtggagcgccagagcatcgtgccactgcgcctcatctaccgctcgggcggcgaagacgaaagtcggcacgacgcgctcgacacgcgggtgcggggcgacctcggtggcccgcagttgactcatgttgaccaagcaagcttccaggttgatgcctttggaacgtcattcattctcgatgtcgtgctaaatcatgatttgctgtcctctgaatacatagagagacacattgaacatggaggcaagactgtggaagttaaaggaggagagcactgttactaccagggccatatccgaggaaaccctgactcatttgttgcattgtcaacatgccacggacttcatgggatgttctatgacgggaaccacacatatctcattgagccagaagaaaatgacactactcaagaggatttccattttcattcagtttacaaatccagactgtttgaattttccttggatgatcttccatctgaatttcagcaagtaaacattactccatcaaaatttattttgaagccaagaccaaaaaggagtaaacggcagcttcgtcgatatcctcgtaatgtagaagaagaaaccaaatacattgaactgatgattgtgaatgatcaccttatgtttaaaaaacatcggctttccgttgtacataccaatacctatgcgaaatctgtggtgaacatggcagatttaatatataaagaccaacttaagaccaggatagtattggttgctatggaaacctgggcgactgacaacaagtttgccatatctgaaaatccattgatcaccctacgtgagtttatgaaatacaggagggattttatcaaagagaaaagtgatgcagttcaccttttttcgggaagtcaatttgagagtagccggagcggggcagcttatattggtgggatttgctcgttgctgaaaggaggaggcgtgaatgaatttgggaaaactgatttaatggctgttacacttgcccagtcattagcccataatattggtattatctcagacaaaagaaagttagcaagtggtgaatgtaaatgcgaggacacgtggtccgggtgcataatgggagacactggctattatcttcctaaaaagttcacccagtgtaatattgaagagtatcatgacttcctgaatagtggaggtggtgcctgccttttcaacaaaccttctaagcttcttgatcctcctgagtgtggcaatggcttcattgaaactggagaggagtgtgattgtggaaccccggccgaatgtgtccttgaaggagcagagtgttgtaagaaatgcaccttgactcaagactctcaatgcagtgacggtctttgctgtaaaaagtgcaagtttcagcctatgggcactgtgtgccgagaagcagtaaatgattgtgatattcgtgaaacgtgctcaggaaattcaagccagtgtgcccctaatattcataaaatggatggatattcatgtgatggtgttcagggaatttgctttggaggaagatgcaaaaccagagatagacaatgcaaatacatttgggggcaaaaggtgacagcatcagacaaatattgctatgagaaactgaatattgaagggacggagaagggtaactgtgggaaagacaaagacacatggatacagtgcaacaaacgggatgtgctttgtggttaccttttgtgtaccaatattggcaatatcccaaggcttggagaactcgatggtgaaatcacatctactttagttgtgcagcaaggaagaacattaaactgcagtggtgggcatgttaagcttgaagaagatgtagatcttggctatgtggaagatgggacaccttgtggtccccaaatgatgtgcttagaacacaggtgtcttcctgtggcttctttcaactttagtacttgcttgagcagtaaagaaggcactatttgctcaggaaatggagtttgcagtaatgagctgaagtgtgtgtgtaacagacactggataggttctgattgcaacacttacttccctcacaatgatgatgcaaagactggtatcactctgtctggcaatggtgttgctggcaccaatatcataataggcataattgctggcaccattttagtgctggccctcatattaggaataactgcgtggggttataaaaactatcgagaacagaggtcaaatgggctctctcattcttggagtgaaaggattccagacacaaaacatatttcagacatctgtgaaaatgggcgacctcgaagtaactcttggcaaggtaacctgggaggcaacaaaaagaaaatcagaggcaaaagatttagacctcggtctaattcaactgagtatttaaacccatggttcaaaagagactataatgtagctaagtgggtagaagatgtgaataaaaacactgaagaaccatactttaggactttatctcctgccaagtctccttcttcatcaactgggtctattgcctccagcagaaaatacccttacccaatgcctccacttcctgatgaggacaagaaagtgaaccgacaaagtgccaggctatgggagacatccatttaagatcaactgtttacatgtgatacatcgaaaactgtttacttcaacttttacttcagacaatacgaagaccctctgagatgctacagaggagaggaagcggagtttcacnnnnnntnaccattttctttttgtcattggcttaggatttaactaaccatgaaaagaactactgaaatattacactataacatggaacaataaaggtactggtatgttaatggataatccgcatgacagataatatgtagaaatattcataaagttaactcacatgacccaaatgtagcaagtttcctaaggtacaatagtggattcagaacttgacgttctgaggcacatcctcactgtaaacagtaatgctatatgcatgaagcttctgtttattgttttccatatttaaggaaacaacatcccataatagaaatgagcatgcagggctaaggcatataggatttttctgcaggactttaaagctttgaaaggccaatatcccataggctaactttaaacatgtatttttatttttgttttgttttttacttttcatatttatattagcatacaaggacaattgtatatatgtaacatttttaaaattttaaaaaaaaaaaaaa

SEQ ID NO: 64 - protein, 899 aa, Homo sapiens [sequence omitted]

SEQ ID NO: 65 - DNA, 495 bp, Homo sapiens
  modified_base (62): a, t, c, g, other or unknown
ttttgcaatgtgacccatgttgggcatttttatataatcaacaactaaatcttttgccaaangcannnnnnnnnnnnatnnnctaananangnnaataacgagcaaaactggttagatttngcatgaaatggttctgaaaggtaagaggaaaacagactttggaggnngtttagttttgaatttctgacagagataaagtagtttaaaatctctcgtacactgataactcaagcttttcattttctcatacagttgtacagatttaactgggaccatcagttttaaactgttgtcaagctaactaataatcatctgctttaagacgcaagattctgaattaaactttatataggtatagatacatctgttgtttctttgtatttcaggaaaggtgatagtagttttatttgatactgataaatattgaattgattttttagttattttttatcattttttcaatggagtagtataggactgtgctttgtcctttt

SEQ ID NO: 66 - DNA, 3360 bp, Homo sapiens
gaattccggctgtgccgcaccgaggcgagcaggagcagggaacaggtgtttaaaattatccaactgccatagagctaaattcttttttggaaaattgaaccgaacttctactgaatacaagatgaaaatgtggttgctggtcagtcatcttgtgataatatctattactacctgtttagcagagtttacatggtatagaagatatggtcatggagtttctgaggaagacaaaggatttggaccaatttttgaagagcagccaatcaataccatttatccagaggaatcactggaaggaaaagtctcactcaactgtagggcacgagccagccctttcccggtttacaaatggagaatgaataatggggacgttgatctcacaagtgatcgatacagtatggtaggaggaaaccttgttatcaacaaccctgacaaacagaaagatgctggaatatactactgtttagcatctaataactacgggatggtcagaagcactgaagcaaccctgagctttggatatcttgatcctttcccacctgaggaacgtcctgaggtcagagtaaaagaagggaaaggaatggtgcttctctgtgaccccccataccattttccagatgatcttagctatcgctggcttctaaatgaatttcctgtatttatcacaatggataaacggcgatttgtgtctcagacaaatggcaatctctacattgcaaatgttgaggcttccgacaaaggcaattattcctgctttgtttccagtccttctattacaaagagcgtgttcagcaaattcatcccactcattccaatacctgaacgaacaacaaaaccatatcctgctgatattgtagttcagttcaaggatgtatatgcattgatgggccaaaatgtgaccttagaatgttttgcacttggaaatcctgttccggatatccgatggcggaaggttctagaaccaatgccaagcactgctgagattagcacctctggggctgttcttaagatcttcaatattcagctagaagatgaaggcatctatgaatgtgaggctgagaacattagaggaaaggataaacatcaagcaagaatttatgttcaagcattccctgagtgggtagaacacatcaatgacacagaggtggacataggcagtgatctctactggccttgtgtggccacaggaaagcccatccctacaatccgatggttgaaaaatggatatgcgtatcataaaggggaattaagactgtatgatgtgacttttgaaaatgccggaatgtatcagtgcatagctgaaaacacatatggagccatttatgcaaatgctgagttgaagatcttggcgttggctccaacttttgaaatgaatcctatgaagaaaaagatcctggctgctaaaggtggaagggtgataattgaatgcaaacctaaagctgcaccgaaaccaaagttttcatggagtaaagggacagagtggcttgtcaatagcagcagaatactcatttgggaagatggtagcttggaaatcaacaacattacaaggaatgatggaggtatctatacatgctttgcagaaaataacagagggaaagctaatagcactggaacccttgttatcacagatcctacgcgaattatattggccccaattaatgccgatatcacagttggagaaaacgccaccatgcagtgtgctgcgtcctttgatcctgccttggatctcacatttgtttggtccttcaatggctatgtgatcgattttaacaaagagaatattcactaccagaggaattttatgctggattccaatggggaattactaatccgaaatgcgcagctgaaacatgctggaagatacacatgcactgcccagacaattgtggacaattcttcagcttcagctgaccttgtagtgagaggccctccaggccctccaggtggtctgagaatagaagacattagagccacttctgtggcacttacttggagccgtggttcagacaatcatagtcctatttctaaatacactatccagaccaagactattctttcagatgactggaaagatgcaaagacagatcccccaattattgaaggaaatatggaggcagcaagagcagtggacttaatcccatggatggagtatgaattccgcgtggtagcaaccaatacactgggtagaggagagcccagtataccatctaacagaattaaaacagacggtgctgcaccaaatgtggctccttcagatgtaggaggtggaggtggaagaaacagagagctgaccataacatgggcgcctttgtcaagagaataccactatggcaacaattttggttacatagtggcatttaagccatttgatggagaagaatggaaaaaagtcacagttactaatcctgatactggccgatatgtccataaagatgaaaccatgagcccttccactgcatttcaagttaaagtcaaggccttcaacaacaaaggagatggaccttacagcctactagcagtcattaattcagcacaagacgctcccagtgaagccccaacagaagtaggtgtaaaagtcttatcatcttctgagatatctgttcattgggaacatgttttagaaaaaatagtggaaagctatcagattcggtattgggctgcccatgacaaagaagaagctgcaaacagagttcaagtcaccagccaagagtactcggccaggctcgagaaccttctgccagacacccagtattttatagaagtcggggcctgcaatagtgcagggtgtggacctccaagtgacatgattgaggctttcaccaagaaagcacctcctagccagcctccaaggatcatcagttcagtaaggtctggttcacgctatataatcacctgggatcatgtcgttgcactatcaaatgaatctacagtgacgggatataaggtactctacagacctgatggccagcatgatggcaagctgtattcaactcacaaacactccatagaagtcccaatccccagagatggagaatacgttgtggaggttcgcgcgcacagtgatggaggagatggagtggtgtctcaagtcaaaatttcaggtgcacccaccctatccccaagtcttctcggcttactgctgcctgcctttggcatccttgtctacttggaattctgaatgtgttgtgacagctgctgttcccatcccagctcagaagacacccttcaaccctgggatgaccacaattccttccaatttctgcggctccatcctaagccaaataaattatactttaacaaactattcaactgatttacaacacacatgatgactgaggcattcaggaaccccttcatcca

SEQ ID NO: 67 - protein, 1018 aa, Homo sapiens [sequence omitted]

SEQ ID NO: 68 - DNA, 522 bp, Homo sapiens
  modified_base (58): a, t, c, g, other or unknown
aagcagaagctgtgacaagtttagtagtcccaaaatgggttatatcccttcccccttnacatcagaatcttgtgaaatgggaaaacaacagaaggaggggatcaaagatagctgatctcacatgcttcccaggcagggcagaggtgggagtcaaacccgggtgacaggtgggtggagagccctgtttgaggttgtggctgatccctctctggtattagtttttcccctgggagcaggaagccctaggaagaggggactgcagggtccccaggggatctttcctccctcccctgcatgaggcagaggcaagctgcctgccaaccccctccctcaaggaatggccttgcccaggaatgcccaccacacataccctcttctttttttctagtcaaactcttgtttattccttggcttgcctccctccttcctcccctctcaacctttacttctgatttctatttcatggaatttgggattgaagttaaactacaacagtgccgccaacaccaagtcttgcaggaa

SEQ ID NO: 69 - DNA, 4278 bp, Homo sapiens
tgggggtctcagtgcatctccttctcctctctgcctgcctcctccctcaccgaagggttagcggacacccatccttttctgcttggggaccccaccaccacccgcaacactgccgctgtctcttcttcaccgtatccttctctacccaccctcttctctcttctcttctccctgcccctttaaatctgcctggcccagcctcccccgtgatgctgggatggagcaaacattgatttgtgctgggatggaatcggaattttgatttatttttcctctcccaaccataagaagaaaaaaataataaaaacaccccctcttgagagccccctccccctttgcatccagctcccagctcttcttccctatctccatccaaggcagattttttcccctacactattctcatcttcccccacccttgccactacctcgcccccccacccagcctgctcctccagctggggagagaggggactctccggactcccccacctttcctctctgggttggagcagtctctccggaaggggagggggcttggcttgtccgggcgaggtgggagtggaggtatcctgccatggatgctgtgccggggaggcagcctgagccccagcccacatgccactcaggatgagggtccggccctgcctgccctcgctggggcccccccgcccggccccggtctaactgcccccgccccgaggcctcgcccggctccaaggcccccagcaggctctccagtcccaggatgcgctgagccgccggggggctgaggccgcgccaactacatgcatgtcccccgggggcaagttcgactttgacgacgggggctgctacgtggggggctgggaggcggggcgggcacatggctacggcgtgtgcacgggccccggcgcccagggcgagtacagcggctgctgggcacacggcttcgagtcactgggcgtcttcacggggcccggcggacacagctaccagggccactggcagcagggcaagcgcgaagggctgggcgtggagcgcaagagccgctggacgtaccgcggcgagtggctgggcgggctgaaggggcgcagcggcgtgtgggaaagcgtgtccggcctgcgctacgccgggctctggaaggacggtttccaggacggctacggcactgagacctactccgacggaggcacctaccagggccagtggcaggccgggaagcgccacggctacggggtacgccagagtgtgccctaccatcaggcggcgctgctgcgctcgccccgccgcacctccctggattccggccacagcgaccccccgacgccacccccgcccctgcccttgccgggcgacgagggaggcagccccgcctcgggctcccggggcggcttcgtgctggccgggcccggggacgccgacggcgcgtcgtcccgaaagcgcactccggcggccggcggattctttcgccgttcgctgctgctcagcgggctccgagcgggcggacgtcgcagctccctgggcagcaagcgaggctccctgcgcagcgaggtgagcagcgaggtgggcagcaccggaccgcccggctcggaggccagcgggcccccggccgcagcgccgcccgccctcatcgagggctcggccacagaggtgtacgcgggcgagtggcgcgcagatcggcgcagcggcttcggcgtcagccagcgctccaacgggctgcgctacgagggcgagtggctgggcaaccggcggcacggctacgggcgcaccacccgccccgacggctcccgcgaggagggcaagtacaagcgcaaccggctggtgcacggcgggcgcgtccgcagtctcctgcctctggcccttcggcggggcaaggttaaggagaaggtggacagggctgtcgagggcgcccgtcgagccgtgagtgctgcccgtcagcgccaggagatcgccgctgccagggcagcagacgccctcctaaaggcagtggcagccagcagtgtcgctgagaaggccgtggaggcagctcgaatggccaaactgatagcccaggacctgcagcccatgctagaggccccaggccgcagacccaggcaggactcagaaggttccgacacggagcccctggatgaggacagccctggggtatatgagaacggactgaccccctcagagggatcccctgaactgcccagcagtcctgcctcctcccgccaaccctggcgaccccctgcctgccggagcccactgcctcctggaggggaccagggtcccttctccagccccaaagcttggcctgaggagtgggggggggcaggcgcacaggcagaggaactagctggctatgaggctgaggatgaggctgggatgcaagggccagggcccagagacggttccccactcctcggaggctgcagcgacagttcaggaagtcttcgagaggaggagggggaggatgaagagcccctgcccccgctgagggccccagcaggcacggagcctgagcccatcgccatgctggtcctgaggggctcgtcctcgaggggtcctgatgctgggtgcctgacagaagagctcggggagcccgctgcaaccgagaggcctgcccagccgggagctgccaaccccctggtggtgggagccgtggccctcctggacctcagcctggcattcctgttctcccagctcctcacctgaggctacttcctggcctggttctggctttggttgcgtgcctcttcacccctttgacctgccttttttctcttctcctcttcctggctgtgttttctcctatctttctttctcttcttcctttcttttctgtgctcctttgtttttttctctcgctttttctttccctgtcttctttcagattatctcatttcttctggatctgtctctgtattcctcactcccttccccatcccaaccccttctttctctagattgtttacatatgaagggcttttctctctcagagttgctgtcttctctgagacacacaaatctaagtcagaccattgctccacgccctcccaccttttctttagacctcaacttcgctgcgggtgggggtttggtgtcctaaggagactcctggaagctgaatggagaggaggaagaaaatgaagaaggagtgattgaatgtcgggcaaggcactggctgagctgctgtggctccctagcctaaggggcctgctgtccctctgaggcctagtgaaaaagctgcaggaggtgcatcctccacctctaatcttggaggctattatcttacctccaagcactgagctgggttactgcccaattccatccttccctgaaggagagaagggaagtgaaaagtagagtaactccccagcatttccctctttttctcctcatcggccagcccctcctccagccccctctggtggcatgccatgccaagagcaacgtgtaaaggaacagagaatatccaatgcagtcaagtccaccctgcccagactttgccactgacttctcccacccttctgtctcccccataatagtttatttggttggtctggactcacttgtggcctttgattaaattcctaaggggcctgaagaagacatttctactgcagagggttagaggcacttgagcaaggcccccacatcccaactctgggagttgtggtgggaggaggcacttctgggggataggaccagacaagataacaggagctcacatggaagcagaagctgtgacaagtttagtagtcccaaaatgggttatatcccttccccctttacatcagaatcttgtgaaatgggaaaacaacagaaggaggggatcaaagatagctgatctcacatgcttcccaggcagggcagaggtgggagtcaaacccgggtgacaggtgggtggagagccctgtttgaggttgtggctgatccctctctggtattagtttttcccctgggagcaggaagccctaggaagaggggactgcagggtccccaggggatctttcctccctcccctgcatgaggcagaggcaagctgcctgccaaccccctccctcaaggaatggccttgcccaggaatgcccaccacacataccctcttctttttttctagtcaaactcttgtttattccttggcttgcctccctccttcctcccctctcaacctttacttctgatttctatttcatggaatttgggattgaagttaaactacaacagtgccgccaacaccaagtcttgcaggaaaaaaatacaaagaaatttaacaaaaaaaatatattaataaaaaagttcaaaaaaggg

SEQ ID NO: 70 - protein, 663 aa, Homo sapiens
LPPPRGLARLQGPQQALQSQDALSRRGAEAAPTTCMSPGGKFDFDDGGCYVGGWEAGRAHGYGVCTGPGAQGEYSGCWAHGFESLGVFTGPGGHSYQGHWQQGKREGLGVERKSRWTYRGEWLGGLKGRSGVWESVSGLRYAGLWKDGFQDGYGTETYSDGGTYQGQWQAGKRHGYGVRQSVPYHQAALLRSPRRTSLDSGHSDPPTPPPPLPLPGDEGGSPASGSRGGFVLAGPGDADGASSRKRTPAAGGFFRRSLLLSGLRAGGRRSSLGSKRGSLRSEVSSEVGSTGPPGSEASGPPAAAPPALIEGSATEVYAGEWRADRRSGFGVSQRSNGLRYEGEWLGNRRHGYGRTTRPDGSREEGKYKRNRLVHGGRVRSLLPLALRRGKVKEKVDRAVEGARRAVSAARQRQEIAAARAADALLKAVAASSVAEKAVEAARMAKLIAQDLQPMLEAPGRRPRQDSEGSDTEPLDEDSPGVYENGLTPSEGSPELPSSPASSRQPWRPPACRSPLPPGGDQGPFSSPKAWPEEWGGAGAQAEELAGYEAEDEAGMQGPGPRDGSPLLGGCSDSSGSLREEEGEDEEPLPPLRAPAGTEPEPIAMLVLRGSSSRGPDAGCLTEELGEPAATERPAQPGAANPLVVGAVALLDLSLAFLFSQLLT

SEQ ID NO: 71 - DNA, 529 bp, Homo sapiens
  modified_base (33): a, t, c, g, other or unknown
taaaatccctatgatctctgtctcacctacttnacagggttgctgtgaagatcgcatactacacacaggaatgctcatcagtttttaaattttatttaatttttatttatttttttttaaatgtaattttttcagagagataaggtcttgctatgttacccagcctagtcttgaactcctggcctcaagtgatcctcctgccttggcctcccatgctgctgggattacaggtgtgaactaccatgcccagccagctcctaagtcttaaggctctgtgttagtgatagatgtggccatggtgtaggcagtgcaatgtcttcgagtgagagtgaaggtggtaactcattgcatggattctagagttctgtttattctaatccaagttcttccacttaaaaacaatgttcttcctctcattgagtctcattcctcatctataggatgggaataagagcatgtacctggcaggttgttgtaaggattaaatggtgtaaaaaaatgtcaagtgcttgcaactttgaataccaaa

SEQ ID NO: 72 - DNA, 4446 bp, Homo sapiens
gcgcgttccctcttggccccaaagcgagtccggcgggcggctcctcggggttgggcgaccgagcggggccggccgggcggggggcgggcccgtgaaggcggcgcagcgcggcgcgggaggcgtgctgggcgcggggctgcggtgcccagaggctgcggcattaggggctcggcgcccccgaccttccgcgtcccggggtggcggcggcggcggcggcggcggcgcgggcggcatatgatgctgagctggctgctccagaatgaaccacagctctgagaaggggaagtagaaacagctggcgccctgccatggcctgtgaaccacaggtggacccgggggccactggcccattgcccccctcctcccctggctggagtgccctgcctggagggagccctcctggctgggggcaagagctccacaatggccaggtcctcactgttctccggattgacaatacctgtgcacccatctccttcgacctgggagccgcagaagagcaactgcaaacttggggcatccaggtcccggctgaccagtacaggagcttggctgagagtgccctcttggagccccaagtgagaagatatatcatctacaactcgaggcctatgcggctggcctttgctgtggttttctatgtggtggtgtgggccaatatctactctaccagtcagatgtttgccttggggaaccactgggctggcatgctgctcgtgaccctggccgcggtgagcctgaccttgactcttgtgctggtctttgaaagacaccagaagaaggccaacaccaacacggacctgaggctggcagctgccaatggagccctcctgagacaccgggtgctgctgggggtgacagacacagtggaaggatgccagagtgtgattcagctttggtttgtctacttcgacctggagaactgtgtgcagtttttgtctgatcatgttcaagaaatgaagactagccaagaggtattgctgagaagcagattgagccagttgtgtgttgtcatggagactggggtgagccctgcaacagcggaggggcctgagaacttggaggatgctcctctcctgcccggcaattcttgtcctaacgagaggccactcatgcagactgagcttcatcagcttgttcctgaggctgagccggaggaaatggcccgccagctgctggcagtgtttggcggctactacatccggcttctagtgacctcccagctccctcaggcaatggggacacgacacacgaactctccgagaattccatgcccctgccagctcatagaagcctacatcctaggcacagggtgctgcccgttcctggcgaggtgacctagggatgaaggtactcatcttccttcaagactgagcagtcaggaaggcttcaggagcccaagatggccaatggggagccccaggtgaggagagaagcatctgggggcactccaaaaggggcctgtgatgtcagccactggggtgttgtgctcacttcagggcccagcacaaaaatccttgtttgacatctcatgctgaccccctggcctttgcagaagctgatggttacagagctagtcccaccaaagctactctctctgctgcttagaactgtggacacgtatggaaagactggacccccattgctttcattgttcagagaacccaggagacatgaagatgaccagactgggcaaattatgtgtccaaaacttggcctcagatgatgtttccatctccaaccccttcatgccagatggggaaactgaggctcagagaggatactgctctatgtggcattgccttgaacccctaaaattatcagacttcctttttccaatataaagaaaaaaagtaagttttcagaattctctcaatttttaagtttttctcccccatattttgtgaaaagcagtggtatgtgtacgtgttgtctaccagtacacaggctgcagaagacagagacagaagaaagagatcaagggcagataactgttgataggaatatttgagaaagattgatcctgtttgacttgaggacttattttgttcacaggcatgcacgcttgtggttgtggttttatattacagatgtagaacaatggttatgtttcccgacatgaacattgtcctggaatgaagtgtgatcagccacttgtggaattctttgaagagctcagaggcttccaagtgatctgctcctgaacaagtttgaagacctattgtttcatagacccaagaccaaacgcatctaaaggatccccagcccccaagacctagcctttgtctgcgattttggcttcatctcccacaaaacccctttatgagttcacgctctttcctggactgacatacctattcctttccatttgttggactcctattcatgcttcaaagtccagctttcttaagcccttctttaggaagccttcccacacagccaaccctgctgctctctgcctcctttaaattcttgatacagctgctgcttgttctgatgttttatggtattgattctgttttcctgtgtatatgccagtttttctagctagactgtaaactccttaaggacagagactacaccttgtactttttgtgcatgacctggacctgctaaggaaaaaaaaatcttgtggattgattgctttgccatccccacagcagcttttgcaaattgctttccaaactcacttgaatgatgacattgctgtggacctgggttctggacctgatctgccacttcaagctgtgtaatttttggcaagttgctttctttgcctggtcctcagtttgcccatcaatataatgggtggattggatgatttttttttttttaattgagatggagtcttgcactgtcacccaggctggagtgcagtggcgcgatcttggctcactgcaacccccgccacctaggttcaagtgattctcatgcctcagcctcccaagtagctgggactacaggtgtgcaccactactcctggatatttttttgtgtttttagtagagatggggtttcgccatgttggccaagctggtcttgaactcctgacctcaggtgatccacccgcctcgggctcccaaagtgctgggattacagacgtgaggcaccacaaccagcctggatgattcttaagggcccttctaggaccaaagttctgggaatttctagcttattctgccccctcatagcccttggcctatctatctttatccacatgcagaaacatctggcaaccccacatggctgagatgacctggtcctaggacacccttggacagaagactggcctacctagcagacctggatttttcttcctgatctgctgcttccaagttgtgtgaccttggctaagtcacttaacctttctgattgtcatttcgctttttaataaagtgggtctggtgaacaagaaatgtaataaacacgtggcttgccattcaagagatgagtctgaccattcactttctgtgtgccagagaagagagatcatgggtatagaccagcccctggaaaggctgctttggtcaaggctgagagcagctttgctcaaggaaattattcacgaaggtgaccactgtctttctgacctggcacagaggaaatgttggctgtgaatgtgaccaatagaaagaagcccgtatttctcagtcagtcctagaaccccggtaagtaattaacagagaataaaaatgtgtttgttaaatgacaaagcagcagtttttcaattgtaaggtctgcttgagagcctttgatgtgtgtttcttttcctgacttttcctttctttagaatttttgatggtctcacctggtgggtggggctttcagggtatgcccacaatgtacatttctcggcatctgtgcctcagtttcctcatttataaaatccctatgatctctgtctcacctactttacagggttgctgtgaagatcgcatactacacacaggaatgctcatcagtttttaaattttatttaatttttatttatttttttttaaatgtaattttttcagagagataaggtcttgctatgttacccagcctagtcttgaactcctggcctcaagtgatcctcctgccttggcctcccatgctgctgggattacaggtgtgaactaccatgcccagccagctcctaagtcttaaggctctgtgttagtgatagatgtggccatggtgtaggcagtgcaatgtcttcgagtgagagtgaaggtggtaactcattgcatggattctagagttctgtttattctaatccaagttcttccacttaaaaacaatgttcttcctctcattgagtctcattcctcatctataggatgggaataagagcatgtacctggcaggttgttgtaaggattaaatggtgtaaaaaaatgtcaagtgcttgcaactttgaataccaaacttgagtgaaagctcaataaattgttacttaaaaaa

SEQ ID NO: 73 - protein, 342 aa, Homo sapiens
MACEPQVDPGATGPLPPSSPGWSALPGGSPPGWGQELHNGQVLTVLRIDNTCAPISFDLGAAEEQLQTWGIQVPADQYRSLAESALLEPQVRRYIIYNSRPMRLAFAVVFYVVVWANIYSTSQMFALGNHWAGMLLVTLAAVSLTLTLVLVFERHQKKANTNTDLRLAAANGALLRHRVLLGVTDTVEGCQSVIQLWFVYFDLENCVQFLSDHVQEMKTSQEVLLRSRLSQLCVVMETGVSPATAEGPENLEDAPLLPGNSCPNERPLMQTELHQLVPEAEPEEMARQLLAVFGGYYIRLLVTSQLPQAMGTRHTNSPRIPCPCQLIEAYILGTGCCPFLAR

SEQ ID NO: 74 - DNA, 498 bp, Homo sapiens
cttcctgcagcacgtggtgctggcggcctgcgccctcctctgcattctcagcattatgctgctgccggagaccaagcgcaagctcctgcccgaggtgctccgggacggggagctgtgtcgccggccttccctgctgcggcagccaccccctacccgctgtgaccacgtcccgctgcttgccacccccaaccctgccctctgagcggcctctgagtaccctggcgggaggctggcccacacagaaaggtggcaagaagatcgggaagactgagtagggaaggcagggctgcccagaagtctcagaggcacctcacgccagccatcgcggagagctcagagggccgtccccaccctgcctcctccctgctgctttgcattcacttccttggccagagtcaggggacagggagggagctccacactgtaaccactgggtctgggctccatcctgcgcccaaagacatccacccagacctcattatttcttgctctatcatt

SEQ ID NO: 75 - DNA, 1460 bp, Homo sapiens
cctccacaggcgtcatggccctccgattcctcttgggctttctgcttgccggtgttgacctgggtgtctacctgatgcgcctggagctgtgcgacccaacccagaggcttcgggtggccctggcaggggagttggtgggggtgggagggcacttcctgttcctgggcctggcccttgtctctaaggattggcgattcctacagcgaatgatcaccgctccctgcatcctcttcctgttttatggctggcctggtttgttcctggagtccgcacggtggctgatagtgaagcggcagattgaggaggctcagtctgtgctgaggatcctggctgagcgaaaccggccccatgggcagatgctgggggaggaggcccaggaggccctgcaggacctggagaatacctgccctctccctgcaacatcctcctcttcctttgcttccctcctcaactaccgcaacatctggaaaaatctgcttatcctgggcttcaccaacttcattgcccatgccattcgccactgctaccagcctgtgggaggaggagggagcccatcggacttctacctgtgctctctgctggccagcggcaccgcagccctggcctgtgtcttcctgggggtcaccgtggaccgatttggccgccggggcatccttcttctctccatgacccttaccggcattgcttccctggtcctgctgggcctgtgggattatctgaacgaggctgccatcaccactttctctgtccttgggctcttctcctcccaagctgccgccatcctcagcaccctccttgctgctgaggtcatccccaccactgtccggggccgtggcctgggcctgatcatggctctaggggcgcttggaggactgagcggcccggcccagcgcctccacatgggccatggagccttcctgcagcacgtggtgctggcggcctgcgccctcctctgcattctcagcattatgctgctgccggagaccaagcgcaagctcctgcccgaggtgctccgggacggggagctgtgtcgccggccttccctgctgcggcagccaccccctacccgctgtgaccacgtcccgctgcttgccacccccaaccctgccctctgagcggcctctgagtaccctggcgggaggctggcccacacagaaaggtggcaagaagatcgggaagactgagtagggaaggcagggctgcccagaagtctcagaggcacctcacgccagccatcgcggagagctcagagggccgtccccaccctgcctcctccctgctgctttgcattcacttccttggccagagtcaggggacagggagggagctccacactgtaaccactgggtctgggctccatcctgcgcccaaagacatccacccagacctcattatttcttgctctatcattctgtttcaataaagacatttggaataaacgagcatatcatagcctggac

SEQ ID NO: 76 - protein, 366 aa, Homo sapiens
MALRFLLGFLLAGVDLGVYLMRLELCDPTQRLRVALAGELVGVGGHFLFLGLALVSKDWRFLQRMITAPCILFLFYGWPGLFLESARWLIVKRQIEEAQSVLRILAERNRPHGQMLGEEAQEALQDLENTCPLPATSSSSFASLLNYRNIWKNLLILGFTNFIAHAIRHCYQPVGGGGSPSDFYLCSLLASGTAALACVFLGVTVDRFGRRGILLLSMTLTGIASLVLLGLWDYLNEAAITTFSVLGLFSSQAAAILSTLLAAEVIPTTVRGRGLGLIMALGALGGLSGPAQRLHMGHGAFLQHVVLAACALLCILSIMLLPETKRKLLPEVLRDGELCRRPSLLRQPPPTRCDHVPLLATPNPAL

SEQ ID NO: 77 - DNA, 1297 bp, Homo sapiens; identical to SEQ ID NO: 49

SEQ ID NO: 78 - protein, 149 aa, Homo sapiens [sequence omitted]

SEQ ID NO: 79 - DNA, 1968 bp, Homo sapiens
atgacttttggacaaaggactggttttaggaatcctgaaagtttctgggagactttaccagtcttatttctgcaagtcatgattaccacatattttgtagctaaacaattgctgttcctacacagtaagatcatcatcttgccctcgcggcctgccgagggagcagggggcgcccgtggaactggctccctgcagctctgcggctacacgcggacctcggctgtgtgcgaggtggcggaggaggctggccgggtgcgaatccgtacccagccccagcatcttccacctgctgaggaccaccgctcagccatgggctaccagaggcaggagcctgtcatcccgccgcagagagatttagatgacagagaaacccttgtttctgaacatgagtataaagagaaaacctgtcagtctgctgctctttttaatgttgtcaactcgattataggatctggtataatagaaagtagtagatggggaagtcattttaaagcttcattaaggctaagagacgactgtgctctgaaagtgcagatagcagggcttcgtgggcaggtgcgtgtgaatgagcaaccttattcagctgttgtttgtggagacttttcccttgttttattgataaaaggaggggccctctctggaacagatacctaccagtctttggtcaataaaactttcggctttccagggtatctgctcctctctgttcttcagtttttgtatccttttatagttgatcctgaaaacgtgtttattggtcgccacttcattattggactttccacagttacctttactctgcctttatccttgtaccgaaatatagcaaagcttggaaaggtctccctcatctctacaggtttaacaactctgattcttggaattgtaatggcaagggcaatttcactgggtccacacataccaaaaacagaagacgcttgggtatttgcaaagcccaatgccattcaagcggtcggggttatgtcttttgcatttatttgccaccataactccttcttagtttacagttctctagaagaacccacagtagctaagtggtcccgccttatccatatgtccatcgtgatttctgtatttatctgtatattctttgctacatgtggatacttgacatttactggcttcacccaaggggacttatttgaaaattactgcagaaatgatgacctggtaacatttggaagattttgttatggtgtcactgtcattttgacataccctatggaatgctttgtgacaagagaggtaattgccaatgtgttttttggtgggaatctttcatcggttttccacattgttgtaacagtgatggtcatcactgtagccacgcttgtgtcattgctgattgattgcctcgggatagttctagaactcaatataggcacatcttccatacaagctcagattccaggaaagaatcagatgacagccttgtcctcaaatgaaagaactatcctgagttgtacaaagactacagacagccttgacttctgtactgatagccaaacaaaagtgaagcaaactcactgccctgttggcgcaccagccttcccgaagcgcagcctagcggtgggaatgggaacacctcgtctgggagctttctttcggttcagcttccccagccggaccccaaagacccgaagccctgggggaaggaaattccaacttgctcccggcccacccccgccccgttcctctctccggctcgctgcttccctcgctccaatgccgccgagctggtccccacttatgtgcggccgtgctgcagaggcggcggcgagctcccggactccgggcagggaaatggggcagggacgccccagccaggtaagcccagagcgccgcgccgcctctcaccggggagggcgaggccggcgaggacagcgaggcctcggccgtttcacctggctggcaactcgctgccctgccggcggcctgactcactga

SEQ ID NO: 80 - protein, 655 aa, Homo sapiens
MTFGQRTGFRNPESFWETLPVLFLQVMITTYFVAKQLLFLHSKIIILPSRPAEGAGGARGTGSLQLCGYTRTSAVCEVAEEAGRVRIRTQPQHLPPAEDHRSAMGYQRQEPVIPPQRDLDDRETLVSEHEYKEKTCQSAALFNVVNSIIGSGIIESSRWGSHFKASLRLRDDCALKVQIAGLRGQVRVNEQPYSAVVCGDFSLVLLIKGGALSGTDTYQSLVNKTFGFPGYLLLSVLQFLYPFIVDPENVFIGRHFIIGLSTVTFTLPLSLYRNIAKLGKVSLISTGLTTLILGIVMARAISLGPHIPKTEDAWVFAKPNAIQAVGVMSFAFICHHNSFLVYSSLEEPTVAKWSRLIHMSIVISVFICIFFATCGYLTFTGFTQGDLFENYCRNDDLVTFGRFCYGVTVILTYPMECFVTREVIANVFFGGNLSSVFHIVVTVMVITVATLVSLLIDCLGIVLELNIGTSSIQAQIPGKNQMTALSSNERTILSCTKTTDSLDFCTDSQTKVKQTHCPVGAPAFPKRSLAVGMGTPRLGAFFRFSFPSRTPKTRSPGGRKFQLAPGPPPPRSSLRLAASLAPMPPSWSPLMCGRAAEAAASSRTPGREMGQGRPSQVSPERRAASHRGGRGRRGQRGLGRFTWLATRCPAGGLTH

SEQ ID NO: 81 - DNA, 1092 bp, Homo sapiens
agagatttagatgacagagaaacccttgtttctgaacatgagtataaagagaaaacctgtcagtctgctgctctttttaatgttgtcaactcgattataggatctggtataataggattgccttattcaatgaagcaagctgggtttcctttgggaatattgcttttattctgggtttcatatgttacagacttttcccttgttttattgataaaaggaggggccctctctggaacagatacctaccagtctttggtcaataaaactttcggctttccagggtatctgctcctctctgttcttcagtttttgtatccttttatagcaatgataagttacaatataatagctggagatactttgagcaaagtttttcaaagaatcccaggagcatttatttgccaccataactccttcttagtttacagttctctagaagaacccacagtagctaagtggtcccgccttatccatatgtccatcgtgatttctgtatttatctgtatattctttgctacatgtggatacttgacatttactggcttcacccaaggggacttatttgaaaattactgcagaaatgatgacctggtaacatttggaagattttgttatggtgtcactgtcattttgacataccctatggaatgctttgtgacaagagaggtaattgccaatgtgttttttggtgggaatctttcatcggttttccacattgttgtaacagtgatggtcatcactgtagccacgcttgtgtcattgctgattgattgcctcgggatagttctagaactcaatggtgtgctctgtgcaactcccctcatttttatcattccatcagcctgttatctgaaactgtctgaagaaccaaggacacactccgataagattatgtcttgtgtcatgcttcccattggtgctgtggtgatggtttttggattcgtcatggctattacaaatactcaagactgcacccatgggcaggaaatgttctactgctttcctgacaatttctctctcacaaatacctcagagtctcatgttcagcagacaacacaactttctactttaaatattagtatctttcaatga

SEQ ID NO: 82 - protein, 363 aa, Homo sapiens
RDLDDRETLVSEHEYKEKTCQSAALFNVVNSIIGSGIIGLPYSMKQAGFPLGILLLFWVSYVTDFSLVLLIKGGALSGTDTYQSLVNKTFGFPGYLLLSVLQFLYPFIAMISYNIIAGDTLSKVFQRIPGAFICHHNSFLVYSSLEEPTVAKWSRLIHMSIVISVFICIFFATCGYLTFTGFTQGDLFENYCRNDDLVTFGRFCYGVTVILTYPMECFVTREVIANVFFGGNLSSVFHIVVTVMVITVATLVSLLIDCLGIVLELNGVLCATPLIFIIPSACYLKLSEEPRTHSDKIMSCVMLPIGAVVMVFGFVMAITNTQDCTHGQEMFYCFPDNFSLTNTSESHVQQTTQLSTLNISIFQ

SEQ ID NO: 83 - DNA, 1668 bp, Homo sapiens
atgaagtttccaacaggtggttgcttcagggaaaagctccagcttcagccatcatgtctctgcattctggccagtgagaaggagcaaaagaaagcatctccgtctccggaggaaaaatacatttgtctgggcgaactccggtggaaaagcgccccaggctgccacagcctagagatcttggggctgcagccctcgcggcctgccgagggagcagggggcgcccgtggaactggctccctgcagctctgcggctacacgcggacctcggctgtgtgcgaggtggcggaggaggctggccgggtgcgaatccgtacccagccccagcatcttccacctgctgaggaccaccgctcagccatgggctaccagaggcaggagcctgtcatcccgccgcagagagatttagatgacagagaaacccttgtttctgaacatgagtataaagagaaaacctgtcagtctgctgctctttttaatgttgtcaactcgattataggatctggtataatagacttttcccttgttttattgataaaaggaggggccctctctggaacagatacctaccagtctttggtcaataaaactttcggctttccagggtatctgctcctctctgttcttcagtttttgtatccttttatagcaatgataagttacaatataatagctggagatactttgagcaaagtttttcaaagaatcccaggagttgatcctgaaaacgtgtttattggtcgccacttcattattggactttccacagttacctttactctgcctttatccttgtaccgaaatatagcaaagcttggaaaggtctccctcatctctacaggtttaacaactctgattcttggaattgtaatggcaagggcaatttcactgggtccacacataccaaaaacagaagacgcttgggtatttgcaaagcccaatgccattcaagcggtcggggttatgtcttttgcatttatttgccaccataactccttcttagtttacagttctctagaagaacccacagtagctaagtggtcccgccttatccatatgtccatcgtgatttctgtatttatctgtatattctttgctacatgtggatacttgacatttactggcttcacccaaggggacttatttgaaaattactgcagaaatgatgacctggtaacatttggaagattttgttatggtgtcactgtcattttgacataccctatggaatgctttgtgacaagagaggtaattgccaatgtgttttttggtgggaatctttcatcggttttccacattgttgtaacagtgatggtcatcactgtagccacgcttgtgtcattgctgattgattgcctcgggatagttctagaactcaatggtgtgctctgtgcaactcccctcatttttatcattccatcagcctgttatctgaaactgtctgaagaaccaaggacacactccgataagattatgtcttgtgtcatgcttcccattggtgctgtggtgatggtttttggattcgtcatggctattacaaatactcaagactgcacccatgggcaggaaatgttctactgctttcctgacaatttctctctcacaaatacctcagagtctcatgttcagcagacaacacaactttctactttaaatattagtatctttcaa

SEQ ID NO: 84 - protein, 556 aa, Homo sapiens
MKFPTGGCFREKLQLQPSCLCILASEKEQKKASPSPEEKYICLGELRWKSAPGCHSLEILGLQPSRPAEGAGGARGTGSLQLCGYTRTSAVCEVAEEAGRVRIRTQPQHLPPAEDHRSAMGYQRQEPVIPPQRDLDDRETLVSEHEYKEKTCQSAALFNVVNSIIGSGIIDFSLVLLIKGGALSGTDTYQSLVNKTFGFPGYLLLSVLQFLYPFIAMISYNIIAGDTLSKVFQRIPGVDPENVFIGRHFIIGLSTVTFTLPLSLYRNIAKLGKVSLISTGLTTLILGIVMARAISLGPHIPKTEDAWVFAKPNAIQAVGVMSFAFICHHNSFLVYSSLEEPTVAKWSRLIHMSIVISVFICIFFATCGYLTFTGFTQGDLFENYCRNDDLVTFGRFCYGVTVILTYPMECFVTREVIANVFFGGNLSSVFHIVVTVMVITVATLVSLLIDCLGIVLELNGVLCATPLIFIIPSACYLKLSEEPRTHSDKIMSCVMLPIGAVVMVFGFVMAITNTQDCTHGQEMFYCFPDNFSLTNTSESHVQQTTQLSTLNISIFQ

SEQ ID NO: 85 - DNA, 1797 bp, Homo sapiens
agcatccccgtcccggaggaaaaaacatttgtctggcgaactccgggtggaaagcgccccaggctgccacagcctagagatcttggggcttcagcccctcgcggcctgccgagggagcagggggcgcccgtggaactggctccctgcagctctgcggctacacgcggacctcggctgtgtgcgaggtggcggaggaggctggccgggtgcgaatccgtacccagccccagcatcttccacctgctgaggaccaccgctcagccatgggctaccagaggcaggagcctgtcatcccgccgcagagagatttagatgacagagaaacccttgtttctgaacatgagtataaagagaaaacctgtcagtctgctgctctttttaatgttgtcaactcgattataggatctggtataataggattgccttattcaatgaagcaagctgggtttcctttgggaatattgcttttattctgggtttcatatgttacagacttttcccttgttttattgataaaaggaggggccctctctggaacagatacctaccagtctttggtcaataaaactttcggctttccagggtatctgctcctctctgttcttcagtttttgtatccttttatagcaatgataagttacaatataatagctggagatactttgagcaaagtttttcaaagaatcccaggagttgatcctgaaaacgtgtttattggtcgccacttcattattggactttccacagttacctttactctgcctttatccttgtaccgaaatatagcaaagcttggaaaggtctccctcatctctacaggtttaacaactctgattcttggaattgtaatggcaagggcaatttcactgggtccacacataccaaaaacagaagacgcttgggtatttgcaaagcccaatgccattcaagcggtcggggttatgtcttttgcatttatttgccaccataactccttcttagtttacagttctctagaagaacccacagtagctaagtggtcccgccttatccatatgtccatcgtgatttctgtatttatctgtatattctttgctacatgtggatacttgacatttactggcttcacccaaggggacttatttgaaaattactgcagaaatgatgacctggtaacatttggaagattttgttatggtgtcactgtcattttgacataccctatggaatgctttgtgacaagagaggtaattgccaatgtgttttttggtgggaatctttcatcggttttccacattgttgtaacagtgatggtcatcactgtagccacgcttgtgtcattgctgattgattgcctcgggatagttctagaactcaatggtgtgctctgtgcaactcccctcatttttatcattccatcagcctgttatctgaaactgtctgaagaaccaaggacacactccgataagattatgtcttgtgtcatgcttcccattggtgctgtggtgatggtttttggattcgtcatggctattacaaatactcaagactgcacccatgggcaggaaatgttctactgctttcctgacaatttctctctcacaaatacctcagagtctcatgttcagcagacaacacaactttctactttaaatattagtatctttcaatgagttgactgctttaaaaatatgtatgttttcatagactttaaaacacataacatttacgcttgctttagtctgtatttatgttatataaaattattattttggcttttatcaagacttggcttttatgagtagtgcaatataaaaa

SEQ ID NO: 86 - protein, 491 aa, Homo sapiens
VCEVAEEAGRVRIRTQPQHLPPAEDHRSAMGYQRQEPVIPPQRDLDDRETLVSEHEYKEKTCQSAALFNVVNSIIGSGIIGLPYSMKQAGFPLGILLLFWVSYVTDFSLVLLIKGGALSGTDTYQSLVNKTFGFPGYLLLSVLQFLYPFIAMISYNIIAGDTLSKVFQRIPGVDPENVFIGRHFIIGLSTVTFTLPLSLYRNIAKLGKVSLISTGLTTLILGIVMARAISLGPHIPKTEDAWVFAKPNAIQAVGVMSFAFICHHNSFLVYSSLEEPTVAKWSRLIHMSIVISVFICIFFATCGYLTFTGFTQGDLFENYCRNDDLVTFGRFCYGVTVILTYPMECFVTREVIANVFFGGNLSSVFHIVVTVMVITVATLVSLLIDCLGIVLELNGVLCATPLIFIIPSACYLKLSEEPRTHSDKIMSCVMLPIGAVVMVFGFVMAITNTQDCTHGQEMFYCFPDNFSLTNTSESHVQQTTQLSTLNISIFQ

SEQ ID NO: 87 - DNA, 1743 bp, Homo sapiens
atgaagtttccaacaggtggttgcttcagggaaaagctccagcttcagccatcatgtctctgcattctggccagtgagaaggagcaaaagaaagcatctccgtctccggaggaaaaatacatttgtctgggcgaactccggtggaaaagcgccccaggctgccacagcctagagatcttggggctgcagccctcgcggcctgccgagggagcagggggcgcccgtggaactggctccctgcagctctgcggctacacgcggacctcggctgtgtgcgaggtggcggaggaggctggccgggtgcgaatccgtacccagccccagcatcttccacctgctgaggaccaccgctcagccatgggctaccagaggcaggagcctgtcatcccgccgcagagagatttagatgacagagaaacccttgtttctgaacatgagtataaagagaaaacctgtcagtctgctgctctttttaatgttgtcaactcgattataggatctggtataataggattgccttattcaatgaagcaagctgggtttcctttgggaatattgcttttattctgggtttcatatgttacagacttttcccttgttttattgataaaaggaggggccctctctggaacagatacctaccagtctttggtcaataaaactttcggctttccagggtatctgctcctctctgttcttcagtttttgtatccttttatagcaatgataagttacaatataatagctggagatactttgagcaaagtttttcaaagaatcccaggagttgatcctgaaaacgtgtttattggtcgccacttcattattggactttccacagttacctttactctgcctttatccttgtaccgaaatatagcaaagcttggaaaggtctccctcatctctacaggtttaacaactctgattcttggaattgtaatggcaagggcaatttcactgggtccacacataccaaaaacagaagacgcttgggtatttgcaaagcccaatgccattcaagcggtcggggttatgtcttttgcatttatttgccaccataactccttcttagtttacagttctctagaagaacccacagtagctaagtggtcccgccttatccatatgtccatcgtgatttctgtatttatctgtatattctttgctacatgtggatacttgacatttactggcttcacccaaggggacttatttgaaaattactgcagaaatgatgacctggtaacatttggaagattttgttatggtgtcactgtcattttgacataccctatggaatgctttgtgacaagagaggtaattgccaatgtgttttttggtgggaatctttcatcggttttccacattgttgtaacagtgatggtcatcactgtagccacgcttgtgtcattgctgattgattgcctcgggatagttctagaactcaatggtgtgctctgtgcaactcccctcatttttatcattccatcagcctgttatctgaaactgtctgaagaaccaaggacacactccgataagattatgtcttgtgtcatgcttcccattggtgctgtggtgatggtttttggattcgtcatggctattacaaatactcaagactgcacccatgggcaggaaatgttctactgctttcctgacaatttctctctcacaaatacctcagagtctcatgttcagcagacaacacaactttctactttaaatattagtatctttcaa

SEQ ID NO: 88 - protein, 581 aa, Homo sapiens
MKFPTGGCFREKLQLQPSCLCILASEKEQKKASPSPEEKYICLGELRWKSAPGCHSLEILGLQPSRPAEGAGGARGTGSLQLCGYTRTSAVCEVAEEAGRVRIRTQPQHLPPAEDHRSAMGYQRQEPVIPPQRDLDDRETLVSEHEYKEKTCQSAALFNVVNSIIGSGIIGLPYSMKQAGFPLGILLLFWVSYVTDFSLVLLIKGGALSGTDTYQSLVNKTFGFPGYLLLSVLQFLYPFIAMISYNIIAGDTLSKVFQRIPGVDPENVFIGRHFIIGLSTVTFTLPLSLYRNIAKLGKVSLISTGLTTLILGIVMARAISLGPHIPKTEDAWVFAKPNAIQAVGVMSFAFICHHNSFLVYSSLEEPTVAKWSRLIHMSIVISVFICIFFATCGYLTFTGFTQGDLFENYCRNDDLVTFGRFCYGVTVILTYPMECFVTREVIANVFFGGNLSSVFHIVVTVMVITVATLVSLLIDCLGIVLELNGVLCATPLIFIIPSACYLKLSEEPRTHSDKIMSCVMLPIGAVVMVFGFVMAITNTQDCTHGQEMFYCFPDNFSLTNTSESHVQQTTQLSTLNISIFQ

SEQ ID NO: 89 - protein, 462 aa, Homo sapiens
MGYQRQEPVIPPQRDLDDRETLVSEHEYKEKTCQSAALFNVVNSIIGSGIIGLPYSMKQAGFPLGILLLFWVSYVTDFSLVLLIKGGALSGTDTYQSLVNKTFGFPGYLLLSVLQFLYPFIAMISYNIIAGDTLSKVFQRIPGVDPENVFIGRHFIIGLSTVTFTLPLSLYRNIAKLGKVSLISTGLTTLILGIVMARAISLGPHIPKTEDAWVFAKPNAIQAVGVMSFAFICHHNSFLVYSSLEEPTVAKWSRLIHMSIVISVFICIFFATCGYLTFTGFTQGDLFENYCRNDDLVTFGRFCYGVTVILTYPMECFVTREVIANVFFGGNLSSVFHIVVTVMVITVATLVSLLIDCLGIVLELNGVLCATPLIFIIPSACYLKLSEEPRTHSDKIMSCVMLPIGAVVMVFGFVMAITNTQDCTHGQEMFYCFPDNFSLTNTSESHVQQTTQLSTLNISIFQ

SEQ ID NO: 90 - DNA, 3025 bp, Homo sapiens
  misc_feature (2271)..(2276): n can represent any nucleotide a, t, c, or g
agtcatgtctgagccacagagatgggcaagatcgagaacaacgagagggtgatcctcaatgtcgggggcacccggcacgaaacctaccgcagcaccctcaagaccctgcctggaacacgcctggcccttcttgcctcctccgagcccccaggcgactgcttgaccacggcgggcgacaagctgcagccgtcgccgcctccactgtcgccgccgccgagagcgcccccgctgtcccccgggccaggcggctgcttcgagggcggcgcgggcaactgcagttcccgcggcggcagggccagcgaccatcccggtggcggccgcgagttcttcttcgaccggcacccgggcgtcttcgcctatgtgctcaattactaccgcaccggcaagctgcactgccccgcagacgtgtgcgggccgctcttcgaggaggagctggccttctggggcatcgacgagaccgacgtggagccctgctgctggatgacctaccggcagcaccgcgacgccgaggaggcgctggacatcttcgagacccccgacctcattggcggcgaccccggcgacgacgaggacctggcggccaagaggctgggcatcgaggacgcggcggggctcgggggccccgacggcaaatctggccgctggaggaggctgcagccccgcatgtgggccctcttcgaagacccctactcgtccagagccgccaggtttattgcttttgcttctttattcttcatcctggtttcaattacaactttttgcctggaaacacatgaagctttcaatattgttaaaaacaagacagaaccagtcatcaatggcacaagtgttgttctacagtatgaaattgaaacggatcctgccttgacgtatgtagaaggagtgtgtgtggtgtggtttacttttgaatttttagtccgtattgttttttcacccaataaacttgaattcatcaaaaatctcttgaatatcattgactttgtggccatcctacctttctacttagaggtgggactcagtgggctgtcatccaaagctgctaaagatgtgcttggcttcctcagggtggtaaggtttgtgaggatcctgagaattttcaagctcacccgccattttgtaggtctgagggtgcttggacatactcttcgagctagtactaatgaatttttgctgctgataattttcctggctctaggagttttgatatttgctaccatgatctactatgccgagagagtgggagctcaacctaacgacccttcagctagtgagcacacacagttcaaaaacattcccattgggttctggtgggctgtagtgaccatgactaccctgggttatggggatatgtacccccaaacatggtcaggcatgctggtgggagccctgtgtgctctggctggagtgctgacaatagccatgccagtgcctgtcattgtcaataattttggaatgtactactccttggcaatggcaaagcagaaacttccaaggaaaagaaagaagcacatccctcctgctcctcaggcaagctcacctactttttgcaagacagaattaaatatggcctgcaatagtacacagagtgacacatgtctgggcaaagacaatcgacttctggaacataacagatcagtgttatcaggtgacgacagtacaggaagtgagccgccactatcacccccagaaaggctccccatcagacgctctagtaccagagacaaaaacagaagaggggaaacatgtttcctactgacgacaggtgattacacgtgtgcttctgatggagggatcaggaaagataactgcaaagaggttgtcattactggttacacgcaagccgaggccagatctcttacttaatgacttgggggaaggcacaaaacatgagagaaagtgttgtacagaatttatcatggattattgactgctgagaaagggacagtggaatttagccatacaaaggactatactggaaacagacttctgctgctgaatgtgccctgatgtgaccaggttgcacttggaagagatcctccgcgtcttcatgaggcacttaaagcttataaaagaactgcggctggaactcatctggtgctccccatgagagtgctctgcttgtagactggccagtgtccatgaaacaactgtaaataccaacatgtgtgcatgggtcaacagtcttggccatttctcatcaaaagaagccaaattcatgatcaacatctctgaagtttcaagtaaggcccacacttctttgaattaactctnnnnnncacattaggttgtgctgtgaattacttaaggcagtgatactgatgtagtatagttttgtcttaatttcccttatttctacttctttggttgaatctatgaacttgattgtataattttcttataaattactgatgtaatcagcttgtcaattatgttgtgaaattgttagtattcatttatcaaaaatgacctatgtttagtcacatatttgtttagttctgggaaattgttatagcttaaatggaactcaccaacattattcatagtttaagtcttttatcattattacctcaattataaatattacaaaaacataattctggcaatgagagtatttttttattcaatgatcaaggagcaatgtcagtatatagtagaatatcaattaaattatatcctaaaatgtatattttgcataaaagagatattctttaatcaattacttttttgtgagttntgtggcgaatgnnnnnnnnnnnnnnnnnnnnnctgttgtagatgaaactgtataaganttttacatcttgcttaatcaatatttncagagnctattagttcccctgggattctgaatataacatatagcctattataaatccctgtatcgtggaccttttgtgaacatttcaaggcgcatgcacaaccttgatgataaccagtggaaatgtaactaactgaaatgaagaatnaaaggcaaatgagctggggataaacttgaatgttatctgattaaattactcaaattatt

SEQ ID NO: 91 - DNA, 1964 bp, Homo sapiens
agtcatgtctgagccacagagatgggcaagatcgagaacaacgagagggtgatcctcaatgtcgggggcacccggcacgaaacctaccgcagcaccctcaagaccctgcctggaacacgcctggcccttcttgcctcctccgagcccccaggcgactgcttgaccacggcgggcgacaagctgcagccgtcgccgcctccactgtcgccgccgccgagagcgcccccgctgtcccccgggccaggcggctgcttcgagggcggcgcgggcaactgcagttcccgcggcggcagggccagcgaccatcccggtggcggccgcgagttcttcttcgaccggcacccgggcgtcttcgcctatgtgctcaattactaccgcaccggcaagctgcactgccccgcagacgtgtgcgggccgctcttcgaggaggagctggccttctggggcatcgacgagaccgacgtggagccctgctgctggatgacctaccggcagcaccgcgacgccgaggaggcgctggacatcttcgagacccccgacctcattggcggcgaccccggcgacgacgaggacctggcggccaagaggctgggcatcgaggacgcggcggggctcgggggccccgacggcaaatctggccgctggaggaggctgcagccccgcatgtgggccctcttcgaagacccctactcgtccagagccgccaggtttattgcttttgcttctttattcttcatcctggtttcaattacaactttttgcctggaaacacatgaagctttcaatattgttaaaaacaagacagaaccagtcatcaatggcacaagtgttgttctacagtatgaaattgaaacggatcctgccttgacgtatgtagaaggagtgtgtgtggtgtggtttacttttgaatttttagtccgtattgttttttcacccaataaacttgaattcatcaaaaatctcttgaatatcattgactttgtggccatcctacctttctacttagaggtgggactcagtgggctgtcatccaaagctgctaaagatgtgcttggcttcctcagggtggtaaggtttgtgaggatcctgagaattttcaagctcacccgccattttgtaggtctgagggtgcttggacatactcttcgagctagtactaatgaatttttgctgctgataattttcctggctctaggagttttgatatttgctaccatgatctactatgccgagagagtgggagctcaacctaacgacccttcagctagtgagcacacacagttcaaaaacattcccattgggttctggtgggctgtagtgaccatgactaccctgggttatggggatatgtacccccaaacatggtcaggcatgctggtgggagccctgtgtgctctggctggagtgctgacaatagccatgccagtgcctgtcattgtcaataattttggaatgtactactccttggcaatggcaaagcagaaacttccaaggaaaagaaagaagcacatccctcctgctcctcaggcaagctcacctactttttgcaagacagaattaaatatggcctgcaatagtacacagagtgacacatgtctgggcaaagacaatcgacttctggaacataacagatcagtgttatcaggtgacgacagtacaggaagtgagccgccactatcacccccagaaaggctccccatcagacgctctagtaccagagacaaaaacagaagaggggaaacatgtttcctactgacgacaggtgattacacgtgtgcttctgatggagggatcaggaaaggatatgaaaaatcccgaagcttaaacaacatagcgggcttggcaggcaatgctctgaggctctctccagtaacatcaccctacaactctccttgtcctctgaggcgctctcgatctcccatcccatctatcttgtaaaccaaaccctcgtgccgaatcttggc

SEQ ID NO: 92 - protein, 613 aa, Homo sapiens
MGKIENNERVILNVGGTRHETYRSTLKTLPGTRLALLASSEPPGDCLTTAGDKLQPSPPPLSPPPRAPPLSPGPGGCFEGGAGNCSSRGGRASDHPGGGREFFFDRHPGVFAYVLNYYRTGKLHCPADVCGPLFEEELAFWGIDETDVEPCCWMTYRQHRDAEEALDIFETPDLIGGDPGDDEDLAAKRLGIEDAAGLGGPDGKSGRWRRLQPRMWALFEDPYSSRAARFIAFASLFFILVSITTFCLETHEAFNIVKNKTEPVINGTSVVLQYEIETDPALTYVEGVCVVWFTFEFLVRIVFSPNKLEFIKNLLNIIDFVAILPFYLEVGLSGLSSKAAKDVLGFLRVVRFVRILRIFKLTRHFVGLRVLGHTLRASTNEFLLLIIFLALGVLIFATMIYYAERVGAQPNDPSASEHTQFKNIPIGFWWAVVTMTTLGYGDMYPQTWSGMLVGALCALAGVLTIAMPVPVIVNNFGMYYSLAMAKQKLPRKRKKHIPPAPQASSPTFCKTELNMACNSTQSDTCLGKDNRLLEHNRSVLSGDDSTGSEPPLSPPERLPIRRSSTRDKNRRGETCFLLTTGDYTCASDGGIRKDNCKEVVITGYTQAEARSLT

SEQ ID NO: 93 - protein, 638 aa, Homo sapiens
MGKIENNERVILNVGGTRHETYRSTLKTLPGTRLALLASSEPPGDCLTTAGDKLQPSPPPLSPPPRAPPLSPGPGGCFEGGAGNCSSRGGRASDHPGGGREFFFDRHPGVFAYVLNYYRTGKLHCPADVCGPLFEEELAFWGIDETDVEPCCWMTYRQHRDAEEALDIFETPDLIGGDPGDDEDLAAKRLGIEDAAGLGGPDGKSGRWRRLQPRMWALFEDPYSSRAARFIAFASLFFILVSITTFCLETHEAFNIVKNKTEPVINGTSVVLQYEIETDPALTYVEGVCVVWFTFEFLVRIVFSPNKLEFIKNLLNIIDFVAILPFYLEVGLSGLSSKAAKDVLGFLRVVRFVRILRIFKLTRHFVGLRVLGHTLRASTNEFLLLIIFLALGVLIFATMIYYAERVGAQPNDPSASEHTQFKNIPIGFWWAVVTMTTLGYGDMYPQTWSGMLVGALCALAGVLTIAMPVPVIVNNFGMYYSLAMAKQKLPRKRKKHIPPAPQASSPTFCKTELNMACNSTQSDTCLGKDNRLLEHNRSVLSGDDSTGSEPPLSPPERLPIRRSSTRDKNRRGETCFLLTTGDYTCASDGGIRKGYEKSRSLNNIAGLAGNALRLSPVTSPYNSPCPLRRSRSPIPSIL